(19) United States
(12) Patent Application Publication
(10) Pub. No.: US 2025/0092437 A1
(43) Pub. Date: Mar. 20, 2025

(54) DISEASE DETECTION WITH COMBINATORIAL BIOMARKERS

(71) Applicant: Sunbird Bio, Inc., Cambridge, MA (US)

(72) Inventors: Wendy Winckler Adamovich, Cambridge, MA (US); Tram Thuy Tran, Cambridge, MA (US); Oscar Alejandro Balbin, Cambridge, MA (US); Sophie Cazanave, Cambridge, MA (US)

(21) Appl. No.: 18/705,899

(22) PCT Filed: Oct. 28, 2022
(86) PCT No.: PCT/US2022/048299

§ 371 Date: Apr. 29, 2024

Related U.S. Application Data
(60) Provisional application No. 63/273,485, filed on Oct. 29, 2021.
(60) Provisional application No. 63/331,520, filed on Apr. 15, 2022.

(51) Int. Cl. C12Q 1/37; G01N 33/542; G01N 33/569; G01N 33/68; G16H 10/40; G16H 50/20

(52) U.S. Cl. CPC C12Q 1/37; G01N 33/542; G01N 33/56983; G01N 33/6893; G16H 10/40; G16H 50/20; G01N 2800/085; G01N 2800/7028

(57) ABSTRACT

The present application provides compositions and methods for determining a disease or condition in a subject. The method comprises contacting a body fluid with a molecule comprising a reporter thereof and the reporter is cleaved by an agent in the body fluid. The rate of formation or amount of the cleaved reporter is measured. This result is input into an algorithm along with other parameters to determine a physiological or clinical condition of a subject. Diseases and conditions that can be determined by the method are also described.

### CROSS-REFERENCE

This application claims the benefit of priority to U.S. Provisional Application No. 63/273,485, filed on Oct. 29, 2021, and U.S. Provisional Application No. 63/331,520, the entirety of which is incorporated herein by reference.

### BRIEF SUMMARY

This application claims the benefit of priority to U.S. Provisional Application No. 63/273,485, filed on Oct. 29, 2021, the entirety of which is incorporated herein by reference.

Provided herein is a method comprising contacting a body fluid sample from a subject with a synthetic molecule. Further provided herein is a method wherein said synthetic molecule comprises a cleavable linker and a reporter, and wherein said cleavable linker is cleaved by an agent from said body fluid sample, releasing said reporter from said synthetic molecule. Further provided herein is a method for detecting a rate of formation or an amount of said reporter, and inputting data from (a) said detection and (b) a parameter that indicates a physiological or clinical condition of said subject into an algorithm, wherein said parameter is not associated with said detection.

Further provided herein is a method further comprising determining and monitoring a disease, a condition, or a change in condition of said subject based on an output from said algorithm. Further provided herein is a method wherein said algorithm comprises a supervised or unsupervised Machine Learning or Deep Learning algorithm, Logistic Regression, Naive Bayes, Support Vector Machine, Decision Tree, Random Forest, Gradient Boosting, Regularizing Gradient Boosting, K-Nearest Neighbors, a continuous regression approach, Ridge Regression, Kernel Ridge Regression, Support Vector Regression, deep learning approach, Neural Networks, Convolutional Neural Network (CNNs), Recurrent Neural Networks (RNNs), Long Short Term Memory Networks (LSTMs), Generative Models, Generative Adversarial Networks (GANs), Deep Belief Networks (DBNs), Feedforward Neural Networks, Autoencoders, Radial Basis Function Networks (RBFNs), Multilayer Perceptrons (MLPs), Stochastic Neural Networks or any combination thereof.

Further provided herein is a method wherein said parameter comprises a result from a clinical test of said subject, a result from a genetic test of said subject, a result from a protein test of said subject, a demographic characteristic of said subject, a clinical characteristic of said subject, a digital profile of said subject or a combination thereof. Further provided herein is a method, wherein said clinical test comprises a tissue biopsy, a blood test, a test for proteins, a test for metabolites, a test for a circulating analyte, a test on any bodily fluid, a test for microbiome, a test for biomarkers, a test for malignancy or tumor, a test for liver function, a test for lipids or any combination thereof. Further provided herein is a method wherein said blood test comprises an enhanced liver fibrosis (ELF) test or any of its components thereof. Further provided herein is a method wherein said circulating analyte comprises a polypeptide, a protein, a glycoprotein, a cytokine, a hormone or a combination thereof. Further provided herein is a method wherein said genetic test comprises analysis of a nucleic acid, a genomic DNA, a mitochondrial DNA (mtDNA), a mRNA, a microRNA (miRNA), a long non-coding RNA (lncRNA), a snoRNA, rRNA, tRNA, a circulating cell-free DNA (cfDNA), a circulating tumor DNA (ctDNA), a cell-free RNA (cfRNA), a cell-free total nucleic acid (cfTNA), an exosomal nucleic acid, a microbial or viral nucleic acid, a DNA methylation marker, or any combination thereof. Further provided herein is a method wherein said protein test comprises analysis of a post-translational modification, total protein, proteomics, protein ratios or any combination thereof. Further provided herein is a method wherein said post-translational modification comprises phosphorylation, glycosylation, ubiquitination, nitrosylation, methylation, acetylation, lipidation, hydroxylation, proteolysis or any combinations thereof. Further provided herein is a method wherein said demographic characteristic comprises age, gender, race, ethnicity, education, occupation, income, geographic area or any combination thereof. Further provided herein is a method wherein said clinical characteristic comprises disease characteristics, symptoms and severity/stage of a disease or condition, body mass index (BMI), age at diagnosis, type 2 diabetes, metabolic syndrome, MELD score, alcohol or other toxin consumption, dyslipidemia, genetic risk profile (PNPLA-3), liver stiffness, controlled attenuation parameter (CAP), or any combination thereof.

Further provided herein is a method wherein said digital profile comprises a digital measurement collected by a wearable device. Further provided herein is a method wherein said wearable device comprises a smart watch, a smart clothing, a smart jewelry, a fitness tracker, an implantable device, a head-mounted display or a combination thereof. Further provided herein is a method wherein said digital measurement comprises heart rate, brainwave, muscle bio-signals, sleep patterns and duration, speed, cadence, distance traveled, calories burned, respiration rate, skin conductivity, biomarkers from a bodily fluid, or any combination thereof.

Further provided herein is a method wherein said parameter comprises at least two parameters, at least five parameters, at least ten parameters, at least twenty parameters or at least fifty parameters.

Further provided herein is a method further comprising contacting a second body fluid sample from the subject with a second synthetic molecule. In some embodiments, the contacting is performed ex vivo. In some embodiments, the contact is performed in vivo. In some embodiments, the contacting is performed in vitro. In some embodiments, the contacting is performed in situ. Further provided herein is a method wherein said second molecule comprises a second cleavable linker and a second reporter, and wherein said second cleavable linker is cleaved by a second agent from said second body fluid sample, releasing said second reporter from said second molecule. Further provided herein is a method for detecting a rate of formation or an amount of said second reporter.

Further provided herein is a method wherein said second body fluid sample is different from said body fluid sample. Further provided herein is a method wherein said second cleavable linker is different from said cleavable linker.

Further provided herein is a method wherein said disease or condition comprises liver disease, cancer, organ transplant rejection, infectious diseases, allergic diseases, an autoimmune disease, a chronic or acute inflammation, or an Alzheimer's disease, neurologic disease or any other protease related disease. Further provided herein is a method wherein said liver disease comprises a Non-alcoholic steatohepatitis (NASH), a non-alcoholic fatty liver disease (NAFLD), a toxin mediated liver injury, a viral hepatitis, a fulminant hepatitis, an alcoholic hepatitis, an autoimmune hepatitis, a cirrhosis of the liver, a hepatocellular carcinoma (HCC), a primary biliary cholangitis (PBC), a cholangiocarcinoma, a primary sclerosing cholangitis, an acute or chronic rejection of a transplanted liver, a drug induced liver injury, an inherited liver disease or a combination thereof.

Further provided herein is a method wherein said body fluid sample is selected from the group consisting of blood, plasma, serum, bone marrow fluid, lymphatic fluid, bile, amniotic fluid, mucosal fluid, saliva, urine, cerebrospinal fluid, spinal fluid, synovial fluid, ascitic fluid, semen, ductal aspirate, feces, stool, vaginal effluent, lachrymal fluid, tissue lysate and patient-derived cell line supernatant. Further provided herein is a method wherein said body fluid sample comprises a rinse fluid, a conditioning media or buffer, a swab viral transport media, a saline, a culture media, or a cell culture supernatant. Further provided herein is a method wherein said rinse fluid is selected from the group consisting of a mouthwash rinse, a bronchioalveolar rinse, a lavage fluid, a hair wash rinse, a nasal spray effluent, a swab of any bodily surface, orifice, organ structure or solid tumor biopsies applied to saline or any media or any derivatives thereof.

Further provided herein is a method wherein said agent is selected from the group consisting of a oxidoreductase, a transferase, a hydrolase, a lyase, a isomerase, a ligase, a protease (peptidase), a hydrolase, an esterase, a β-glycosidase, a phospholipase and a phosphodiesterase, peroxidase, lipase, amylase a nucleophilic reagent, a reducing reagent, a electrophilic/acidic reagent, an organometallic/metal catalyst, an oxidizing reagent, a hydroxyl ion, a thiols nucleophile, a nitrogen nucleophile, a sodium dithionite and a sodium periodate. Further provided herein is a method wherein said agent is a protease. Further provided herein is a method wherein said protease is an endopeptidase or an exopeptidase.

Further provided herein is a method wherein said protease is selected from the group consisting of an A20 (TNFa-induced protein 3), an abhydrolase domain containing 4, an abhydrolase domain containing 12, an abhydrolase domain containing 12B, an abhydrolase domain containing 13, an acrosin, an acylaminoacyl-peptidase, a disintegrin and metalloproteinase (ADAM), an ADAM1a, an ADAM2 (Fertilin-b), an ADAM3B, an ADAM4, an ADAM4B, an ADAM5, an ADAM6, an ADAM7, an ADAM8, an ADAM9, an ADAM10, an ADAM11, an ADAM12 metalloprotease, an ADAM15, an ADAM17, an ADAM18, an ADAM19, an ADAM20, an ADAM21, an ADAM22, an ADAM23, an ADAM28, an ADAM29, an ADAM30, an ADAM32, an ADAM33, a disintegrin and metalloproteinase with thrombospondin motifs (ADAMTS), an ADAMTS1, an ADAMTS2, an ADAMTS3, an ADAMTS4, an ADAMTS5/11, an ADAMTS6, an ADAMTS7, an ADAMTS8, an ADAMTS9, an ADAMTS10, an ADAMTS12, an ADAMTS13, an ADAMTS14, an ADAMTS15, an ADAMTS16, an ADAMTS17, an ADAMTS18, an ADAMTS19, an ADAMTS20, an adipocyte-enh. binding protein 1, an Afg3-like protein 1, an Afg3-like protein 2, an airway-trypsin-like protease, an aminoacylase, an aminopeptidase A, an aminopeptidase B, an aminopeptidase B-like 1, an aminopeptidase MAMS/L-RAP, an aminopeptidase N, an aminopeptidase O, an aminopeptidase P homologue, an aminopeptidase P1, an aminopeptidase PILS, an aminopeptidase Q, an aminopeptidase-like 1, an AMSH/STAMBP, an AMSH-LP/STAMBPL1, an angiotensin-converting enzyme 1 (ACE1), an angiotensin-converting enzyme 2 (ACE2), an angiotensin-converting enzyme 3 (ACE3), an anionic trypsin (II), an apolipoprotein (a), an archaemetzincin-1, an archaemetzincin-2, an aspartoacylase, an aspartoacylase-3, an aspartyl aminopeptidase, an ataxin-3, an ataxin-3 like, an ATP/GTP binding protein 1, an ATP/GTP binding protein-like 2, an ATP/GTP binding protein-like 3, an ATP/GTP binding protein-like 4, an ATP/GTP binding protein-like 5, an ATP23 peptidase, an autophagin-1, an autophagin-2, an autophagin-3, an autophagin-4, an azurocidin, a beta lactamase, a beta-secretase 1, a beta-secretase 2, a bleomycin hydrolase, a brain serine proteinase 2, a BRCC36 (BRCA2-containing complex, sub 3), a calpain, a calpain 1, a calpain 2, a calpain 3, a calpain 4, a calpain 5, a calpain 6, a calpain 7, a calpain 7-like, a calpain 8, a calpain 9, a calpain 10, a calpain 11, a calpain 12, a calpain 13, a calpain 14, a calpain 15 (Solh protein), a cysteine protease, a carboxypeptidase A1, a carboxypeptidase A2, a carboxypeptidase A3, a carboxypeptidase A4, a carboxypeptidase A5, a carboxypeptidase A6, a carboxypeptidase B, a carboxypeptidase D, a carboxypeptidase E, a carboxypeptidase M, a carboxypeptidase N, a carboxypeptidase O, a carboxypeptidase U, a carboxypeptidase X1, a carboxypeptidase X2, a carboxypeptidase Z, a carnosine dipeptidase 1, a carnosine dipeptidase 2, a caspase recruitment domain family, member 8, a caspase, a caspase-1, a caspase-2, a caspase-3, a caspase-4/11, a caspase-5, a caspase-6, a caspase-7, a caspase-8, a caspase-9, a caspase-10, a caspase-12, a caspase-14, a caspase-14-like, a casper/FLIP, a cathepsin, a cathepsin A (CTSA), a cathepsin B (CTSB), a cathepsin C (CTSC), a cathepsin D (CTSD), a cathepsin E (CTSE), a cathepsin F, a cathepsin G, a cathepsin H (CTSH), a cathepsin K (CTSK), a cathepsin L (CTSL), a cathepsin L2, a cathepsin O, a cathepsin S (CTSS), a cathepsin V (CTSV), a cathepsin W, a cathepsin Z (CTSZ), a cationic trypsin, a cezanne/OTU domain containing 7B, a cezanne-2, a CGI-58, a chymase, a chymopasin, a chymosin, a chymotrypsin B, a chymotrypsin C, a coagulation factor IXa, a coagulation factor VIIa, a coagulation factor Xa, a coagulation factor XIa, a coagulation factor XIIa, a collagenase 1, a collagenase 2, a collagenase 3, a complement protease C1r serine protease, a complement protease C1s serine protease, a complement C1r-homolog, a complement component 2, a complement component C1ra, a complement component C1sa, a complement factor B, a complement factor D, a complement factor D-like, a complement factor I, a COPS6, a corin, a CSN5 (JAB1), a cylindromatosis protein, a cytosol alanyl aminopep.-like 1, a cytosol alanyl aminopeptidase, a DDI-related protease, a DECYSIN, a Der1-like domain family, member 1, a Der1-like domain family, member 2, a Der1-like domain family, member 3, a DESC1 protease, a desert hedgehog protein, a desumoylating isopeptidase 1, a desumoylating isopeptidase 2, a dihydroorotase, a dihydropyrimidinase, a dihydropyrimidinase-related protein 1, a dihydropyrimidinase-related protein 2, a dihydropyrimidinase-related protein 3, a dihydropyrimidinase-related protein 4, a dihydropyrimidinase-related protein 5, a DINE peptidase, a dipeptidyl peptidase (DPP), a dipeptidyl peptidase (DPP1), a dipeptidyl-peptidase 4 (DPP4), a dipeptidyl-peptidase 6 (DPP6), a dipeptidyl-peptidase 8 (DPP8), a dipeptidyl-peptidase 9 (DPP9), a dipeptidyl-peptidase II, a dipeptidyl-peptidase III, a dipeptidyl-peptidase 10 (DPP10), a DJ-1, a DNA-damage inducible protein, a DNA-damage inducible protein 2, a DUB-1, a DUB-2, a DUB2a, a DUB2a-like, a DUB2a-like2, a DUB6, or a combination thereof.

Further provided herein is a method wherein said protease is selected from the group consisting of a T cell protease, a complement protease, a fibrosis protease, and an inflammation-related protease. Further provided herein is a method wherein said cleavable linker is a peptide, a carbohydrate, a nucleic acid, a lipid, an ester, a glycoside, a phospholipid, a phosphodiester, a nucleophile/base sensitive linker, a reduction sensitive linker, an electrophile/acid sensitive linker, a metal cleavable linker, an oxidation sensitive linker or a combination thereof.

Further provided herein is a method wherein said cleavable linker is a peptide. Further provided herein is a method wherein said peptide comprises an amino acid sequence selected from the group consisting of SEQ ID Nos: 1-677 or a sequence comprising a mimetic of any one of SEQ ID Nos: 1-677.

Further provided herein is a method wherein said mimetic is a beta amino acid or a peptoid. Further provided herein is a method wherein said amino acid comprises a chemical modification. Further provided herein is a method wherein said chemical modification comprises phosphorylation, alkylation, arylation, amination, amidation, sulfonylation, halogenation, borylation, glycosylation, cyclization, linearization, hydration, hydrogenation, nitration, nitrosylation, reduction, oxidation, esterification, hydrolysis, dephosphorylation, dealkylation, dearylation, deamination, deamidation, desulfonylation, dehalogenation, deborylation, deglycosylation, decyclization, delinearization, dehydration, dehydrogenation, denitration, denitrosylation, deesterification, dehydrolysis or any combination thereof.

Further provided herein is a method wherein said cleavable linker is directly connected to said reporter through a covalent bond. Further provided herein is a method wherein said reporter comprises a fluorescent label, a mass tag, a chromophore, an electrochemically active molecule, a bio-Layer interferometry or surface plasmon resonance detectable molecule, a precipitating substance, a mass spectrometry and liquid chromatography substrate, a magnetically active molecule, a gel forming and/or viscosity changing molecule, an immunoassay detectable molecule, a cell-based amplification detectable or a nucleic acid barcode, or any combinations thereof. Further provided herein is a method wherein said reporter comprises a fluorescent label. Further provided herein is a method wherein said fluorescent label is selected from a group consisting of a 5-carboxyfluorescein (5-FAM), a 7-amino-4-carbamoylmethylcoumarin (ACC), a 7-Amino-4-methylcoumarin (AMC), a 2-Aminobenzoyl (Abz), a Cy7, a Cy5, a Cy3 and a (5-((2-Aminoethyl)amino)naphthalene-1-sulfonic acid) (EDANS). Further provided herein is a method wherein said molecule further comprises a fluorescent quencher. Further provided herein is a method wherein said fluorescent quencher is selected from the group consisting of BHQ0, BHQ1, BHQ2, BHQ3, BBQ650, ATTO 540Q, ATTO 580Q, ATTO 612Q, CPQ2, QSY-21, QSY-35, QSY-7, QSY-9, DABCYL (4-([4′-dimethylamino)phenyl] azo)benzoyl), Dnp (2,4-dinitrophenyl) and Eclipse. Further provided herein is a method wherein said fluorescent quencher is directly connected to said cleavable linker through a covalent bond.

Further provided herein is a method wherein said molecule further comprises a carrier. Further provided herein is a method wherein said carrier comprises a native, labeled or synthetic protein, a synthetic chemical polymer of precisely known chemical composition or with a distribution around a mean molecular weight, an oligonucleotide, a phosphorodiamidate morpholino oligomer (PMO), a foldamer, a lipid, a lipid micelle, a nanoparticle, a solid support made of polystyrene, polypropylene or any other type of plastic, or any combination thereof.

Further provided herein is a method wherein said subject is a mammal. Further provided herein is a method wherein said mammal is a human.

Further provided herein is a method wherein said detection comprises fluorescent detection, spectroscopic detection, mass spectrometry, immunological detection, DNA unique molecular identifier barcode detection or imaging detection. Further provided herein is a method wherein said detection comprises fluorescent detection. Further provided herein is a method wherein said fluorescent detection is fluorescence resonance energy transfer (FRET). Further provided herein is a method wherein the contacting is conducted ex vivo. Further provided herein is a method wherein said cleavable linker comprises a nucleic acid. Further provided herein is a method wherein said subject comprises a mammal. Further provided herein is a method wherein said mammal comprises a human.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the present disclosure are set forth with particularity in the appended claims. A better understanding of the features and advantages of the present disclosure will be obtained by reference to the following detailed description that sets forth illustrative embodiments, in which the principles of the present disclosure are utilized, and the accompanying drawings (“FIGURE.” or “FIGURES.” herein), of which:

FIG. 1 shows a plurality of probes according to the current application. Each probe 101 includes a reporter 103, shown as a star in FIG. 1. The reporters 103, are linked to a cleavable linker 105, which is a cleavable substrate for an agent 107.

FIG. 2 shows cleavage of the reporter in a plurality of the probes. As shown, cleavage by the agent 107 of the cleavable linker 105 results in the reporters 103 being cleaved from the probe 101. Once cleaved, the cleaved reporters 203 can be detected and/or distinguished from un-cleaved reporters 103. The presence and detection of cleaved reporters 203 indicates that the agents 107 are present and active in a sample. In addition, the absence of an agent activity may be used for detection associated with a decrease in activity. The activity of the agents can be quantified based on, for example, the rate at which the cleavage reaction takes place or the amount of cleaved reporters in a sample or by other means such as a ratio of rates against an appropriate control or a ratio of cleaved reporters against an appropriate control.

FIG. 3 illustrates a method 301 of evaluating a biological condition in a subject using the probes 101.

FIG. 4 shows the selection of probes to use in a composition to analyze the activities of agents to analyze one or more particular, biological conditions or disease states. The activity of one or more agents may be associated with a biological condition or disease state. This may include the progression of a particular condition or state over time. Thus, to evaluate a biological condition or disease state in a subject, probes that can be cleaved by agents of interest are selected from the library for inclusion in a condition-specific panel 403. The selected probes 405 of the condition-specific panel are differentially labeled so that the activity of the predetermined proteases can be measured 305. The different probes 101, including those included in library 401, may include features that confer properties to the fragments that ensure accurate, multiplex detection of agent activity. Such properties include, for example improved cleavage, detection, solubility, stability, reproducibility, robustness and/or expanded compatibility with different types of reporter.

FIG. 5 shows a schematic of a probe 501 that includes a spacer 507, a solubility tag 509, a quencher and a covalent or non-covalent attachment site 511. The respective positions of these components can, in principle, be interconverted.

FIG. 6A-C shows cleavage of the probe. FIG. 6A shows that the probe 601 includes a fluorescent reporter 603 and a quencher 605. The probe 601 may also include a spacer 507, a solubility tag 509, and/or a covalent or non-covalent attachment site 511. FIG. 6B shows the cleavage process of two components probe. FIG. 6C shows the cleavage process of three components probe.

FIG. 7A-C shows reaction processes for HPQ fluorophore. FIG. 7A shows a probe 701 with an auto-immolative spacer 705 and precipitating fluorescent reporter 703. The spacer 705 connects the precipitating fluorophore reporter to an exopeptidase substrate 707, which is surrounded by the rectangle for clarity. A specific, predetermined exopeptidase cleaves the exopeptidase substrate 707. As a result, the auto-immolative spacer 705 dissociates from the precipitating fluorophore reporter 703. This allows establishment of a particular hydrogen bond 709 in the reporter 703, such that it enters a solid state, precipitates from the fluid sample, and provides an intense fluorescent signal. FIG. 7B shows de detailed process. FIG. 7C shows the reaction process with both endopeptidase and exopeptidase.

FIG. 8 shows a method using a probe 801 with an auto-immolative spacer 807, precipitating or non precipitating fluorescent reporter 805, and an enzyme/protease substrate 809 cleaved by a predetermined enzyme/endoprotease 803. The probe includes an enzyme/protease substrate 809 that is cleaved by two predetermined enzymes/proteases. The first of these enzymes/proteases, is the enzyme/endoprotease 803 of interest in the sample. The enzyme/endoprotease 803 in the fluid sample cleaves the enzyme/protease substrate 809. However, because 803, cannot cleave completely/the terminal or penultimate amino acids in the protease substrate from the spacer 807. Thus, a predetermined exopeptidase/enzyme 811 is introduced to the sample. The exopeptidase/enzyme can be spiked into the fluid sample, before, after, or during incubation with the endoprotease/enzyme 803. The enzyme/protease substrate 805 is engineered such that cleavage by the enzyme/endoprotease 803 results in a second enzyme/protease substrate 813 that can be cleaved by the predetermined enzyme/exopeptidase 811. Cleavage by 811 causes the spacer 807 to dissociate from the precipitating/non-precipitating fluorophore reporter 805, such the reporter 805 provides an intense fluorescent signal.

FIG. 9 shows the progression of NASH.

FIG. 10 shows in vivo probes used to detect protease activity.

FIG. 11 shows the protease activities measured using the in vivo probes.

FIG. 12 outlines an experiment of present application.

FIG. 13 outlines an experiment of present application.

FIG. 14 shows that the probes can accurately detect and differentiate between samples from patients diagnosed with NASH via liver biopsy and healthy patient samples when encountering NASH-related proteases in mice K2 EDTA plasma.

FIG. 15A-B provide experimental results showing that a specific peptide linker of the present application can differentiate between NASH-related protease activity in healthy mice and NASH+ samples from K2 EDTA mice plasma. FIG. 15A shows the results from healthy samples.

FIG. 15B shows results from NASH+ samples.

FIG. 16 provides experimental results comparing the probes and their ability to distinguish between NASH (CDHFD) samples (the right data point) and healthy (CD) samples (the left data point).

FIG. 17 provides raw experimental results showing that the measured rate of fluorescence increase for Probe #492 can be ascribed to protease activity and to NASH disease in K2 EDTA mice plasma. The average rate of fluorescence increase over n=10 samples matches pooled plasma (n=10) increase of fluorescence in both disease and healthy conditions.

FIG. 18 provides experimental results showing that the measure rate of fluorescence increase for Probe #102 can be ascribed to protease activity and to NASH disease in K2 EDTA mice plasma. The average rate of fluorescence increase over n=10 samples matches pooled plasma (n=10) increase of fluorescence in both disease and healthy conditions.

FIG. 19A-B provides experimental results showing that activity, not abundance, is responsible for determination of disease-based protease activity differences in K2 EDTA mouse plasma samples. FIG. 19A shows the results of testing for protease abundance levels and FIG. 19B shows the results of testing for protease activity levels.

FIG. 20 outlines an experimental design of the present application.

FIG. 21A-F provide experimental results showing that several probes can differentiate among healthy K2 EDTA plasma samples (left), regression samples (center), and NASH samples (right). FIG. 21A shows the results of Probe #428, FIG. 21B shows the results of Probe #520, FIG. 21C shows the results of Probe #96, FIG. 21D shows the results of Probe #102, FIG. 21E shows the results of Probe #492, and FIG. 21F shows the results of Probe #647.

FIG. 22 provides experimental results showing the probes can distinguish between healthy and the JO2 mouse model of fulminant hepatitis samples. The Jo2 antibody shows cytolytic activity against cell lines expressing mouse Fas by inducing apoptosis.

FIG. 23 provides experimental results showing the probes can distinguish between healthy and fulminant hepatitis samples in vivo in a mice model. +/++ group denotes mild hepatitis symptoms and +++/++++ group denotes fulminant hepatitis based on physio-pathological examination of mice. The Jo2 antibody shows cytolytic activity against cell lines expressing mouse Fas by inducing apoptosis.

FIG. 24 shows that peptide fragments can distinguish between two different preclinical models of liver disease due to their distinct biological mechanisms.

FIG. 25 outlines an experimental design of the present application.

FIG. 26 provides experimental results showing the probes can distinguish between healthy, Obese and NASH human samples.

FIG. 27 provides experimental results that show reproducibility among independent sample cohorts with various collection dates, collection protocols, shipment etc.

FIG. 28 provides experimental results showing the peptide fragments can distinguish between different stages of NASH disease progression in specific assay conditions.

FIG. 29 provides experimental results showing the multiplicity of the peptide fragments able to distinguish between NASH and Healthy human K2 EDTA plasma.

FIG. 30A-F provide experimental results demonstrating the association of specific proteases in the detection of disease-specific activity differences in NASH samples in mice K2 EDTA plasma. FIG. 30A shows the results when testing with a pan-protease inhibitor. FIG. 30B shows the results when testing with a cysteine protease family inhibitor. FIG. 30C shows the results when testing with a cathepsin family inhibitor. FIG. 30D shows the results when testing with a CTSB specific inhibitor. FIG. 30E shows the results when testing with a CTSK specific inhibitor. FIG. 30F shows the results when testing with a CTSL specific inhibitor. These results show that this substrate is cleaved by CTSL.

FIG. 31A-B provides experimental results showing that two common promiscuous proteases abundant in plasma are not responsible for determination of disease-based protease activity differences in NASH samples in K2 EDTA mice plasma. FIG. 31A shows the results of testing with a trypsin specific inhibitor and FIG. 31B shows the results when testing with a thrombin specific inhibitor.

FIG. 32A-B provides experimental results showing that activity, not abundance, is responsible for determination of disease-based protease activity differences in human samples. FIG. 32A shows the results of testing pooled samples of healthy and NASH plasma when comparing protease activity. FIG. 32B shows the quantitation ratio for protease activity between healthy and NASH samples.

FIG. 33A-B shows that although Cathepsin-L is equally abundant in both healthy and NASH human samples, the differences in its activity levels allow for the differentiation between healthy and NASH samples. FIG. 33A shows the results of testing for CTSL abundance levels and FIG. 33B shows that testing for CTSL activity levels is superior to testing for CTSL abundance.

FIG. 34A-B provides experimental evidence that the probes can detect both host response and presence of the COVID virus in plasma under two different conditions of plasma collection. FIG. 34A shows the results from the K2 EDTA plasma cohort while FIG. 34B shows the results from the LiHeparin plasma cohort. Probe #18 is a Neutrophil elastase substrate. Probe #409 is a SARS-COV2 3C protease. Probe #462 is a MMP8 substrate. Probe #84 is a Furin substrate. Probe #26 is a Cathepsin K/B, Trypsin, Thrombin, Tryptase substrate.

FIG. 35 provides experimental data that the probes can differentiate between healthy swab samples and COVID swab samples.

FIG. 36A-B provides experimental data showing that 3Cl protease from SARS-COV2 can be detected when spiked in saliva or swab samples. FIG. 36A shows the results from saliva samples while FIG. 36B shows the results from swab samples conditioned in VTM (Viral Transport Media containing up to 10% FBS).

FIG. 37 shows several probes that are capable of differentiating between healthy and COVID samples.

FIG. 38A provides experimental evidence that the Probe #647 can detect the activity of COVID-related proteases to differentiate between healthy and COVID pooled swab samples conditioned in saline. FIG. 38B shows that there are significant differences (p=0.029) between COVID+ (n=18) and COVID− (n−19) samples. FIG. 38C shows the adjusted RFU across timepoints for COVID+ (7 samples were active) and COVID− (1 sample was active) samples.

FIG. 39A-B provides experimental evidence that Granzyme B, a protease linked to other autoimmune diseases, is the protease that allows Probe #647 to differentiate between healthy and COVID samples. FIG. 39A shows the results of inhibition experiments involving Granzyme B while FIG. 39B shows the results of inhibition experiments involving caspases. Differential protease activity is more sensitive to the GzmB specific inhibitor than the caspase inhibitor, implicating GzmB, a hallmark of T-cell activity, in the disease signal detected in swabs.

FIG. 40 shows a paper strip test capable of monitoring Granzyme B activity.

FIG. 41A-B provides experimental evidence showing that the peptide fragments can distinguish between healthy and pancreatic ductal adenocarcinoma (PDAC) samples. FIG. 41A shows the results of first set of experiments, while FIG. 41B shows the results of second set of experiments.

FIG. 42 provides experimental evidence showing that the peptide fragments can distinguish between healthy samples, PDAC samples, and pancreatitis samples.

FIG. 43 shows a schematic diagram for detection of Chlorination and peroxidation activity of MPO using the EnzChek® Myeloperoxidase Activity Assay Kit. AH represents the nonfluorescent Amplex® UltraRed substrate, and A represents its fluorescent oxidation product. Hydrogen peroxide converts MPO to MPO-I and MPO is inactive without the presence of hydrogen peroxide. Amplex® UltraRed is then oxidized by MPO-I and creates the fluorescent oxidation product A which can be read at Ex/Em=530/590.

FIG. 44A-C shows the results for detecting peroxidases. FIG. 44A shows that MPO activities are different between healthy mice and mice with NASH. FIG. 44B shows that MPO activities are different between mice fed on a standard ChowDiet (CD), mice feed on a choline-deficient, L-amino acid-defined, high-fat diet (CDAHFD). FIG. 44C shows that MPO activities are different between healthy human subject and subjects with rheumatoid arthritis.

FIG. 45A-B shows the pooled results of spiked recombinant protease in human plasma using resorufin oleate as substrate. FIG. 46A shows result of 3 recombinant enzymes-carboxylesterase 1, phospholipase A2 and lipoprotein lipase. FIG. 46B shows the result of various concentrations of lipoprotein lipase.

FIG. 46A-C shows general designs of the exemplary cleavable linkers for FRET substrates. FIG. 46A shows general designs for endopeptidase, aminopeptidase and carboxypeptidase substrates. FIG. 46B shows an example that reporter and quencher can be inverted. FIG. 46C shows the generalized substrate designs for aminopeptidase and carboxypeptidase.

FIG. 47A shows the testing result of using LBx-NASH classifier only. FIG. 47B shows the testing result of using LBx-NASH staging while incorporating aspartate aminotransferase (AST) test and AST/alanine aminotransferase (ALT) test classifier information.

FIG. 48A shows the testing result of using AST test, ALT test, and Glympse Liver Test (GLT) classifiers alone or in combination with samples from mouse Cohort 1 to differentiate between positive diagnoses and false positive diagnoses. FIG. 48B shows the same testing with mouse Cohort 2.

FIG. 49A shows the testing result of using AST test, ALT test, and GLT classifiers alone or in combination with samples from mouse Cohort 2 to differentiate between positive diagnoses and false positive diagnoses. FIG. 49B shows the testing result of using GLT classifiers in combination with a subject's enhanced liver fibrosis score (ELF) on samples from rat Cohort 2 to differentiate between positive diagnoses and false positive diagnoses.

FIG. 50A-50B show results of AFP-protease biosensor panel assays. FIG. 10A shows results of the classifier cross-validated in low AFP patients (AFP<10 ng/ml). FIG. 10B shows results of the combined classifier using protease panel+AFP.

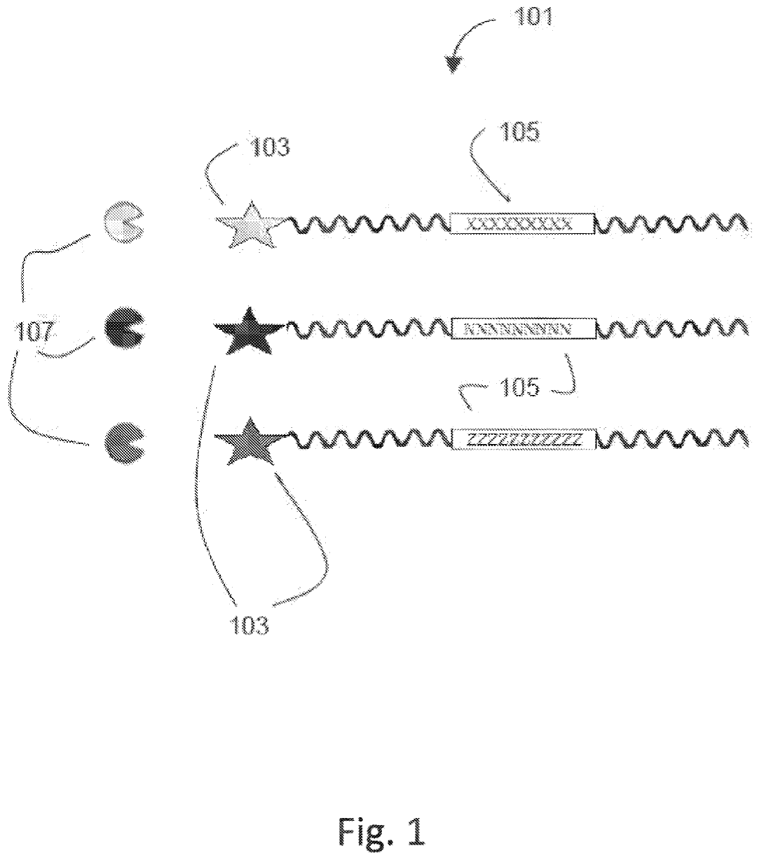

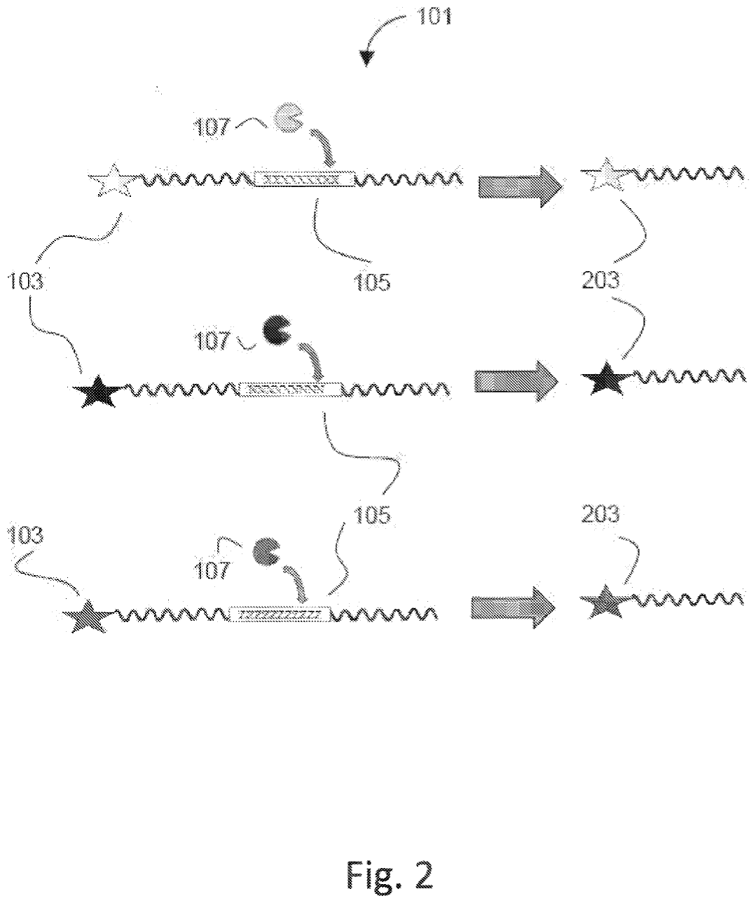

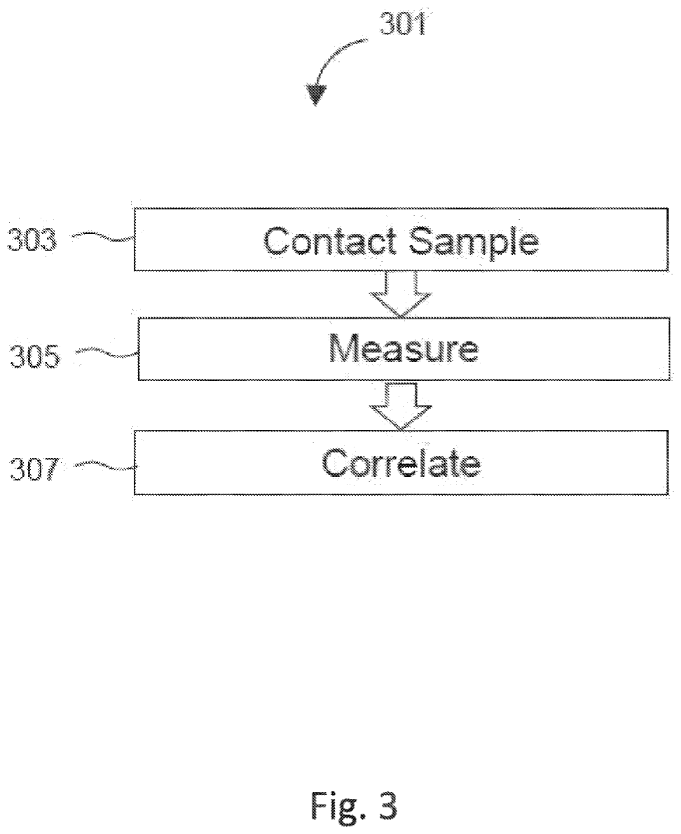

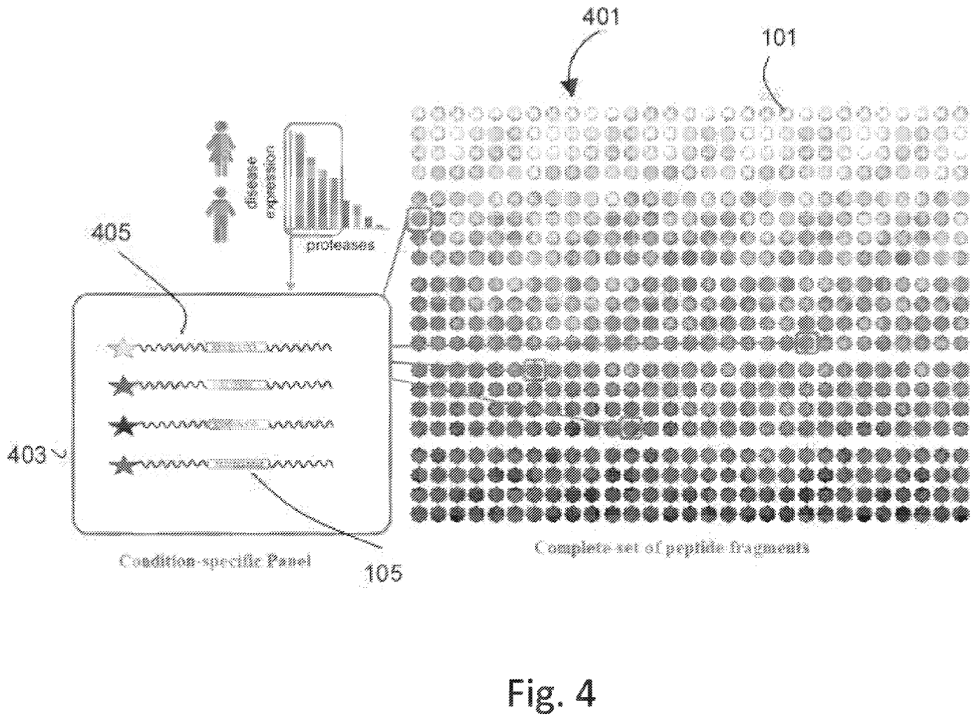

### DETAILED DESCRIPTION

Provided herein are methods comprising contacting a body fluid sample from a subject with a molecule. In some embodiments, the molecule comprises a reporter, and the agent from the body fluid reacts with the molecule, forming a detectable signal. In some embodiments, the method further comprises detecting a rate of formation or an amount of the detectable signal. In some embodiments, the rate of formation or amount of the detectable signal is significantly different from a healthy subject. In some embodiments, the body fluid may be plasma. In some embodiments, the method further comprises determining a disease, a condition, or change in condition of the subject based on the detection. In some embodiments, the method further comprises determining a disease or condition of the subject based on the detection and a parameter that indicates a physiological or clinical condition of the subject, wherein the parameter is not associated with the detection.

Provided herein are methods comprising contacting a body fluid sample from a subject with a molecule. In some embodiments, the molecule comprises a reporter, and the agent from the body fluid reacts with the molecule, forming a detectable signal. In some embodiments, the method further comprises detecting a rate of formation or an amount of the detectable signal. In some embodiments, the rate of formation or amount of the detectable signal is significantly different from a healthy subject. In some embodiments, the body fluid may be plasma. In some embodiments, the method further comprises inputting data from the detection and a parameter, which indicates a physiological or clinical condition of a subject, into an algorithm, wherein the parameter is not associated with the detection.

Provided herein are methods comprising contacting a body fluid sample from a subject with a molecule. In some embodiments, the molecule comprises a cleavable linker and a reporter, and the cleavable linker is cleaved by an agent from the body fluid, releasing the reporter from the molecule. In some embodiments, the method further comprises detecting a rate of formation or an amount of the released reporter. In some embodiments, the rate of formation or amount of the released report is significantly different from a healthy subject. In some embodiments, the body fluid may be plasma. In some embodiments, the method further comprises determining a disease, a condition, or change in condition of the subject based on the detection. In some embodiments, the method further comprises determining a disease or condition of the subject based on the detection and a parameter that indicates a physiological or clinical condition of the subject, wherein the parameter is not associated with the detection.

Provided herein are methods comprising contacting a body fluid sample from a subject with a molecule. In some embodiments, the molecule comprises a cleavable linker and a reporter. In some embodiments, the cleavable linker is cleaved by an agent from the body fluid sample, releasing the reporter from the molecule. In some embodiments, the method further comprises detecting a rate of formation or an amount of the reporter. In some embodiments, the method further comprises inputting data from the detection and a parameter that indicates physiological or clinical condition of a subject into an algorithm, wherein the parameter is not associated with the detection.

In some embodiments, the method further comprises determining or monitoring a disease, a condition, or a change in condition of the subject based on an output from the algorithm. In some embodiments, the algorithm comprises a supervised or unsupervised Machine Learning or Deep Learning algorithm, Logistic Regression, Naive Bayes, Support Vector Machine, Decision Tree, Random Forest, Gradient Boosting, Regularizing Gradient Boosting, K-Nearest Neighbors, Bayesian Network, a continuous regression approach, Ridge Regression, Kernel Ridge Regression, Least Absolute Shrinkage and Selection Operator (LASSO), Elastic Nets, Support Vector Regression, a deep learning approach, Neural Networks, Convolutional Neural Network (CNNs), Recurrent Neural Networks (RNNs), Long Short Term Memory Networks (LSTMs), Restricted Boltzmann Machines (RBMs), Generative Models, Generative Adversarial Networks (GANs), Deep Belief Networks (DBNs), Feedforward Neural Networks, Autoencoders, Radial Basis Function Networks (RBFNs), Multilayer Perceptrons (MLPs), Stochastic Neural Networks or any combination thereof. In some embodiments, the parameter comprises a result or data set from a clinical test of the subject, a data set or result from a genetic test of the subject, a result or data set from a protein test of the subject, a demographic characteristic of the subject, a clinical characteristic of the subject, a digital profile of the subject, or a combination thereof. In some embodiments, the parameter comprises at least two parameters, at least five parameters, at least ten parameters, at least twenty parameters or at least fifty parameters. In some embodiments, the result(s) or data set(s) can be statistically significant. In some embodiments, the statistical significance comprises a probability value (p-value). P-values less than 0.05 are statistically significant. In some embodiments, the parameter comprises a plurality of data sets or results. In some embodiments, the parameter or the plurality of parameters can comprise a concentration. The concentration can be from 0.1 to 10%, from 10% to 20%, from 20% to 30%, from 30% to 40%, from 40% to 50%, from 50% to 60%, from 60% to 70%, from 70% to 80%, from 80% to 90%, from 90% to 100%, or a combination thereof. In some embodiments, the parameter comprises an activity measurement (e.g., rate), a count, a binary call (e.g., Y/N), a value (e.g., height, weight), interpretation of a numerical value (e.g., risk level as high, medium or low), or a disease characteristic or diagnosis.

In some embodiments, the clinical test comprises a tissue biopsy, a blood test, a test for proteins, a test for metabolites, a test for a circulating analyte, a test on any bodily fluid, a test for microbiome, a test for biomarkers, a test for malignancy or tumor, a test for liver function, a test for lipids or any combination thereof. In some embodiments, the circulating analyte comprises a polypeptide, a protein, a glycoprotein, a cytokine, a hormone or a combination thereof. In some embodiments, the genetic test comprises analysis of a nucleic acid, a genomic DNA, a mitochondrial DNA (mtDNA), a mRNA, a microRNA (miRNA), a long non-coding RNA (lncRNA), a snoRNA, rRNA, tRNA, a circulating cell-free DNA (cfDNA), a circulating tumor DNA (ctDNA), a cell-free RNA (cfRNA), a cell-free total nucleic acid (cfTNA), an exosomal nucleic acid, a microbial or viral nucleic acid, a DNA methylation marker, or any combination thereof. In some embodiments, the protein test comprises analysis of a post-translational modification, total protein, proteomics, protein ratios or any combination thereof. In some embodiments, the post-translational modification comprises phosphorylation, glycosylation, ubiquitination, nitrosylation, methylation, acetylation, lipidation, hydroxylation, proteolysis or any combinations thereof. In some embodiments, the demographic characteristic comprises age, gender, race, ethnicity, education, occupation, income, geographic area or any combination thereof. In some embodiments, the clinical characteristic comprises disease characteristics, symptoms and severity/stage of a disease or condition, body mass index (BMI), age at diagnosis, type 2 diabetes, metabolic syndrome, MELD score, alcohol or other toxin consumption, dyslipidemia, genetic risk profile (PNPLA-3), liver stiffness, controlled attenuation parameter (CAP), or any combination thereof.

In some embodiments, the digital profile comprises a digital metric, data set or measurement collected by a wearable device. In some embodiments, the wearable device comprises a smart watch, a smart clothing, a smart jewelry, a fitness tracker, an implantable device, a head-mounted display or a combination thereof. In some embodiments, the digital measurement comprises heart rate, brainwave, muscle bio-signals, sleep patterns and duration, speed, cadence, distance traveled, calories burned, respiration rate, skin conductivity, biomarkers from a bodily fluid, or any combination thereof.

In one aspect, a second body fluid sample is contacted by a second synthetic molecule with a second cleavable linker and a second reporter. In some embodiments, the second cleavable linker is cleaved by a second agent from the body fluid, and the cleavage releases the second reporter from the second synthetic molecule. In some embodiments, the method further comprises detecting a rate of formation or an amount of the second released reporter. In some embodiments, the method further comprises determining a disease or condition of the subject based on the detection of the first released reporter and the detection of the second released reporter. In some embodiments, the rate of formation or the amount of the first released reporter differs (e.g., more than or less than) from the rate of formation or the amount of the second released reporter. In some embodiments, the rate of formation or the amount of the first released reporter is the same as the rate of formation or the amount of the second released reporter.

In some embodiments, the second body fluid sample is different from the first body fluid sample. In some embodiments, the second cleavable linker is different from the first cleavable linker. In some embodiments, the second cleavable linker can be a peptide comprising an amino acid sequence. In some embodiments, the amino acid sequence is selected from SEQ ID Nos: 1-677 or a sequence comprising a mimetic of any one of SEQ ID Nos: 1-677.

### Detection Probe/Molecule

Determination of the disease or condition is based on the rate of formation or amount of the released reporter detected in the sample. A probe/molecule is introduced to the body fluid samples. In some embodiments, the probe/molecule, as disclosed herein, refers to a synthetic probe/molecule. The probe/molecule comprises a cleavable linker and a reporter, and an agent of from the body fluid cleave the cleavable linker, releasing a cleaved reporter. The probe/molecule may have any structure that can fulfill this function. In some embodiments, the reporter may be covalently linked to a cleavable linker. In some embodiments, the reporter may be a fluorescent label, a mass tag, a chromophore, an electrochemically active molecule, a bio-Layer interferometry or surface plasmon resonance detectable molecule, a precipitating substance, a mass spectrometry and liquid chromatography substrate (including size exclusion, reverse phase, isoelectric point, etc.), a magnetically active molecule, a gel forming and/or viscosity changing molecule, an immunoassay detectable molecule, a cell-based amplification detectable molecule, a nucleic acid barcode, or any combinations thereof.

In some embodiments, the reporter may be a fluorescent label and the molecule also comprises a quencher. In some embodiments, the quencher is covalently linked to the cleavable linker. In some embodiments an internally quenched fluorophore is linked to the cleavable linker. In some embodiments, the molecule further comprises a self-immolative spacer. In some other embodiments, the molecule further comprises a carrier.

### Cleavable Linker

In some aspects, the probe/molecule described herein comprises a cleavable linker. The cleavable linker as described herein may be in any structure that is capable of being cleaved by an agent. In some embodiments, the cleavable linker may be a peptide, a carbohydrate, a nucleic acid, a lipid, an ester, a glycoside, a phospholipid, a phosphodiester, a nucleophile/base sensitive linker, a reduction sensitive linker, an electrophile/acid sensitive linker, a metal cleavable linker, an oxidation sensitive linker, an auto-immolable linker (three component probe=enzyme substrate+linker+reporter) or a combination thereof. In some embodiments, the reporter can be in an inactive form and under disease activity becomes detectable. Geoffray Leriche, Louise Chisholm, Alain Wagner, Cleavable linkers in chemical biology, Bioorganic & Medicinal Chemistry, Volume 20, Issue 2, 2012, Pages 571-582, ISSN 0968-0896, https://doi.org/10.1016/j.bmc.2011.07.048.

Cross-linking agents aim to form a covalent bond between two spatially adjacent residues within one or two polymer chains. To identify protein binding partners, the cross-linking agents need to be able to detect and stabilize transient interactions. The crosslinking agents frequently form covalent links between lysine or cysteine residues in the proteins. Alternatively, the cross-linking agent can be photoreactive. Cross-linking cleavable linkers can be used to distinguish between inter- and intra-protein interactions of receptors, signaling cascades, and the structure of multi-protein complexes.

In some embodiments, the cleavable linker may be a peptide. The core structure of a peptide linker sometimes comprises of either a di-peptide or a tetra-peptide that is recognized and cleaved by lysosomal enzymes. Proteases (also called peptidases) catalyze the breakdown of peptide bonds by hydrolysis and is restricted to a specific sequence of amino acids recognizable by the proteases. Commonly used proteases comprise pepsin, trypsin or chymotrypsin. Since proteases have key roles in many diseases, peptide linkers are widely used in drug release systems or in diagnostic tools. In some embodiments, the peptide linkers comprise a short peptide sequence. In some embodiments, the peptide linkers may include at least one non-naturally occurring amino acid.

In some embodiments, the peptide linkers may be less than about 20 amino acids in length. In some embodiments, the peptide linkers may be between 10 and 100 amino acids in length. In some embodiments, the peptide linkers may be 1 to 5, 1 to 10, 1 to 20, 1 to 30, 1 to 50, 1 to 70, 1 to 90, 1 to 100, 5 to 10, 5 to 20, 5 to 30, 5 to 50, 5 to 70, 5 to 90, 5 to 100, 10 to 20, 10 to 30, 10 to 50, 10 to 70, 10 to 90, 10 to 100, 20 to 30, 20 to 50, 20 to 70, 20 to 90, 20 to 100, 30 to 50, 30 to 70, 30 to 90, 30 to 100, 50 to 70, 50 to 90, 50 to 100, 70 to 90, 70 to 100, or 90 to 100 amino acids in length.

TABLE 1
Exemplary sequences for peptide linkers and corresponding probe construct designs

| SEQ ID | | Exemplary | |
|---|---|---|---|
| NO | Sequence | probe name | Exemplary probe construct |
| 1 | SGRSG | Probe #1 | 5-FAM-GSGRSGGK(CPQ2)-PEG2-kk-GC |
| 2 | PGPREG | Probe #2 | 5-FAM-GPGPREGGK(CPQ2)-PEG2-kk-GC |
| 3 | IEPDSGSQ | Probe #3 | 5-FAM-GIEPDSGSQGK(CPQ2)-PEG2-kk-GC |
| 4 | VVADSSMES | Probe #4 | 5-FAM-GVVADSSMESGK(CPQ2)-PEG2-kk-GC |
| 5 | PTSY | Probe #5 | 5-FAM-GPTSYGK(CPQ2)-PEG2-kk-GC |
| 6 | YRFK | Probe #6 | 5-FAM-GYRFKGK(CPQ2)-PEG2-kk-GC |
| 7 | KVPL | Probe #7 | 5-FAM-GKVPLGK(CPQ2)-PEG2-kk-GC |
| 8 | VDVAD | Probe #8 | 5-FAM-GVDVADGK(CPQ2)-PEG2-kk-GC |
| 9 | LETD | Probe #9 | 5-FAM-GLETDGK(CPQ2)-PEG2-kk-GC |
| 10 | LEHD | Probe #10 | 5-FAM-GLEHDGK(CPQ2)-PEG2-kk-GC |
| 11 | REQD | Probe #11 | 5-FAM-GREQDGK(CPQ2)-PEG2-kk-GC |
| 12 | DEVD | Probe #12 | 5-FAM-GDEVDGK(CPQ2)-PEG2-kk-GC |
| 13 | VEID | Probe #13 | 5-FAM-GVEIDGK(CPQ2)-PEG2-kk-GC |
| 14 | VQVDGW | Probe #14 | 5-FAM-GVQVDGWGK(CPQ2)-PEG2-kk-GC |
| 15 | YEVDGW | Probe #15 | 5-FAM-GYEVDGWGK(CPQ2)-PEG2-kk-GC |
| 16 | LEVD | Probe #16 | 5-FAM-GLEVDGK(CPQ2)-PEG2-kk-GC |
| 17 | IEVE | Probe #17 | 5-FAM-GIEVEGK(CPQ2)-PEG2-kk-GC |
| 18 | AAPV | Probe #18 | 5-FAM-GAAPVGK(CPQ2)-PEG2-kk-GC |
| 19 | FFKF | Probe #19 | 5-FAM-GFFKFGK(CPQ2)-PEG2-kk-GC |
| 20 | GRRGKGG | Probe #20 | 5-FAM-GGRRGKGGGK(CPQ2)-PEG2-kk-GC |
| 21 | VKKR | Probe #21 | 5-FAM-GVKKRGK(CPQ2)-PEG2-kk-GC |
| 22 | FAAF(NO2)FVL | Probe #22 | 5-FAM-GFAAF(NO2)FVL GK(CPQ2)-PEG2-kk-GC |
| 23 | VVR | Probe #23 | 5-FAM-GVVRGK(CPQ2)-PEG2-kk-GC |
| 24 | KQKLR | Probe #24 | 5-FAM-GKQKLRGK(CPQ2)-PEG2-kk-GC |
| 25 | RPPGFSAF | Probe #25 | 5-FAM-GRPPGFSAFGK(CPQ2)-PEG2-kk-GC |
| 26 | GPR | Probe #26 | 5-FAM-GGPRGK(CPQ2)-PEG2-kk-GC |
| 27 | FR | Probe #27 | 5-FAM-GFRGK(CPQ2)-PEG2-kk-GC |
| 28 | LPLGL | Probe #28 | 5-FAM-GLPLGLGK(CPQ2)-PEG2-kk-GC |
| 29 | KPLGL | Probe #29 | 5-FAM-GKPLGLGK(CPQ2)-PEG2-kk-GC |
| 30 | (Gaba)PQGLE | Probe #30 | 5-FAM-G(Gaba)PQGLE GK(CPQ2)-PEG2-kk-GC |
| 31 | PKPLAL | Probe #31 | 5-FAM-GPKPLALGK(CPQ2)-PEG2-kk-GC |
| 32 | GPSGIHV | Probe #32 | 5-FAM-GGPSGIHVGK(CPQ2)-PEG2-kk-GC |
| 33 | WAHRTTFYRRGA | Probe #33 | 5-FAM-GWAHRTTFYRRGAGK(CPQ2)-PEG2-kk-GC |
| 34 | WKLRSSKQ | Probe #34 | 5-FAM-GWKLRSSKQGK(CPQ2)-PEG2-kk-GC |
| 35 | PFR | Probe #35 | 5-FAM-GPFRGK(CPQ2)-PEG2-kk-GC |
| 36 | SYRIF | Probe #36 | 5-FAM-GSYRIFGK(CPQ2)-PEG2-kk-GC |
| 37 | RPY | Probe #37 | 5-FAM-GRPYGK(CPQ2)-PEG2-kk-GC |
| 38 | TAFRSAYG | Probe #38 | 5-FAM-GTAFRSAYGGK(CPQ2)-PEG2-kk-GC |
| 39 | WAAFRFSQA | Probe #39 | 5-FAM-GWAAFRFSQAGK(CPQ2)-PEG2-kk-GC |
| 40 | VPR | Probe #40 | 5-FAM-GVPRGK(CPQ2)-PEG2-kk-GC |
| 41 | G | Probe #41 | 5-FAM-GGK(CPQ2)-PEG2-kk-GC |
| 42 | KLRSSKQ | Probe #42 | 5-FAM-GKLRSSKQGK(CPQ2)-PEG2-kk-GC |
| 43 | YASR | Probe #43 | 5-FAM-GYASRGK(CPQ2)-PEG2-kk-GC |
| 44 | RFAQAQQQLP | Probe #44 | 5-FAM-GRFAQAQQQLPGK(CPQ2)-PEG2-kk-GC |
| 45 | KPAKFFRL | Probe #45 | 5-FAM-GKPAKFFRLGK(CPQ2)-PEG2-kk-GC |
| 46 | PRAAA(hF)TSP | Probe #46 | 5-FAM-GPRAAA(hF)TSPGK(CPQ2)-PEG2-kk-GC |
| 47 | VGPQRFSGAP | Probe #47 | 5-FAM-GVGPQRFSGAPGK(CPQ2)-PEG2-kk-GC |
| 48 | FFLAQA(hF)RS | Probe #48 | 5-FAM-GFFLAQA(hF)RS GK(CPQ2)-PEG2-kk-GC |
| 49 | PLAQAV | Probe #49 | 5-FAM-GPLAQAVGK(CPQ2)-PEG2-kk-GC |
| 50 | RTAAVFRP | Probe #50 | 5-FAM-GRTAAVFRPGK(CPQ2)-PEG2-kk-GC |
| 51 | DVQEFRGVTAVIR | Probe #51 | 5-FAM-GDVQEFRGVTAVIRGK(CPQ2)-PEG2-kk-GC |
| 52 | TEGEARGSVI | Probe #52 | 5-FAM-GTEGEARGSVIGK(CPQ2)-PEG2-kk-GC |
| 53 | 1-TR | Probe #53 | 5-FAM-G-1-TRGK(CPQ2)-PEG2-kk-GC |
| 54 | PLFAERK | Probe #54 | 5-FAM-GPLFAERKGK(CPQ2)-PEG2-kk-GC |
| 55 | LLVY | Probe #55 | 5-FAM-GLLVYGK(CPQ2)-PEG2-kk-GC |
| 56 | QQKRKIVL | Probe #56 | 5-FAM-GQQKRKIVLGK(CPQ2)-PEG2-kk-GC |
| 57 | ASHLGLAR | Probe #57 | 5-FAM-GASHLGLARGK(CPQ2)-PEG2-kk-GC |
| 58 | LPSRSSKI | Probe #58 | 5-FAM-GLPSRSSKIGK(CPQ2)-PEG2-kk-GC |
| 59 | STGRNGFK | Probe #59 | 5-FAM-GSTGRNGFKGK(CPQ2)-PEG2-kk-GC |
| 60 | SLLRSEET | Probe #60 | 5-FAM-GSLLRSEETGK(CPQ2)-PEG2-kk-GC |
| 61 | HRGRTLEI | Probe #61 | 5-FAM-GHRGRTLEIGK(CPQ2)-PEG2-kk-GC |
| 62 | YLGRSYKV | Probe #62 | 5-FAM-GYLGRSYKVGK(CPQ2)-PEG2-kk-GC |
| 63 | EKQRIIGG | Probe #63 | 5-FAM-GEKQRIIGGGK(CPQ2)-PEG2-kk-GC |
| 64 | QRQRIIGG | Probe #64 | 5-FAM-GQRQRIIGGGK(CPQ2)-PEG2-kk-GC |
| 65 | LORIYK | Probe #65 | 5-FAM-GLQRIYKGK(CPQ2)-PEG2-kk-GC |
| 66 | SLGRKIQI | Probe #66 | 5-FAM-GSLGRKIQIGK(CPQ2)-PEG2-kk-GC |
| 67 | HAAPRSADIQIDI | Probe #67 | 5-FAM-GHAAPRSADIQIDIGK(CPQ2)-PEG2-kk-GC |
| 68 | FGR | Probe #68 | 5-FAM-GFGRGK(CPQ2)-PEG2-kk-GC |
| 69 | SLGR | Probe #69 | 5-FAM-GSLGRGK(CPQ2)-PEG2-kk-GC |
| 70 | GLQR | Probe #70 | 5-FAM-GGLQRGK(CPQ2)-PEG2-kk-GC |
| 71 | SVARTLLV | Probe #71 | 5-FAM-GSVARTLLVGK(CPQ2)-PEG2-kk-GC |
| 72 | GRIFG | Probe #72 | 5-FAM-GGRIFGGK(CPQ2)-PEG2-kk-GC |
| 73 | APK | Probe #73 | 5-FAM-GAPKGK(CPQ2)-PEG2-kk-GC |
| 74 | GFSPY | Probe #74 | 5-FAM-GGFSPYGK(CPQ2)-PEG2-kk-GC |
| 75 | WELRHAGH | Probe #75 | 5-FAM-GWELRHAGHGK(CPQ2)-PEG2-kk-GC |
| 76 | RQSRIVGGE | Probe #76 | 5-FAM-GRQSRIVGGEGK(CPQ2)-PEG2-kk-GC |
| 77 | EQAVYQTI | Probe #77 | 5-FAM-GEQAVYQTIGK(CPQ2)-PEG2-kk-GC |
| 78 | VAYSGENTFGF | Probe #78 | 5-FAM-GVAYSGENTFGFGK(CPQ2)-PEG2-kk-GC |
| 79 | GGR | Probe #79 | 5-FAM-GGGRGK(CPQ2)-PEG2-kk-GC |
| 80 | ATAD | Probe #80 | 5-FAM-GATADGK(CPQ2)-PEG2-kk-GC |
| 81 | RPLESNAV | Probe #81 | 5-FAM-GRPLESNAVGK(CPQ2)-PEG2-kk-GC |
| 82 | RPLGLAR | Probe #82 | 5-FAM-GRPLGLARGK(CPQ2)-PEG2-kk-GC |
| 83 | AAFF | Probe #83 | 5-FAM-GAAFFGK(CPQ2)-PEG2-kk-GC |
| 84 | RVKRGLA | Probe #84 | 5-FAM-GRVKRGLAGK(CPQ2)-PEG2-kk-GC |
| 85 | AAL | Probe #85 | 5-FAM-GAALGK(CPQ2)-PEG2-kk-GC |
| 86 | CGGmeGVndneeGFFsAr | Probe #86 | 5-FAM-CGGmeGVndneeGFFsArGK(CPQ2) |
| 87 | GPQGIWGQ | Probe #87 | 5FAM-GGPQGIWGQK(CPQ2)-PEG2-C |
| 88 | GLVPRGS | Probe #88 | 5FAM-GGLVPRGSGK(CPQ2)-PEG2-C |
| 89 | GPVGLI | Probe #89 | 5FAM-GGPVGLIGK(CPQ2)-PEG2-C |
| 90 | GPWGIWGQ | Probe #90 | 5FAM-GGPWGIWGQGK(CPQ2)-PEG2-C |
| 91 | GPVPLSLVM | Probe #91 | 5FAM-GGPVPLSLVMK(CPQ2)-PEG2-C |
| 92 | Gf-Pip-RSGG | Probe #92 | 5FAM-GGf-Pip-RSGGGK(CPQ2)-PEG2-C |
| 93 | PLGMRG | Probe #93 | 5FAM-GGf-Pip-KSGGGK(CPQ2)-PEG2-C |
| 94 | PLGMRG | Probe #94 | (FAM)-GPLGMRGG-K(CPQ2)-PEG2-k-GC |
| 95 | P-(Cha)-G- | Probe #95 | (FAM)-GP-(Cha)-G-Cys(Me)-HAG-K(CPQ2)- |
| | Cys(Me)-HA | | PEG2-kk-GC |
| 96 | RPLALWESQ | Probe #96 | (FAM)-GRPLALWESQG-K(CPQ2)-PEG2-k-GC |
| 97 | SGKGPRQITA | Probe #97 | (FAM)-SGKGPRQITA-K(CPQ2)-PEG2-k-GC |
| 98 | SGPLFYSVTA | Probe #98 | (FAM)-SGPLFYSVTA-K(CPQ2)-PEG2-kk-GC |
| 99 | SGRIFLRTA | Probe #99 | (FAM)-SGRIFLRTA-K(CPQ2)-PEG2-GC |
| 100 | SGRSENIRTA | Probe #100 | (FAM)-SGRSENIRTA-K(CPQ2)-PEG2-GC |
| 101 | GSGGS | Probe #101 | (FAM)-GGSGGS-K(CPQ2)-PEG2-kk-GC |
| 102 | KPILFFRLKG | Probe #102 | (FAM)-GKPILFFRLKG-K(CPQ2)-PEG2-kk-GC |
| 103 | AWESR(Nle) | Probe #103 | (FAM)-GAWESR(Nle)GK(CPQ2)-NH2 |
| 104 | NEKSG(Nle) | Probe #104 | (FAM)-GNEKSG(Nle)GK(CPQ2)-NH2 |
| 105 | NATIVY | Probe #105 | (FAM)-GNATIVYGK(CPQ2)-PEG2-k-NH2 |
| 106 | DPFVVS | Probe #106 | (FAM)-GDPFVVSGK(CPQ2)-PEG2-k-NH2 |
| 107 | FH(Nle)FTK | Probe #107 | (FAM)-GFH(Nle)FTKGK(CPQ2)-PEG2-k-NH2 |
| 108 | (Nle)NWHKH | Probe #108 | (FAM)-G(Nle)NWHKHGK(CPQ2)-NH2 |
| 109 | FARRWG | Probe #109 | (FAM)-GFARRWGGK(CPQ2)-PEG2-k-NH2 |
| 110 | PGKWSK | Probe #110 | (FAM)-GPGKWSKGK(CPQ2)-PEG2-k-NH2 |
| 111 | YEEAQP | Probe #111 | (FAM)-GYEEAQPGK(CPQ2)-PEG2-k-NH2 |
| 112 | YGAIKK | Probe #112 | (FAM)-GYGAIKKGK(CPQ2)-PEG2-k-NH2 |
| 113 | TS(Nle)EGY | Probe #113 | (FAM)-GTS(Nle)EGYGK(CPQ2)-PEG2-k |
| 114 | PNNFGS | Probe #114 | (FAM)-GPNNFGSGK(CPQ2)-PEG2-k-NH2 |
| 115 | EDTRNT | Probe #115 | (FAM)-GEDTRNTGK(CPQ2)-NH2 |
| 116 | KDLEQS | Probe #116 | (FAM)-GKDLEQSGK(CPQ2)-NH2 |
| 117 | AALHND | Probe #117 | (FAM)-GAALHNDGK(CPQ2)-PEG2-kk-NH2 |
| 118 | ADSFFK | Probe #118 | (FAM)-GADSFFKGK(CPQ2)-NH2 |
| 119 | ITFWRA | Probe #119 | (FAM)-GITFWRAGK(CPQ2)-NH2 |
| 120 | LSD(Nle)RL | Probe #120 | (FAM)-GLSD(Nle)RLGK(CPQ2)-NH2 |
| 121 | EVGWTY | Probe #121 | (FAM)-GEVGWTYGK(CPQ2)-PEG2-k-NH2 |
| 122 | IAFRQ(Nle) | Probe #122 | (FAM)-GIAFRQ(Nle)GK(CPQ2)-NH2 |
| 123 | YNIHT(Nle) | Probe #123 | (FAM)-GYNIHT(Nle)GK(CPQ2)-PEG2-kk-NH2 |
| 124 | (Nle)LWANH | Probe #124 | (FAM)-G(Nle)LWANHGK(CPQ2)-PEG2-kk-NH2 |
| 125 | LYSVQV | Probe #125 | (FAM)-GLYSVQVGK(CPQ2)-PEG2-k-NH2 |
| 126 | SHI(Nle)SN | Probe #126 | (FAM)-GSHI(Nle)SNGK(CPQ2)-PEG2-kk-NH2 |
| 127 | KLLIDV | Probe #127 | (FAM)-GKLLIDVGK(CPQ2)-NH2 |
| 128 | E(Nle)GVFD | Probe #128 | (FAM)-GE(Nle)GVFDGK(CPQ2)-PEG2-k-NH2 |
| 129 | HQAYTL | Probe #129 | (FAM)-GHQAYTLGK(CPQ2)-PEG2-kk-NH2 |
| 130 | YVRKIQ | Probe #130 | (FAM)-GYVRKIQGK(CPQ2)-PEG2-k-NH2 |
| 131 | DRENSP | Probe #131 | (FAM)-GDRENSPGK(CPQ2)-NH2 |
| 132 | KYDKPR | Probe #132 | (FAM)-GKYDKPRGK(CPQ2)-NH2 |
| 133 | RPWKQL | Probe #133 | (FAM)-GRPWKQLGK(CPQ2)-PEG2-k-NH2 |
| 134 | APLQRY | Probe #134 | (FAM)-GAPLQRYGK(CPQ2)-NH2 |
| 135 | YQGQK(Nle) | Probe #135 | (FAM)-GYQGQK(Nle)GK(CPQ2)-NH2 |
| 136 | GRISSI | Probe #136 | (FAM)-GGRISSIGK(CPQ2)-NH2 |
| 137 | HSLTNV | Probe #137 | (FAM)-GHSLTNVGK(CPQ2)-PEG2-kk-NH2 |
| 138 | EWDFPE | Probe #138 | (FAM)-GEWDFPEGK(CPQ2)-PEG2-k-NH2 |
| 139 | YLA(Nle)DG | Probe #139 | (FAM)-GYLA(Nle)DGGK(CPQ2)-PEG2-k-NH2 |
| 140 | FIY(Nle)PT | Probe #140 | (FAM)-GFIY(Nle)PTGK(CPQ2)-PEG2-k-NH2 |
| 141 | GHETWV | Probe #141 | (FAM)-GGHETWVGK(CPQ2)-PEG2-kk-NH2 |
| 142 | DYIGDE | Probe #142 | (FAM)-GDYIGDEGK(CPQ2)-PEG2-k-NH2 |
| 143 | AGTAHP | Probe #143 | (FAM)-GAGTAHPGK(CPQ2)-PEG2-kk-NH2 |
| 144 | V(Nle)TEIW | Probe #144 | (FAM)-GV(Nle)TEIWGK(CPQ2)-PEG2-k-NH2 |
| 145 | PDDWQN | Probe #145 | (FAM)-GPDDWQNGK(CPQ2)-PEG2-k-NH2 |
| 146 | GLNQEY | Probe #146 | (FAM)-GGLNQEYGK(CPQ2)-PEG2-k-NH2 |
| 147 | YRDAVA | Probe #147 | (FAM)-GYRDAVAGK(CPQ2)-NH2 |
| 148 | TGPKGN | Probe #148 | (FAM)-GTGPKGNGK(CPQ2)-NH2 |
| 149 | DHVPQI | Probe #149 | (FAM)-GDHVPQIGK(CPQ2)-PEG2-kk-NH2 |
| 150 | NKEPIL | Probe #150 | (FAM)-GNKEPILGK(CPQ2)-NH2 |
| 151 | VWN(Nle)VH | Probe #151 | (FAM)-GVWN(Nle)VHGK(CPQ2)-PEG2-kk-NH2 |
| 152 | PVIIEH | Probe #152 | (FAM)-GPVIIEHGK(CPQ2)-PEG2-kk-NH2 |
| 153 | FQTDNL | Probe #153 | (FAM)-GFQTDNLGK(CPQ2)-PEG2-k-NH2 |
| 154 | RF(Nle)HGI | Probe #154 | (FAM)-GRF(Nle)HGIGK(CPQ2)-PEG2-k-NH2 |
| 155 | YAERTT | Probe #155 | (FAM)-GYAERTTGK(CPQ2)-NH2 |
| 156 | NRGELP | Probe #156 | (FAM)-GNRGELPGK(CPQ2)-NH2 |
| 157 | HHYFNY | Probe #157 | (FAM)-GHHYFNYGK(CPQ2)-PEG2-k-NH2 |
| 158 | STPYYH | Probe #158 | (FAM)-GSTPYYHGK(CPQ2)-PEG2-kk-NH2 |
| 159 | WFYPSA | Probe #159 | (FAM)-GWFYPSAGK(CPQ2)-PEG2-k-NH2 |
| 160 | SEFLFS | Probe #160 | (FAM)-GSEFLFSGK(CPQ2)-PEG2-k-NH2 |
| 161 | WYKTQY | Probe #161 | (FAM)-GWYKTQYGK(CPQ2)-NH2 |
| 162 | VTHLKV | Probe #162 | (FAM)-GVTHLKVGK(CPQ2)-PEG2-k-NH2 |
| 163 | INGGFS | Probe #163 | (FAM)-GINGGFSGK(CPQ2)-PEG2-k-NH2 |
| 164 | TVLGLD | Probe #164 | (FAM)-GTVLGLDGK(CPQ2)-PEG2-k-NH2 |
| 165 | SYWP(Nle)Q | Probe #165 | (FAM)-GSYWP(Nle)QGK(CPQ2)-PEG2-k-NH2 |
| 166 | ASQQHR | Probe #166 | (FAM)-GASQQHRGK(CPQ2)-PEG2-k-NH2 |
| 167 | KNPAKA | Probe #167 | (FAM)-GKNPAKAGK(CPQ2)-PEG2-k-NH2 |
| 168 | (Nle)YWLVE | Probe #168 | (FAM)-G(Nle)YWLVEGK(CPQ2)-PEG2-k-NH2 |
| 169 | SWWIFE | Probe #169 | (FAM)-GSWWIFEGK(CPQ2)-PEG2-k-NH2 |
| 170 | VNYEQD | Probe #170 | (FAM)-GVNYEQDGK(CPQ2)-PEG2-k-NH2 |
| 171 | HFF(Nle)AE | Probe #171 | (FAM)-GHFF(Nle)AEGK(CPQ2)-PEG2-kk-NH2 |
| 172 | DIPPHW | Probe #172 | (FAM)-GDIPPHWGK(CPQ2)-PEG2-kk-NH2 |
| 173 | VDQW(Nle)W | Probe #173 | (FAM)-GVDQW(Nle)WGK(CPQ2)-PEG2-k-NH2 |
| 174 | LRSL(Nle)K | Probe #174 | (FAM)-GLRSL(Nle)KGK(CPQ2)-PEG2-k-NH2 |
| 175 | (Nle)(Nle)IRHA | Probe #175 | (FAM)-G(Nle)(Nle)IRHAGK(CPQ2)-PEG2-k-NH2 |
| 176 | HDVKFI | Probe #176 | (FAM)-GHDVKFIGK(CPQ2)-PEG2-kk-NH2 |
| 177 | KRVQFL | Probe #177 | (FAM)-GKRVQFLGK(CPQ2)-PEG2-k-NH2 |
| 178 | RD(Nle)YAE | Probe #178 | (FAM)-GRD(Nle)YAEGK(CPQ2)-NH2 |
| 179 | L(Nle)IYFE | Probe #179 | (FAM)-GL(Nle)IYFEGK(CPQ2)-PEG2-k-NH2 |
| 180 | LRTKQS | Probe #180 | (FAM)-GLRTKQSGK(CPQ2)-PEG2-k-NH2 |
| 181 | WHGQQY | Probe #181 | (FAM)-GWHGQQYGK(CPQ2)-PEG2-kk-NH2 |
| 182 | GPEGTI | Probe #182 | (FAM)-GGPEGTIGK(CPQ2)-PEG2-k-NH2 |
| 183 | ELDPIP | Probe #183 | (FAM)-GELDPIPGK(CPQ2)-PEG2-k-NH2 |
| 184 | GRAADF | Probe #184 | (FAM)-GGRAADFGK(CPQ2)-NH2 |
| 185 | HFIDYI | Probe #185 | (FAM)-GHFIDYIGK(CPQ2)-PEG2-kk-NH2 |
| 186 | S(Nle)(Nle)RVH | Probe #186 | (FAM)-GS(Nle)(Nle)RVHGK(CPQ2)-PEG2-k-NH2 |
| 187 | SFRKII | Probe #187 | (FAM)-GSFRKIIGK(CPQ2)-PEG2-k-NH2 |
| 188 | TYE(Nle)FS | Probe #188 | (FAM)-GTYE(Nle)FSGK(CPQ2)-PEG2-k-NH2 |
| 189 | HLLGFY | Probe #189 | (FAM)-GHLLGFYGK(CPQ2)-PEG2-kk-NH2 |
| 190 | (Nle)WTALT | Probe #190 | (FAM)-G(Nle)WTALTGK(CPQ2)-PEG2-k-NH2 |
| 191 | IWN(Nle)VY | Probe #191 | (FAM)-GIWN(Nle)VYGK(CPQ2)-PEG2-k-NH2 |
| 192 | RRNPLW | Probe #192 | (FAM)-GRRNPLWGK(CPQ2)-PEG2-k-NH2 |
| 193 | RWYGGI | Probe #193 | (FAM)-GRWYGGIGK(CPQ2)-NH2 |
| 194 | KTGDAR | Probe #194 | (FAM)-GKTGDARGK(CPQ2)-PEG2-k-NH2 |
| 195 | NYWEAN | Probe #195 | (FAM)-GNYWEANGK(CPQ2)-PEG2-k-NH2 |
| 196 | (Nle)QFDTS | Probe #196 | (FAM)-G(Nle)QFDTSGK(CPQ2)-PEG2-k-NH2 |
| 197 | KRGAVE | Probe #197 | (FAM)-GKRGAVEGK(CPQ2)-PEG2-k-NH2 |
| 198 | SLKPTE | Probe #198 | (FAM)-GSLKPTEGK(CPQ2)-NH2 |
| 199 | ENDRLP | Probe #199 | (FAM)-GENDRLPGK(CPQ2)-NH2 |
| 200 | NSYQVQ | Probe #200 | (FAM)-GNSYQVQGK(CPQ2)-PEG2-k-NH2 |
| 201 | YPKEYL | Probe #201 | (FAM)-GYPKEYLGK(CPQ2)-NH2 |
| 202 | INNKWQ | Probe #202 | (FAM)-GINNKWQGK(CPQ2)-NH2 |
| 203 | (Nle)EFQGW | Probe #203 | (FAM)-G(Nle)EFQGWGK(CPQ2)-PEG2-k-NH2 |
| 204 | PVRSTN | Probe #204 | (FAM)-GPVRSTNGK(CPQ2)-NH2 |
| 205 | SQAIKV | Probe #205 | (FAM)-GSQAIKVGK(CPQ2)-NH2 |
| 206 | WA(Nle)LYH | Probe #206 | (FAM)-GWA(Nle)LYHGK(CPQ2)-PEG2-kk-NH2 |
| 207 | ISWIHA | Probe #207 | (FAM)-GISWIHAGK(CPQ2)-PEG2-kk-NH2 |
| 208 | AHDIV | Probe #208 | (FAM)-GAHDIVNGK(CPQ2)-PEG2-kk-NH2 |
| 209 | RHNVAS | Probe #209 | (FAM)-GRHNVASGK(CPQ2)-PEG2-k-NH2 |
| 210 | SVFVIE | Probe #210 | (FAM)-GSVFVIEGK(CPQ2)-PEG2-k-NH2 |
| 211 | FAKYYK | Probe #211 | (FAM)-GFAKYYKGK(CPQ2)-PEG2-k-NH2 |
| 212 | PYNTLQ | Probe #212 | (FAM)-GPYNTLQGK(CPQ2)-PEG2-k-NH2 |
| 213 | (Nle)DWGH(Nle) | Probe #213 | (FAM)-G(Nle)DWGH(Nle)GK(CPQ2)-PEG2-kk-NH2 |
| 214 | SNREWF | Probe #214 | (FAM)-GSNREWFGK(CPQ2)-NH2 |
| 215 | GKSEHT | Probe #215 | (FAM)-GGKSEHTGK(CPQ2)-PEG2-kk-NH2 |
| 216 | FP(Nle)TDQ | Probe #216 | (FAM)-GFP(Nle)TDQGK(CPQ2)-PEG2-k-NH2 |
| 217 | WSKFW(Nle) | Probe #217 | (FAM)-GWSKFW(Nle)GK(CPQ2) |
| 218 | RFTRPH | Probe #218 | (FAM)-GRFTRPHGK(CPQ2)-NH2 |
| 219 | QET(Nle)KD | Probe #219 | (FAM)-GQET(Nle)KDGK(CPQ2)-NH2 |
| 220 | HWWDVL | Probe #220 | (FAM)-GHWWDVLGK(CPQ2)-PEG2-kk-NH2 |
| 221 | FNL V(Nle)S | Probe #221 | (FAM)-GFNLV(Nle)SGK(CPQ2)-PEG2-k-NH2 |
| 222 | SAWRQR | Probe #222 | (FAM)-GSAWRQRGK(CPQ2)-PEG2-k-NH2 |
| 223 | TFHIFL | Probe #223 | (FAM)-GTFHIFLGK(CPQ2)-PEG2-kk-NH2 |
| 224 | WPQHVK | Probe #224 | (FAM)-GWPQHVKGK(CPQ2)-PEG2-k-NH2 |
| 225 | LI(Nle)HKN | Probe #225 | (FAM)-GLI(Nle)HKNGK(CPQ2)-PEG2-k-NH2 |
| 226 | QDLEQP | Probe #226 | (FAM)-GQDLEQPGK(CPQ2)-PEG2-k-NH2 |
| 227 | HQKK(Nle)P | Probe #227 | (FAM)-GHQKK(Nle)PGK(CPQ2)-NH2 |
| 228 | GVTWLN | Probe #228 | (FAM)-GGVTWLNGK(CPQ2)-PEG2-k-NH2 |
| 229 | AGEPFK | Probe #229 | (FAM)-GAGEPFKGK(CPQ2)-NH2 |
| 230 | SR(Nle)ATT | Probe #230 | (FAM)-GSR(Nle)ATTGK(CPQ2)-NH2 |
| 231 | LAF(Nle)NH | Probe #231 | (FAM)-GLAF(Nle)NHGK(CPQ2)-PEG2-kk-NH2 |
| 232 | PPSGLS | Probe #232 | (FAM)-GPPSGLSGK(CPQ2)-PEG2-k-NH2 |
| 233 | YTHSSP | Probe #233 | (FAM)-GYTHSSPGK(CPQ2)-PEG2-kk-NH2 |
| 234 | DGSHYR | Probe #234 | (FAM)-GDGSHYRGK(CPQ2)-PEG2-kk-NH2 |
| 235 | Y(Nle)GNGY | Probe #235 | (FAM)-GY(Nle)GNGYGK(CPQ2)-PEG2-k-NH2 |
| 236 | DSITVS | Probe #236 | (FAM)-GDSITVSGK(CPQ2)-PEG2-k-NH2 |
| 237 | QTPNIQ | Probe #237 | (FAM)-GQTPNIQGK(CPQ2)-PEG2-k-NH2 |
| 238 | KLFFGY | Probe #238 | (FAM)-GKLFFGYGK(CPQ2)-NH2 |
| 239 | TQNFNW | Probe #239 | (FAM)-GTQNFNWGK(CPQ2)-PEG2-k-NH2 |
| 240 | YSDHEV | Probe #240 | (FAM)-GYSDHEVGK(CPQ2)-PEG2-kk-NH2 |
| 241 | RYVVPA | Probe #241 | (FAM)-GRYVVPAGK(CPQ2)-NH2 |
| 242 | ILHRIR | Probe #242 | (FAM)-GILHRIRGK(CPQ2)-NH2 |
| 243 | ESDNQ(Nle) | Probe #243 | (FAM)-GESDNQ(Nle)GK(CPQ2)-PEG2-k-NH2 |
| 244 | YDDKG(Nle) | Probe #244 | (FAM)-GYDDKG(Nle)GK(CPQ2)-NH2 |
| 245 | QLS(Nle)VW | Probe #245 | (FAM)-GQLS(Nle)VWGK(CPQ2)-PEG2-k-NH2 |
| 246 | PGGER(Nle) | Probe #246 | (FAM)-GPGGER(Nle)GK(CPQ2)-NH2 |
| 247 | WKHHPD | Probe #247 | (FAM)-GWKHHPDGK(CPQ2)-NH2 |
| 248 | QWVDED | Probe #248 | (FAM)-GQWVDEDGK(CPQ2)-PEG2-k-NH2 |
| 249 | NAYNEI | Probe #249 | (FAM)-GNAYNEIGK(CPQ2)-PEG2-k-NH2 |
| 250 | EEKAPR | Probe #250 | (FAM)-GEEKAPRGK(CPQ2)-PEG2-kk-NH2 |
| 251 | PWQIGK | Probe #251 | (FAM)-GPWQIGKGK(CPQ2)-NH2 |
| 252 | IAQVGN | Probe #252 | (FAM)-GIAQVGNGK(CPQ2)-PEG2-k-NH2 |
| 253 | V(Nle)RQSE | Probe #253 | (FAM)-GV(Nle)RQSEGK(CPQ2)-NH2 |
| 254 | TERVDA | Probe #254 | (FAM)-GTERVDAGK(CPQ2)-NH2 |
| 255 | WLRWRL | Probe #255 | (FAM)-GWLRWRLGK(CPQ2)-PEG2-k-NH2 |
| 256 | WKTKGQ | Probe #256 | (FAM)-GWKTKGQGK(CPQ2)-PEG2-k-NH2 |
| 257 | QSNGDV | Probe #257 | (FAM)-GQSNGDVGK(CPQ2)-PEG2-k-NH2 |
| 258 | TLFYAL | Probe #258 | (FAM)-GTLFYALGK(CPQ2)-PEG2-k-NH2 |
| 259 | TVTLNP | Probe #259 | (FAM)-GTVTLNPGK(CPQ2)-PEG2-k-NH2 |
| 260 | YAFGRK | Probe #260 | (FAM)-GYAFGRKGK(CPQ2)-PEG2-k-NH2 |
| 26 | DYNYWD | Probe #261 | (FAM)-GDYNYWDGK(CPQ2)-PEG2-k-NH2 |
| 262 | EWHEII | Probe #262 | (FAM)-GEWHEIIGK(CPQ2)-PEG2-kk-NH2 |
| 263 | QKAAWD | Probe #263 | (FAM)-GQKAAWDGK(CPQ2)-NH2 |
| 264 | DNTSAD | Probe #264 | (FAM)-GDNTSADGK(CPQ2)-PEG2-k-NH2 |
| 265 | HEGEYV | Probe #265 | (FAM)-GHEGEYVGK(CPQ2)-PEG2-kk-NH2 |
| 266 | WSPSFK | Probe #266 | (FAM)-GWSPSFKGK(CPQ2)-NH2 |
| 267 | HDEHWT | Probe #267 | (FAM)-GHDEHWTGK(CPQ2)-PEG2-kk-NH2 |
| 268 | YVW(Nle)RD | Probe #268 | (FAM)-GYVW(Nle)RDGK(CPQ2)-NH2 |
| 269 | (Nle)DP(Nle)KF | Probe #269 | (FAM)-G(Nle)DP(Nle)KFGK(CPQ2)-NH2 |
| 270 | (Nle)R(Nle)FWD | Probe #270 | (FAM)-G(Nle)R(Nle)FWDGK(CPQ2)-NH2 |
| 271 | DIAIT(Nle) | Probe #271 | (FAM)-GDIAIT(Nle)GK(CPQ2)-PEG2-k-NH2 |
| 272 | PI(Nle)RFH | Probe #272 | (FAM)-GPI(Nle)RFHGK(CPQ2)-PEG2-k-NH2 |
| 273 | VWQGYI | Probe #273 | (FAM)-GVWQGYIGK(CPQ2)-PEG2-k-NH2 |
| 274 | KK(Nle)SNP | Probe #274 | (FAM)-GKK(Nle)SNPGK(CPQ2)-PEG2-k-NH2 |
| 275 | GHPLSP | Probe #275 | (FAM)-GGHPLSPGK(CPQ2)-PEG2-kk-NH2 |
| 276 | VRQHKP | Probe #276 | (FAM)-GVRQHKPGK(CPQ2)-NH2 |
| 277 | AQNFYR | Probe #277 | (FAM)-GAQNFYRGK(CPQ2)-NH2 |
| 278 | VAGKSI | Probe #278 | (FAM)-GVAGKSIGK(CPQ2)-NH2 |
| 279 | LVGQVN | Probe #279 | (FAM)-GLVGQVNGK(CPQ2)-PEG2-k-NH2 |
| 280 | QVKHFT | Probe #280 | (FAM)-GQVKHFTGK(CPQ2)-PEG2-k-NH2 |
| 281 | QKSVVS | Probe #281 | (FAM)-GQKSVVSGK(CPQ2)-NH2 |
| 282 | Y(Nle)QEWL | Probe #282 | (FAM)-GY(Nle)QEWLGK(CPQ2)-PEG2-k-NH2 |
| 283 | G(Nle)YIDE | Probe #283 | (FAM)-GG(Nle)YIDEGK(CPQ2)-PEG2-k-NH2 |
| 284 | NAGSKF | Probe #284 | (FAM)-GNAGSKFGK(CPQ2)-NH2 |
| 285 | EFVHNP | Probe #285 | (FAM)-GEFVHNPGK(CPQ2)-PEG2-kk-NH2 |
| 286 | WE(Nle)VKI | Probe #286 | (FAM)-GWE(Nle)VKIGK(CPQ2)-NH2 |
| 287 | WVGASH | Probe #287 | (FAM)-GWVGASHGK(CPQ2)-PEG2-kk-NH2 |
| 288 | ITTLY(Nle) | Probe #288 | (FAM)-GITTLY(Nle)GK(CPQ2)-PEG2-k-NH2 |
| 289 | GHIDEY | Probe #289 | (FAM)-GGHIDEYGK(CPQ2)-PEG2-kk-NH2 |
| 290 | KV(Nle)DYG | Probe #290 | (FAM)-GKV(Nle)DYGGK(CPQ2)-NH2 |
| 291 | QEKQT(Nle) | Probe #291 | (FAM)-GQEKQT(Nle)GK(CPQ2)-NH2 |
| 292 | EVGHEA | Probe #292 | (FAM)-GEVGHEAGK(CPQ2)-PEG2-kk-NH2 |
| 293 | AWEGQY | Probe #293 | (FAM)-GAWEGQYGK(CPQ2)-PEG2-k-NH2 |
| 294 | FLVQWT | Probe #294 | (FAM)-GFLVQWTGK(CPQ2)-PEG2-k-NH2 |
| 295 | SKWGYW | Probe #295 | (FAM)-GSKWGYWGK(CPQ2)-NH2 |
| 296 | TWIS(Nle)Q | Probe #296 | (FAM)-GTWIS(Nle)QGK(CPQ2)-PEG2-k-NH2 |
| 297 | VIDKDF | Probe #297 | (FAM)-GVIDKDFGK(CPQ2)-NH2 |
| 298 | VKFAIY | Probe #298 | (FAM)-GVKFAIYGK(CPQ2)-NH2 |
| 299 | HNQ(Nle)KS | Probe #299 | (FAM)-GHNQ(Nle)KSGK(CPQ2)-PEG2-k-NH2 |
| 300 | QYVFF(Nle) | Probe #300 | (FAM)-GQYVFF(Nle)GK(CPQ2)-PEG2-k-NH2 |
| 301 | YNPRE(Nle) | Probe #301 | (FAM)-GYNPRE(Nle)GK(CPQ2)-NH2 |
| 302 | KHG(Nle)PE | Probe #302 | (FAM)-GKHG(Nle)PEGK(CPQ2)-PEG2-kk-NH2 |
| 303 | WSREYW | Probe #303 | (FAM)-GWSREYWGK(CPQ2)-NH2 |
| 304 | IDRVDK | Probe #304 | (FAM)-GIDRVDKGK(CPQ2)-PEG2-kk-NH2 |
| 305 | GDRENSPK(CPQ2) | Probe #305 | (FAM)-kkGDRENSPK(CPQ2)L-OH |
| | L-OH | | |
| 306 | GDRENSPLK | Probe #306 | (FAM)-kkGDRENSPLK(CPQ2)-OH |
| | (CPQ2)-OH | | |
| 307 | NAGSKFK(CPQ2) | Probe #307 | (FAM)-GNAGSKFK(CPQ2)Q-OH |
| | Q-OH | | |
| 308 | NAGSKFQK | Probe #308 | (FAM)-GNAGSKFQK(CPQ2)-OH |
| | (CPQ2)-OH | | |
| 309 | GHLLGFYK | Probe #309 | (FAM)-kkGHLLGFYK(CPQ2)V-OH |
| | (CPQ2)V-OH | | |
| 310 | GHLLGFYVK | Probe #310 | (FAM)-kkGHLLGFYVK(CPQ2)-OH |
| | (CPQ2)-OH | | |
| 311 | GQEKQT(Nle)K | Probe #311 | (FAM)-kkGQEKQT(Nle)K(CPQ2)(Nle)-OH |
| | (CPQ2)(Nle)-OH | | |
| 312 | GQEKQT(Nle) | Probe #312 | (FAM)-kkGQEKQT(Nle)(Nle)K(CPQ2)-OH |
| | (Nle)K(CPQ2)-OH | | |
| 313 | kGDPFVVSK | Probe #313 | (FAM)-kGDPFVVSK(CPQ2)W-OH |
| | (CPQ2)W-OH | | |
| 314 | kGDPFVVSWK | Probe #314 | (FAM)-kGDPFVVSWK(CPQ2)-OH |
| | (CPQ2)-OH | | |
| 315 | NAYNEIK(CPQ2) | Probe #315 | (FAM)-GNAYNEIK(CPQ2)R-OH |
| | R-OH | | |
| 316 | NAYNEIRK | Probe #316 | (FAM)-GNAYNEIRK(CPQ2)-OH |
| | (CPQ2)-OH | | |
| 317 | V(Nle)RQSEK | Probe #317 | (FAM)-GV(Nle)RQSEK(CPQ2)N-OH |
| | (CPQ2)N-OH | | |
| 318 | V(Nle)RQSENK | Probe #318 | (FAM)-GV(Nle)RQSENK(CPQ2) |
| | (CPQ2)-OH | | |
| 319 | YNPRE(Nle)K | Probe #319 | (FAM)-GYNPRE(Nle)K(CPQ2)I-OH |
| | (CPQ2)I-OH | | |
| 320 | YNPRE(Nle)IK | Probe #320 | (FAM)-GYNPRE(Nle)IK(CPQ2)-OH |
| | (CPQ2)-OH | | |
| 321 | EFVHNPK(CPQ2) | Probe #321 | (FAM)-kGEFVHNPK(CPQ2)K-OH |
| | K-OH | | |
| 322 | EFVHNPKK | Probe #322 | (FAM)-kGEFVHNPKK(CPQ2)-OH |
| | (CPQ2)-OH | | |
| 323 | KRVQFLK(CPQ2) | Probe #323 | (FAM)-GKRVQFLK(CPQ2)H-OH |
| | H-OH | | |
| 324 | KRVQFLHK | Probe #324 | (FAM)-GKRVQFLHK(CPQ2)-OH |
| | (CPQ2)-OH | | |
| 325 | LI(Nle)HKNK | Probe #325 | (FAM)-kGLI(Nle)HKNK(CPQ2)G-OH |
| | (CPQ2)G-OH | | |
| 326 | LI(Nle)HKNGK | Probe #326 | (FAM)-kGLI(Nle)HKNGK(CPQ2)-OH |
| | (CPQ2)-OH | | |
| 327 | WA(Nle)LYHK | Probe #327 | (FAM)-kkGWA(Nle)LYHK(CPQ2)S-OH |
| | (CPQ2)S-OH | | |
| 328 | WA(Nle)LYHS | Probe #328 | (FAM)-kkGWA(Nle)LYHSK(CPQ2)-OH |
| | K(CPQ2)-OH | | |
| 329 | AHDIVNK(CPQ2) | Probe #329 | (FAM)-kkGAHDIVNK(CPQ2)Y-OH |
| | Y-OH | | |
| 330 | AHDIVNYK | Probe #330 | (FAM)-kkGAHDIVNYK(CPQ2)-OH |
| | (CPQ2)-OH | | |
| 331 | SVFVIEK(CPQ2) | Probe #331 | (FAM)-kGSVFVIEK(CPQ2)P-OH |
| | P-OH | | |
| 332 | SVFVIEPK | Probe #332 | (FAM)-kGSVFVIEPK(CPQ2)-OH |
| | (CPQ2)-OH | | |
| 333 | PPSGLSK(CPQ2) | Probe #333 | (FAM)-kGPPSGLSK(CPQ2)E-OH |
| | E-OH | | |
| 334 | PPSGLSEK | Probe #334 | (FAM)-kGPPSGLSEK(CPQ2)-OH |
| | (CPQ2)-OH | | |
| 335 | RWYGGIK | Probe #335 | (FAM)-kkGRWYGGIK(CPQ2)F-OH |
| | (CPQ2)F-OH | | |
| 336 | RWYGGIFK | Probe #336 | (FAM)-kkGRWYGGIFK(CPQ2)-OH |
| | (CPQ2)-OH | | |
| 337 | QYVFF(Nle)K | Probe #337 | (FAM)-kGQYVFF(Nle)K(CPQ2)D-OH |
| | (CPQ2)D-OH | | |
| 338 | QYVFF(Nle)DK | Probe #338 | (FAM)-kGQYVFF(Nle)DK(CPQ2)-OH |
| | (CPQ2)-OH | | |
| 339 | FAKYYKK | Probe #339 | (FAM)-kGFAKYYKK(CPQ2)T-OH |
| | (CPQ2)T-OH | | |
| 340 | FAKYYKTK | Probe #340 | (FAM)-KGFAKYYKTK(CPQ2)-OH |
| | (CPQ2)-OH | | |
| 341 | QVKHFTK(CPQ2) | Probe #341 | (FAM)-kGQVKHFTK(CPQ2)A-OH |
| | A-OH | | |
| 342 | QVKHFTAK | Probe #342 | (FAM)-kGQVKHFTAK(CPQ2)-OH |
| | (CPQ2)-OH | | |
| 343 | APK(CPQ2)-OH | Probe #343 | FAM-APK(CPQ2)-OH |
| 344 | NH2-HK(FAM) | Probe #344 | NH2-HK(FAM)DRENSPGK(CPQ2)-NH2 |
| | DRENSP | | |
| 345 | NH2-K(FAM) | Probe #345 | NH2-K(FAM)HDRENSPGK(CPQ2)-NH2 |
| | HDRENSP | | |
| 346 | NH2-WK(FAM) | Probe #346 | NH2-WK(FAM)NAGSKFGKK(CPQ2)-NH2 |
| | NAGSKF | | |
| 347 | NH2-K(FAM) | Probe #347 | NH2-K(FAM)WNAGSKFGKK(CPQ2)-NH2 |
| | WNAGSKF | | |
| 348 | NH2-SK(FAM) | Probe #348 | NH2-SK(FAM)HLLGFYGKK(CPQ2)-NH2 |
| | HLLGFY | | |
| 349 | NH2-K(FAM) | Probe #349 | NH2-K(FAM)SHLLGFYGKK(CPQ2)-NH2 |
| | SHLLGFY | | |
| 350 | NH2-KK(FAM) | Probe #350 | NH2-KK(FAM)QEKQT(Nle)GK(CPQ2)-NH2 |
| | QEKQT(Nle) | | |
| 35 | NH2-K(FAM) | Probe #351 | NH2-K(FAM)KQEKQT(Nle)GK(CPQ2)-NH2 |
| | KQEKQT(Nle) | | |
| 352 | NH2-GK(FAM) | Probe #352 | NH2-GK(FAM)DPFVVSGK(CPQ2)-NH2 |
| | DPFVVS | | |
| 353 | NH2-K(FAM) | Probe #353 | NH2-K(FAM)GDPFVVSGK(CPQ2)-NH2 |
| | GDPFVVS | | |
| 354 | NH2-PK(FAM) | Probe #354 | NH2-PK(FAM)NAYNEIGK(CPQ2)-NH2 |
| | NAYNEI | | |
| 355 | NH2-K(FAM) | Probe #355 | NH2-K(FAM)PNAYNEIGK(CPQ2)-NH2 |
| | PNAYNEI | | |
| 356 | NH2-DK(FAM)V | Probe #356 | NH2-DK(FAM)V(Nle)RQSEGkK(CPQ2)-NH2 |
| | (Nle)RQSE | | |
| 357 | NH2-K(FAM) | Probe #357 | NH2-K(FAM)DV(Nle)RQSEGKK(CPQ2)-NH2 |
| | DV(Nle)RQSE | | |
| 358 | NH2-EK(FAM) | Probe #358 | NH2-EK(FAM)YNPRE(Nle)GkK(CPQ2)-NH2 |
| | YNPRE(Nle) | | |
| 359 | NH2-K(FAM) | Probe #359 | NH2-K(FAM)EYNPRE(Nle)GkK(CPQ2)-NH2 |
| | EYNPRE(Nle) | | |
| 360 | NH2-TK(FAM) | Probe #360 | NH2-TK(FAM)EFVHNPGkK(CPQ2)-NH2 |
| | EFVHNP | | |
| 361 | NH2-TK(FAM) | Probe #361 | NH2-K(FAM)TEFVHNPGkK(CPQ2)-NH2 |
| | EFVHNP | | |
| 362 | NH2-QK(FAM) | Probe #362 | NH2-QK(FAM)KRVQFLGK(CPQ2)-NH2 |
| | KRVQFL | | |
| 363 | NH2-K(FAM) | Probe #363 | NH2-K(FAM)QKRVQFLGK(CPQ2)-NH2 |
| | QKRVQFL | | |
| 364 | NH2-YK(FAM) | Probe #364 | NH2-YK(FAM)LI(Nle)HKNGK(CPQ2)-NH2 |
| | LI(Nle)HKN | | |
| 365 | NH2-K(FAM) | Probe #365 | NH2-K(FAM)YLI(Nle)HKNGK(CPQ2)-NH2 |
| | YLI(Nle)HKN | | |
| 366 | NH2-FK(FAM) | Probe #366 | NH2-FK(FAM)WA(Nle)LYHGkK(CPQ2)-NH2 |
| | WA(Nle)LYH | | |
| 367 | NH2-K(FAM) | Probe #367 | NH2-K(FAM)FWA(Nle)LYHGkK(CPQ2)-NH2 |
| | FWA(Nle)LYH | | |
| 368 | NH2-IK(FAM) | Probe #368 | NH2-IK(FAM)AHDIVNGKK(CPQ2)-NH2 |
| | AHDIVN | | |
| 369 | NH2-K(FAM) | Probe #369 | NH2-K(FAM)IAHDIVNGKK(CPQ2)-NH2 |
| | IAHDIVN | | |
| 370 | NH2-VK(FAM) | Probe #370 | NH2-VK(FAM)SVFVIEGK(CPQ2)-NH2 |
| | SVFVIE | | |
| 371 | NH2-K(FAM) | Probe #371 | NH2-K(FAM)VSVFVIEGK(CPQ2)-NH2 |
| | VSVFVIE | | |
| 372 | NH2-(Nle)K | Probe #372 | NH2-(Nle)K(FAM)PPSGLSGK(CPQ2)-NH2 |
| | (FAM)PPSGLS | | |
| 373 | NH2-K(FAM) | Probe #373 | NH2-K(FAM)(Nle)PPSGLSGK(CPQ2)-NH2 |
| | (Nle)PPSGLS | | |
| 374 | NH2-LK(FAM) | Probe #374 | NH2-LK(FAM)RWYGGIGKK(CPQ2)-NH2 |
| | RWYGGI | | |
| 375 | NH2-K(FAM) | Probe #375 | NH2-K(FAM)LRWYGGIGKK(CPQ2)-NH2 |
| | LRWYGGI | | |
| 376 | NH2-NK(FAM) | Probe #376 | NH2-NK(FAM)QYVFF(Nle)GK(CPQ2)-NH2 |
| | QYVFF(Nle) | | |
| 377 | NH2-K(FAM) | Probe #377 | NH2-K(FAM)NQYVFF(Nle)GK(CPQ2)-NH2 |
| | NQYVFF(Nle) | | |
| 378 | NH2-AK(FAM) | Probe #378 | NH2-AK(FAM)FAKYYKGK(CPQ2)-NH2 |
| | FAKYYK | | |
| 379 | NH2-K(FAM) | Probe #379 | NH2-K(FAM)AFAKYYKGK(CPQ2)-NH2 |
| | AFAKYYK | | |
| 380 | NH2-RK(FAM) | Probe #380 | NH2-RK(FAM)QVKHFTGK(CPQ2)-NH2 |
| | QVKHFT | | |
| 381 | NH2-K(FAM) | Probe #381 | NH2-K(FAM)RQVKHFTGK(CPQ2)-NH2 |
| | RQVKHFT | | |
| 382 | NH2-K(FAM)PP | Probe #382 | NH2-K(FAM)PPK(CPQ2)-NH2 |
| 383 | kpilffrlk | Probe #383 | 5FAM-GkpilffrlkGK(CPQ2)-PEG2-kk-NH2 |
| 384 | LRR | Probe #384 | Boc-Leu-Arg-Arg-AMC |
| 385 | R | Probe #385 | Arg-AMC |
| 386 | VR | Probe #386 | Boc-Val-Arg-AMC |
| 387 | RR | Probe #387 | Z-Arg-Arg-AMC |
| 388 | GR | Probe #388 | Gly-Arg-AMC |
| 389 | FR | Probe #389 | Z-Phe-Arg-AMC |
| 390 | RGK | Probe #390 | Ac-Arg-Gly-Lys-AMC |
| 391 | GGR | Probe #391 | Z-Gly-Gly-Arg-AMC |
| 392 | F | Probe #392 | Glutary1-Phe-AMC |
| 393 | D | Probe #393 | H-Asp-AMC |
| 394 | RR | Probe #394 | H-Arg-Arg-AMC |
| 395 | R | Probe #395 | Z-Arg-AMC |
| 396 | Bz-R | Probe #396 | Bz-Arg-AMC |
| 397 | Bz-R | Probe #397 | Bz-Arg-AMC |
| 398 | PR | Probe #398 | Z-Pro-Arg-AMC |
| 399 | GPR | Probe #399 | Z-Gly-Pro-Arg-AMC |
| 400 | LR | Probe #400 | Z-Leu-Arg-AMC |
| 401 | PFR | Probe #401 | H-Pro-Phe-Arg-AMC |
| 402 | LLR | Probe #402 | Z-Leu-Leu-Arg-AMC |
| 403 | QRR | Probe #403 | Boc-Gln-Arg-Arg-AMC |
| 404 | GR | Probe #404 | Glutaryl-Gly-Arg-AMC |
| 405 | GRR | Probe #405 | Boc-Gly-Arg-Arg-AMC |
| 406 | LRGG | Probe #406 | Z-Leu-Arg-Gly-Gly-AMC |
| 407 | RLRGG | Probe #407 | 5-FAM-GRLRGGGK(CPQ2)-PEG2-kk-GC |
| 408 | RELNGGAPI | Probe #408 | 5-FAM-GRELNGGAPIGK(CPQ2)-PEG2-kk-GC |
| 409 | TSAVLQSGFRK | Probe #409 | 5-FAM-GTSAVLQSGFRKGK(CPQ2)-PEG2-kk-GC |
| 410 | SGVTFQGKFKK | Probe #410 | 5-FAM-GSGVTFQGKFKKGK(CPQ2)-PEG2-kk-GC |
| 411 | AAFA | Probe #411 | 5-FAM-GAAFAGK(CPQ2)-PEG2-kk-GC |
| 412 | HGDQMAQKS | Probe #412 | 5FAM-GHGDQMAQKS-K(CPQ2)-PEG2- |
| | | | DLys-DLys-GC-NH2 |
| 413 | GPLGMR | Probe #413 | 5FAM-GGPLGMRG-K(CPQ2)-PEG2-DLys- |
| | | | DLys-GC-NH2 |
| 414 | FFLAQA- | Probe #414 | 5FAM-GFFLAQA-HomoPhe-RSK-K(CPQ2)- |
| | HomoPhe-RSK | | PEG2-DLys-DLys-GC-NH2 |
| 415 | AHAVSRIRIYLLPAK | Probe #415 | 5FAM-GAHAVSRIRIYLLPAK-K(CPQ2)- |
| | | | PEG2-DLys-DLys-GC-NH2 |
| 416 | PLALWAR | Probe #416 | 5FAM-GPLALWAR-K(CPQ2)-PEG2-DLys- |
| | | | DLys-GC-NH2 |
| 417 | PLA- | Probe #417 | 5FAM-GPLA-C(OMeBzl)-WAR-K(CPQ2)- |
| | C(OMeBzl)-WAR | | PEG2-DLys-DLys-GC-NH2 |
| 418 | APRWIQD | Probe #418 | 5FAM-GAPRWIQD-K(CPQ2)-PEG2-DLys- |
| | | | DLys-GC-NH2 |
| 419 | LREQQRLKS | Probe #419 | 5FAM-GLREQQRLKS-K(CPQ2)-PEG2- |
| | | | DLys-DLys-GC-NH2 |
| 420 | EFPIYVFLPAKK | Probe #420 | 5FAM-GEFPIYVFLPAKK-K(CPQ2)-PEG2- |
| | | | DLys-DLys-GC-NH2 |
| 421 | GAANLVRGG | Probe #421 | 5FAM-GGAANLVRGG-K(CPQ2)-PEG2- |
| | | | DLys-DLys-GC-NH2 |
| 422 | GYAELRMG | Probe #422 | 5FAM-GGYAELRMGG-K(CPQ2)-PEG2- |
| | | | DLys-DLys-GC-NH2 |
| 423 | AAGAMFLEA | Probe #423 | 5FAM-GAAGAMFLEA-K(CPQ2)-PEG2- |
| | | | DLys-DLys-GC-NH2 |
| 424 | LGGSGQRGRKALE | Probe #424 | (FAM)-GLGGSGQRGRKALEG-K(CPQ2)- |
| | | | (PEG2)-DLys-DLys-GC |
| 425 | LGGSGHYGRSGLE | Probe #425 | (FAM)-GLGGSGHYGRSGLEG-K(CPQ2)- |
| | | | (PEG2)-DLys-DLys-GC |
| 426 | YGRS | Probe #426 | (FAM)-GYGRSG-K(CPQ2)-(PEG2)-DLys- |
| | | | DLys-GC |
| 427 | FRGRK | Probe #427 | (FAM)-GFRGRKG-K(CPQ2)-(PEG2)-DLys- |
| | | | DLys-GC |
| 428 | DRRKKLTQ | Probe #428 | (FAM)-GDRRKKLTQG-K(CPQ2)-(PEG2)- |
| | | | DLys-DLys-GC |
| 429 | HPGGPQ | Probe #429 | (FAM)-GHPGGPQG-K(CPQ2)-(PEG2)-DLys- |
| | | | DLys-GC |
| 430 | KLRFSKQ | Probe #430 | (FAM)-GKLRFSKQG-K(CPQ2)-(PEG2)- |
| | | | DLys-DLys-GC |
| 43 | AIKFFSAQ | Probe #431 | (FAM)-GAIKFFSAQG-K(CPQ2)-(PEG2)- |
| | | | DLys-DLys-GC |
| 432 | AIKFFVRQ | Probe #432 | (FAM)-GAIKFFVRQG-K(CPQ2)-(PEG2)- |
| | | | DLys-DLys-GC |
| 433 | RPPGFSAFK | Probe #433 | (FAM)-GRPPGFSAFKG-K(CPQ2)-(PEG2)- |
| | | | DLys-DLys-GC |
| 434 | FAP-QLS | Probe #434 | (FAM)-GFAP-QLSG-K(CPQ2)-(PEG2)-DLys- |
| | | | DLys-GC |
| 435 | FAA-QMA | Probe #435 | (FAM)-GFAA-QMAG-K(CPQ2)-(PEG2)- |
| | | | DLys-DLys-GC |
| 436 | GMP-ANQ | Probe #436 | (FAM)-GGMP-ANQG-K(CPQ2)-(PEG2)- |
| | | | DLys-DLys-GC |
| 437 | LSGRSDNH | Probe #437 | (FAM)-GLSGRSDNHG-K(CPQ2)-(PEG2)- |
| | | | DLys-DLys-GC |
| 438 | MAALITRPDF | Probe #438 | (FAM)-GMAALITRPDFG-K(CPQ2)-(PEG2)- |
| | | | DLys-DLys-GC |
| 439 | MAAAITRPRF | Probe #439 | (FAM)-GMAAAITRPRFG-K(CPQ2)-(PEG2)- |
| | | | DLys-DLys-GC |
| 440 | MAALIVRPDL | Probe #440 | (FAM)-GMAALIVRPDLG-K(CPQ2)-(PEG2)- |
| | | | DLys-DLys-GC |
| 441 | TSGPNQEQE | Probe #441 | (FAM)-GTSGPNQEQEG-K(CPQ2)-(PEG2)- |
| | | | DLys-DLys-GC |
| 442 | TAGPNQEQE | Probe #442 | (FAM)-GTAGPNQEQEG-K(CPQ2)-(PEG2)- |
| | | | DLys-DLys-GC |
| 443 | GPGPNQA | Probe #443 | (FAM)-GGPGPNQAG-K(CPQ2)-(PEG2)- |
| | | | DLys-DLys-GC |
| 444 | ASGPAGPA | Probe #444 | (FAM)-GASGPAGPAG-K(CPQ2)-(PEG2)- |
| | | | DLys-DLys-GC |
| 445 | ERGETGPSG | Probe #445 | (FAM)-GERGETGPSGG-K(CPQ2)-(PEG2)- |
| | | | DLys-DLys-GC |
| 446 | VSQELGQR | Probe #446 | (FAM)-GVSQELGQRG-K(CPQ2)-(PEG2)- |
| | | | DLys-DLys-GC |
| 447 | TGPPGYPTG | Probe #447 | (FAM)-GTGPPGYPTGG-K(CPQ2)-(PEG2)- |
| | | | DLys-DLys-GC |
| 448 | TRLPVYQ | Probe #448 | (FAM)-GTRLPVYQG-K(CPQ2)-(PEG2)- |
| | | | DLys-DLys-GC |
| 449 | RQARVVGG | Probe #449 | (FAM)-GRQARVVGGG-K(CPQ2)-(PEG2)- |
| | | | DLys-DLys-GC |
| 450 | RQRRVVGG | Probe #450 | (FAM)-GRQRRVVGGG-K(CPQ2)-(PEG2)- |
| | | | DLys-DLys-GC |
| 451 | RQARAVGG | Probe #451 | (FAM)-GRQARAVGGG-K(CPQ2)-(PEG2)- |
| | | | DLys-DLys-GC |
| 452 | RKRRGSRG | Probe #452 | (FAM)-GRKRRGSRGG-K(CPQ2)-(PEG2)- |
| | | | DLys-DLys-GC |
| 453 | KQSRKFVP | Probe #453 | (FAM)-GKQSRKFVPG-K(CPQ2)-(PEG2)- |
| | | | DLys-DLys-GC |
| 454 | VTGRS | Probe #454 | (FAM)-GVTGRSG-K(CPQ2)-(PEG2)-DLys- |
| | | | DLys-GC |
| 455 | LKSRVK | Probe #455 | (FAM)-GLKSRVKG-K(CPQ2)-(PEG2)-DLys- |
| | | | DLys-GC |
| 456 | GIGAVLKVLT | Probe #456 | (FAM)-GGIGAVLKVLTG-K(CPQ2)-(PEG2)- |
| | | | DLys-DLys-GC |
| 457 | GLPALISWIK | Probe #457 | (FAM)-GGLPALISWIKG-K(CPQ2)-(PEG2)- |
| | | | DLys-DLys-GC |
| 458 | SEVNLDAEF | Probe #458 | (FAM)-GSEVNLDAEFG-K(CPQ2)-(PEG2)- |
| | | | DLys-DLys-GC |
| 459 | EEKPICFFRLGKE | Probe #459 | (FAM)-GEEKPICFFRLGKEG-K(CPQ2)- |
| | | | (PEG2)-DLys-DLys-GC |
| 460 | EEKPILFFRLGKE | Probe #460 | (FAM)-GEEKPILFFRLGKEG-K(CPQ2)- |
| | | | (PEG2)-DLys-DLys-GC |
| 461 | APSSVIAA | Probe #461 | (FAM)-GAPSSVIAAG-K(CPQ2)-(PEG2)- |
| | | | DLys-DLys-GC |
| 462 | KKAKRNAL | Probe #462 | (FAM)-GKKAKRNALG-K(CPQ2)-(PEG2)- |
| | | | DLys-DLys-GC |
| 463 | WTNTSANYNL | Probe #463 | (FAM)-GWTNTSANYNLG-K(CPQ2)- |
| | | | (PEG2)-DLys-DLys-GC |
| 464 | RVRR | Probe #464 | (FAM)-GRVRRG-K(CPQ2)-(PEG2)-DLys-DLys-GC |
| 465 | ERTKR | Probe #465 | (FAM)-GERTKRG-K(CPQ2)-(PEG2)-DLys-DLys-GC |
| 466 | RYQIKPLKSTDE | Probe #466 | (FAM)-GRYQIKPLKSTDEG-K(CPQ2)- |
| | | | (PEG2)-DLys-DLys-GC |
| 467 | WELRHQA- | Probe #467 | (FAM)-GWELRHQA-(Hfe)-RSKG-K(CPQ2)- |
| | (Hfe)-RSK | | (PEG2)-DLys-DLys-GC |
| 468 | SGAFK-C(Me)- | Probe #468 | (FAM)-GSGAFK-C(Me)-LKDGAGG- |
| | LKDGAG | | K(CPQ2)-(PEG2)-DLys-DLys-GC |
| 469 | YVADGW | Probe #469 | (FAM)-GYVADGWG-K(CPQ2)-(PEG2)-DLys-DLys-GC |
| 470 | WEHDGW | Probe #470 | (FAM)-GWEHDGWG-K(CPQ2)-(PEG2)-DLys-DLys-GC |
| 471 | YVADAPV | Probe #471 | (FAM)-GYVADAPVG-K(CPQ2)-(PEG2)-DLys-DLys-GC |
| 472 | RPPGFSA | Probe #472 | (FAM)-GRPPGFSAG-K(CPQ2)-(PEG2)-DLys-DLys-GC |
| 473 | GSPAFLA | Probe #473 | (FAM)-GGSPAFLAG-K(CPQ2)-(PEG2)-DLys-DLys-GC |
| 474 | AGFSLPA | Probe #474 | (FAM)-GAGFSLPAG-K(CPQ2)-(PEG2)-DLys-DLys-GC |
| 475 | RWHTVGLRWE | Probe #475 | (FAM)-GRWHTVGLRWEG-K(CPQ2)- |
| | | | (PEG2)-DLys-DLys-GC |
| 476 | LEQ | Probe #476 | (FAM)-GLEQG-K(CPQ2)-(PEG2)-DLys-DLys-GC |
| 477 | RWPPMGLPWE | Probe #477 | (FAM)-GRWPPMGLPWEG-K(CPQ2)- |
| | | | (PEG2)-DLys-DLys-GC |
| 478 | RPKPVE | Probe #478 | (FAM)-GRPKPVEG-K(CPQ2)-(PEG2)-DLys-DLys-GC |
| 479 | IETD | Probe #479 | (FAM)-GIETDG-K(CPQ2)-(PEG2)-DLys-DLys-GC |
| 480 | VGPDFGR | Probe #480 | (FAM)-GVGPDFGRG-K(CPQ2)-(PEG2)- |
| | | | DLys-DLys-GC |
| 481 | GIEFDSGGC | Probe #481 | (FAM)-GGIEFDSGGCG-K(CPQ2)-(PEG2)- |
| | | | DLys-DLys-GC |
| 482 | GDFLRRV | Probe #482 | (FAM)-GGDFLRRVG-K(CPQ2)-(PEG2)- |
| | | | DLys-DLys-GC |
| 483 | AAL | Probe #483 | (FAM)-GAALG-K(CPQ2)-(PEG2)-DLys-DLys-GC |
| 484 | YATWSMIAAH | Probe #484 | (FAM)-GYATWSMIAAHG-K(CPQ2)- |
| | | | (PEG2)-DLys-DLys-GC |
| 485 | VIMWRLTVGT | Probe #485 | (FAM)-GVIMWRLTVGTG-K(CPQ2)- |
| | | | (PEG2)-DLys-DLys-GC |
| 486 | RRVLALQQEL | Probe #486 | (FAM)-GRRVLALQQELG-K(CPQ2)-(PEG2)- |
| | | | DLys-DLys-GC |
| 487 | LATWPLSGLW | Probe #487 | (FAM)-GLATWPLSGLWG-K(CPQ2)- |
| | | | (PEG2)-DLys-DLys-GC |
| 488 | NTPNWLVNAV | Probe #488 | (FAM)-GNTPNWLVNAVG-K(CPQ2)- |
| | | | (PEG2)-DLys-DLys-GC |
| 489 | SPLAQAVRSSSRK | Probe #489 | (FAM)-GSPLAQAVRSSSRKG-K(CPQ2)- |
| | | | (PEG2)-DLys-DLys-GC |
| 490 | QMPGRLSMAF | Probe #490 | (FAM)-GQMPGRLSMAFG-K(CPQ2)- |
| | | | (PEG2)-DLys-DLys-GC |
| 491 | PLGLR | Probe #491 | (FAM)-GPLGLRG-K(CPQ2)-(PEG2)-DLys-DLys-GC |
| 492 | QRANSIRVTW | Probe #492 | (FAM)-GQRANSIRVTWG-K(CPQ2)-(PEG2)- |
| | | | DLys-DLys-GC |
| 493 | PLAVR | Probe #493 | (FAM)-GPLAVRG-K(CPQ2)-(PEG2)-DLys-DLys-GC |
| 494 | LLAVPAANTV | Probe #494 | (FAM)-GLLAVPAANTV G-K(CPQ2)- |
| | | | (PEG2)-DLys-DLys-GC |
| 495 | GPQGLRGQ | Probe #495 | (FAM)-GGPQGLRGQG-K(CPQ2)-(PEG2)- |
| | | | DLys-DLys-GC |
| 496 | RTGLYLYNST | Probe #496 | (FAM)-GRTGLYLYNSTG-K(CPQ2)-(PEG2)- |
| | | | DLys-DLys-GC |
| 497 | RKKLTQSKFVGGAE | Probe #497 | (FAM)-GRKKLTQSKFVGGAEG-K(CPQ2)- |
| | | | (PEG2)-DLys-DLys-GC |
| 498 | KHYR | Probe #498 | (FAM)-GKHYRG-K(CPQ2)-(PEG2)-DLys- |
| | | | DLys-GC |
| 499 | QAR | Probe #499 | (FAM)-GQARG-K(CPQ2)-(PEG2)-DLys- |
| | | | DLys-GC |
| 500 | PRPFNYL | Probe #500 | (FAM)-GPRPFNYLG-K(CPQ2)-(PEG2)- |
| | | | DLys-GC |
| 501 | APFEMSA | Probe #501 | (FAM)-GAPFEMSAG-K(CPQ2)-(PEG2)- |
| | | | DLys-DLys-GC |
| 502 | APFEFSA | Probe #502 | (FAM)-GAPFEFSAG-K(CPQ2)-(PEG2)- |
| | | | DLys-DLys-GC |
| 503 | PLGFRV | Probe #503 | (FAM)-GPLGFRVG-K(CPQ2)-(PEG2)-DLys-GC |
| 504 | RPLALWRS | Probe #504 | (FAM)-GRPLALWRSG-K(CPQ2)-(PEG2)-GC |
| 505 | RPLALEESQ | Probe #505 | (FAM)-GRPLALEESQG-K(CPQ2)-(PEG2)-DLys-GC |
| 506 | RPLALWRSQ | Probe #506 | (FAM)-GRPLALWRSQG-K(CPQ2)-(PEG2)-GC |
| 507 | RNALAVERTAS | Probe #507 | (FAM)-GRNALAVERTASG-K(CPQ2)-(PEG2)-GC |
| 508 | RPKPQQFW | Probe #508 | (FAM)-GRPKPQQFWG-K(CPQ2)-(PEG2)-DLys-GC |
| 509 | SGSNPYKYTA | Probe #509 | (FAM)-SGSNPYKYTA-K(CPQ2)-(PEG2)- |
| | | | DLys-DLys-GC |
| 510 | SGSNPYGYTA | Probe #510 | (FAM)-SGSNPYGYTA-K(CPQ2)-(PEG2)- |
| | | | DLys-DLys-GC |
| 511 | SGTLSELHTA | Probe #511 | (FAM)-SGTLSELHTA-K(CPQ2)-(PEG2)- |
| | | | DLys-DLys-GC |
| 512 | SGTISHLHTA | Probe #512 | (FAM)-SGTISHLHTA-K(CPQ2)-(PEG2)- |
| | | | DLys-DLys-GC |
| 513 | SG-(Orn)-RSHP- | Probe #513 | (FAM)-SG-(Orn)-RSHP-(Hfe)-TLYTA- |
| | (Hfe)-TLYTA | | K(CPQ2)-(PEG2)-DLys-GC |
| 514 | SG-(Orn)- | Probe #514 | (FAM)-SG-(Orn)-RSHG-(Hfe)-FLYTA- |
| | RSHG-(Hfe)- | | K(CPQ2)-(PEG2)-DLys-GC |
| | FLYTA | | |
| 515 | SGESLAYYTA | Probe #515 | (FAM)-SGESLAYYTA-K(CPQ2)-(PEG2)- |
| | | | DLys-DLys-GC |
| 516 | SGHMHAALTA | Probe #516 | (FAM)-SGHMHAALTA-K(CPQ2)-(PEG2)- |
| | | | DLys-DLys-GC |
| 517 | ILSR-(DIle)- | Probe #517 | (FAM)-GILSR-(DIle)-VGGG-K(CPQ2)- |
| | VGG | | (PEG2)-DLys-GC |
| 518 | ILS-(DArg)- | Probe #518 | (FAM)-GILS-(DArg)-(DIle)-(DVal)-GGG- |
| | (DIle)-(DVal)-GG | | K(CPQ2)-(PEG2)-DLys-GC |
| 519 | RQRRALEK | Probe #519 | 5FAM-GRQRRALEKG-K(CPQ2)-PEG2-GC |
| 520 | KPISLISS | Probe #520 | 5FAM-GKPISLISSG-K(CPQ2)-PEG2-GC |
| 521 | QKGRYKQE | Probe #521 | 5FAM-GQKGRYKQEG-K(CPQ2)-PEG2-GC |
| 522 | GPLGLRSW | Probe #522 | 5FAM-GGPLGLRSWK(CPQ2)-PEG2-C |
| 523 | GPLGVRGK | Probe #523 | 5FAM-GGPLGVRGKK(CPQ2)-PEG2-C |
| 524 | GfPRSGG | Probe #524 | 5FAM-GGfPRSGGGK(CPQ2)-PEG2-C |
| 525 | Py | Probe #525 | Pyr-AMC |
| 526 | SY | Probe #526 | H-Ser-Tyr-AMC |
| 527 | GF | Probe #527 | H-Gly-Phe-AMC |
| 528 | Y | Probe #528 | H-Tyr-AMC |
| 529 | Cit | Probe #529 | H-Cit-AMC Hydrobromide salt |
| 530 | GP | Probe #530 | Suc-Gly-Pro-AMC |
| 531 | T | Probe #531 | H-Thr-AMC |
| 532 | I | Probe #532 | H-Ile-AMC |
| 533 | GA | Probe #533 | H-Gly-Ala-AMC hydrochloride salt |
| 534 | Cys(Bzl) | Probe #534 | H-Cys(Bzl)-AMC |
| 535 | A | Probe #535 | H-Ala-AMC |
| 536 | K | Probe #536 | Ac-Lys-AMC acetate salt |
| 537 | GLF | Probe #537 | MeOSuc-Gly-Leu-Phe-AMC |
| 538 | L | Probe #538 | H-Leu-AMC |
| 539 | VAN | Probe #539 | Z-Val-Ala-Asn-AMC |
| 540 | AAA | Probe #540 | Suc-Ala-Ala-Ala-AMC |
| 541 | K | Probe #541 | H-Lys-AMC acetate salt |
| 542 | F | Probe #542 | H-Phe-AMC trifluoroacetate salt |
| 543 | FSR | Probe #543 | Boc-Phe-Ser-Arg-AMC |
| 544 | VVR | Probe #544 | Z-Val-Val-Arg-AMC hydrochloride salt |
| 545 | KA | Probe #545 | H-Lys-Ala-AMC hydrochloride salt |
| 546 | PR | Probe #546 | H-Pro-Arg-AMC hydrochloride salt |
| 547 | MGP | Probe #547 | H-Met-Gly-Pro-AMC hydrochloride salt |
| 548 | KP | Probe #548 | H-Lys-Pro-AMC hydrochloride salt |
| 549 | QGR | Probe #549 | Boc-Gln-Gly-Arg-AMC hydrochloride salt |
| 550 | Glu(OBzl)-AR | Probe #550 | Boc-Glu(OBzl)-Ala-Arg-AMC hydrochloride |
| | | | salt |
| 551 | WEHD | Probe #551 | Ac-Trp-Glu-His-Asp-AMC |
| 552 | QAR | Probe #552 | Boc-Gln-Ala-Arg-AMC hydrochloride salt |
| 553 | AAF | Probe #553 | H-Ala-Ala-Phe-AMC (free base) |
| 554 | GPK | Probe #554 | Tos-Gly-Pro-Lys-AMC trifluoroacetate salt |
| 555 | AAPM | Probe #555 | MeOSuc-Ala-Ala-Pro-Met-AMC |
| 556 | AEPF | Probe #556 | Suc-Ala-Glu-Pro-Phe-AMC |
| 557 | GG | Probe #557 | H-Gly-Gly-AMC hydrochloride salt |
| 558 | VLK | Probe #558 | Boc-Val-Leu-Lys-AMC acetate salt |
| 559 | EKK | Probe #559 | Boc-Glu-Lys-Lys-AMC acetate salt |
| 560 | VPR | Probe #560 | Boc-Val-Pro-Arg-AMC hydrochloride salt |
| 561 | GKR | Probe #561 | Boc-Gly-Lys-Arg-AMC hydrochloride salt |
| 562 | Glu(OBzl)-GR | Probe #562 | Boc-Glu(OBzl)-Gly-Arg-AMC hydrochloride |
| | | | salt |
| 563 | LR | Probe #563 | Z-Leu-Arg-AMC hydrochloride salt |
| 564 | AFK | Probe #564 | MeOSuc-Ala-Phe-Lys-AMC trifluoroacetate |
| | | | salt |
| 565 | LGR | Probe #565 | Boc-Leu-Gly-Arg-AMC acetate salt |
| 566 | PFR | Probe #566 | H-Pro-Phe-Arg-AMC acetate salt |
| 567 | AAPV | Probe #567 | Suc-Ala-Ala-Pro-Val-AMC |
| 568 | AFK | Probe #568 | H-Ala-Phe-Lys-AMC trifluoroacetate salt |
| 569 | VKM | Probe #569 | Z-Val-Lys-Met-AMC acetate salt |
| 570 | GPLGP | Probe #570 | Suc-Gly-Pro-Leu-Gly-Pro-AMC |
| 571 | KQKER | Probe #571 | Ac-Lys-Gln-Lys-Leu-Arg-AMC |
| | | | trifluoroacetate salt |
| 572 | RVRR | Probe #572 | Boc-Arg-Val-Arg-Arg-AMC acetate salt |
| 573 | IEGR | Probe #573 | Boc-Ile-Glu-Gly-Arg-AMC acetate salt |
| 574 | GP | Probe #574 | H-Gly-Pro-AMC HBr |
| 575 | AAPV | Probe #575 | MeOSuc-Ala-Ala-Pro-Val-AMC |
| 576 | RPFHLLVY | Probe #576 | Suc-Arg-Pro-Phe-His-Leu-Leu-Val-Tyr-AMC |
| | | | trifluoroacetate salt |
| 577 | Anb-WS-Gnf-TVF | Probe #577 | H-Anb-Trp-Ser-Gnf-Thr-Val-Phe-AMC |
| 578 | HSSKLQ | Probe #578 | Mu-His-Ser-Ser-Lys-Leu-Gln-AMC |
| 579 | RPY | Probe #579 | MeO-Succ-Arg-Pro-Tyr-AMC |
| 580 | DRENSPK(Dnp) | Probe #580 | (ACC)-kkDRENSPK(Dnp)L |
| | L-OH | | |
| 581 | kkDRENSPLK | Probe #581 | (ACC)-kkDRENSPLK(Dnp) |
| | (Dnp)-OH | | |
| 582 | NAGSKFK | Probe #582 | (ACC)-NAGSKFK(Dnp)Q |
| | (Dnp)Q-OH | | |
| 583 | NAGSKFQK | Probe #583 | (ACC)-NAGSKFQK(Dnp) |
| | (Dnp)-OH | | |
| 584 | HLLGFYK(Dnp) | Probe #584 | (ACC)-kkHLLGFYK(Dnp)V |
| | V-OH | | |
| 585 | HLLGFYVK | Probe #585 | (ACC)-kkHLLGFYVK(Dnp) |
| | (Dnp)-OH | | |
| 586 | QEKQT(Nle)K | Probe #586 | (ACC)-kkQEKQT(Nle)K(Dnp)(Nle) |
| | (Dnp)(Nle)-OH | | |
| 587 | QEKQT(Nle) | Probe #587 | (ACC)-kkQEKQT(Nle)(Nle)K(Dnp) |
| | (Nle)K(Dnp)-OH | | |
| 588 | DPFVVSK(Dnp) | Probe #588 | (ACC)-kDPFVVSK(Dnp)W |
| | W-OH | | |
| 589 | DPFVVSWK | Probe #589 | (ACC)-kDPFVVSWK(Dnp) |
| | (Dnp)-OH | | |
| 590 | NAYNEIK(Dnp) | Probe #590 | (ACC)-NAYNEIK(Dnp)R |
| | R-OH | | |
| 591 | NAYNEIRK | Probe #591 | (ACC)-NAYNEIRK(Dnp) |
| | (Dnp)-OH | | |
| 592 | V(Nle)RQSEK | Probe #592 | (ACC)-V(Nle)RQSEK(Dnp)N |
| | (Dnp)N-OH | | |
| 593 | V(Nle)RQSENK | Probe #593 | (ACC)-V(Nle)RQSENK(Dnp) |
| | (Dnp)-OH | | |
| 594 | YNPRE(Nle)K | Probe #594 | (ACC)-YNPRE(Nle)K(Dnp)I |
| | (Dnp)I-OH | | |
| 595 | YNPRE(Nle)IK | Probe #595 | (ACC)-YNPRE(Nle)IK(Dnp) |
| | (Dnp)-OH | | |
| 596 | EFVHNPK(Dnp) | Probe #596 | (ACC)-KEFVHNPK(Dnp)K |
| | K-OH | | |
| 597 | EFVHNPKK | Probe #597 | (ACC)-KEFVHNPKK(Dnp) |
| | (Dnp)-OH | | |
| 598 | KRVQFLK(Dnp) | Probe #598 | (ACC)-KRVQFLK(Dnp)H |
| | H-OH | | |
| 599 | KRVQFLHK | Probe #599 | (ACC)-KRVQFLHK(Dnp) |
| | (Dnp)-OH | | |
| 600 | LI(Nle)HKNK | Probe #600 | (ACC)-KLI(Nle)HKNK(Dnp)G |
| | (Dnp)G-OH | | |
| 601 | LI(Nle)HKNGK | Probe #601 | (ACC)-KLI(Nle)HKNGK(Dnp) |
| | (Dnp)-OH | | |
| 602 | WA(Nle)LYHK | Probe #602 | (ACC)-kkWA(Nle)LYHK(Dnp)S |
| | (Dnp)S-OH | | |
| 603 | WA(Nle)LYHS | Probe #603 | (ACC)-kkWA(Nle)LYHSK(Dnp) |
| | K(Dnp)-OH | | |
| 604 | AHDIVNK(Dnp) | Probe #604 | (ACC)-kkAHDIVNK(Dnp)Y |
| | Y-OH | | |
| 605 | AHDIVNYK | Probe #605 | (ACC)-kkAHDIVNYK(Dnp) |
| | (Dnp)-OH | | |
| 606 | SVFVIEK(Dnp) | Probe #606 | (ACC)-kSVFVIEK(Dnp)P |
| | P-OH | | |
| 607 | SVFVIEPK | Probe #607 | (ACC)-kSVFVIEPK(Dnp) |
| | (Dnp)-OH | | |
| 608 | PPSGLSK(Dnp) | Probe #608 | (ACC)-kPPSGLSK(Dnp)E |
| | E-OH | | |
| 609 | PPSGLSEK | Probe #609 | (ACC)-kPPSGLSEK(Dnp) |
| | (Dnp)-OH | | |
| 610 | RWYGGIK | Probe #610 | (ACC)-kkRWYGGIK(Dnp)F |
| | (Dnp)F-OH | | |
| 611 | RWYGGIFK | Probe #611 | (ACC)-kkRWYGGIFK(Dnp) |
| | (Dnp)-OH | | |
| 612 | QYVFF(Nle)K | Probe #612 | (ACC)-kQYVFF(Nle)K(Dnp)D |
| | (Dnp)D-OH | | |
| 613 | QYVFF(Nle)DK | Probe #613 | (ACC)-kQYVFF(Nle)DK(Dnp) |
| | (Dnp)-OH | | |
| 614 | FAKYYKK | Probe #614 | (ACC)-kFAKYYKK(Dnp)T |
| | (Dnp)T-OH | | |
| 615 | FAKYYKTK | Probe #615 | (ACC)-kFAKYYKTK(Dnp) |
| | (Dnp)-OH | | |
| 616 | QVKHFTK | Probe #616 | (ACC)-kQVKHFTK(Dnp)A |
| | (Dnp)A-OH | | |
| 617 | QVKHFTAK | Probe #617 | (ACC)-kQVKHFTAK(Dnp) |
| | (Dnp)-OH | | |
| 618 | YVADAPK | Probe #618 | (ACC)-KYVADAPK(Dnp) |
| | (Dnp)-OH | | |
| 619 | KGISSQY | Probe #619 | ACC-GKGISSQYK(Dnp)-NH2 |
| 620 | ALPALQN | Probe #620 | ACC-GALPALQNK(Dnp)-PEG2-Dlys-Dlys-NH2 |
| 621 | HRFRG | Probe #621 | ACC-GHRFRGK(Dnp)-NH2 |
| 622 | APEEIMDQQ | Probe #622 | ACC-GAPEEIMDQQK(Dnp)-PEG2-Dlys-Dlys-NH2 |
| 623 | SRKSQQY | Probe #623 | ACC-GSRKSQQYK(Dnp)-NH2 |
| 624 | SKGRSLI | Probe #624 | ACC-GSKGRSLIGK(Dnp)-NH2 |
| 625 | FAQSIPK | Probe #625 | ACC-GFAQSIPKK(Dnp)-PEG2-Dlys-Dlys-NH2 |
| 626 | RQRRVVG | Probe #626 | ACC-GRQRRVVGGK(Dnp)-NH2 |
| 627 | ERGETGPS | Probe #627 | ACC-GERGETGPSGK(Dnp)-NH2 |
| 628 | ASGPSS | Probe #628 | ACC-GASGPSSGK(Dnp)-PEG2-Dlys-Dlys-NH2 |
| 629 | YRFR | Probe #629 | ACC-GYRFRGK(Dnp)-NH2 |
| 630 | KLFSSKQ | Probe #630 | ACC-GKLFSSKQK(Dnp)-NH2 |
| 631 | IVPRG | Probe #631 | ACC-GIVPRGK(Dnp)-NH2 |
| 632 | IRRSSYFK | Probe #632 | ACC-GIRRSSYFKK(Dnp)-NH2 |
| 633 | His(Bzl)-Tle- | Probe #633 | ACC-Gly-His(Bzl)-Tle-Pro-Ser-Asp-Met(O)- |
| | PSD-Met(O) | | Gly-K(Dnp)-Gly-PEG2-Dlys-Dlys-NH2 |
| 634 | Nva-IE-Oic- | Probe #634 | ACC-Nva-Ile-Glu-Oic-Asp-Phe-Gly-Arg- |
| | DFGR | | Lys(Dnp)-NH2 |
| 635 | H-DThr- | Probe #635 | Ac-His-DThr-Phe(F5)-Arg-ACC |
| | Phe(F5)-R | | |
| 636 | Dap-Orn- | Probe #636 | Ac-Dap-Orn-Phe(3Cl)-Cys(MeOBzl)-ACC |
| | Phe(3Cl)- | | |
| | Cys(MeOBzl) | | |
| 637 | Cha-L- | Probe #637 | Ac-Cha-Leu-hSer(Bzl)-Arg-ACC |
| | hSer(Bzl)-R | | |
| 638 | His(Bzl)-Tle- | Probe #638 | ACC-Gly-His(Bzl)-Tle-Pro-Ser-Asp-Met(O)- |
| | PSD-Met(O) | | Gly-K(Dnp)-Gly-PEG2-Dlys-Dlys-NH2 |
| 639 | hCha-Phe(guan)- | Probe #639 | Ac-hCha-Phe(guan)-Oic-Arg-ACC |
| | Oic-R | | |
| 640 | Abu-Nle(O-Bzl) | Probe #640 | NH2-Abu-Nle(O-Bzl)-ACC |
| 641 | Nle(O-Bzl)- | Probe #641 | Ac-Nle(O-Bzl)-Met(O)2-Oic-Abu-ACC |
| | Met(O)2-Oic- | | |
| | Abu | | |
| 642 | Dap-Orn- | Probe #642 | ACC-G-Dap-Orn-Phe(3Cl)-Cys(MeOBz)-G- |
| | Phe(3Cl)- | | K(Dnp)-NH2 |
| | Cys(MeOBz) | | |
| 643 | Cha-L-hSer-R | Probe #643 | ACC-Gly-Cha-Leu-hSer-Arg-Gly-K(Dnp)-NH2 |
| 644 | FVT-Gnf-SW | Probe #644 | ACC-Phe-Val-Thr-Gnf-Ser-Trp-K(Dnp)-NH2 |
| 645 | hCha-Phe(guan)- | Probe #645 | ACC-Gly-hCha-Phe(guan)-Oic-Arg-Gly- |
| | Oic-R | | K(Dnp)-NH2 |
| 646 | Nle(OBz)- | Probe #646 | ACC-Gly-Nle(OBz)-Met(02)-Oic-Abu-Gly- |
| | Met(O2)-Oic-Abu | | K(Dnp)-NH2 |
| 647 | AIEPDSG | Probe #647 | 5FAM-GAIEPDSGG-Lys(CPQ2)-PEG2-Dlys- |
| | | | Dlys-GC-NH2 |
| 648 | AIEFDSG | Probe #648 | 5FAM-GAIEFDSGG-Lys(CPQ2)-Dlys-Dlys-GC-NH2 |
| 649 | AAEAISD | Probe #649 | 5FAM-GGAAEAISDAK(CPQ2)-kk-PEG2-C |
| 650 | AGGAQMGA | Probe #650 | 5FAM-GGAGGAQMGAK(CPQ2)-kk-PEG2-C |
| 651 | AQPDALNV | Probe #651 | 5FAM-GGAQPDALNVK(CPQ2)-kk-PEG2-C |
| 652 | ATDVTTTP | Probe #652 | 5FAM-GGATDVTTTPK(CPQ2)-kk-PEG2-C |
| 653 | DIVTVANA | Probe #653 | 5FAM-GGDIVTVANAK(CPQ2)-kk-PEG2-C |
| 654 | DLGLKSVP | Probe #654 | 5FAM-GGDLGLKSVPK(CPQ2)-kk-PEG2-C |
| 655 | DVMASNKR | Probe #655 | 5FAM-GGDVMASNKRK(CPQ2)-kk-PEG2-C |
| 656 | ESDELNTI | Probe #656 | 5FAM-GGESDELNTIK(CPQ2)-kk-PEG2-C |
| 657 | FHPLHSKI | Probe #657 | 5FAM-GGFHPLHSKIK(CPQ2)-kk-PEG2-C |
| 658 | HARLVHV | Probe #658 | 5FAM-GGGHARLVHVK(CPQ2)-kk-PEG2-C |
| 659 | HIANVERV | Probe #659 | 5FAM-GGHIANVERVK(CPQ2)-kk-PEG2-C |
| 660 | KAAATQKK | Probe #660 | 5FAM-GGKAAATQKKK(CPQ2)-kk-PEG2-C |
| 661 | LATASTMD | Probe #661 | 5FAM-GGLATASTMDK(CPQ2)-kk-PEG2-C |
| 662 | LGPKGQT | Probe #662 | 5FAM-GGLGPKGQTGK(CPQ2)-kk-PEG2-C |
| 663 | LSLPETGE | Probe #663 | 5FAM-GGLSLPETGEK(CPQ2)-kk-PEG2-C |
| 664 | NLAGILKE | Probe #664 | 5FAM-GGNLAGILKEK(CPQ2)-kk-PEG2-C |
| 665 | NPGMSEPV | Probe #665 | 5FAM-GGNPGMSEPVK(CPQ2)-kk-PEG2-C |
| 666 | PFGCHAK | Probe #666 | 5FAM-GGPFGCHAKK(CPQ2)-kk-PEG2-C |
| 667 | PLGLRWW | Probe #667 | 5FAM-GGPLGLRWWK(CPQ2)-kk-PEG2-C |
| 668 | QMGVMQGV | Probe #668 | 5FAM-GGQMGVMQGVK(CPQ2)-kk-PEG2-C |
| 669 | QTCKCSCK | Probe #669 | 5FAM-GGQTCKCSCKK(CPQ2)-kk-PEG2-C |
| 670 | QWAGLVEK | Probe #670 | 5FAM-GGQWAGLVEKK(CPQ2)-kk-PEG2-C |
| 671 | RPAVMTSP | Probe #671 | 5FAM-GGRPAVMTSPK(CPQ2)-kk-PEG2-C |
| 672 | TLRELHLD | Probe #672 | 5FAM-GGTLRELHLDK(CPQ2)-kk-PEG2-C |
| 673 | TPPPSQGK | Probe #673 | 5FAM-GGTPPPSQGKK(CPQ2)-kk-PEG2-C |
| 674 | TSEDL VVQ | Probe #674 | 5FAM-GGTSEDLVVQK(CPQ2)-kk-PEG2-C |
| 675 | VWAAEAIS | Probe #675 | 5FAM-GGVWAAEAISK(CPQ2)-kk-PEG2-C |
| 676 | R | Probe #676 | H-R-AMC |
| 677 | GC | Probe #677 | FAM-GGC-PEG8 |

| | |
|---|---|
| Nle = norleucine | Oic = L-octahydroindole-2-carboxylic acid |
| K(FAM) = carboxy-fluorescein-L-lysine | Nva = norvaline (click to see farther down list) |
| HomoPhe = Hfe = L-homophenylalanine | DThr = d-threonine |
| Cys(OMeBzl) = C(OMeBzl) = S-para- | Phe(F5) = 2,3,4,5,6-pentafluoro-L-penylalanine |
| methoxybenzyl cysteine | Phe(3Cl) = 3-chloro-L-phenylalanine |
| DIle = d-isoleucine | hSer(Bzl) = benzyl homoserine |
| DArg = D-arginine | hCha = homocyclohexylalnine |
| DVal = D-valine | Phe(guan) = phenylalanine derivative with a |
| Pyr = pyroglutamic acid | guanidine group in the para position |
| Cit = citrulline | Nle(O-Bzl) = Nle(OBz) = benzyloxy-L- |
| C(Bzl) = S-benzyl-L-cysteine | norleucine |
| Glu(OBzl) = benzyl-L-glutamate | Met(O)2 = L-methionine sulfone |
| Anb = amino-n-butyric acid | Dap = 2,3-diaminopropionic acid |
| Gnf = guamidine-L-phenylalanine | hSer = homoserine |
| K(Dnp) = dinitrobenzylation of lysine | Met(O2) = methylsulfonylbutanoic acid |
| His(Bzl) = benzyl-L-histidine | Abu = L-alpha-aminobutyric acid |
| Tle = L-tert-leucine | Cha = L-cyclohexylalanine |
| Met(O) = L-methionine-sulfoxide | Cys(Me) = L- Methyl cysteine |
| Bz = Benzoyl | Orn = L-Ornithine |
| | hF = L-Homophenylalanine |
| | GABA = gamma aminobutyric acid |
| | Pip = piperidine carboxylic acid |
| | lower case = D-amino acids |

The peptide linkers described herein for endoproteases may follow a design: XₘAYₙ or AXₙB, wherein respectively, A is a single amino acid and A and B are amino acid pairs recognized by a particular endoprotease, X and Y are any amino acid labeled or not with a reporter, and m, n are zero or any integer. This design is for exemplification only and should not be construed as the only possible design for the peptide linker.

The peptide linkers described herein for exoproteases may follow a design: XₘAYₙ, wherein A is amino acid pairs recognized by a particular exoprotease, X and Y are any amino acid labeled or not with a reporter, and n is zero or any integer. This design is for exemplification only and should not be construed as the only possible design for the peptide linker.

TABLE 2
Exemplary peptide linker designs.

| | | | | | | | | Critical |
|---|---|---|---|---|---|---|---|---|
| | | | | | | | | amino |
| amino | amino | amino | amino | amino | Example | | | acid |
| acid | acid in | acid | acid in | acid | probe | | Protease | (single |
| in P1′ | P1 | in P2 | P3 | in P4 | name | Example prob design | family | or pair) |
| | R/K | | | | Probe | (FAM)-GWYKTQYGK(CPQ2)- | Endo | Single |
| | | | | | #161 | NH2 | | |
| | R/K | | | | Probe | (FAM)-GFARRWGGK(CPQ2)- | Endo | Single |
| | | | | | #109 | PEG2-k-NH2 | | |
| | F/Y/L/W | | | | Probe | (FAM)- | Endo | Single |
| | | | | | #165 | GSYWP(Nle)QGK(CPQ2)- | | |
| | | | | | | PEG2-k-NH2 | | |
| | F/Y | | | | Probe | (FAM)-GFIY(Nle)PTGK(CPQ2)- | Endo | Single |
| | | | | | #140 | PEG2-k-NH2 | | |
| | P | | | | Probe | (FAM)-GTGPKGNGK(CPQ2)- | Endo | Single |
| | | | | | #148 | NH2 | | |
| F | K | | | | Probe | (FAM)- | Endo | Pair |
| | | | | | #217 | GWSKFW(Nle)GK(CPQ2) | | (AB) |
| D | G | | | | Probe | (FAM)-GKTGDARGK(CPQ2)- | Endo | Pair |
| | | | | | #194 | PEG2-k-NH2 | | (AB) |
| L | P | | | | Probe | (FAM)-GGHPLSPGK(CPQ2)- | Endo | Pair |
| | | | | | #275 | PEG2-kk-NH2 | | (AB) |
| | D | T/I/V | | | Probe | (FAM)-GVIDKDFGK(CPQ2)- | Endo | Pair |
| | | | | | #297 | NH2 | | (AB) |
| | R | K/R | | | Probe | (FAM)-GFARRWGGK(CPQ2)- | Endo | Pair |
| | | | | | #109 | PEG2-k-NH2 | | (AB) |
| S | R | | | | Probe | (FAM)-GPVRSTNGK(CPQ2)- | Endo | Pair |
| | | | | | #204 | NH2 | | (AB) |
| | D | | E | | Probe | (FAM)-GENDRLPGK(CPQ2)- | Endo | Pair |
| | | | | | #199 | NH2 | | (near |
| | | | | | | | | neighbor |
| | | | | | | | | AXB) |
| | D | | V | | Probe | (FAM)-GQWVDEDGK(CPQ2)- | Endo | Pair |
| | | | | | #248 | PEG2-k-NH2 | | (near |
| | | | | | | | | neighbor |
| | | | | | | | | AXXB) |
| | K/R at | | | | Probe | (FAM)-kGEFVHNPK(CPQ2)K- | Exo | Single |
| | C- | | | | #321 | OH | | |
| | terminus | | | | | | | |
| | K/R/H at | | | | Probe | (FAM)-GNAYNEIK(CPQ2)R- | Exo | Single |
| | C- | | | | #315 | OH | | |
| | terminus | | | | | | | |
| | W/G/F | | | | Probe | NH2- | Exo | Single |
| | at N- | | | | #346 | WK(FAM)NAGSKFGKK(CPQ2)- | | |
| | terminus | | | | | NH2 | | |
| | Q/K at | | | | | NH2- | | |
| | N- | | | | Probe | QK(FAM)KRVQFLGK(CPQ2)- | | |
| | terminus | | | | #362 | NH2 | Exo | Single |

In some embodiments, the cleavable linker comprises an amino acid sequence selected from the group consisting of SEQ ID Nos: 1-677 or a sequence comprising a mimetic of any one of SEQ ID Nos: 1-677. In some embodiments, the mimetic is a beta amino acid or a peptoid.

In some embodiments, the cleavable linker may be a carbohydrate. Tung et al. reported a conjugate of β-galactoside and 7-hydroxy-9H-(1,3-dichloro-9,9-dimethylacridin-2-one), which has far-red fluorescence properties after a cleavage by β-galactosidase. Tung C H, Zeng Q, Shah K, Kim D E, Schellingerhout D, Weissleder R. In vivo imaging of beta-galactosidase activity using far red fluorescent switch. Cancer Res. 2004 Mar. 1; 64(5):1579-83. Ho et al. reported combining β-galactosidase substrate with p-benzyloxycarbonyl as a self-immolative linker. β-D-Galactopyranoside, the substrate of β-galactosidase, was conjugated to an optical probe through a para-substituted benzyloxycarbonyl group (serves as a first self-immolative linker) and a glycine residue (serves as a quencher and a second self-immolative linker). Enzymatic cleavage of the β-D-Galactopyranoside triggered a series of spontaneous reactions that resulted in a release of optically active probe. Ho, N.-H., Weissleder, R. and Tung, C.-H. (2007), A Self-Immolative Reporter For β-Galactosidase Sensing. ChemBioChem, 8:560-566. Some carbohydrate linkers are commercially available.

In some embodiments, the cleavable linker may be a nucleic acid. In some embodiments, the nucleic acid comprises a nucleic acid sequence encoding any one of the amino acids of SEQ ID Nos: 1-677 or a mimetic of any one of the amino acids of SEQ ID Nos. 1-677. The effect of a DNA linker on the behavior of its conjugate both reduces the toxicity of the free drug by reducing its cell penetration, which is positive in case of premature deconjugation in the bloodstream and increases the off-target toxicity on low antigen-expressing cells, presumably due to nonspecific interaction of the nucleic acid-based linker with the cell surface. For example, in an antibody-drug conjugates, the antibody and drug can be non-covalently connected using complementary DNA linkers. Dovgan, I., Ehkirch, A., Lehot, V. et al. On the use of DNA as a linker in antibody-drug conjugates: synthesis, stability and in vitro potency. Sci Rep 10, 7691 (2020). Dovgan et al. disclosed a trastuzumab to be connected to monomethyl auristatin E (MMAE) through a 37-mer oligonucleotide.

In some embodiments, the cleavable linker may be a lipid. In some embodiments, the cleavable linker may be a phospholipid. The insertion of phospholipid groups between two fluorescent dyes or a dye/quencher pair allows the detection of phospholipase cleavage activity. In some embodiments, the cleavable linker may be a phosphodiester. The insertion of phosphodiester groups between two fluorescent dyes or a dye/quencher pair allows the detection of phosphodiesterase cleavage activity. In some embodiments, the lipid is directly attached to the fluorophore: once the covalent bond between the lipid and fluorophore is cleaved, the increase of fluorescent activity allows for the detection of the enzyme presence

In some embodiments, the cleavable linker may be an ester. Ester groups are often cleaved by saponification. The reactivity of the ester to cleavage can be enhanced by the use of electron-withdrawing groups or stabilized by the use of auto-immolative spacers to precluded spontaneous hydrolysis. In chemical biology, ester-based cleavable compounds were initially used for protein purification and in structural biology. FRET-based probes were designed to image esterase activities.

In some embodiments, the cleavable linker may be a glycoside. For example, cellulase enzymes deconstruct cellulose to glucose, and are often comprised of glycosylated linkers connecting glycoside hydrolases (GHs) to carbohydrate-binding modules (CBMs).

In some embodiments, the cleavable linker may be a nucleophile/base sensitive linker. These can include, but are not limited to, halogen nucleophiles, oxygen nucleophiles, safety-catch linkers, thiol nucleophiles, nitrogen nucleophiles, and phenacyl ester derivatives.

In some embodiments, the cleavable linker may be sensitive to activity from all enzyme families, including but is not limited to oxidoreductases, transferases, hydrolases, lyases, isomerases, and ligases.

Fluoridolyzable linkers are widely used in organic chemistry as silicon-based protecting groups for alcohols. The high thermodynamic affinity of fluorine for silicon allows their removal in orthogonal and mild conditions using a fluorine source. In this reaction a fluoride ion reacts with silicon as nucleophilic species and the cleavage conditions depend on the steric hindrance of the silicon's alkyl group. Fluoride ions can also trigger bond cleavage due to their basic properties.

Oxygen nucleophiles include sulfone and ester linkers while safety-catch linkers allow greater control over the timing of the bond breakage, because the linker will remain stable until it is activated for cleavage by a chemical modification.

A chemical modification can be any one of a number of processes that alter the chemical constitution or structure of a molecule. A chemical modification can include, but is not limited to, phosphorylation, alkylation, arylation, amination, amidation, sulfonylation, halogenation, borylation, glycosylation, cyclization, linearization, hydration, hydrogenation, nitration, nitrosylation, reduction, oxidation, esterification, hydrolysis, dephosphorylation, dealkylation, dearylation, deamination, deamidation, desulfonylation, dehalogenation, deborylation, deglycosylation, decyclization, delinearization, dehydration, dehydrogenation, denitration, denitrosylation, deesterification, dehydrolysis or any combination thereof.

In secondary amine synthesis or solid phase synthesis, nitrobenzenesulfonamides are known to be cleaved with a thiol nucleophile, like b-mercaptoethanol. Cysteines can be modified by electron-deficient alkynes to form a vinyl sulfide linkage.

Displacement reactions involving a specific nitrogen species as a nucleophile can occur in mild cleavable conditions. These reactions can be classified into two groups; cleavage by aminolysis or exchange reaction. For aminolysis cleavage, examples include the cleavage of a malondialdehyde (MDA) indole derivative by either pyrrolidine or hydrazine, and the cleavage of an ester linker by hydroxylamine or hydrazine. Acylhydrazones44 and hydrazones45,156 can be used as cleavable linkers through transimination in a mildly acidic medium. An amine catalyst (e.g., aniline, p-anisidine or hydroxylamine) accelerates hydrolysis and enables the effective transition between stable and dynamic states, which is required for cleavage and exchange.

In some embodiments, the cleavable linker may be a reduction sensitive linker. Reduction sensitive linkages have been used in chemical biology for a long time and it is a commonly used class of cleavable linker. Examples of cleavable linkers sensitive to reductive conditions include: nitroreductases, disulfide bridges and azo compounds. Karan et al. reported a fluorescent probe to detect nitroreductase. Sanu Karan, Mi Young Cho, Hyunseung Lee, Hwunjae Lee, Hye Sun Park, Mahesh Sundararajan, Jonathan L. Sessler, and Kwan Soo Hong. Near-Infrared Fluorescent Probe Activated by Nitroreductase for In Vitro and In Vivo Hypoxic Tumor Detection. Journal of Medicinal Chemistry 2021 64 (6), 2971-2981. In naturally occurring proteins, disulfide bridges generally play a role in maintaining the protein structure. They are known to be efficiently and rapidly cleaved by mild reducing agents like dithiothreitol (DTT), b-mercaptoethanol or tris(2-carboxyethyl) phosphine (TCEP). In chemical biology, disulfide bridges have been used in a wide range of applications including functional and structural proteomics, drug delivery, tumor imaging, DNA and protein-DNA complex purifications. The disulfide-based cleavable linker is commonly used due to its straightforward synthesis and rapid cleavage. Azo linkers are very appealing to chemical biologists since they are able to undergo cleavage following treatment with sodium dithionite, a mild and potentially bio-orthogonal reducing agent. The azo compound is reduced into two aniline moieties via an electrochemical reduction mechanism and this allows the use of reducing agents that are commonly used in many biological protocols, such as TCEP, DTT. In chemical biology, azo compounds have been used to cross-link proteins for over a decade and more recently for protein affinity purification.

In some embodiments, the cleavable linker may be an electrophile/acid sensitive linker. Acid sensitive linkers can be combined with other type of linkers. For example, a first β-galactosidase cleavage of the β-D-Galactopyranoside triggers the self-immolation of a benzyloxycarbonyl group, resulting in a release of optically active probe. Ho, N.-H., Weissleder, R. and Tung, C.-H. (2007), A Self-Immolative Reporter For β-Galactosidase Sensing. ChemBioChem, 8:560-566. Two different modes of electrophilic cleavage are used in chemical biology: acidic sensitive linkers that are sensitive to proton sources, and alkyl 2-(diphenylphosphino)benzoate derivatives sensitive to azide compounds. Proton sensitive bonds are among the most frequently used cleavable functions in organic chemistry; illustrated by the development of the BOC group which protects amines, or the Merrifield resin used in solid phase synthesis. In organic chemistry, the cleavage conditions that can be tolerated are very flexible regarding the acids' reagents, solvents, temperatures and pH. In contrast, biocompatible acid cleavable linkers must be responsive to minor changes in pH. Strong acidic conditions can lead to the denaturation of proteins and DNA. Biocompatible acid cleavable linkers are chosen for their instability near physiological pH and are often different from the classical protecting groups, which are cleaved with strong acids. Chemical reactions that can break or form bonds in water can be used as the basis of a cleavable linker, for example the Staudinger ligation. This reaction is proceeded by the nucleophilic attack of an alkyl 2-(diphenylphosphino)benzoate derivative on an azide, to form an aza-ylide intermediate. Then the ester traps the aza-ylide, which leads to the formation of an amide. In this process, the ester acts as a cleavable linker, and the azide as a bioorthogonal chemical agent, which guarantees a chemoselective and bioorthogonal cleavage.

In some embodiments, the cleavable linker may be a metal cleavable linker. Organometallic compounds are used to catalyze the modification of proteins containing non-natural amino acids, but their use as cleavage reagent in chemical biology has only been reported a few times. The allyl function is a commonly used protecting group for alcohols in organic synthesis and it is also used as a cleavable linker in DNA sequencing by synthesis Metal cleavable linkers were also used in the design of peptide nucleic acids (PNAs), which were developed for enzyme-independent DNA/RNA hybridization methods.

In some embodiments, the cleavable linker may be an oxidation sensitive linker. Sodium periodate is undoubtedly the most frequently used biocompatible oxidizing agent due to its ability to cleave vicinal diols to form two aldehydes compounds. One example of this type of cleavable linker consists of a vicinal diol with a tartaric acid spacer and two functional groups at both ends. Selenium based linkers also contain cleavable bonds sensitive to oxidizing agents, such as sodium periodate or N-chlorobenzenesulfonamide immobilized on polystyrene beads (iodo-beads). The trigger agent oxidizes the labile bond to selenium oxide, which is then cleaved directly via intramolecular b-elimination or rearrangement.

### Reporter and Detection Methods

In some aspects, the probe/molecule described herein comprises a reporter. The reporter as described herein may be in any structure that may be capable of being detected by any method, including but not limited to fluorescent detection, spectroscopic detection, immunological detection or imaging detection. In some embodiments, the reporter may be a fluorescent label, a mass tag or a nucleic acid barcode.

In some embodiments, the reporter may be a fluorescent label. Labels, tags and probes containing small compounds such as florescence can be used to label proteins and nucleic acids. Bio-affinity towards other molecules (biotin, digoxygenin), enzymatic (AP, HRP) or chemiluminescent (esters or acridine) can be used as well. Genetically encoded markers like the fluorescent proteins of the GFP family have become a reporter of choice for gene expression studies and protein localization. In combination with subcellular tags, GFP can be used to label subcellular structures like synapses allowing novel approaches to study developmental processes like synapse formation. Other fluorescent labels include but are not limited to small organic dyes and lipophilic dyes. The fluorescence label may serve itself as the activity substrate without addition of linkers.

Some reporters are “internally quenched”, thus does not require a quencher, wherein the cleavage of a bond linking the internally quenched fluorophore to the substrate linker directly yields a fluorescent molecule. Many described probes for proteases, esterases, peroxidases and others function this way.

In some embodiments, the reporter may be a mass tag. Mass tag reagents are designed to enable identification and quantitation of proteins in different samples using mass spectrometry (MS). Mass tagging reagents within a set typically have the same nominal mass (i.e., are isobaric) and chemical structure composed of an amine-reactive NHS ester group, a spacer arm (mass normalizer), and a mass reporter.

In some embodiments, the reporter may be a nucleic acid barcode. For example, DNA barcoding is a system for species identification focused on the use of a short, standardized genetic region acting as a “barcode” in a similar way that Universal Product Codes are used by supermarket scanners to distinguish commercial products.

In some embodiments, the reporter may be detected using a ligand binding assay. A ligand binding assay often involves a detection step, such as an ELISA, including fluorescent, colorimetric, bioluminescent and chemiluminescent ELISAs, a paper test strip or lateral flow assay, or a bead-based fluorescent assay. In some embodiments, a paper-based ELISA test may be used to detect the cleaved reporter in the fluid sample. The paper-based ELISA may be created inexpensively, such as by reflowing wax deposited from a commercial solid ink printer to create an array of test spots on a single piece of paper. When the solid ink is heated to a liquid or semi-liquid state, the printed wax permeates the paper, creating hydrophobic barriers. The space between the hydrophobic barriers may then be used as individual reaction wells. The ELISA assay may be performed by drying the detection antibody on the individual reaction wells, constituting test spots on the paper, followed by blocking and washing steps. Fluid from a sample taken from the subject may then be added to the test spots. Then, for example, a streptavidin alkaline phosphate (ALP) conjugate may be added to the test spots, as the detection antibody. Bound ALP may then be exposed to a color reacting agent, such as BCIP/NBT (5-bromo-4-chloro-3′-indolyphosphate p-toluidine salt/nitro-blue tetrazolium chloride), which causes a purple colored precipitate, indicating presence of the reporter.

In some embodiments, the reporter can be detected using volatile organic compounds. Volatile organic compounds may be detected by analysis platforms such as gas chromatography instrument, a breathalyzer, a mass spectrometer, or use of optical or acoustic sensors. Gas chromatography may be used to detect compounds that can be vaporized without decomposition (e.g., volatile organic compounds). A gas chromatography instrument includes a mobile phase (or moving phase) that is a carrier gas, for example, an inert gas such as helium or an unreactive gas such as nitrogen, and a stationary phase that is a microscopic layer of liquid or polymer on an inert solid support, inside a piece of glass or metal tubing called a column. The column is coated with the stationary phase and the gaseous compounds analyzed interact with the walls of the column, causing them to elute at different times (i.e., have varying retention times in the column). Compounds may be distinguished by their retention times.

Mass spectrometry and enrichment/chromatography methods may be used to separate and distinguish/detect cleaved from intact reporters used in the present disclosure based on differences in mass and or presence of a label. For example, enzymatic reactions can result in the fragmentation of a parent molecule resulting in a mass shift of the starting substrate, this can be exploited in different chromatography/enrichment methods such as size exclusion chromatography and affinity enrichments. In mass spectrometry, a sample is ionized, for example by bombarding it with electrons. The sample may be solid, liquid, or gas. By ionizing the sample, some of the sample's molecules are broken into charged fragments. These ions may then be separated according to their mass-to-charge ratio. This is often performed by accelerating the ions and subjecting them to an electric or magnetic field, where ions having the same mass-to-charge ratio will undergo the same amount of deflection. When deflected, the ions may be detected by a mechanism capable of detecting charged particles, for example, an electron multiplier. The detected results may be displayed as a spectrum of the relative abundance of detected ions as a function of the mass-to-charge ratio. The molecules in the sample can then be identified by correlating known masses, such as the mass of an entire molecule to the identified masses or through a characteristic fragmentation pattern.

When the reporter includes a nucleic acid, the reporter may be detected by various sequencing methods known in the art, for example, traditional Sanger sequencing methods or by next-generation sequencing (NGS). NGS generally refers to non-Sanger-based high throughput nucleic acid sequencing technologies, in which many (i.e., thousands, millions, or billions) of nucleic acid strands can be sequenced in parallel. Examples of such NGS sequencing includes platforms produced by Illumina (e.g., HiSeq, MiSeq, NextSeq, MiniSeq, and iSeq 100), Pacific Biosciences (e.g., Sequel and RSII), and Ion Torrent by ThermoFisher (e.g., Ion S5, Ion Proton, Ion PGM, and Ion Chef systems). It is understood that any suitable NGS sequencing platform may be used for NGS to detect nucleic acid of the detectable analyte as described herein.

Analysis may be performed directly on the biological sample or the detectable cleaved reporters may be purified to some degree first. For example, a purification step may involve isolating the detectable analyte from other components in the biological sample. Purification may include methods such as affinity chromatography. The isolated or purified detectable analyte does not need to be 100% pure or even substantially pure prior to analysis. Detecting the cleaved reporters may provide a qualitative assessment (e.g., whether the detectable cleaved reporters, and thus the predetermined protease is present or absent) or a quantitative assessment (e.g., the amount of the detectable cleaved reporters present) to indicate a comparative activity level of the predetermined proteases in the fluid sample. The quantitative value may be calculated by any means, such as, by determining the percent relative amount of each fraction present in the sample. Methods for making these types of calculations are known in the art.

The cleaved reporters may be detected by any detection method that may be suitable for the particular reporter. In some aspects, the detection method comprises fluorescent detection, spectroscopic detection, mass spectrometry, immunological detection or imaging detection. In some aspects, the detection method may be fluorescence resonance energy transfer (FRET).

In some embodiments, the detection method may be spectroscopic detection. Spectroscopic methods of detection are very commonly employed in ion chromatography (IC) and are second only to conductivity detection in their frequency of usage. These methods can be divided broadly into the categories of molecular spectroscopic techniques and atomic spectroscopic techniques. Molecular spectroscopy includes UV-visible spectrophotometry, refractive index measurements, and photoluminescence techniques (fluorescence and phosphorescence). Atomic spectroscopy includes atomic emission spectroscopy (using various excitation sources) and atomic absorption spectroscopy. Many of the spectroscopic detection methods can operate in a direct or indirect mode. The definitions of these terms are the same as those used to describe the electrochemical detection modes. That is, direct spectroscopic detection results when the solute ion has a greater value of the measured detection parameter than does the eluent ion. Indirect detection results when the reverse is true.

In some embodiments, the detection method may be mass spectrometry. Mass spectrometry (MS) is an analytical technique that is used to measure the mass-to-charge ratio of ions. The results are typically presented as a mass spectrum, a plot of intensity as a function of the mass-to-charge ratio.

In some embodiments, the detection method may be fluorescence resonance energy transfer (FRET). FRET (Fluorescence Resonance Energy Transfer) is a distance dependent dipole-dipole interaction without the emission of a photon, which results in the transfer of energy from an initially excited donor molecule to an acceptor molecule. It allows the detection of molecular interactions in the nanometer range. FRET peptides are labeled with a donor molecule and an acceptor (quencher) molecule. In most cases, the donor and acceptor pairs are two different dyes. The transferred energy from a fluorescent donor is converted into molecular vibrations if the acceptor is a non-fluorescent dye (quencher). When the FRET is terminated (by separating donor and acceptor), an increase of donor fluorescence can be detected. When both the donor and acceptor dyes are fluorescent, the transferred energy is emitted as light of longer wavelength so that the intensity ratio change of donor and acceptor fluorescence can be measured. In order for efficient FRET quenching to take place, the fluorophore and quencher molecules must be close to each other (approximately 10-100 Å) and the absorption spectrum of the quencher must overlap with the emission spectrum of the fluorophore.

### Precipitating Fluorophore

In some aspects, the cleaved reporter may be a precipitating fluorophore. In some embodiments, the precipitating fluorophore may be HPQ, CI-HPQ, HTPQ, HTPQA, HBPQ, or HQPQ.

In some embodiments, the precipitating fluorophore may be HPQ, also known as 2-(2′-hydroxyphenyl)-4(3H)-quinazolinone. HPQ is a small organic dye known for its classic luminescence mechanism through excited-state intramolecular proton transfer (ESIPT), shows strong light emission in the solid state, but no emission in solution. HPQ is found to be strictly insoluble in water and exhibits intense solid-state fluorescence similar to that of tetraphenyl ethylene. Moreover, its essential properties of insolubility and intense solid-state fluorescence can be countered and reversed, by prohibiting the establishment of an internal hydrogen bond between the imine nitrogen and phenolic hydroxyl group.

In some embodiments, the precipitating fluorophore may be Cl-HPQ. C1-HPQ is released when HPQF, a water soluble and non-fluorescent molecule, reacts with furin. Cl-HPQ starts to precipitate near the enzyme activity site, and the precipitates emit bright solid-state fluorescence with more than 60-fold fluorescence enhancement. Li et al. In Situ Imaging of Furin Activity with a Highly Stable Probe by Releasing of Precipitating Fluorochrome. Anal. Chem. 2018, 90, 19, 11680-11687.

In some embodiments, the precipitating fluorophore may be HTPQ. HTPQ is found to be strictly insoluble in water and shows intense fluorescence in the solid state with maximum excitation and emission wavelengths at 410 nm and 550 nm respectively. This makes it far better suited to the use with a confocal microscope. The large Stokes shift of HTPQ contributes additional and highly desirable advantages: increased sensitivity, minimized background fluorescence and enhanced bioimaging contrast. Liu et al. In Situ Localization of Enzyme activity in Live Cells by a Molecular Probe Releasing a Precipitating Fluorochrome. Angew Chem Int Ed Engl. 2017 Sep. 18; 56(39):11788-11792.

In some embodiments, the precipitating fluorophore may be HTPQA. HTPQA is another enzyme-responsive fluorogenic probe derived from HTPQ. When converted by ALP, the probe releases free HTPQ which starts to precipitate after a very short delay; the precipitate emits bright solid-state fluorescence with more than 100-fold fluorescence enhancement.

In some embodiments, the precipitating fluorophore may be HBPQ. HBPQ is completely insoluble in water and shows strong yellow solid emission when excited with a 405 nm laser. Liu et al. Precipitated Fluorophore-Based Molecular Probe for In Situ Imaging of Aminopeptidase N in Living Cells and Tumors. Anal. Chem. 2021, 93, 16, 6463-6471, Publication Date: Apr. 14, 2021.

In some embodiments, the precipitating fluorophore may be HQPQ. HQPQ is, a novel solid-state fluorophore that is insoluble in water. Li et al. Precipitated Fluorophore-Based Probe for Accurate Detection of Mitochondrial Analytes. Anal. Chem. 2021, 93, 4, 2235-2243. Publication Date: Jan. 5, 2021.

The precipitating and non-precipitating fluorophores can be separated from the enzyme substrate by a self-immolative substrate to stabilize the initial probe and ensure that the enzymatic cleavage is transduced via the immolative spacer into the formation of the precipitating fluorophore or the non-internally quenched soluble fluorophore.

### Fluorescent Quencher

In some aspects, the probe/molecule described herein comprises a fluorescent quencher. The fluorescent quencher as described herein may be in any structure that is capable of decreasing the fluorescence intensity of a given substance. In some embodiments, the fluorescent quencher may be BHQ0, BHQ1, BHQ2, BHQ3, BBQ650, ATTO 540Q, ATTO 580Q, ATTO 612Q, CPQ2, QSY-21, QSY-35, QSY-7, QSY-9, DABCYL (4-([4′-dimethylamino)phenyl] azo)benzoyl), Dnp (2,4-dinitrophenyl) or Eclipse®.

In some embodiments, the fluorescent quencher may be a BHQ quencher including, but not limited to, BHQ0, BHQ1, BHQ2, BHQ3, or BBQ650. BHQ, or black hole quencher, dyes work through a combination of FRET and static quenching to enable avoidance of the residual background signal common to fluorescing quenchers such as TAMRA, or low signal-to-noise ratio. The different types of BHQ dyes are used to quench different colored dyes with BHQ1 used to quench green and yellow dyes such as FAM, TET, or HEX and BHQ2 used for quenching orange and red dyes. BHQ dyes are true dark quenchers with no native emission due to their polyacromatic-azo backbone. Substituting electron-donating and withdrawing groups on the aromatic rings produces a complete series of quenchers with broad absorption curves that span the visible spectrum.

In some embodiments, the fluorescent quencher may be an ATTO quencher including, but not limited to ATTO 540Q, ATTO 580Q, or ATTO 612Q. ATTO quenchers have characteristic properties of strong absorption (high extinction coefficient) and high photo-stability. ATTO quenchers are often utilized as fluorescent quenchers on amine-labeled nucleotides for FRET experiments.

In some embodiments, the fluorescent quencher may be CPQ2. The quencher CPQ2 is often used as a pair with the fluorescent donor 5-carboxylfluorescein.

In some embodiments, the fluorescent quencher may be a QSY quencher including but not limited to QSY-21, QSY-35, QSY-7, or QSY-9. QSY probes are dark quenchers, substances that absorb excitation energy from a fluorophore and dissipate the energy as heat.

In some embodiments, the fluorescent quencher may be DABCYL (4-([4′-dimethylamino)phenyl]azo)benzoyl). DABCYL is one of the most popular acceptors for developing FRET-based nucleic acid probes and protease substrates. DABCYL dyes are often paired with EDANS in FRET-based fluorescent probes. DABCYL has a broad and intense visible absorption but no fluorescence.

In some embodiments, the fluorescent quencher may be Dnp (2,4-dinitrophenyl). Dnp is a stable quencher and its absorption spectrum does not change with pH, which makes this group a convenient marker for substrate quantitation in solutions.

In some embodiments, the fluorescent quencher may be Eclipse®. Eclipse® is a non-fluorescent chromophore and a dark quencher often used in dual-labelled probes. As dark quenchers, Eclipse® absorbs energy without emitting fluorescence. Eclipse® has an absorption range from 390 nm to 625 nm and is capable of effective performance in a wide range of colored FRET probes.

### Carrier

In some aspects, the probe/molecule described herein comprises a carrier. The fluorescent quencher as described herein may be in any structure. In some embodiments, the carrier may be a native, labeled or synthetic protein, a synthetic chemical polymer of precisely known chemical composition or with a distribution around a mean molecular weight (e.g. a linear or branched PEG polymers), an oligonucleotide, a phosphorodiamidate morpholino oligomer (PMO), or a foldamer, a lipid, a lipid micelle, a nanoparticle (e.g., iron oxide, gold, and non-metallic nanoparticles), a solid support made of polystyrene, polypropylene or any other type of plastic or polymer. In some embodiments, the carrier may be a peptide longer than the peptide linker. A carrier can be covalently or non-covalently attached to the cleavable linker.

In some embodiments, the carrier may be a nanoparticle. The transport of insoluble drugs via nanoparticles is improving because of their small particle size. Nanoparticle carrier is a kind of sub-micro particle delivery system, which belongs to a nanoscale microscope. Drugs encapsulated in sub-particles can adjust the speed of drug release, increase the permeability of biofilm, change the distribution in vivo, and improve the bioavailability. Nanoparticles are solid colloidal particles ranging in size from 10 to 100 nm used as a core in functionalization systems. They are generally composed of natural or synthetic macromolecule substances and can be used as carriers for conducting or transporting drugs. Nanospheres and nanocapsules can be formed. The chemical materials of nanomaterials are chitosan, gelatin, branched polymers, carbon-based carriers, etc. Gold nanoparticles consist of a core of gold atoms that can be functionalized by addition of a monolayer of moieties containing a thiol (SH) group.

In some embodiments, the carrier may be a native, labeled or synthetic protein. Proteins can be used as carriers for the delivery of chemicals and biomolecular drugs, such as anticancer drugs and therapeutic proteins. Protein nanoparticles have several advantages as a drug delivery system, such as biodegradability, stability, surface modification of particles, ease of particle size control, and they have less problems associated with toxicity issues, such as immunogenicity. Protein nanoparticles can be generated using proteins, such as fibroins, albumin, gelatin, gliadine, legumin, 30Kc19, lipoprotein, and ferritin proteins, and are prepared through emulsion, electrospray, and desolvation methods. Hong S, Choi D W, Kim H N, Park C G, Lee W, Park H H. Protein-Based Nanoparticles as Drug Delivery Systems. Pharmaceutics. 2020; 12(7):604. Published 2020 Jun. 29. For example, albumin, a plasma protein with a molecular weight of 66 kDa, has been extensively investigated as a drug carrier.

In some embodiments, the carrier may be a synthetic chemical polymer. Polymeric nanoparticles have been extensively investigated as drug nanocarriers. Drug loading is achieved either by (i) entrapment of an aqueous drug phase using the polymer to form nanoscale structures such as cages and capsules or (ii) chemical linking of the drug molecules to the polymer backbone by means of a simple ester or amide bond that can be hydrolyzed in vivo. The most widely researched synthetic polymers include polylactide (PLA), poly(D,L-lactide-co-glycolide) (PLGA) and PEG. All three polymers are hydrolyzed in vivo and are biodegradable. Malam Y, Loizidou M, Seifalian A M. Liposomes and nanoparticles: nanosized vehicles for drug delivery in cancer. Trends Pharmacol Sci. 2009 November; 30(11):592-9.

In some embodiments, the carrier may be a polyethylene glycol (PEG). PEG has been studied comprehensively as a carrier because it is soluble in both organic and hydrophilic solvents. Unlike many other synthetic polymers, PEG is relatively hydrophilic. Conjugation with PEG increases the solubility of hydrophobic molecules and prolongs the circulation time in the organism. PEG also minimizes the nonspecific absorption of a molecule, such as a drug, provides specific affinity toward the targeted tissue, and increases the drug accumulation in malignant tissue. PEG can be conjugated to other polymers to make them less hydrophobic (i.e., PEGylation). The changes in surface hydrophilicity prevent protein adsorption, thereby enabling cell adhesion and proliferation on biomaterial scaffolds. The PMO backbone is made of morpholino rings with phosphorodiamidate linkage, which protects them from nuclease degradation while still maintaining the complementary base pairing. The potential application of PMO-based antisense technology targeting bacterial pathogens is being explored for the development of a new class of antibacterial drugs. Panchal R G, Geller B L, Mellbye B, Lane D, Iversen P L, Bavari S. Peptide conjugated phosphorodiamidate morpholino oligomers increase survival of mice challenged with Ames Bacillus anthracis. Nucleic Acid Ther. 2012; 22(5):316-322. Fluorescein-tagged Morpholinos combined with fluorescein-specific antibodies can be used as probes for in-situ hybridization to miRNAs.

In some embodiments, the carrier may be an oligonucleotide. Biostable, high-payload DNA nano assemblies of various structures, including cage-like DNA nanostructure, DNA particles, DNA polypods, and DNA hydrogel, have been reported. Cage-like DNA structures hold drug molecules firmly inside the structure and leave a large space within the cavity. These DNA nanostructures use their unique structure to carry abundant CpG, and their biocompatibility and size advantages to enter immune cells to achieve immunotherapy for various diseases. Part of the DNA nanostructures can also achieve more effective treatment in conjunction with other functional components such as aPD1, RNA, TLR ligands. DNA-based nanoparticles, such as spherical nucleic acids, hybrid DNA-based nanoparticles, polypod-like DNA nanostructure, DNA hydrogels have been reported. Chi Q, Yang Z, Xu K, Wang C and Liang H (2020) DNA Nanostructure as an Efficient Drug Delivery Platform for Immunotherapy. Front. Pharmacol. 10:1585.

In some embodiments, the carrier may be a phosphorodiamidate Morpholino oligomer (PMO). Antisense phosphorodiamidate morpholino oligomers (PMOs) and their derivatives downregulate target gene expression in a sequence-dependent manner by interfering with the binding of ribosome to mRNA and thereby inhibiting protein translation.

In some embodiments, the carrier may be a lipid or a lipid micelle. The liposome bilayer can be composed of either synthetic or natural phospholipids. The predominant physical and chemical properties of a liposome are based on the net properties of the constituent phospholipids, including permeability, charge density and steric hindrance. The lipid bilayer closes in on itself due to interactions between water molecules and the hydrophobic phosphate groups of the phospholipids. This process of liposome formation is spontaneous because the amphiphilic phospholipids self-associate into bilayers. Drug loading into liposomes can be achieved through (i) liposome formation in an aqueous solution saturated with soluble drug; (ii) the use of organic solvents and solvent exchange mechanisms; (iii) the use of lipophilic drugs; and (iv) pH gradient methods. Malam Y, Loizidou M, Seifalian A M. Liposomes and nanoparticles: nanosized vehicles for drug delivery in cancer. Trends Pharmacol Sci. 2009 November; 30(11):592-9.

In some embodiments, the carrier may be a solid support made of polystyrene, polypropylene or any other type of plastic. For example, drug delivery properties of microporous polystyrene solid foams have been reported by Canal et al. These materials were obtained by polymerization in the continuous phase of highly concentrated emulsions prepared by the phase inversion temperature method. Their porosity, specific surface and surface topography are associated with drug incorporation and release characteristics. Canal, Cristina & Aparicio, Rosa & Vílchez, Alejandro & Esquena, Jordi & García-Celma, Maria. (2012). Drug Delivery Properties of Macroporous Polystyrene Solid Foams. Journal of pharmacy & pharmaceutical sciences: a publication of the Canadian Society for Pharmaceutical Sciences, Société canadienne des sciences pharmaceutiques. 15. 197-207.

In some embodiments, the carrier may be a foldamer. Foldamer, is a folded oligomer or polymer with a well-defined conformation. The conformation of foldamers is highly predictable from their primary sequences, therefore, it is possible to arrange functional groups at target positions and it may be possible to design functional foldamers, such as for efficient cellular uptake. For example, Cell-penetrating peptide (CPP) foldamers are peptide-based foldamers equipped with cell membrane permeabilities. Peptide foldamers contain unnatural amino acids, non-proteinogenic amino acids, which make the peptide adopt a stable secondary structure, especially helical structures, even in short sequences. This property is helpful for the design of amphipathic CPPs with a stable helical structure. Furthermore, peptides containing unnatural amino acids generally exhibit resistance to hydrolysis by proteases, which are abundant throughout the body and in the cells. High stability of the peptide foldamers against enzymatic degradation can lead to their prolonged function in vivo. Makoto Oba, Cell-Penetrating Peptide Foldamers: Drug Delivery Tools. ChemBioChem 10.1002/cbic.201900204.

### Self-Immolative Spacer

In some aspects, the probe/molecule described herein comprises a self-immolative spacer. In some embodiments, the self-immolative spacer comprise a disulfide, a p-amino benzyl alcohol, an a-quinone methide spacer, a hetheroaminebifuncional disulfide, a thiol-based pirydazinediones, a p-aminebenzyloxycarbonyl, a dipeptide, a Gly-Pro, a L-Phe-Sar, a trans-cyclooctene tetrazine, a ortho Hydroxy-protected Aryl sulfate, a phosphoramidate-based spacer, a hydroxybenzyl, a trimethyl carbamate, a quinone methide-based spacer, a cyclizing spacer, a Trimethyl lock, a 2-amino methyl piperidine or an ethylene diamine derived cyclizing spacer. Gonzaga et al. Perspective about self-immolative drug delivery systems. Journal of Pharmaceutical Sciences 109 (2020) 3262-3281.

Cleavage of the cleavable linker by a predetermined protease or enzyme makes the self-immolative spacer dissociate from the precipitating fluorescent or non-fluorescent reporter, thereby resulting in a detectable signal. The cleavable linker of the plurality of probes/molecules may be cleavable by a predetermined endoprotease in the body fluid sample resulting in auto immolation and reporter release or results in a protease substrate that can be cleaved by a predetermined exopeptidase. In some embodiments, the predetermined exopeptidase is added to the body fluid sample. In some embodiments, the predetermined exopeptidase cleaves the protease substrate, thereby causing the self-immolative spacer to dissociate from the precipitating fluorescent reporter, thereby resulting in a detectable signal.

### Body Fluid Samples

Determination of the disease or condition is based on the rate of formation or amount of the released reporter detected in the body fluid sample. In some embodiments, the body fluid sample may be blood, serum, plasma, bone marrow fluid, lymphatic fluid, bile, amniotic fluid, mucosal fluid, saliva, urine, cerebrospinal fluid, synovial fluid, ascitic fluid, semen, ductal aspirate, feces, vaginal effluent, cyst fluid, tissue homogenate, tissue-derived fluid, lachrymal fluid and patient-derived cell line supernatant. In some embodiments, the body fluid sample comprises a rinse fluid. In some embodiments, the rinse fluid may be a mouthwash rinse, a bronchioalveolar rinse, a lavage fluid, a hair wash rinse, a nasal spray effluent, a swab of any bodily surface, orifice or organ structure applied to saline or any media or any derivatives thereof.

In some embodiments, the body fluid sample may be blood. Blood is a constantly circulating fluid providing the body with nutrition, oxygen, and waste removal. Blood is mostly liquid, with numerous cells and proteins suspended in it. Blood is made of several main factors including plasma, red blood cells, white blood cells, and platelets.

In some embodiments, the body fluid sample may be a plasma. Plasma is the liquid that remains when clotting is prevented with the addition of an anticoagulant. Serum is the conventional term in the art for the fluid that remains when clotting factors are removed from plasma. Anticoagulants are medicines that help prevent blood clots. Examples of anticoagulants include, but are not limited to, an ethylenediamine tetra acetic acid (EDTA), a citrate, a heparin, an oxalate, any salt, solvate, enantiomer, tautomer and geometric isomer thereof, or any mixtures thereof.

In some embodiments, the anticoagulant may be EDTA. The main property of EDTA, a polyprotic acid containing four carboxylic acid groups and two amine groups with lone pair electrons, is the ability to chelate or complex metal ions in 1:1 metal-EDTA complexes. Owing to its strong complexation with metal ions that are cofactors for enzymes, EDTA is widely used as a sequestering agent to prevent some enzyme reactions from occurring. When blood is collected with no additives within an appropriate container (blood tube), it clots fairly quickly. As calcium ions are necessary for this process, the specific association between the carboxylic groups of EDTA and calcium is a reliable solution to prevent clotting, stabilizing whole blood in a fluid form, as required for some laboratory analyses. Moreover, EDTA showed optimal extended stabilization of blood cells and particles. Three EDTA formulations can be employed as anticoagulants: Na₂EDTA, K2 EDTA and K3 EDTA, choice of which mostly depends on the type of analyses to be performed.

In some embodiments, the anticoagulant may be a citrate. Citrate (C6H707) is a small negatively charged molecule with a molecular weight of 191 Daltons. Citrate can be used as the anticoagulant of choice for stored blood products, typically as acid citrate dextrose (ACD), (3.22% citrate, 112.9 mmol/l citrate, 123.6 mmol/l glucose, 224.4 mmol/l sodium and 114.2 mmol/l hydrogen ions), or trisodium citrate (TCA) Na₃C₃H₅O(COO)₃, (4% TCA, 136 mmol/l citrate, 420 mmol/l sodium). Citrate chelates calcium, and at a concentration of 4-6 mmol/l with an ionized calcium of <0.2 mmol/l prevents activation of both coagulation cascades and platelets. As such, citrate has been the standard anticoagulant used by hematologists and blood transfusion services for stored blood products and also as an extracorporeal anticoagulant for centrifugal platelet and leukapheresis techniques and plasma exchange.

In some embodiments, the anticoagulant may be a heparin. The molecular basis for the anticoagulant action of heparin lies in its ability to bind to and enhance the inhibitory activity of the plasma protein antithrombin against several serine proteases of the coagulation system, most importantly factors IIa (thrombin), Xa and IXa. Two major mechanisms underlie heparin's potentiation of antithrombin. The conformational changes induced by heparin binding cause both expulsion of the reactive loop and exposure of exosites of the surface of antithrombin, which bind directly to the enzyme target; and a template mechanism exists in which both inhibitor and enzyme bind to the same heparin molecule. The relative importance of these two modes of action varies between enzymes. In addition, heparin can act through other serine protease inhibitors such as heparin co-factor II, protein C inhibitor and tissue factor plasminogen inhibitor. The antithrombotic action of heparin in vivo, though dominated by anticoagulant mechanisms, is more complex, and interactions with other plasma proteins and cells play significant roles in the living vasculature.

In some embodiments, the anticoagulant may be an oxalate. Sodium, potassium, ammonium, and lithium oxalates inhibit blood coagulation by forming insoluble complex with calcium. Potassium oxalate at concentration of 1-2 mg/ml of blood is widely used. Combined ammonium and/or potassium oxalate does not cause shrinkage of erythrocytes. It consists of three parts by weight of ammonium oxalate, which causes swelling of the erythrocytes, balanced by two parts of potassium oxalate which causes shrinkage. NH4+ & K+ oxalate mixture in the ratio of 3:2, and 2 mg/ml of blood is the required amount.

In some embodiments, the body fluid sample may be bone marrow fluid. Bone marrow is found in the center of most bones and has many blood vessels. There are two types of bone marrow: red and yellow. Red marrow contains blood stem cells that can become red blood cells, white blood cells, or platelets. Yellow marrow is made mostly of fat.

In some embodiments, the body fluid sample may be lymphatic fluid. Lymphatic fluid, also called lymph, is a collection of the extra fluid that drains from cells and tissues, that is not reabsorbed into the capillaries.

In some embodiments, the body fluid sample may be bile. Bile is a digestive fluid produced by the liver and stored in the gallbladder. During bile reflux, digestive fluid backs up into the stomach and, in some cases, the esophagus.

In some embodiments, the body fluid sample may be amniotic fluid. Amniotic fluid is a clear, slightly yellowish liquid that surrounds the unborn baby (fetus) during pregnancy. It is contained in the amniotic sac.

In some embodiments, the body fluid sample may be mucosal fluid. Mucosal fluid, also called mucus, is a thick protective fluid that is secreted by mucous membranes and used to stop pathogens and dirt from entering the body. Mucus is also used to prevent bodily tissues from being dehydrated.

In some embodiments, the body fluid sample may be saliva. Saliva is an extracellular fluid produced and secreted by salivary glands in the mouth.

In some embodiments, the body fluid sample may be urine. Urine is a liquid by-product of metabolism in humans and in many other animals. Urine flows from the kidneys through the ureters to the urinary bladder.

In some embodiments, the body fluid sample may be cerebrospinal fluid. Cerebrospinal fluid is a clear fluid that surrounds the brain and spinal cord. It cushions the brain and spinal cord from injury and also serves as a nutrient delivery and waste removal system for the brain.

In some embodiments, the body fluid sample may be synovial fluid. Synovial fluid, also known as joint fluid, is a thick liquid located between your joints. The fluid cushions the ends of bones and reduces friction when joints are moved.

In some embodiments, the body fluid sample may be ascitic fluid. Ascitic fluid is fluid that comes from ascites, a condition in which abnormal amounts of fluid collect in the abdominal space. Ascitic fluid is often related to liver disease.

In some embodiments, the body fluid sample may be semen. Semen is the male reproductive fluid which contains spermatozoa in suspension.

In some embodiments, the body fluid sample may be ductal aspirate. Ductal aspirate, also known as ductal lavage, ductal fluid, or lavage fluid, is fluid collected from a duct, such as the milk duct of the breast.

In some embodiments, the body fluid sample may be feces. Feces, also known as excrement or stool is waste matter discharged from the bowels after food has been digested.

In some embodiments, the body fluid sample may be vaginal effluent. Vaginal effluent, also known as vaginal discharge, is a clear or whitish fluid that comes out of the vagina.

In some embodiments, the body fluid sample may be lachrymal fluid. Lachrymal fluid, also known as lacrimal fluid, is secreted by the lacrimal glands to lubricate the eye and fight bacteria.

In some embodiments, the body fluid sample may be tissue homogenate. A tissue homogenate is obtained through mechanical micro-disruption of fresh tissue and the cell membranes are mechanically permeabilized.

### Proteases and Other Agents

The probe/molecule described herein may be cleaved by a protease from the body fluid. In some embodiments, the protease comprises an endopeptidase or an exopeptidase.

In some embodiments, the protease comprises an endopeptidase. An endopeptidase is an enzyme which breaks peptide bonds other than terminal ones in a peptide chain.

In some embodiments, the protease comprises an exopeptidase. An exopeptidase is an enzyme that catalyzes the cleavage of the terminal or penultimate peptide bond; the process releases a single amino acid or dipeptide from the peptide chain.

In some embodiments, the protease comprises an A20 (TNFa-induced protein 3), an abhydrolase domain containing 4, an abhydrolase domain containing 12, an abhydrolase domain containing 12B, an abhydrolase domain containing 13, an acrosin, an acylaminoacyl-peptidase, a disintegrin and metalloproteinase (ADAM), an ADAM1a, an ADAM2 (Fertilin-b), an ADAM3B, an ADAM4, an ADAM4B, an ADAM5, an ADAM6, an ADAM7, an ADAM8, an ADAM9, an ADAM10, an ADAM11, an ADAM12 metalloprotease, an ADAM15, an ADAM17, an ADAM18, an ADAM19, an ADAM20, an ADAM21, an ADAM22, an ADAM23, an ADAM28, an ADAM29, an ADAM30, an ADAM32, an ADAM33, a disintegrin and metalloproteinase with thrombospondin motifs (ADAMTS), an ADAMTS1, an ADAMTS2, an ADAMTS3, an ADAMTS4, an ADAMTS5/11, an ADAMTS6, an ADAMTS7, an ADAMTS8, an ADAMTS9, an ADAMTS10, an ADAMTS12, an ADAMTS13, an ADAMTS14, an ADAMTS15, an ADAMTS16, an ADAMTS17, an ADAMTS18, an ADAMTS19, an ADAMTS20, an adipocyte-enh. binding protein 1, an Afg3-like protein 1, an Afg3-like protein 2, an airway-trypsin-like protease, an aminoacylase, an aminopeptidase A, an aminopeptidase B, an aminopeptidase B-like 1, an aminopeptidase MAMS/L-RAP, an aminopeptidase N, an aminopeptidase O, an aminopeptidase P homologue, an aminopeptidase P1, an aminopeptidase PILS, an aminopeptidase Q, an aminopeptidase-like 1, an AMSH/STAMBP, an AMSH-LP/STAMBPL1, an angiotensin-converting enzyme 1 (ACE1), an angiotensin-converting enzyme 2 (ACE2), an angiotensin-converting enzyme 3 (ACE3), an anionic trypsin (II), an apolipoprotein (a), an archaemetzincin-1, an archaemetzincin-2, an aspartoacylase, an aspartoacylase-3, an aspartyl aminopeptidase, an ataxin-3, an ataxin-3 like, an ATP/GTP binding protein 1, an ATP/GTP binding protein-like 2, an ATP/GTP binding protein-like 3, an ATP/GTP binding protein-like 4, an ATP/GTP binding protein-like 5, an ATP23 peptidase, an autophagin-1, an autophagin-2, an autophagin-3, an autophagin-4, an azurocidin, or a combination hereof.

In some embodiments, the protease comprises a beta lactamase, a beta-secretase 1, a beta-secretase 2, a bleomycin hydrolase, a brain serine proteinase 2, a BRCC36 (BRCA2-containing complex, sub 3), a calpain, a calpain 1, a calpain 2, a calpain 3, a calpain 4, a calpain 5, a calpain 6, a calpain 7, a calpain 7-like, a calpain 8, a calpain 9, a calpain 10, a calpain 11, a calpain 12, a calpain 13, a calpain 14, a calpain 15 (Solh protein), or a combination hereof.

In some embodiments, the protease comprises a cysteine protease, a carboxypeptidase A1, a carboxypeptidase A2, a carboxypeptidase A3, a carboxypeptidase A4, a carboxypeptidase A5, a carboxypeptidase A6, a carboxypeptidase B, a carboxypeptidase D, a carboxypeptidase E, a carboxypeptidase M, a carboxypeptidase N, a carboxypeptidase O, a carboxypeptidase U, a carboxypeptidase X1, a carboxypeptidase X2, a carboxypeptidase Z, a carnosine dipeptidase 1, a carnosine dipeptidase 2, a caspase recruitment domain family, member 8, a caspase, a caspase-1, a caspase-2, a caspase-3, a caspase-4/11, a caspase-5, a caspase-6, a caspase-7, a caspase-8, a caspase-9, a caspase-10, a caspase-12, a caspase-14, a caspase-14-like, a casper/FLIP, a cathepsin, a cathepsin A (CTSA), a cathepsin B (CTSB), a cathepsin C (CTSC), a cathepsin D (CTSD), a cathepsin E (CTSE), a cathepsin F, a cathepsin G, a cathepsin H (CTSH), a cathepsin K (CTSK), a cathepsin L (CTSL), a cathepsin L2, a cathepsin O, a cathepsin S (CTSS), a cathepsin V (CTSV), a cathepsin W, a cathepsin Z (CTSZ), a cationic trypsin, a cezanne/OTU domain containing 7B, a cezanne-2, a CGI-58, a chymase, a chymopasin, a chymosin, a chymotrypsin B, a chymotrypsin C, a coagulation factor IXa, a coagulation factor VIIa, a coagulation factor Xa, a coagulation factor XIa, a coagulation factor XIIa, a collagenase 1, a collagenase 2, a collagenase 3, a complement protease C1r serine protease, a complement protease C1s serine protease, a complement C1r-homolog, a complement component 2, a complement component C1ra, a complement component C1sa, a complement factor B, a complement factor D, a complement factor D-like, a complement factor I, a COPS6, a corin, a CSN5 (JAB1), a cylindromatosis protein, a cytosol alanyl aminopep.-like 1, a cytosol alanyl aminopeptidase, or a combination hereof.

In some embodiments, the protease comprises a DDI-related protease, a DECYSIN, a Der1-like domain family, member 1, a Der1-like domain family, member 2, a Der1-like domain family, member 3, a DESC1 protease, a desert hedgehog protein, a desumoylating isopeptidase 1, a desumoylating isopeptidase 2, a dihydroorotase, a dihydropyrimidinase, a dihydropyrimidinase-related protein 1, a dihydropyrimidinase-related protein 2, a dihydropyrimidinase-related protein 3, a dihydropyrimidinase-related protein 4, a dihydropyrimidinase-related protein 5, a DINE peptidase, a dipeptidyl peptidase (DPP), a dipeptidyl peptidase (DPP1), a dipeptidyl-peptidase 4 (DPP4), a dipeptidyl-peptidase 6 (DPP6), a dipeptidyl-peptidase 8 (DPP8), a dipeptidyl-peptidase 9 (DPP9), a dipeptidyl-peptidase II, a dipeptidyl-peptidase III, a dipeptidyl-peptidase 10 (DPP10), a DJ-1, a DNA-damage inducible protein, a DNA-damage inducible protein 2, a DUB-1, a DUB-2, a DUB2a, a DUB2a-like, a DUB2a-like2, a DUB6, or a combination hereof.

In some embodiments, the protease comprises an enamelysin, an endopeptidase Clp, an endoplasmic reticulum metallopeptidase 1, an endothelin-converting enzyme 1, an endothelin-converting enzyme 2, an enteropeptidase, an epidermis-specific SP-like, an epilysin, an epithelial cell transforming sequence 2 oncogene-like, an epitheliasin, an epoxide hydrolase, an epoxyde hydrolase related protein, an eukar. translation initiation F3SF, an eukar. translation initiation F3SH, or a combination hereof.

In some embodiments, the protease comprises a Factor VII activating protease, a FACE-1/ZMPSTE24, a FACE-2/RCE1, a family with sequence similarity 108, member A1, a family with sequence similarity 108, member B1, a family with sequence similarity 108, member C1, a family with sequence similarity 111, A, a family with sequence similarity 111, B, a furin, or a combination hereof.

In some embodiments, the protease comprises a gamma-glutamyl hydrolase, a gamma-glutamyltransferase 1, a gamma-glutamyltransferase 2, a gamma-glutamyltransferase 5, a gamma-glutamyltransferase 6, a gamma-glutamyltransferase m-3, a gamma-glutamyltransferase-like 3, a GCDFP15, a gelatinase A, a gelatinase B, a Gln-fructose-6-P transamidase 1, a Gln-fructose-6-P transamidase 2, a Gln-fructose-6-P transamidase 3, a Gln-PRPP amidotransferase, a glutamate carboxypeptidase II, a glutaminyl cyclase, a glutaminyl cyclase 2, a glycosylasparaginase, a glycosylasparaginase-2, a granzyme, a granzyme A, a granzyme B, a granzyme H, a granzyme K, a granzyme M, a haptoglobin-1, or a combination hereof.

In some embodiments, the protease comprises a histone deacetylase (HDAC), a haptoglobin-related protein, a HAT-like 2, a HAT-like 3, a HAT-like 4, a HAT-like 5, a HAT-related protease, HSP90AA1? (a heat shock 90 kDa protein 1, alpha), HSP90AB1? (a heat shock 90 kDa protein 1, beta), a heat shock protein 75, a heat shock protein 90 kDa beta (Grp94), member 1/tumor rejection antigen (gp96), a hepatocyte growth factor, a hepsin, a HetF-like, a HGF activator, a hGPI8, a Hin-1/OTU domain containing 4, a homologue ICEY, a HP43.8 KD, a HTRA1 serine protease, a HTRA2, a HTRA3, a HTRA4, a hyaluronan-binding ser-protease, a implantation serine protease 2, a indian hedgehog protein, a insulysin, a intestinal serine protease 1, a josephin-1, a josephin-2, or a combination hereof.

In some embodiments, the protease comprises a Kallikrein (KLK), a kallikrein hK1, a kallikrein hK2, a kallikrein hK3, a kallikrein hK4, a kallikrein hK5, a kallikrein hK6, a kallikrein hK7, a kallikrein hK8, a kallikrein hK9, a kallikrein hK10, a kallikrein hK11, a kallikrein hK12, a kallikrein hK13, a kallikrein hK14, a kallikrein hK15, a Kell blood-group protein, a KHNYN KH and NYN domain containing, a lactotransferrin, a legumain, a leishmanolysin-2, a leucyl aminopeptidase, a leucyl-cystinyl aminopeptidase, a leukotriene A4 hydrolase, a lysosomal carboxypeptidase A, a lysosomal Pro-X C-peptidase, or a combination hereof.

In some embodiments, the protease comprises a membrane metallo-endopeptidase (MME), a macrophage elastase, a macrophage-stimulating protein, a mammalian tolloid-like 1 protein, a mammalian tolloid-like 2 protein, a MAP1D methione aminopeptidase 1D, a marapsin, a marapsin 2, a MASP1/3 (a MBL associated serine protease 3), a MBL associated serine protease 2 (MASP2), a mastin, a matrilysin, a matrilysin-2, a matriptase, a matriptase-2, a matriptase-3, a membrane dipeptidase, a membrane dipeptidase 2, a membrane dipeptidase 3, a membrane-type mosaic Ser-protein, a meprin alpha subunit, a meprin beta subunit, a mesoderm-specific transcript, a mesotrypsin, a methionyl aminopeptidase I, a methionyl aminopeptidase II, a methionyl aminopeptidase II-like, a mitochondrial inner membrane protease 2, a mitochondrial Intermediate peptidase, a mitochondrial Proc. peptidase b-subunit, a mitochondrial proc. protease, a mitochondrial signal peptidase, a matrix metalloproteinase (MMP), a MMP19, a MMP21, a MMP23A, a MMP23B, a MMP27, a MPND, a MT1-MMP, a MT2-MMP, a MT3-MMP, a MT4-MMP, a MT5-MMP, a MT6-MMP, a MYSM1, or a combination hereof.

In some embodiments, the protease comprises a NAALADASE II, a NAALADASE like 2, a NAALADASE like1, a napsin A, a napsin B, a nardilysin, a nasal embryonic LHRH factor, a NEDD4 binding protein 1, a neprilysin, a neprilysin-2, a neurolysin, a neurotrypsin, a neutrophil elastase (ELANE, ELA2), a NLRP1 self-cleaving protein, a nuclear recept. interacting protein 2, a nuclear recept. interacting protein 3, a nucleoporin 98, a NYN domain and retroviral integrase containing, a NY-REN-60, an OMA1, an O-sialoglycoprotein endopeptidase, an O-sialoglycoprotein endopeptidase like 1, an osteoblast serine protease, an OTU domain containing 6B, an OTU domain containing-1, an OTU domain containing-3, an OTU domain containing-5, an OTU domain containing-6A, an otubain-1, an otubain-2, an OTUD2/YOD1, an ovastacin, an oviductin-like/ovochymase-2, an ovochymase-like, or a combination hereof.

In some embodiments, the protease comprises a proteinase 3 (PRTN3), a papain, a PACE4 proprotein convertase, a pancreatic elastase, a pancreatic elastase II (IIA), a pancreatic elastase II form B, a pancreatic endopeptidase E (A), a pancreatic endopeptidase E (B), a pappalysin-1, a pappalysin-2, a paracaspase, a paraplegin, a pepsin A, a pepsin C, a PHEX endopeptidase, a PIDD auto-processing protein unit 1, a PIM1 endopeptidase, a PIM2 endopeptidase, a pitrilysin metalloproteinase 1, a plasma Glu-carboxypeptidase, a plasma kallikrein, a plasma-kallikrein-like 2, a plasma-kallikrein-like 3, a plasma-kallikrein-like 4, a plasmin (plasminogen), a PM20D2 peptidase, a POH1/PSMD14, a polyserase-2, a polyserase-3, a polyserase-I, a Ppnx, a presenilin 1, a presenilin 2, a presenilin homolog 1/SPPL3, a presenilin homolog 2, a presenilin homolog 3/SPP, a presenilin homolog 4/SPPL2B, a presenilin homolog 5, a presenilins-assoc. rhomboid like, a procollagen C-proteinase, a proliferation-association protein 1, a prolyl oligopeptidase, a prolyl oligopeptidase-like, a proprotein convertase 1, a proprotein convertase 2, a proprotein convertase 4, a proprotein convertase 5, a proprotein convertase 7, a proprotein convertase 9 (a proprotein convertase subtilisin/kexin type 9, PCSK9), a prostasin, (a protease, serine, 56), a proteasome alpha 1 subunit, a proteasome alpha 2 subunit, a proteasome alpha 3 subunit, a proteasome alpha 3-like subunit, a proteasome alpha 4 subunit, a proteasome alpha 5 subunit, a proteasome alpha 6 subunit, a proteasome alpha 7 subunit, a proteasome alpha 8 subunit, a proteasome b subunit LMP7-like, a proteasome beta 1 subunit, a proteasome beta 2 subunit, a proteasome beta 3 subunit, a proteasome beta 3-like subunit, a proteasome beta 4 subunit, a proteasome catalytic sub. 1-like, a proteasome catalytic subunit 1, a proteasome catalytic subunit 1i, a proteasome catalytic subunit 2, a proteasome catalytic subunit 2i, a proteasome catalytic subunit 3, a proteasome catalytic subunit 3i, a protein C, a protein C-like, a protein Z, a proteinase 3, a PRPF8, a PSMD7, a pyroglutamyl-peptidase I, a pyroglutamyl-peptidase II, or a combination hereof.

In some embodiments, the protease comprises a reelin, a renin, a retinol binding protein 3, a rhomboid 5 homolog 1, a rhomboid 5 homolog 2, a rhomboid domain containing 1, a rhomboid domain containing 2, a rhomboid, veinlet-like 2, a rhomboid, einlet-like 3, a rhomboid-like protein 1, or a combination hereof.

In some embodiments, the protease comprises a serine protease, a serine protease 3 (PRSS3), a S2P protease, a SAD1, a secernin-1, a secernin-2, a secernin-3, a sentrin (SUMO protease 1), a sentrin (SUMO protease 2), a sentrin (SUMO protease 3), a sentrin (SUMO protease 5), a sentrin (SUMO protease 5-like 1), a sentrin (SUMO protease 6), a sentrin (SUMO protease 7), a sentrin (SUMO protease 8), a sentrin (SUMO protease 9), a sentrin (SUMO protease 11), a sentrin (SUMO protease 12), a sentrin (SUMO protease 13), a sentrin (SUMO protease 14), a sentrin (SUMO protease 15), a sentrin (SUMO protease 16), a sentrin (SUMO protease 17), a sentrin (SUMO protease 18), a sentrin (SUMO protease 19), a separase, a seprase, a serine carboxypeptidase 1, a signalase 18 kDa component, a signalase 21 kDa component, a signalase-like 1, a similar to Arabidopsis Ser-prot., a similar to SPUVE, a site-1 protease, a sonic hedgehog protein, a spinesin, a SprT-like N-terminal domain, a stromelysin 1, a stromelysin 2, a stromelysin 3, a suppressor of Ty 16 homolog, or a combination hereof.

In some embodiments, the protease comprises a taspase, a TBP-associated factor 2, a TESP2, a TESP3, a testase 2, a testis serine protease 2, a testis serine protease 3, a testis serine protease 4, a testis serine protease 5, a testis serine protease 6, a testisin, a testis-specific protein tsp50, a thimet oligopeptidase, a thrombin, a thymus-specific serine peptidase, a TINAG related protein, a TMPRSS11A, a t-plasminogen activator, a TRAF-binding protein domain, a transferrin receptor 2 protein, a transferrin receptor protein, a transmembrane Ser-protease 3, a transmembrane Ser-protease 4, a transthyretin, a TRH-degrading ectoenzyme, a tripeptidyl-peptidase I, a tripeptidyl-peptidase II, a trypsin, a trypsin 10, a trypsin 15, a trypsin C, a trypsin X2, a tryptase, a tryptase alpha/beta 1, a tryptase beta 2, a tryptase delta 1, a tryptase gamma 1, a tryptase homolog 2/EOS, a tryptase homolog 3, a tubulointerstitial nephritis antigen, or a combination hereof.

In some embodiments, the protease comprises a ubiquitin C-term. hydrolase BAP1, a ubiquitin C-terminal hydrolase 1, a ubiquitin C-terminal hydrolase 3, a ubiquitin C-terminal hydrolase 4, a ubiquitin C-terminal hydrolase 5, a ubiquitin specific peptidase like 1, a UCR1, a UCR2, a UDP-N-acetylglucosaminyltransferase subunit, a Ufm-1 specific protease 1, a Ufm-1 specific protease 2, a urokinase (PLAU, uPA) a umbelical vein proteinase, a u-plasminogen activator, a USP1, a USP2, a USP3, a USP4, a USP5, a USP6, a USP7, a USP8, a USP9X, a USP9Y, a USP10, a USP11, a USP12, a USP13, a USP14, a USP15, a USP16, a USP17, a USP17-like, a USP18, a USP19, a USP20, a USP21, a USP22, a USP24, a USP25, a USP26, a USP27, a USP28, a USP29, a USP30, a USP31, a USP34, a USP35, a USP36, a USP37, a USP40, a USP41, a USP42, a USP43, a USP44, a USP45, a USP46, a USP47, a USP48, a USP49, a USP50, a USP51, a USP52, a USP53, a USP54, or a combination hereof.

In some embodiments, the protease comprises a VCP (p97)/p47-interacting protein, a VDU1, a vitellogenic carboxypeptidase-L, a X-Pro dipeptidase, a X-prolyl aminopeptidase 2, a YME1-like 1, a zinc finger CCCH-type containing 12A, a zinc finger CCCH-type containing 12B, a zinc finger CCCH-type containing 12C, a zinc finger CCCH-type containing 12D, a Zinc finger containing ubiquitin peptidase 1, or a combination hereof.

In some embodiments, the protease comprises an A20 (Tumor necrosis factor, alpha-induced protein 3, TNF a-induced protein 3). A20 is a zinc finger protein and a deubiquitinating enzyme. A20 has been shown to inhibit NF-kappa B activation as well as TNF-mediated apoptosis, limit inflammation.

In some embodiments, the protease comprises an Angiotensin-converting enzyme 2 (ACE2). ACE2 is an enzyme attached to the membrane cells located to the membrane of cells located in the intestines, kidney, testis, gallbladder, and heart. ACE2 counters the activity of the related angiotensin-converting enzyme, ACE, by reducing the amount of angiostatin II.

In some embodiments, the protease comprises a cathepsin. The cathepsin may be, but is not limited to, a cathepsin A (CTSA), a cathepsin B (CTSB), a cathepsin C (CTSC), a cathepsin D (CTSD), a cathepsin E (CTSE), a cathepsin H (CTSH), a cathepsin K (CTSK), a cathepsin L (CTSL), a cathepsin S (CTSS), a cathepsin V (CTSV), and a cathepsin Z (CTSZ). Cathepsins are a subset of proteases, many of which become activated in low pH. Cathepsins comprise serine proteases, cysteine proteases, and aspartyl proteases, among others. Cathepsins have been implicated in cancer, Alzheimer's disease, arthritis, Ebola, pancreatitis, glaucoma, COPD, and other diseases.

In some embodiments, the protease comprises a caspase. The caspase may be, but is not limited to, a caspase 1, a caspase 2, a caspase 3, a caspase 4, a caspase 5, a caspase 6, a caspase 7, a caspase 8, a caspase 9, a caspase 10, a caspase 11, a caspase 12, a caspase 13, and a caspase 14.

In some embodiments, the protease comprises a calpain. The calpain may be, but is not limited to a calpain 1, a calpain 2, a calpain 3, a calpain 4, a calpain 5, a calpain 6, a calpain 7, a calpain 8, a calpain 9, a calpain 10, a calpain 11, a calpain 12, a calpain 13, a calpain 14, and a calpain 15. Caspases are a family of protease enzymes that play essential roles in programmed cell death and cell homeostasis.

In some embodiments, the protease comprises a cysteine protease. Cysteine proteases, also known as thiol proteases, are hydrolase enzymes that degrade proteins. These proteases share a common catalytic mechanism that involves a nucleophilic cysteine thiol in a catalytic triad or dyad. The cysteine protease family comprises Papain (Carica papaya), bromelain (Ananas comosus), cathepsin K (liverwort), calpain (Homo sapiens), aspase-1 (Rattus norvegicus), separase (Saccharomyces cerevisiae), Adenain (human adenovirus type 2), Pyroglutamyl-peptidase I (Bacillus amyloliquefaciens), Sortase A (Staphylococcus aureus), Hepatitis C virus peptidase 2 (hepatitis C virus), Sindbis virus-type nsP2 peptidase (sindbis virus), Dipeptidyl-peptidase VI (Lysinibacillus sphaericus), DeSI-1 peptidase (Mus musculus), TEV protease (tobacco etch virus), Amidophosphoribosyltransferase precursor (Homo sapiens), Gamma-glutamyl hydrolase (Rattus norvegicus), Hedgehog protein (Drosophila melanogaster) and DmpA aminopeptidase (Ochrobactrum anthropi), etc.

In some embodiments, the protease comprises a complement C1r serine protease (Complement component 1r). In some embodiments, the protease comprises a complement C1s serine protease (Complement component 1s). C1r along with C1q and C1s form the C1 complex. C1r has very narrow trypsin-like specificity that is responsible for activation of the C1 complex. C1 activation is a two-step process involving (1) C1r intramolecular autoactivation and (2) C1s cleavage by activated C1r. C1r contains a chymotrypsin-like serine protease domain at its C-terminal, and cleaves a single Arg-Ile bond in C1r and in C1s. Zvi Fishelson, in xPharm: The Comprehensive Pharmacology Reference, 2007.

In some embodiments, the protease comprises a chymotrypsin (chymotrypsins A and B, alpha-chymar ophth, avazyme, chymar, chymotest, enzeon, quimar, quimotrase, alpha-chymar, alpha-chymotrypsin A, alpha-chymotrypsin)). Chymotrypsin is a digestive enzyme component of pancreatic juice acting in the duodenum, where it performs proteolysis, the breakdown of proteins and polypeptides. Chymotrypsin preferentially cleaves peptide amide bonds where the side chain of the amino acid N-terminal to the scissile amide bond is a large hydrophobic amino acid (tyrosine, tryptophan, and phenylalanine).

In some embodiments, the protease comprises a chymase (mast cell protease 1, skeletal muscle protease, skin chymotryptic proteinase, mast cell serine proteinase, skeletal muscle protease). Chymases are a family of serine proteases found in mast cells, basophil granulocytes. Chymases show broad peptidolytic activity and are involved in inflammatory response, hypertension and atherosclerosis.

In some embodiments, the protease comprises a dipeptidyl peptidase (DPP). DPP comprises cathepsin C (DPP1), DPP2, DPP3, DPP4, DPP 6, DPP7, DPP8, DPP9, DPP10.

In some embodiments, the protease comprises a DPP4 (adenosine deaminase complexing protein 2, CD26). DPP4 is expressed on cell surface and is associated with immune regulation, signal transduction, and apoptosis. DPP4 is a serine exopeptidase that cleaves X-proline or X-alanine dipeptides from the N-terminus of polypeptides. DPP-4 is known to cleave a broad range of substrates including growth factors, chemokines, neuropeptides, and vasoactive peptides. DPP4 plays a major role in glucose metabolism, is responsible for the degradation of incretins such as GLP-1, and appears to work as a suppressor in the development of some tumors.

In some embodiments, the protease comprises a DPP1 (Cathepsin C, CTSC). DPP1 is a lysosomal exo-cysteine protease belonging to the peptidase C1 family. Cathepsin C appears to be a central coordinator for activation of many serine proteases in immune/inflammatory cells. Cathepsin C catalyzes excision of dipeptides from the N-terminus of protein and peptide substrates.

In some embodiments, the protease comprises a disintegrin and metalloproteinase (ADAM). ADAMs are a family of single-pass transmembrane and secreted metalloendopeptidases. Not all human ADAMs have a functional protease domain. Those ADAMs which are active proteases are classified as sheddases because they cut off or shed extracellular portions of transmembrane proteins.

In some embodiments, the protease comprises an ADAM12 metalloprotease. ADAM12 binds insulin growth factor binding protein-3 (IGFBP-3), appears to be an early Down syndrome marker, and has been implicated in a variety of biological processes involving cell-cell and cell-matrix interactions, including fertilization, muscle development, and neurogenesis.

In some embodiments, the protease comprises a disintegrin and metalloproteinase with thrombospondin motifs (ADAMTS). ADAMTS is a family of multidomain extracellular protease enzymes, comprising ADAMTS1, ADAMTS2, ADAMTS3, ADAMTS4, ADAMTS5 (=ADAMTS11), ADAMTS6, ADAMTS7, ADAMTS8 (or METH-2), ADAMTS9, ADAMTS10, ADAMTS12, ADAMTS13, ADAMTS14, ADAMTS15, ADAMTS16, ADAMTS17, ADAMTS18, ADAMTS19, and ADAMTS20. Known functions of the ADAMTS proteases include processing of procollagens and von Willebrand factor as well as cleavage of aggrecan, versican, brevican and neurocan, making them key remodeling enzymes of the extracellular matrix. They have been demonstrated to have important roles in connective tissue organization, coagulation, inflammation, arthritis, angiogenesis and cell migration.

In some embodiments, the protease comprises an ADAMTS1. ADAMTS1 is a member of the ADAMTS protein family. The expression of ADAMTS1 may be associated with various inflammatory processes, development of cancer cachexia, normal growth, fertility, and organ morphology and function.

In some embodiments, the protease comprises a Factor VII activating protease (FSAP). FSAP is a circulating serine protease with high homology to fibrinolytic enzymes, and may be associated with the regulation of coagulation and fibrinolysis.

In some embodiments, the protease comprises a furin. Furin belongs to the subtilisin-like proprotein convertase family, and is a calcium-dependent serine endoprotease. Furin's substrates includes: proparathyroid hormone, transforming growth factor beta 1 precursor, proalbumin, pro-beta-secretase, membrane type-1 matrix metalloproteinase, beta subunit of pro-nerve growth factor and von Willebrand factor.

In some embodiments, the protease comprises a histone deacetylase (HDAC). HDACs are a class of enzymes that remove acetyl groups (O═C—CH3) from an ε-N-acetyl lysine amino acid on a histone, allowing the histones to wrap the DNA more tightly.

In some embodiments, the protease comprises a HTRA1 serine protease. HTRA1 is a secreted enzyme that is proposed to regulate the availability of insulin-like growth factors (IGFs) by cleaving IGF-binding proteins. It has also been suggested to be a regulator of cell growth.

In some embodiments, the protease comprises a granzyme. Granzymes are serine proteases released by cytoplasmic granules within cytotoxic T cells and natural killer (NK) cells. Granzymes induce programmed cell death in the target cell. Granzymes also kill bacteria and inhibit viral replication.

In some embodiments, the protease comprises, a Kallikrein (KLK). Kallikreins are a subgroup of serine proteases. Kallikreins are responsible for the coordination of various physiological functions including blood pressure, semen liquefaction and skin desquamation.

In some embodiments, the protease comprises a matrix metalloproteinase (MMP, matrix metallopeptidases, matrixins). MPPs are calcium-dependent zinc-containing endopeptidases. MMPs have been implicated in cleavage of cell surface receptors, the release of apoptotic ligands, chemokine/cytokine inactivation, cell proliferation and cell migration.

In some embodiments, the protease comprises a membrane metallo-endopeptidase (MME). MME is a zinc-dependent metalloprotease that cleaves peptides at the amino side of hydrophobic residues and inactivates several peptide hormones including glucagon, enkephalins, substance P, neurotensin, oxytocin, and bradykinin. MME is expressed in a wide variety of tissues and is particularly abundant in kidney. MME is also a common acute lymphocytic leukemia antigen.

In some embodiments, the protease comprises a mannose-binding protein-associated serine protease 2 (MASP2, Mannan-binding lectin serine protease 2, MBL associated serine protease 2). MASP2 is involved in the complement system, cleaves complement components C4 and C2 into C4a, C4b, C2a, and C2b.

In some embodiments, the protease comprises a mannose-binding protein-associated serine protease 3 (MBL associated serine protease 3, MASP3). MASP3 originates from the MASP1 gene through differential splicing, it circulates in high serum concentrations predominantly in complex with Ficolin-3 and regulates Ficolin-3 mediated complement activation.

In some embodiments, the protease comprises a neutrophil elastase (ELANE, ELA2). ELANE is a serine proteinase secreted by neutrophils and microphages during inflammation and destroys bacteria and host tissue.

In some embodiments, the protease comprises a proteinase 3 (PRTN3). PRTN3 is a serine protease enzyme expressed mainly in neutrophil granulocytes and contributes to the proteolytic generation of antimicrobial peptides.

In some embodiments, the protease comprises a plasmin (a.k.a. plasminogen). Plasmin is a proteolytic enzyme derived from an inert plasma precursor known as plasminogen. It is present in blood that degrades many blood plasma proteins, including fibrin clots. In human, plasmin is encoded by PLG gene.

In some embodiments, the protease comprises a pepsin. Pepsin is an endopeptidase that cleaves proteins into smaller peptides. It is an aspartic protease, using a catalytic aspartate in its active site.

In some embodiments, the protease comprises a presenilin-1 (PS-1). PS-1 is a presenilin protein that is one of the four core proteins in the gamma secretase complex, which is considered to play an important role in generation of amyloid beta from amyloid precursor protein.

In some embodiments, the protease comprises a proprotein convertase subtilisin/kexin type 9 (PCSK9). PCSK9 is a member of the peptidase S8 family.

In some embodiments, the protease comprises a serine protease. Serine protease cleaves peptide bonds in proteins, in which serine serves as the nucleophilic amino acid at the enzyme's active site. Serine protease includes many subfamilies.

In some embodiments, the protease comprises a tryptase. Tryptase is a the most abundant secretory granule-derived serine proteinase contained in mast cells and has been used as aa marker for mast cell activation. It is released from mask cells when they are activated as part of a normal immune response as well as in allergic responses.

In some embodiments, the protease comprises, a trypsin. Trypsin is a serine protease from the PA clan superfamily, found in the digestive system. Trypsin cuts peptide chains mainly at the carboxyl side of the amino acids lysine or arginine.

In some embodiments, the protease comprises a urokinase (PLAU, uPA). Urokinase is a serine protease present in humans and other animals. It is present in human urine, blood and in the extracellular matrix of many tissues. It is involved in degradation of the extracellular matrix and possibly tumor cell migration and proliferation. Urokinase is a 411-residue protein, consisting of three domains: the serine protease domain, the kringle domain, and the EGF-like domain. Urokinase is synthesized as a zymogen form (prourokinase or single-chain urokinase), and is activated by proteolytic cleavage between Lys158 and Ile159. The two resulting chains are kept together by a disulfide bond.

In some embodiments, the protease is a T cell protease, a complement protease, a fibrosis protease, or an inflammation-related protease.

Described herein are agents to be detected including but are not limited to a oxidoreductase, a transferase, a hydrolase, a lyase, a isomerase, a ligase, a protease, a hydrolase, an esterase, a β-glycosidase, a phospholipase and a phosphodiesterase, peroxidase, lipase, amylase a nucleophilic reagent, a reducing reagent, a electrophilic/acidic reagent, an organometallic/metal catalyst, an oxidizing reagent, a hydroxyl ion, a thiols nucleophile, a nitrogen nucleophile, a sodium dithionite and a sodium periodate.

As described herein, the activity detection of some agents does not rely on cleavage. For example, some oxidoreductases, transferases, hydrolases, lyases, isomerases, and ligases lead to the substrate linker modification and release or formation of a reporter molecule that can be detected. As a way of illustration, a certain oxidation processes can modify an inactive fluorophore and render it fluorescent/detectable without the need of a substrate linker or binding events (for non-covalent processes) can change magnetic/fluorescent physical-chemical properties of certain reporters and render them detectable.

### Disease and Condition

The methods described herein comprise determining a disease or condition of the subject. In some aspects, the disease or condition comprises a liver disease, a cancer, a metabolic disease, a fibrotic disease, an organ transplant rejection, an infectious disease, an allergic disease, an autoimmunity, Alzheimer's, a chronic inflammation, neurologic disease or any other protease related disease. In some embodiments, the liver disease may be a non-alcoholic steatohepatitis (NASH), a non-alcoholic fatty liver disease (NAFLD), a toxin mediated liver injury (drug/medication, alcohol, environmental), a viral hepatitis (HAV, HBV, HCV, HDV, HEV, other virus infecting the liver), an autoimmune hepatitis, a primary biliary cholangitis, a primary sclerosing cholangitis, a fulminant hepatitis, a cirrhosis of the liver, a hepatocellular carcinoma (HCC), a cholangiocarcinoma, an acute or chronic rejection of a transplanted liver, an inherited liver disease (e.g. Wilson disease, hemochromatosis, or alpha-1 antitrypsin) or a combination thereof.

In some embodiments, the cancer comprises adenoid cystic carcinoma, adrenal gland tumors, amyloidosis, anal cancer, appendix cancer, astrocytoma, ataxia-telangiectasia, Beckwith-Wiedemann syndrome, bile duct cancer (cholangiocarcinoma), Birt-Hogg-Dubé Syndrome, bladder cancer, bone cancer (sarcoma of the bone), brain stem glioma, brain tumors, breast cancer, Carney complex, central nervous system tumors, cervical cancer, colorectal cancer, Cowden Syndrome, craniopharyngioma, Desmoid tumors, desmoplastic infantile ganglioglioma, ependymoma, esophageal cancer, Ewing sarcoma, eye cancer, eyelid cancer, familial adenomatous polyposis, familial GIST, familial malignant melanoma, familial pancreatic cancer, gallbladder cancer, gastrointestinal stromal tumors (GIST), germ cell tumors, gestational trophoblastic disease, head and neck cancer, breast and ovarian cancer, diffuse gastric cancer, leiomyosarcoma and renal cell cancer, mixed polyposis syndrome, papillary renal carcinoma, juvenile polyposis syndrome, kidney cancer, lacrimal gland tumors, laryngeal and hypopharyngeal cancer, leukemia, myeloid leukemia, lymphoblastic leukemia, eosinophilic leukemia, Li-Fraumeni syndrome, liver cancer, lung cancer, Hodgkin lung cancer, non-Hodgkin lung cancer, Lynch syndrome, mastocytosis, medulloblastoma, melanoma, meningioma, mesothelioma, multiple endocrine neoplasia, multiple myeloma, myelodysplastic syndrome, nasal cavity and paranasal sinus cancer, nasopharyngeal cancer, neuroblastoma, neuroendocrine tumors, neurofibromatosis, nevoid basal cell carcinoma syndrome, oral and oropharyngeal cancer, osteosarcoma, ovarian cancer, fallopian tube cancer, peritoneal cancer, pancreatic cancer, parathyroid cancer, penile cancer, Peutz-Jeghers syndrome, phenochromocytoma, paraganglioma, pituitary gland tumors, pleuropulmonary blastoma, prostate cancer, retinoblastoma, rhabdomyosarcoma, salivary gland cancer, Kaposi sarcoma, soft tissue sarcoma, sarcoma, non-melanoma skin cancer, small bowel cancer, stomach cancer, testicular cancer, thymoma and thymic carcinoma, thyroid cancer, tuberous sclerosis complex, uterine cancer, vaginal cancer, von Hippel-Lindau syndrome, vulvar cancer, Waldenstrom macroglobulinemia, Werner syndrome, Wilms tumors, or xeroderma pigmentosum.

In some embodiments, the disease may be NASH. Non-alcoholic steatohepatitis, also called NASH, is a more active inflammatory form of non-alcoholic fatty liver disease (NAFLD). NAFLD is caused by buildup of fat in the liver. When this buildup causes inflammation and damage, it is known as NASH, which can lead to scarring of the liver. There are often no outward signs or symptoms associated with NASH, although the most common symptoms are fatigue or mild pain in the upper right abdomen. NASH may lead to cirrhosis of the liver, causing one or more of the following symptoms as the condition progresses: bleeding easily, bruising easily, itchy skin, jaundice, abdominal fluid accumulation, loss of appetite, nausea, leg swelling, confusion, drowsiness, slurred speech, or spider-like blood vessels.

NASH is most common in patients who are overweight or obese; other risk factors include diabetes, high cholesterol, high triglycerides, poor diet, metabolic syndrome, polycystic ovary syndrome, sleep apnea, and hyperthyroidism.

NAFLD encompasses the entire spectrum of fatty liver disease in individuals without significant alcohol consumption, ranging from fatty liver to steatohepatitis to cirrhosis. Non-alcoholic fatty liver is the presence of >5% hepatic steatosis without evidence of hepatocellular injury in the form of ballooning of the hepatocytes or evidence of fibrosis. The risk of progression to cirrhosis and liver failure is considered minimal. NASH is the presence of >5% hepatic steatosis with inflammation and hepatocyte injury (ballooning) with or without fibrosis. This can progress to cirrhosis, liver failure, and rarely liver cancer. NASH cirrhosis is presence of cirrhosis with current or previous histological evidence of steatosis or steatohepatitis.

NAS is an unweighted composite of steatosis, lobular inflammation, and ballooning scores. NAS is a useful tool to measure changes in liver histology in patients with NAFLD/NASH and higher risk of liver cancer in clinical trials. Fibrosis is scored separately and can be classified as F1 through F4; specifically, stage 1 is zone 3 (perivenular), perisinusoidal, or periportal fibrosis; stage 2 is both zone 3 and periportal fibrosis; stage 3 is bridging fibrosis with nodularity; and stage 4 is cirrhosis.

TABLE 3
The histological scoring system for nonalcoholic fatty liver disease:
components of NAFLD activity score (NAS) and fibrosis staging.

| | | | |
|---|---|---|---|
| Item | Score | Extent | Definition and Comment |

NAS Components (see scoring interpretation)

| | | | |
|---|---|---|---|
| Steatosis | 0 | <5% | Refers to amount of surface area involved by steatosis |
| | 1 | 5-33% | as evaluated on low to medium power examination. |
| | 2 | >33-66% | |
| | 3 | >66% | |
| Lobular | 0 | No foci | Acidophil bodies are not included in this assessment, |
| Inflammation | 1 | <2 foci/200x | nor is portal inflammation |
| | 2 | 2-4 foci/200x | |
| | 3 | >4 foci/200x | |
| Hepatocyte | 0 | None | |
| Ballooning | 1 | Few ballooned | “Few” means rare but definite ballooned hepatocytes as |
| | | cells | well as cases that are diagnostically borderline |
| | 2 | Many | Most cases with prominent ballooning also had |
| | | cells/prominent | Mallory's hyalin, but Mallory's hyaline is not scored |
| | | ballooning | separately for the NAS |

Fibrosis Stage (Evaluated separately from NAS)

| | | | |
|---|---|---|---|
| Fibrosis | 0 | None | |
| | 1 | Perisinusoidal or | |
| | | periportal | |
| | 1A | Mild, zone 3, | “delicate” fibrosis |
| | | perisinusoidal | |
| | 1B | Moderate, zone 3, | “dense” fibrosis |
| | | perisinusoidal | |
| | 1C | Portal/periportal | This category is included to accommodate cases with |
| | | | portal and/or peri portal fibrosis without accompanying |
| | | | pericellular/perisinusoidal fibrosis |
| | 2 | Perisinusoidal and | |
| | | portal/periportal | |
| | 3 | Bridging fibrosis | |
| | 4 | Cirrhosis | |

Scoring interpretation: Total NAS score represents the sum of scores for steatosis, lobular inflammation, and ballooning, and ranges from 0-8. Diagnosis of NASH (or, alternatively, fatty liver not diagnostic of NASH) should be made first, then NAS is used to grade activity. In the reference study, NAS scores of 0-2 occurred in cases largely considered not diagnostic of NASH, scores of 3-4 were evenly divided among those considered not diagnostic, borderline, or positive for NASH. Scores of 5-8 occurred in cases that were largely considered diagnostic of NASH

In some embodiments, the disease may be NAFLD. Nonalcoholic fatty liver disease (NAFLD) is an umbrella term for a range of liver conditions affecting people who drink little to no alcohol. As the name implies, the main characteristic of NAFLD is too much fat stored in liver cells. There are often no outward signs or symptoms associated with NAFLD, although the most common symptoms are fatigue or mild pain in the upper right abdomen.

In some embodiments, the disease may be fulminant hepatitis. Fulminant hepatitis, or fulminant hepatic failure, is defined as a clinical syndrome of severe liver function impairment, which causes hepatic coma and the decrease in synthesizing capacity of liver. Then they rapidly develop severe, often life-threatening liver failure. This can happen within hours, days, or sometimes weeks. Symptoms of severe liver failure include confusion, extreme irritability, altered consciousness, blood clotting defects, and buildup of fluid in the abdominal cavity and multiorgan system failure.

In some embodiments, the disease may be a hepatocellular carcinoma (HCC). HCC is the most common type of primary liver cancer. HCC occurs most often in people with chronic liver diseases leading to advanced fibrosis or cirrhosis and less commonly in patients with little or no fibrosis. The most common liver diseases associated with HCC are viral hepatitis B or C, alcohol related liver disease and NASH.

In some embodiments, the disease may be a primary biliary cholangitis (PBC). Primary biliary cholangitis, previously called primary biliary cirrhosis, is a chronic disease in which the bile ducts in the liver are slowly destroyed. Bile is a fluid made in the liver. Chronic inflammation in the liver can lead to bile duct damage, irreversible scarring of liver tissue (cirrhosis) and eventually, liver failure. PBC is considered an autoimmune disease, which means the body's immune system is mistakenly attacking healthy cells and tissue. Researchers think a combination of genetic and environmental factors triggers the disease. It usually develops slowly. At this time, there's no cure for primary biliary cholangitis, but medication can slow liver damage, especially if treatment begins early.

In some embodiments, the liver disease may be a toxin mediated liver injury (e.g., from drug/medication, alcohol, environmental). Toxin mediated liver injury is an inflammation of liver in reaction to certain substances, such as alcohol, chemicals, drugs/medication, environmental factors or nutritional supplements. The liver normally removes and breaks down most drugs and chemicals from the bloodstream, which creates byproducts that can damage the liver. Although the liver has a great capacity for regeneration, acute or chronic exposure to toxic substances can cause serious, sometimes irreversible harm.

In some embodiments, the liver disease may be a viral hepatitis (HAV, HBV, HCV, HDV, HEV, other virus infecting the liver). Viral hepatitis is a liver inflammation due to a viral infection. It may present in acute form as a recent infection with relatively rapid onset, or in chronic form. The most common causes of viral hepatitis are the five unrelated hepatotropic viruses hepatitis A, B, C, D, and E. Other viruses can also cause liver inflammation, including cytomegalovirus, Epstein-Barr virus, and yellow fever. There also have been scores of recorded cases of viral hepatitis caused by herpes simplex virus. Viral hepatitis is either transmitted through contaminated food or water (A, E) or via blood and body fluids (B, C). Hepatitis A and hepatitis B can be prevented by vaccination. Effective treatments for hepatitis C are available but costly.

In some embodiments, liver disease detection using a protease panel can be combined with additional markers of liver disease, for example, alpha feto-protein (AFP). AFP, also known as alpha feto-globin, is a major plasma protein produced by the fetus and is thought to be the fetal analog of serum albumin. A sustained increase in AFP serum levels can be a diagnostic marker for chronic liver conditions, for example, HCC.

In some embodiments, the liver disease may be an autoimmune hepatitis. Autoimmune hepatitis is liver inflammation that occurs when the immune system attacks liver cells. The exact cause of autoimmune hepatitis is unclear, but genetic and environmental factors appear to interact over time in triggering the disease. Untreated autoimmune hepatitis can lead to scarring of the liver (cirrhosis) and eventually to liver failure. When diagnosed and treated early, autoimmune hepatitis often can be controlled with drugs that suppress the immune system. A liver transplant may be an option when autoimmune hepatitis does not respond to drug treatments or in cases of advanced liver disease. There are two main forms of autoimmune hepatitis: (1) Type 1 autoimmune hepatitis. Type I autoimmune hepatitis is the most common type and can occur at any age. About half the people with type 1 autoimmune hepatitis have other autoimmune disorders, such as celiac disease, rheumatoid arthritis or ulcerative colitis; (2) Type 2 autoimmune hepatitis. Although adults can develop type 2 autoimmune hepatitis, it's most common in children and young people. Other autoimmune diseases may accompany type 2 autoimmune hepatitis.

In some embodiments, the liver disease may be a primary sclerosing cholangitis. Primary sclerosing cholangitis is a disease of the bile ducts, believed to be immune related injury. In primary sclerosing cholangitis, inflammation causes scars within the bile ducts. These scars make the ducts hard and narrow and gradually cause serious liver damage. A majority of people with primary sclerosing cholangitis also have inflammatory bowel disease, such as ulcerative colitis or Crohn's disease. In most cases of primary sclerosing cholangitis, the disease progresses slowly. It can eventually lead to liver failure, repeated infections, and tumors of the bile duct (cholangiocarcinoma) or liver.

In some embodiments, the liver disease may be a cirrhosis of the liver. Cirrhosis is a late stage of scarring (fibrosis) of the liver caused by many forms of liver diseases and conditions, such as hepatitis and chronic alcoholism. In the process of liver self-repair, scar tissue forms. As cirrhosis progresses, more and more scar tissue forms, making it difficult for the liver to function (decompensated cirrhosis).

In some embodiments, the liver disease may be a cholangiocarcinoma. Cholangiocarcinoma (bile duct cancer) is a type of cancer that forms in the bile ducts. Risk factors for cholangiocarcinoma include primary sclerosing cholangitis (an inflammatory disease of the bile ducts), ulcerative colitis, cirrhosis, hepatitis C, hepatitis B, infection with certain liver flukes, and some congenital liver malformations. Cholangiocarcinoma can be categorized based on the location of the cancer occurs in the bile ducts: intrahepatic cholangiocarcinoma, hilar cholangiocarcinoma, distal cholangiocarcinoma. Cholangiocarcinoma is often diagnosed when it is advanced, making successful treatment difficult to achieve.

In some embodiments, the liver disease may be an inherited liver disease (e.g., Wilson disease, hemochromatosis, or alpha-1 antitrypsin, etc.) Inherited liver diseases are genetic disorders that can cause severe liver disease and other health problems. Wilson disease is a rare inherited disorder that causes copper to accumulate in your liver, brain and other vital organs. Hemochromatosis is a disease in which deposits of iron collect in the liver and other organs. The primary form of hemochromatosis is one of the most common inherited diseases in the U.S. The alpha-1 antitrypsin protein is synthesized mainly in the liver by hepatocytes, secreted into the blood stream, and acts as an inhibitor of neutrophil elastase released primarily in the lung during inflammation. Alpha-1 antitrypsin deficiency is caused when alpha-1 antitrypsin protein is either lacking or exists in lower than normal levels in the blood and is accumulated in the liver.

In some embodiments, the disease may be an organ transplant rejection. Transplant rejection occurs when transplanted tissue is rejected by the recipient's immune system, which destroys the transplanted tissue. Transplant rejection can be lessened by determining the molecular similitude between donor and recipient and by use of immunosuppressant drugs after transplant.

In some embodiments, the disease may be an infectious disease, Infectious diseases are disorders caused by organisms—such as bacteria, viruses, fungi or parasites. Bacteria are one-cell organisms responsible for illnesses such as streptococcal upper respiratory infection, urinary tract infections and tuberculosis. Viruses cause a multitude of diseases ranging from the common cold to AIDS. Many skin diseases, such as ringworm and athlete's foot, are caused by fungi. Other types of fungi can infect the lungs or nervous system. Malaria is caused by a tiny parasite that is transmitted by a mosquito bite. Other parasites may be transmitted to humans from animal feces. In some embodiments, the infectious disease is COVID-19.

In some embodiments, the disease may be an allergic disease. Allergic diseases are caused by allergen-induced unfavorable immune responses initiating various symptoms in different organs, which often cannot be completely controlled by modern medicine. The immunologic basis of allergic diseases is observed in two phases: sensitization and development of memory T and B cell responses, and IgE production and effector functions, which are related to eosinophils, innate lymphoid cells, dendritic cell subsets, epithelial cells and tissue inflammation/injury, epithelial barrier, tissue remodeling and chronicity in asthma, atopic dermatitis (AD) and allergic rhinitis (AR). Different disease phenotypes and endotypes may become apparent with different dominant molecular mechanisms, related biomarkers and responses to biologic therapy. Multiple mechanistic factors are complexly involved in the pathogenesis of allergic inflammations In some embodiments, the disease may be an autoimmune disease/autoimmunity. An autoimmune disease is a condition in which the immune system mistakenly attacks one's own body. Normally, the immune system can tell the difference between foreign cells and one's own cells. In an autoimmune disease, the immune system mistakes part of the body, like the joints or skin, as foreign. It releases proteins called autoantibodies that attack healthy cells. Some autoimmune diseases target only one organ. Type 1 diabetes damages the pancreas. Other diseases, like systemic lupus erythematosus (SLE), affect many different organ systems. In some embodiments, the autoimmune disease may be Rheumatoid arthritis, Crohns disease, Multiple sclerosis (MS) or psoriatic arthritis (PsA).

In some embodiments, the disease may be a chronic inflammation. Chronic inflammation is also referred to as slow or fluctuating, long-term inflammation lasting for prolonged periods of several months to years. Generally, the extent and effects of chronic inflammation vary with the cause of the injury and the ability of the body to repair and overcome the damage. Most of the features of acute inflammation continue as the inflammation becomes chronic, including the expansion of blood vessels (vasodilation), increase in blood flow, capillary permeability and migration of neutrophils into the infected tissue through the capillary wall (diapedesis). However, the composition of blood the white cells changes soon and the macrophages and lymphocytes begin to replace short-lived neutrophils. Thus the hallmarks of chronic inflammation are the infiltration of the primary inflammatory cells such as macrophages, lymphocytes, and plasma cells in the tissue site, producing inflammatory cytokines, growth factors, enzymes and hence contributing to the progression of tissue damage and secondary repair including fibrosis and granuloma formation, etc.

In some embodiments, the disease may be a fibrotic disease. Fibrotic disease is defined by the overgrowth, hardening, and/or scarring of various tissues and is attributed to excess deposition of extracellular matrix components including collagen. Fibrosis is the end result of chronic inflammatory reactions induced by a variety of stimuli including persistent infections, autoimmune reactions, allergic responses, chemical insults, radiation, and tissue injury. The fibrotic disorders include but are not limited to systemic fibrotic diseases such as systemic sclerosis (SSc), sclerodermatous graft vs. host disease, idiopathic pulmonary fibrosis (IPF), nephrogenic systemic fibrosis, and organ-specific disorders including radiation-induced fibrosis and cardiac, pulmonary, liver, and kidney fibrosis.

In some embodiments, the disease may be a metabolic disease. A metabolic disorder/disease occurs when abnormal chemical reactions in the body disrupt metabolism. When this happens, one might have too much of some substances or too little of other ones that an individual needs to stay healthy. There are different groups of disorders. Some affect the breakdown of amino acids, carbohydrates, or lipids. Another group, mitochondrial diseases, affects the parts of the cells that produce the energy. one can develop a metabolic disorder when some organs, such as the liver or pancreas, become diseased or do not function normally. Diabetes is an example.

In some embodiments, the disease may be Alzheimer's. Alzheimer's is a type of dementia that affects memory, thinking and behavior. Symptoms eventually grow severe enough to interfere with daily tasks. Alzheimer's changes typically begin in the part of the brain that affects learning. As Alzheimer's advances through the brain, it leads to increasingly severe symptoms, including disorientation, mood and behavior changes; deepening confusion about events, time and place; unfounded suspicions about family, friends and professional caregivers; more serious memory loss and behavior changes; and difficulty speaking, swallowing and walking.

### Algorithms

In some embodiments, the method described herein can be used in a multiplexed format, such that a single body fluid sample can be used to ascertain the activity of multiple, select agents. This allows diagnostic panels to be created for specific pathologies and conditions, which leverage the activity of multiple agents to provide a more complete and accurate assessment of a certain condition. These panels can be used to correlate the activity of multiple agents with a particular condition or disease-state. These signatures can be saved, for example, in a database and used to assess the conditions or disease-state for subsequent individuals assessed by a particular protease activity panel.

In some embodiments, a classification tool is used in the analysis to differentiate between healthy and diseased patients, or between discrete stages of disease. The classification tool may be a supervised or unsupervised Machine Learning or Deep Learning algorithm, Logistic Regression, Naive Bayes, Support Vector Machine, Decision Tree, Random Forest, Gradient Boosting, Regularizing Gradient Boosting, K-Nearest Neighbors, Bayesian Network, a continuous regression approach, Ridge Regression, Kernel Ridge Regression, Least Absolute Shrinkage and Selection Operator (LASSO), Elastic Nets, Support Vector Regression, deep learning approach, Neural Networks, Convolutional Neural Network (CNNs), Recurrent Neural Networks (RNNs), Long Short Term Memory Networks (LSTMs), Restricted Boltzmann Machines (RBMs), Generative Models, Generative Adversarial Networks (GANs), Deep Belief Networks (DBNs), Feedforward Neural Networks, Autoencoders, Radial Basis Function Networks (RBFNs), Multilayer Perceptrons (MLPs), Stochastic Neural Networks or any combination thereof.

In some embodiments, the method will detect information from probe measurements (e.g. the rate of formation of a reporter, the amount of a reporter), wherein the information can be input into a plurality of algorithms. These algorithms can operate on the multi-dimensional feature space defined by the measurements of multiple probes (or a mathematical function of those measurements such as probe ratios) in order to learn the relationship between probe measurements and disease status. Alternatively, or in addition to, alternate parameters can be input into the plurality of algorithms. For example, alternative parameters (e.g., age, gender, patients' comorbid status) can be combined with probe measurements. In that case, one could either incorporate clinical features in the classifier directly or, alternatively, learn a second-order classifier which combines a probe-only prediction with clinical features to produce a result that is calibrated for those variables.

### Other Parameters

In some embodiments, the information detected from probe measurements will be multiplexed with alternate parameters that do not come from the detection. Such parameters may be any physical or clinical characteristic related to the subject, and can include, but are not limited to results of clinical tests, clinical characteristics, genetic profiles, molecular profiles, digital profiles, and demographic characteristics. The probe measurement can be multiplexed with at least about 1, 2, 3, 4, 5, 6, 7, 8, 9, 10, 15, 20, 30, 40, 50, 60, 70, 80, 90, 100, or more parameters.

Clinical tests, also known as medical tests, are medical procedures performed to detect, diagnose, or monitor diseases, disease processes, susceptibility, or to determine a course of treatment. Clinical tests can include, but are not limited to, a physical exam, a visual exam, performing an electrocardiogram, taking an x-ray, newborn screenings, a Pap smear, a tissue biopsy, genetic testing, calcium testing, blood tests, tests for reactive proteins, tests for circulating analytes, urine tests, stool tests, liver function tests, microbiological culturing, gastric fluid analysis, performing a lumbar puncture, malabsorption tests, pregnancy testing, toxicology, angiocardiography, brain scanning, magnetic resonance imaging (MRI), CT scan, body mass index (BMI), mammography, ultrasound, urography, endoscopy, laparoscopy, or esophagogastroduodenoscopy, colonoscopy.

In some embodiments, a clinical test can be a tissue biopsy. A tissue biopsy is a medical test that involves extraction of sample cells or tissues for examination to determine the presence or extent of a disease (e.g. cancer). A biopsy can consist of a needle biopsy, a CT-guided biopsy, an ultrasound-guided biopsy, a bone biopsy, a bone marrow biopsy, a liver biopsy, a kidney biopsy, as aspiration biopsy, a prostate biopsy, a skin biopsy, or a surgical biopsy or an endoscopic or colonoscopic biopsy of gastrointestinal tissue.

In some embodiments, a clinical test can be a blood test. A blood test is a laboratory analysis performed on a blood sample that is usually extracted from a subject. Multiple tests for specific blood components are often grouped together into one test panel called a blood panel or blood work. Blood tests are often used to determine physiological and biochemical states such as disease, mineral content, pharmaceutical drug effectiveness, or organ function. Blood tests can include, but are not limited to a basic or comprehensive metabolic panel, a complete blood count, a blood gas test, a blood film, measuring blood lead levels, a Schumm test, liver function tests, coagulation tests, protein electrophoresis, Western Blot, Northern Blot, blood typing, bone marrow aspiration, cephalin-cholesterol flocculation, enzyme analysis, epinephrine tolerance test, glucose tolerance test, hematocrit, immunologic blood test, inulin clearance, serological test, or thymol turbidity.

In some embodiments, a blood test can be a liver function test. A liver function test, also known as a hepatic panel, are groups of blood tests that provide information about the state of a patient's liver. Liver function tests can comprise AST, ALT, alkaline phosphatase, activated Partial Thromboplastin Time (aPTT), albumin, direct bilirubin, indirect bilirubin, or prothrombin time (PT/INR).

In some embodiments, a liver fibrosis test can be an enhanced liver fibrosis (ELF) test. An ELF test is a blood test that measures three molecules involved in liver matrix metabolism to give a score reflecting the severity of liver fibrosis. The three molecules measured are tissue inhibitor of metalloproteinases 1 (TIMP-1), amino-terminal propeptide of type III procollagen (PIIINP), and hyaluronic acid (HA), which all show strong correlations with fibrosis stages in chronic liver disease.

In some embodiments, a clinical test can be a test for reactive proteins. A test for reactive proteins, also called a C-reactive protein (CRP) test or a high-sensitivity C-reactive protein (hs-CRP) test, is a test which can detect slight increases within the range of standard reactive protein levels. A CRP test can test for inflammation, help diagnose a chronic inflammatory disease, or determine risk of heart disease.

In some embodiments, a clinical test can be a test for erythrocyte sedimentation rate (ESR). An ESR test measures how quickly red blood cells settle in a test tube. An ESR test detects inflammation associated with infections, cancers, and autoimmune diseases.

In some embodiments, a clinical test can test for circulating analytes. A test for circulating analytes can identify components other than blood components that are circulating in the blood. These components can include nucleic acids (e.g., DNA, RNA, microRNA, circulating tumor DNA), polypeptides, proteins, glycoproteins, cytokines, or hormones.

In some embodiments, a clinical characteristic can be disease characteristics, symptoms and severity/stage of a disease or condition, body mass index (BMI), age at diagnosis, type 2 diabetes, metabolic syndrome, MELD score, alcohol or other toxin consumption, dyslipidemia, genetic risk profile (PNPLA-3), liver stiffness/elastography, a controlled attenuation parameter (CAP), NFS score, FIB4 score, or any combination thereof.

A genetic profile is information about specific genes, including sequence variations and gene expression, in an individual or in a certain type of tissue. A genetic profile can be used to help diagnose a disease or learn how a disease may progress or respond to treatment with drugs or radiation. A genetic profile can include information from a genome, an epigenome, or a transcriptome. A genetic test can comprise analysis of a nucleic acid, a genomic DNA, a mitochondrial DNA (mtDNA), a mRNA, a microRNA (miRNA), a long non-coding RNA (lncRNA), a snoRNA, rRNA, tRNA, a circulating cell-free DNA (cfDNA), a circulating tumor DNA (ctDNA), a cell-free RNA (cfRNA), a cell-free total nucleic acid (cfTNA), an exosomal nucleic acid, a microbial or viral nucleic acid, a DNA methylation marker, or any combination thereof.

A molecular profile is information about specific proteins or other molecules (e.g. lipids, sugars, hormones) present in an individual or sample. A molecular profile can include information from a proteome, metabolome, lipidome, or glycome. Information about proteins can also include analysis of a post-translational modification, total protein, proteomics, protein ratios or any combination thereof. A post-translational modification can be phosphorylation, glycosylation, ubiquitination, nitrosylation, methylation, acetylation, lipidation, hydroxylation, proteolysis, or any combination thereof.

A digital profile is a digital measurement collected by a wearable device. A wearable device can be a smart watch, a smart clothing, a smart jewelry, a fitness tracker, an implantable device, a head-mounted display or a combination thereof. A digital measurement can be heart rate, brainwave, muscle bio-signals, sleep patterns and duration, speed, cadence, distance traveled, calories burned, respiration rate, skin conductivity, biomarkers from a bodily fluid, or any combination thereof.

Demographic characteristics are classifiable or measurable characteristics of a given population. Demographic characteristics are used in healthcare to assist in medical diagnosis by understanding how life events, choices, and circumstances may affect health outcomes. Examples of demographic characteristics can include age, race, gender, ethnicity, religion, income, education, home ownership, sexual orientation, marital status, family size, health and disability status, geographic area, psychiatric diagnosis, or occupation.

### EXAMPLES

These examples are provided for illustrative purposes only and not to limit the scope of the claims provided herein. It will be appreciated that variations in proportions and alternatives in elements of the components shown will be apparent to those skilled in the art and are within the scope of the embodiments presented herein.

### Example 1. Diagnosing NASH Using Probes in Mice

In this experiment, the probes of the present application were shown to accurately detect the activity levels of proteases associated with non-alcoholic steatohepatitis (NASH) in a fluid sample to diagnose NASH in a subject.

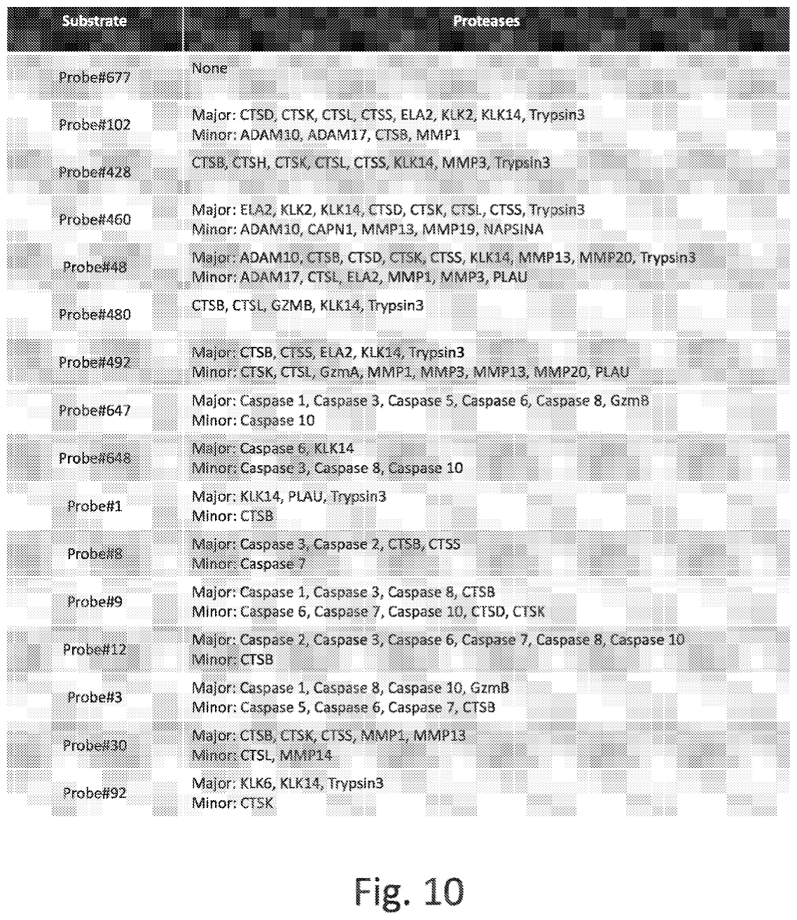

Protease activity levels associated with NASH were assessed in vivo in two mice populations, one healthy and one with NASH. The probes used in vivo are shown in FIG. 10.

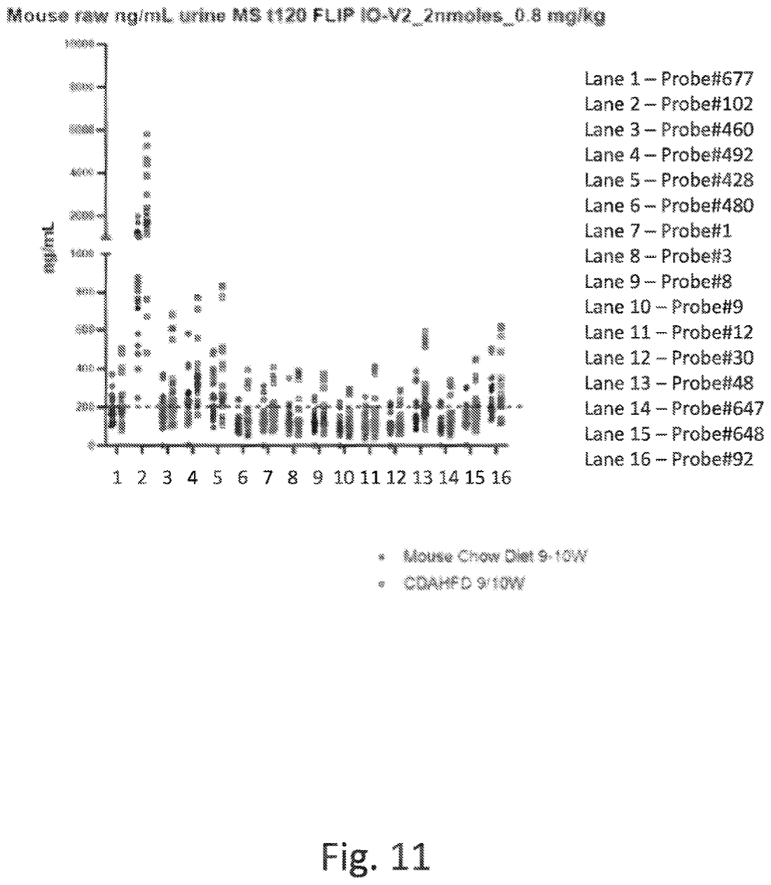

Mass-barcoded reporters urinary concentration levels obtained from proteolytic cleavage of these probes by proteases in healthy mice, which were fed on a standard Chow Diet (CD), and NASH mice, which were fed a choline-deficient, L-amino acid-defined, high-fat diet (CDAHFD) are shown in FIG. 11. NASH-related probes, cleaved by increased NASH-related protease activity, associated with higher mass-barcoded reporters accumulation in urine from NASH mice compared to healthy mice.

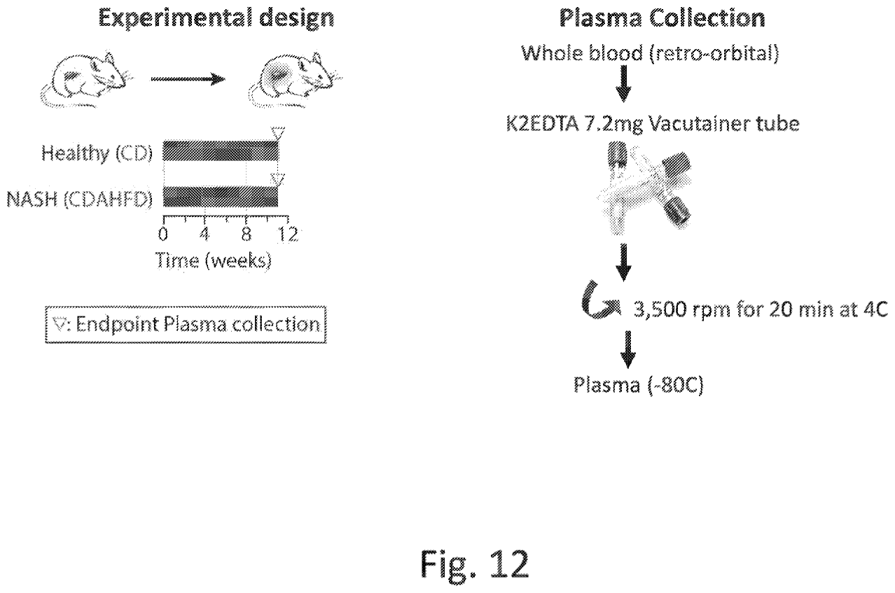

As shown in FIG. 12, blood samples were collected in K2 EDTA tubes from mice that were either healthy (CD) or had NASH (CDAHFD) after 12 weeks on their respective diet. All animals were used in accordance with animal care guidelines. Plasma was obtained from these blood samples by centrifugation at 3,500 RPM for 20 min at 4° C. The plasma was stored at −80° C. until it was needed for experimental purposes.

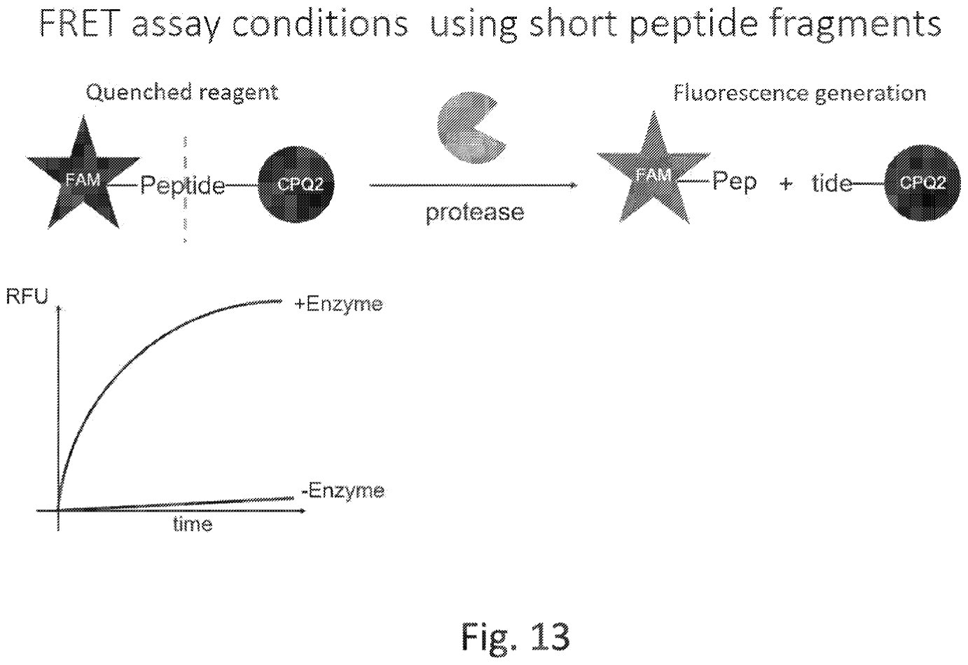

As shown in FIG. 13, thawed plasma samples were pooled and contacted with probes with fluorescent quenchers and protease-cleavable fluorescent reporters at various peptide and serum concentrations. Samples were mixed with protease substrates and quenchers/reporters in 96-well plates. The 96-well plates were read on a Biotech Synergy H1, using 465,535 excitation/emission settings.

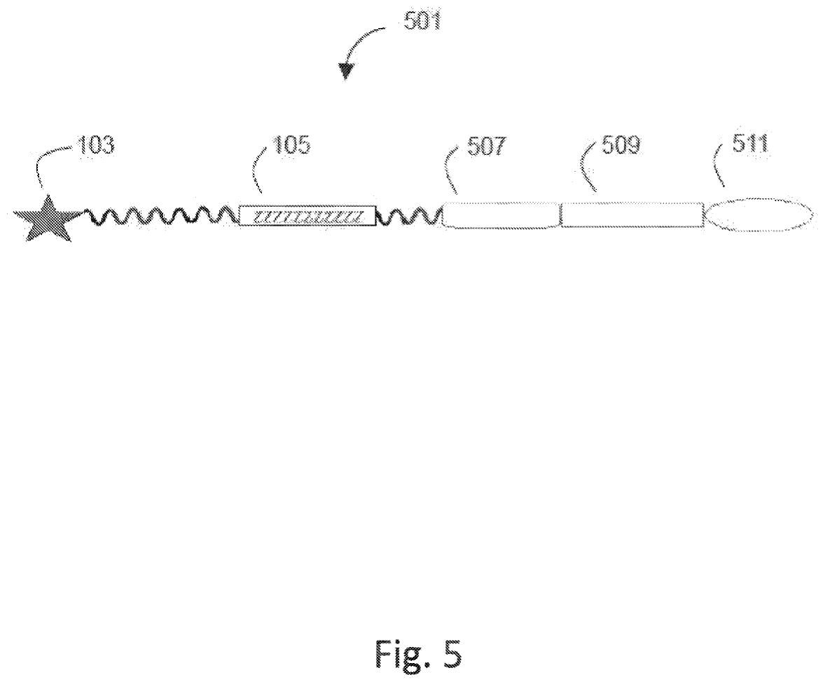

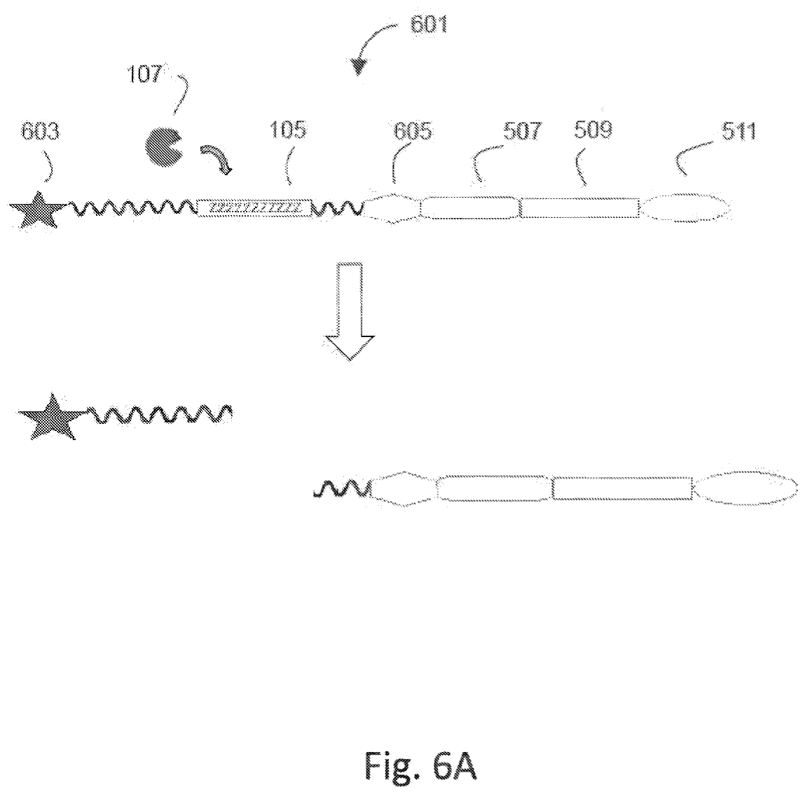

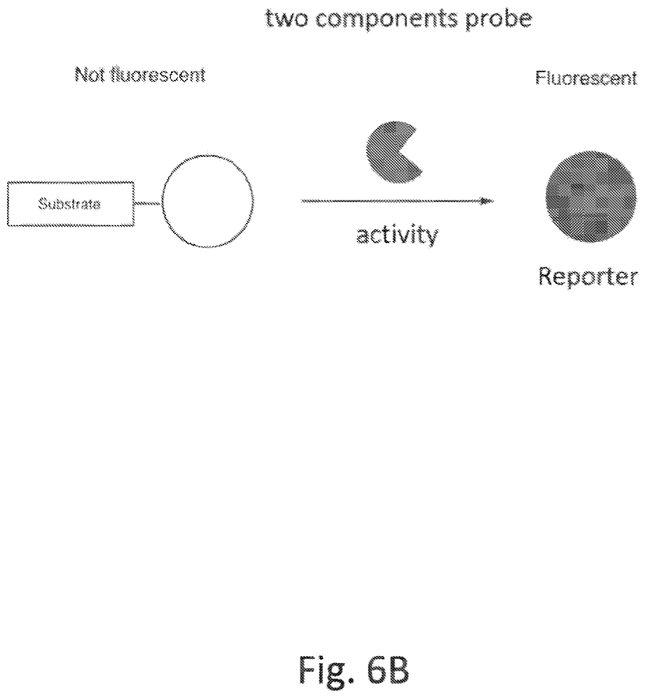

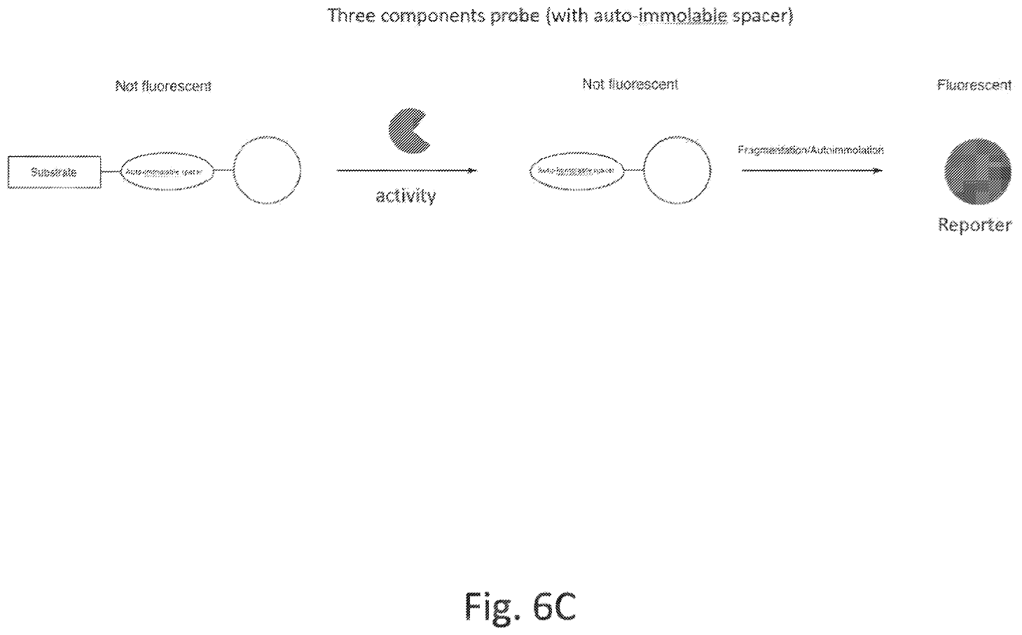

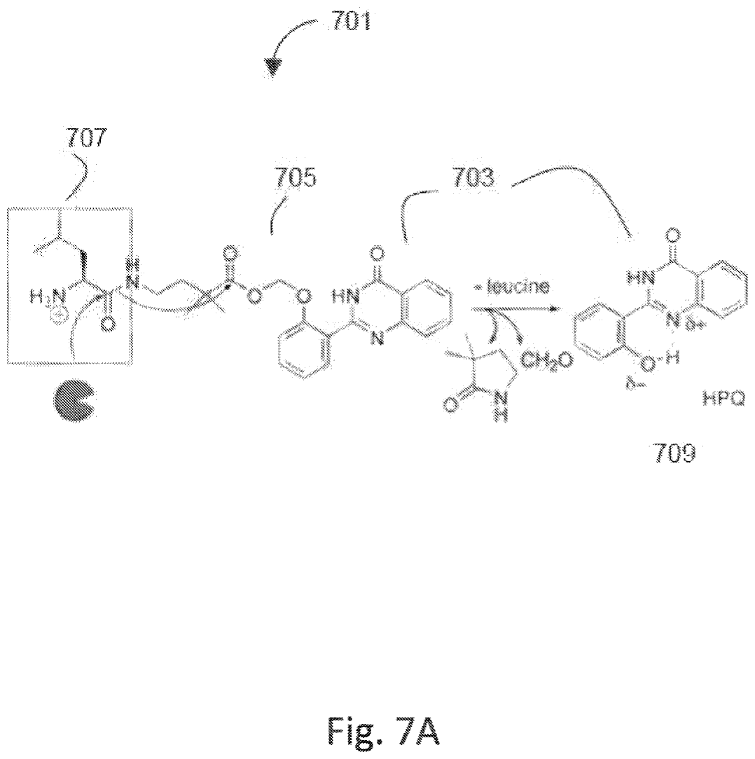

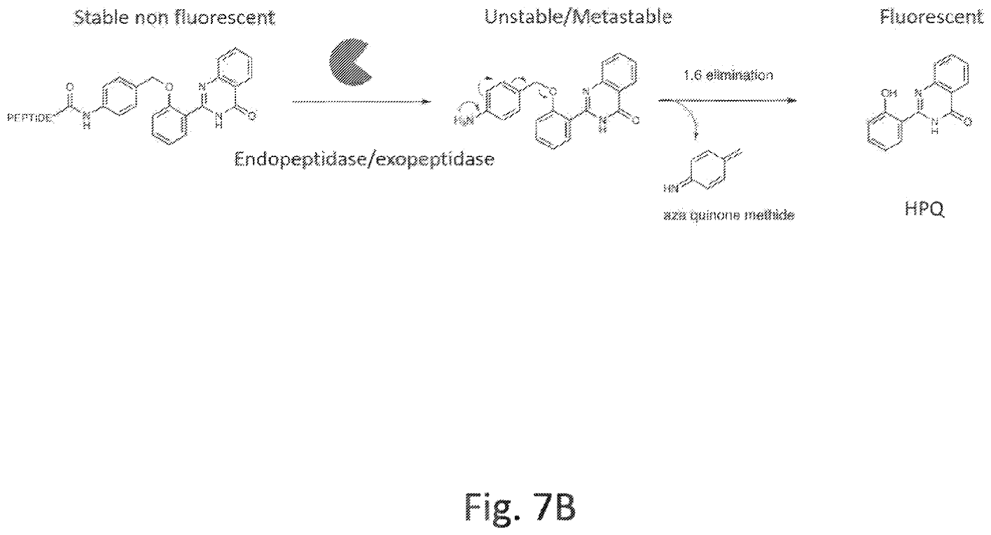

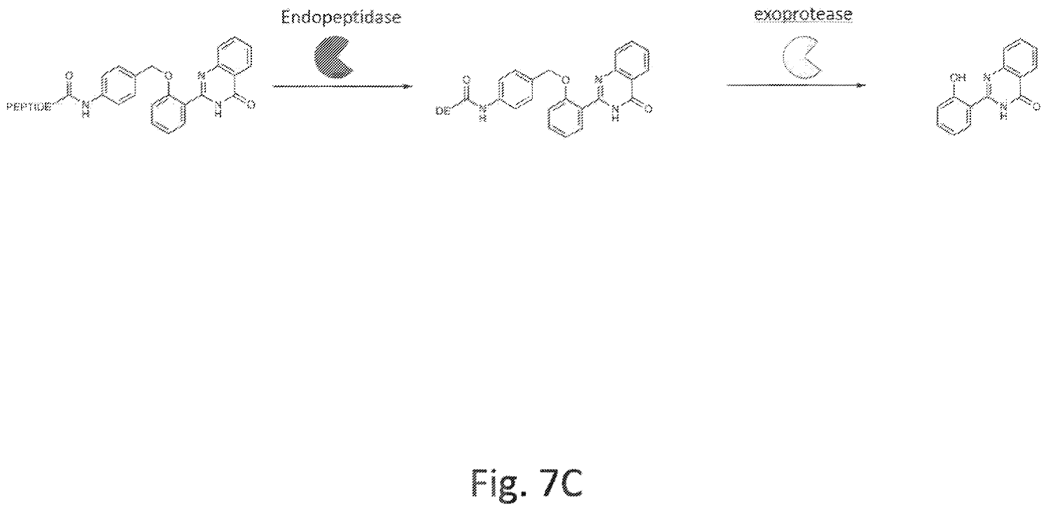

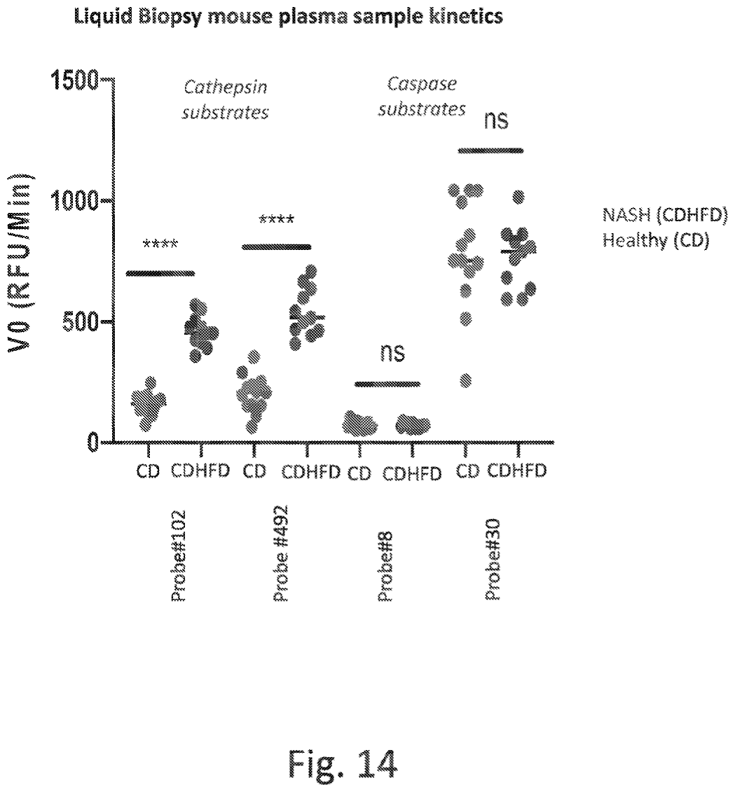

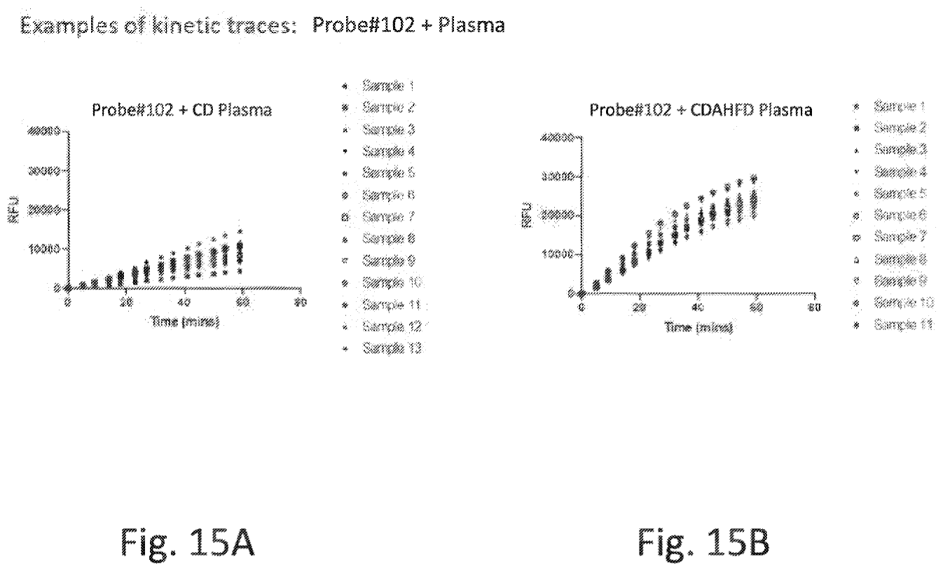

As shown in FIG. 14, the probes of the present application were able to measure the activity of NASH-related proteases as expressed in Relative Fluorescent Unit (RFU) per minute in the two mouse populations. Probes measuring cathepsin activity were 3-fold higher in protease cleavage kinetics in mice with NASH compared to healthy mice. In contrast, probes sensing caspase activity showed no change in detectable activity between healthy and NASH mice. FIG. 15A and FIG. 15B show the subset of results for one probe, Probe #102, in detecting NASH-related protease activity; here, the use of the fluorescent reporter and quencher, like those discussed in FIG. 5, were shown to accurately measure the activity levels of NASH-related proteases in the plasma of healthy mice (FIG. 15A) and NASH mice (FIG. 15B).

Thus, probes of the present application can accurately detect the activity levels of proteases associated with a biological condition or disease-state in a subject, using a body fluid sample.

### Example 2: Detection of NASH Protease Activity in Plasma in Mice

As shown in FIG. 14, the probes of the present application are able to accurately detect protease activity of NASH related proteases in the plasma samples taken from two mice populations, as explained in Example 1 and FIG. 13, in a multiplex format. A single plasma sample was contacted with the probes for each predetermined protease to provide a multiplex assessment of protease activity in the sample.

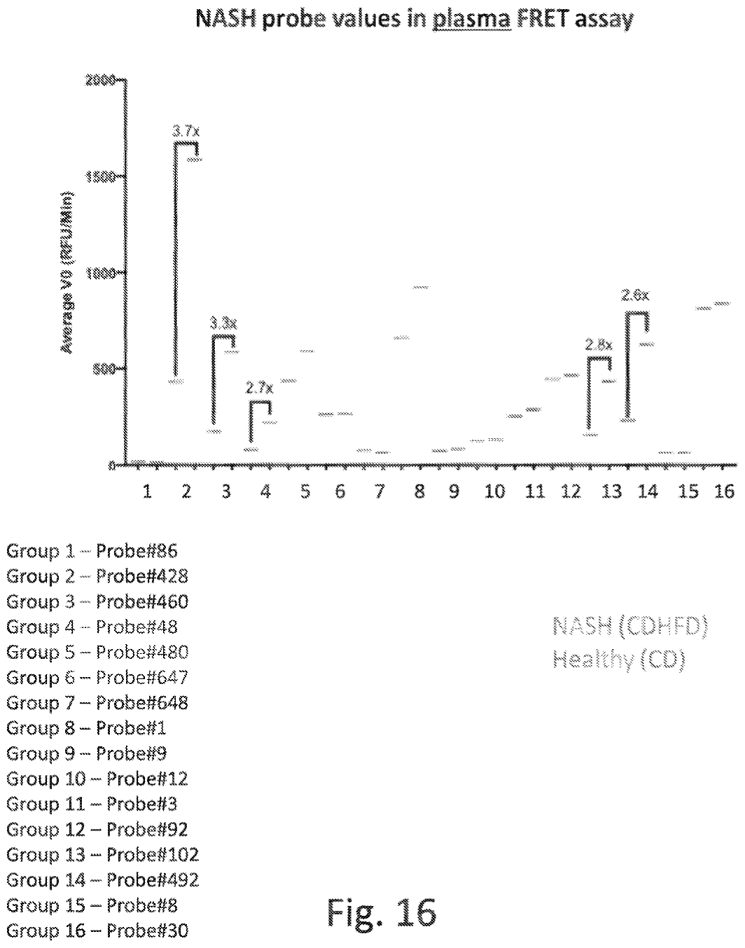

In FIG. 16, for each set of probes, the protease activity in healthy mice is shown on the left, while the protease activity in NASH mice is shown on the right. As shown, the probes of the present application were able to measure increases in NASH-related protease activity.

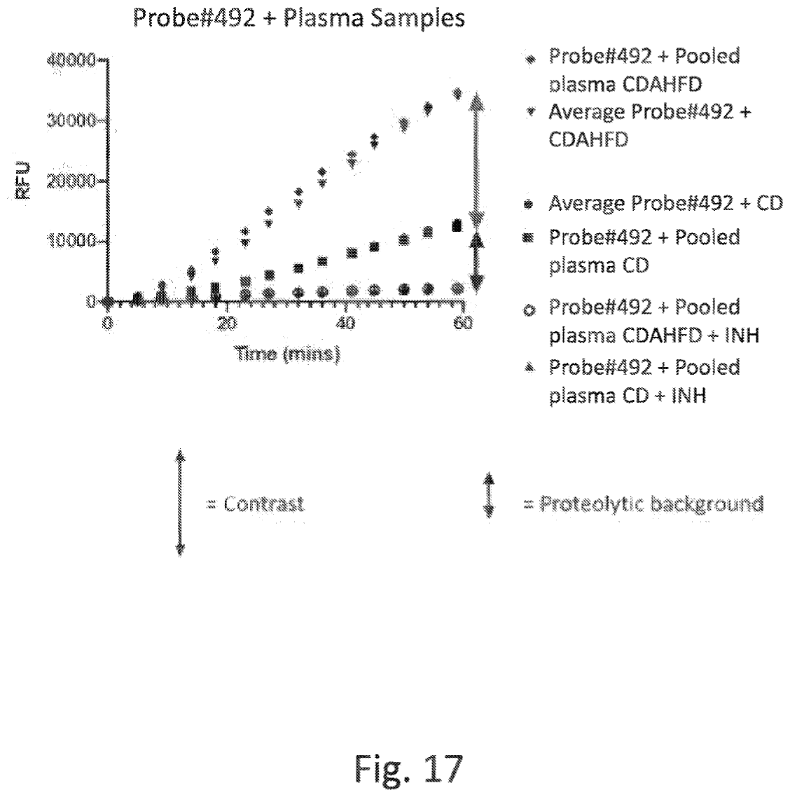

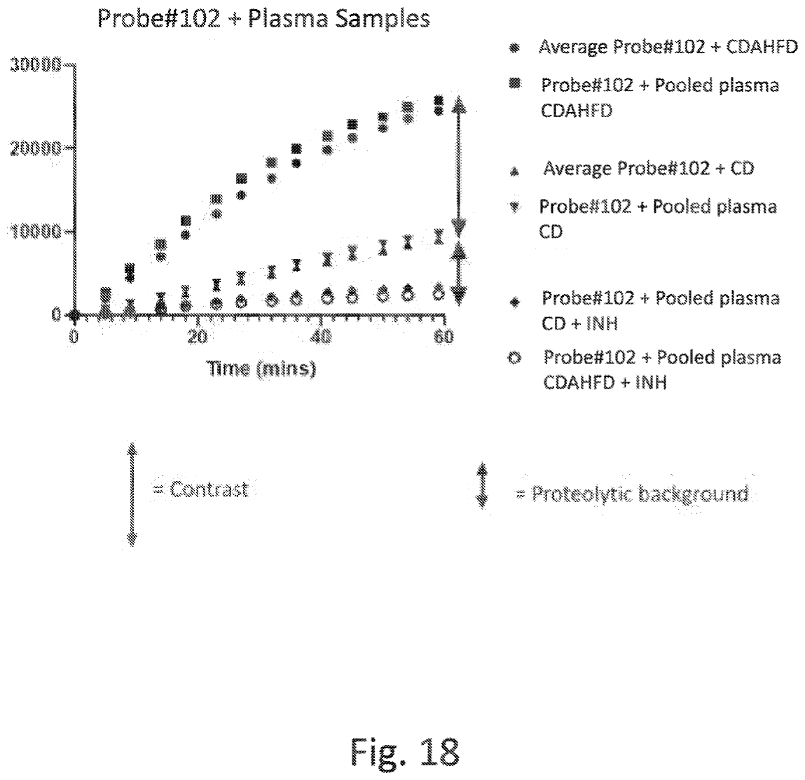

As shown in FIG. 17 and FIG. 18, protease activity measured as RFU/min was similar in pooled plasma samples within the same group of animals than the average of protease activity from each animal from that group. Furthermore, adding a broad protease inhibitor cocktail (INH) completely abrogated protease activity in both healthy and NASH animal groups, providing evidence that the fluorescent signal measured over time depends on proteolytic activities.

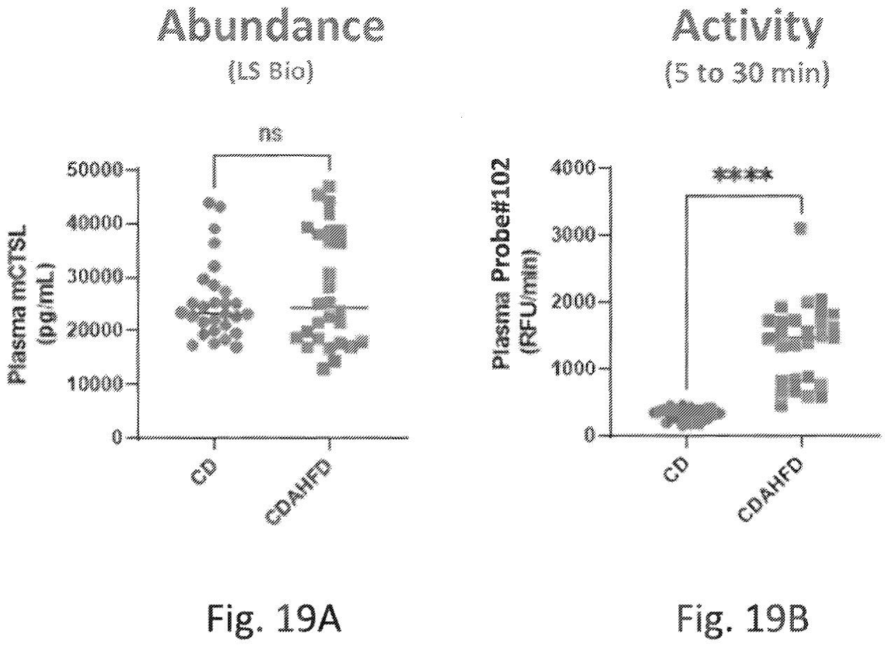

FIG. 19A and FIG. 19B show that, when studying samples of mouse plasma, activity, not abundance, is more important in differentiating between healthy samples and NASH samples. Although abundance of NASH-related proteases (here cathepsin L, or CTSL) may be comparable between healthy CD mice and NASH CDAHFD mice (FIG. 19A), the activity levels of these proteases are not (FIG. 19B). In this experiment, protease abundance was measured using an ELISA kit from LS Bio while activity was measured using the Probe #102 (a CTSL sensing probe) fluorescence assay described in Example 1.

Thus, probes of the present application can accurately detect the activity levels of proteases associated with a biological condition or disease-state in a subject, using a body fluid sample such as plasma in a multiplex format.

### Example 3: Liquid Biopsy Determines Progression Versus Regression of NASH

In this experiment, the probes of the present application were able to differentiate among healthy mice, NASH mice, and NASH mice that were undergoing disease regression.

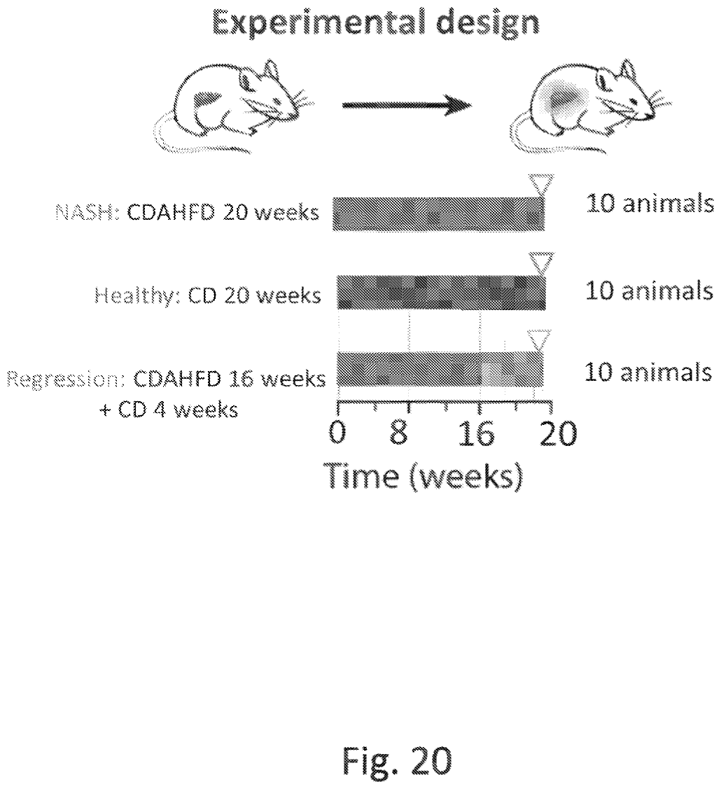

FIG. 20 shows the experimental design including three groups of mice: CDAHFD NASH mice for 20 weeks (NASH progression), healthy CD mice for 20 weeks, and mice fed a CDAHFD for 16 weeks before being switched to a chow diet for 4 weeks (NASH regression). Plasma samples were collected from all animals at 20 weeks.

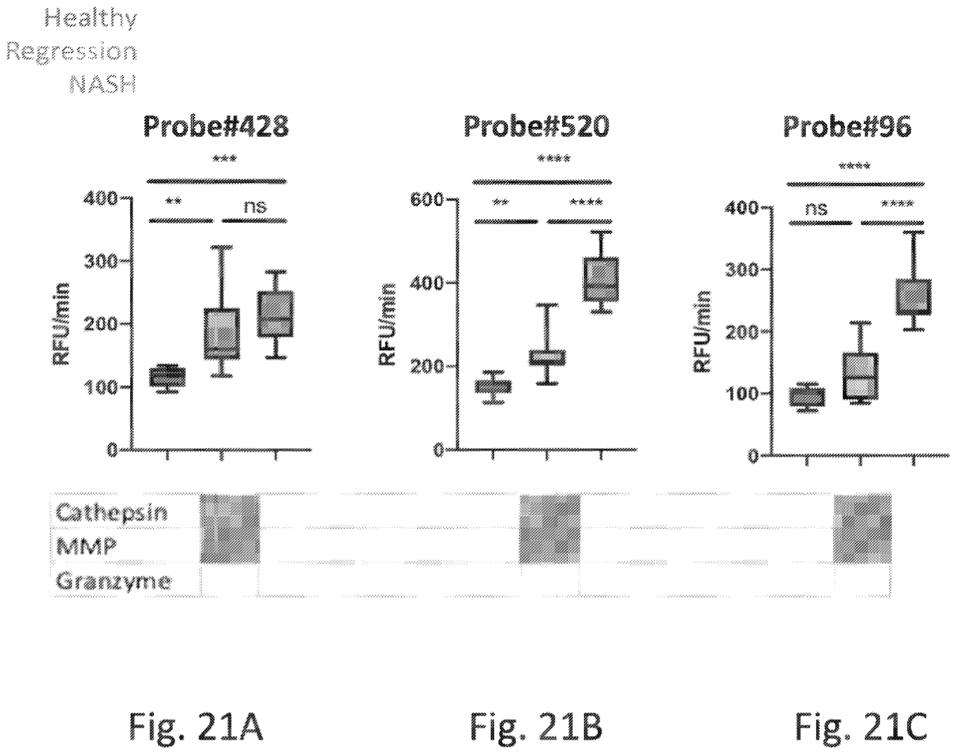

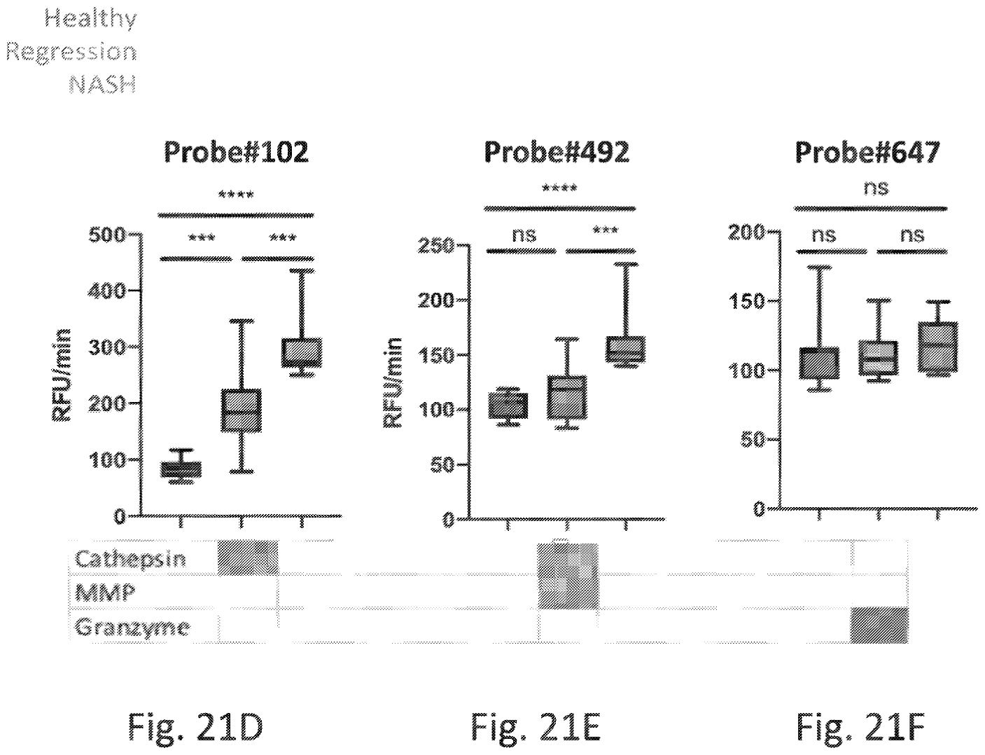

As seen in FIGS. 21A-F, several probes were used to contact the thawed plasma, as described in Example 1, and this resulted in clear differentiation between the healthy, regression, and NASH samples. The probes showing the most differentiation in NASH were linked to cathepsin and/or MMP protease activities.

This experiment showed that not only can we differentiate between healthy and diseased samples, but it can also differentiate among healthy, disease-progressing, and disease-regressing samples.

### Example 4: Liquid Biopsy Applications Towards Fulminant Hepatitis in Mice

In this experiment, another mouse liver-disease model—that for fulminant hepatitis—was studied to determine the wider uses of the present application. This experiment served to develop the ex vivo assay technology for applications in hepatitis models using plasma and existing sensors in the FRET substrate library.

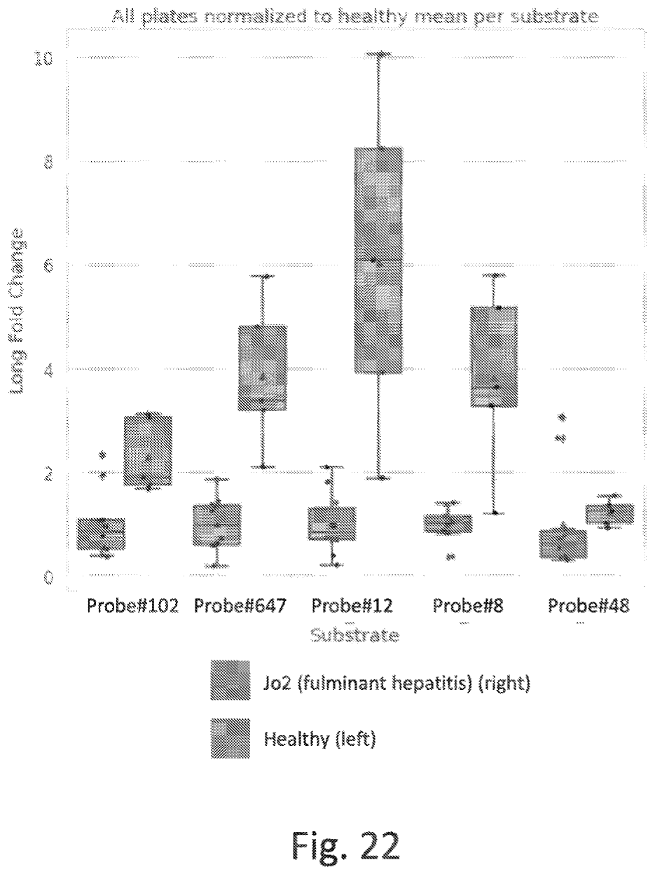

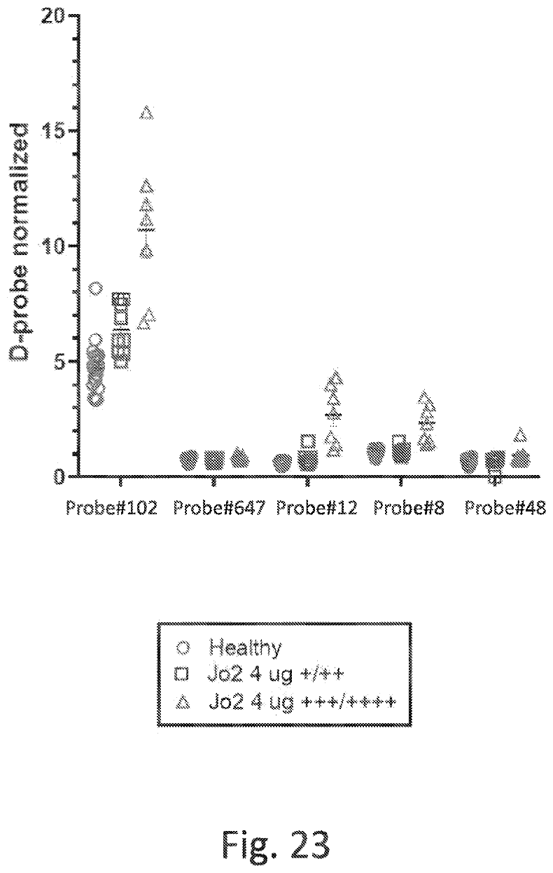

Fulminant hepatitis is induced after injection intraperitoneal of monoclonal antibody anti-CD95 (Jo2, BD biosciences, 4 ug/animal), and mouse plasma samples were collected 3 hours after Jo2 injection. As shown in FIG. 22, when the probes contacted the mouse plasma samples using the method described previously in Example 1, the probes were able to differentiate between healthy and Jo2 samples ex vivo. FIG. 23 shows the same results in vivo, with the same mice receiving the injectable probe formulation for direct comparison with the ex vivo approach.

The Jo2 hepatitis model demonstrates differential probe cleavage compared to NASH liver model data in mice. Predominantly Caspase centric probes (Probe #647, Probe #8, Probe #12) show contrast that is specific and sensitive to the Jo2 model. The comparison with mass spectrometry data also aligns and confirms high concordance with the ex vivo approach, which is reassuring to confirm the existence of a biologically relevant signal.

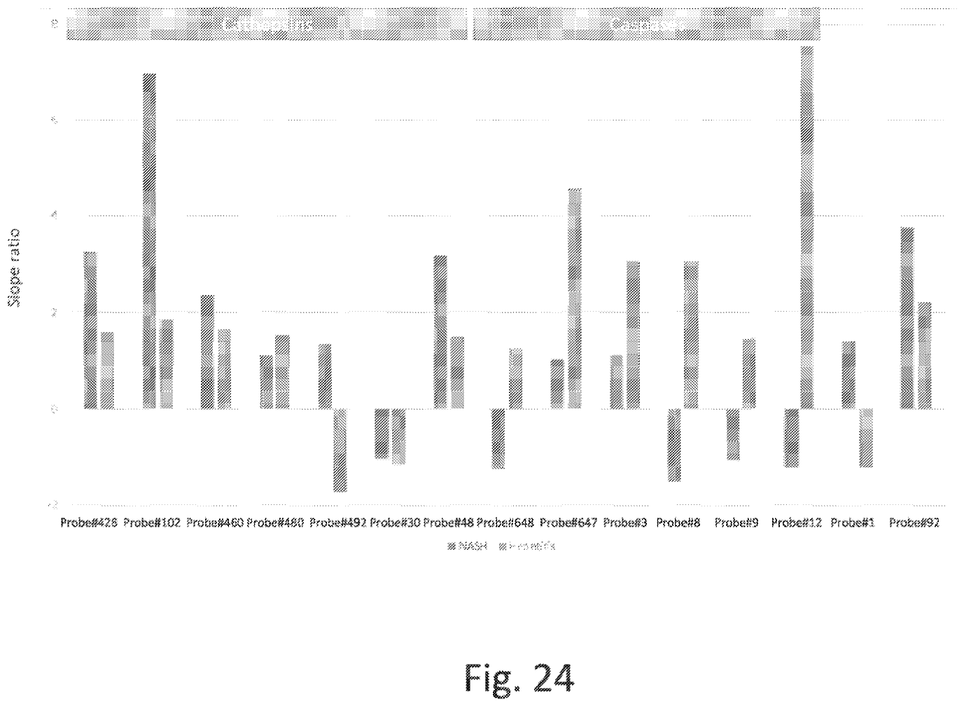

FIG. 24 demonstrates that for two preclinical models of liver disease, the application can distinctly identify each disease due to the distinct biological mechanisms underlying protease activity of each disease (i.e., cathepsin activity in NASH and caspase activity in hepatitis).

### Example 5: Detecting NASH in Human Plasma

This experiment relates to the detection of NASH in humans.

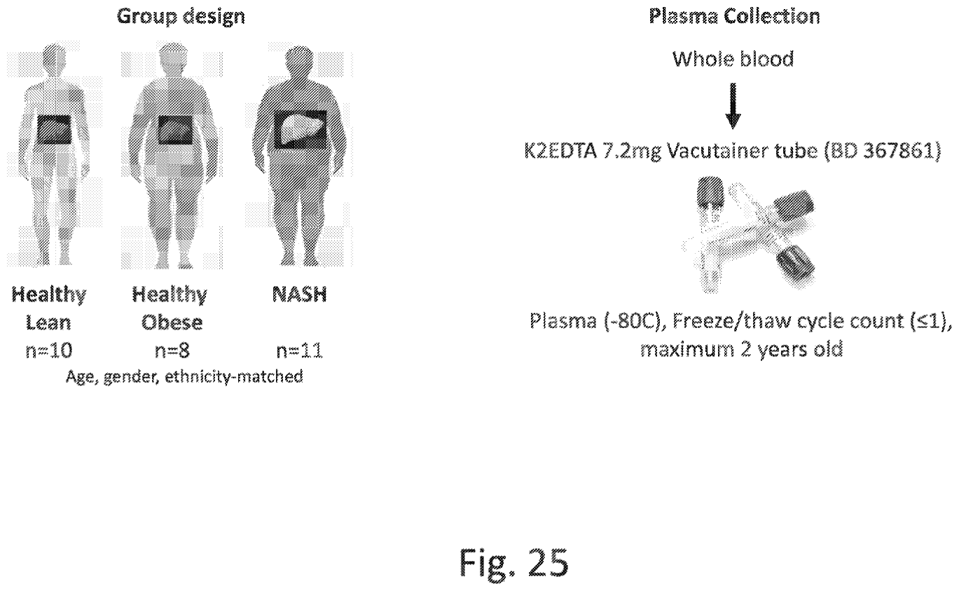

As shown in FIG. 25, blood samples were collected from human subjects that were diagnosed as healthy/lean, healthy/obese, or NASH. Plasma was obtained from these blood samples in the same method as used in Example 1. The plasma was stored at −80 C for no more than 2 years and with a freeze/thaw cycle count of ≤1 for each sample.

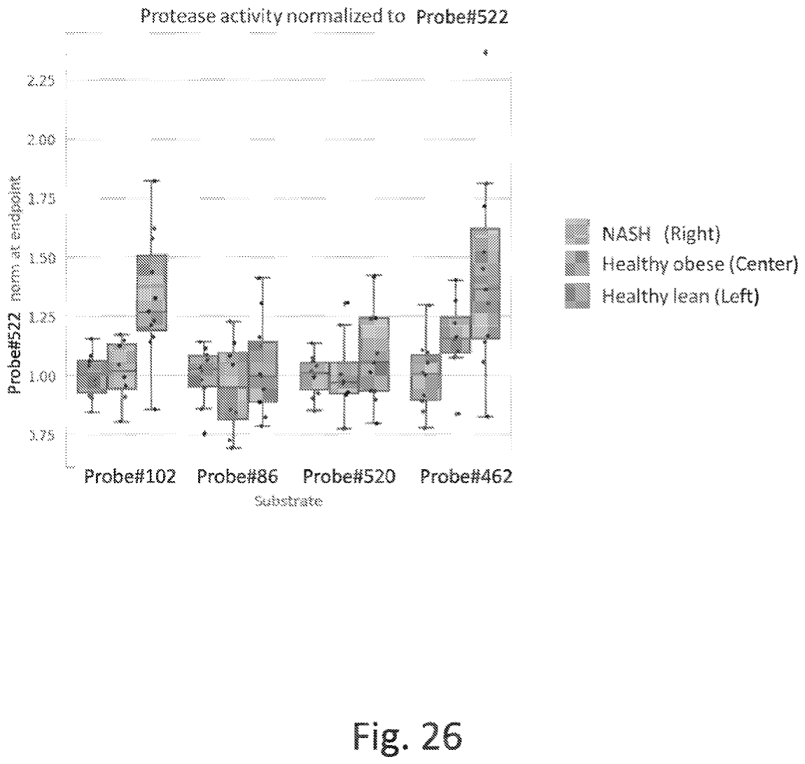

As shown in FIG. 26, when the probes contacted the human plasma samples using the method described in Example 1, increased fluorescence levels over time were observed in NASH samples when compared to healthy, allowing differentiation between the protease activity levels of healthy and NASH samples.

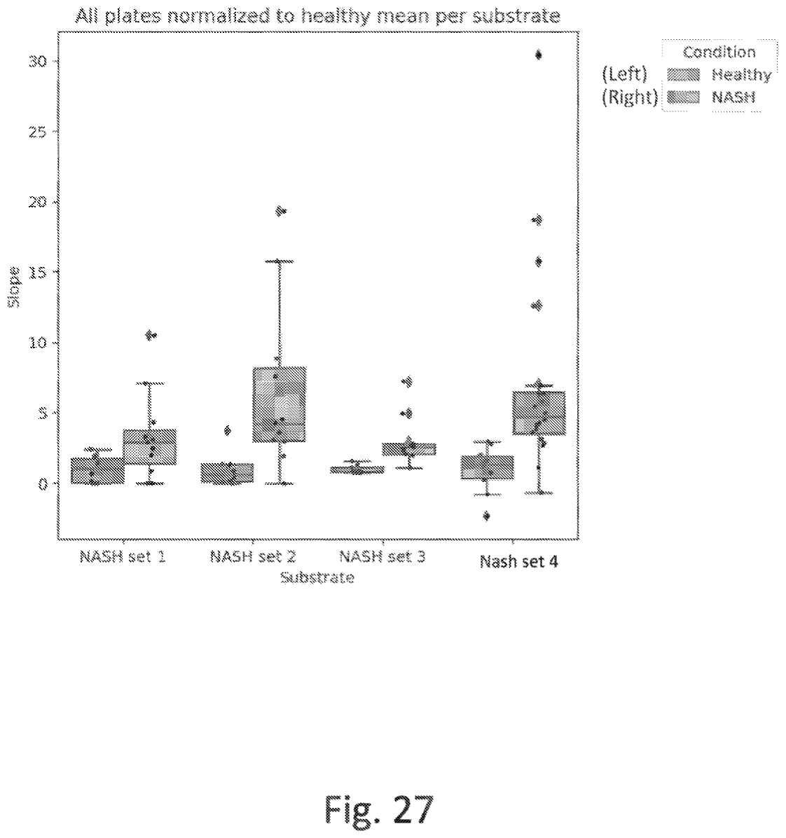

FIG. 27 shows high levels of reproducibility in the application's ability to differentiate between healthy and NASH samples when independent sample cohorts were tested.

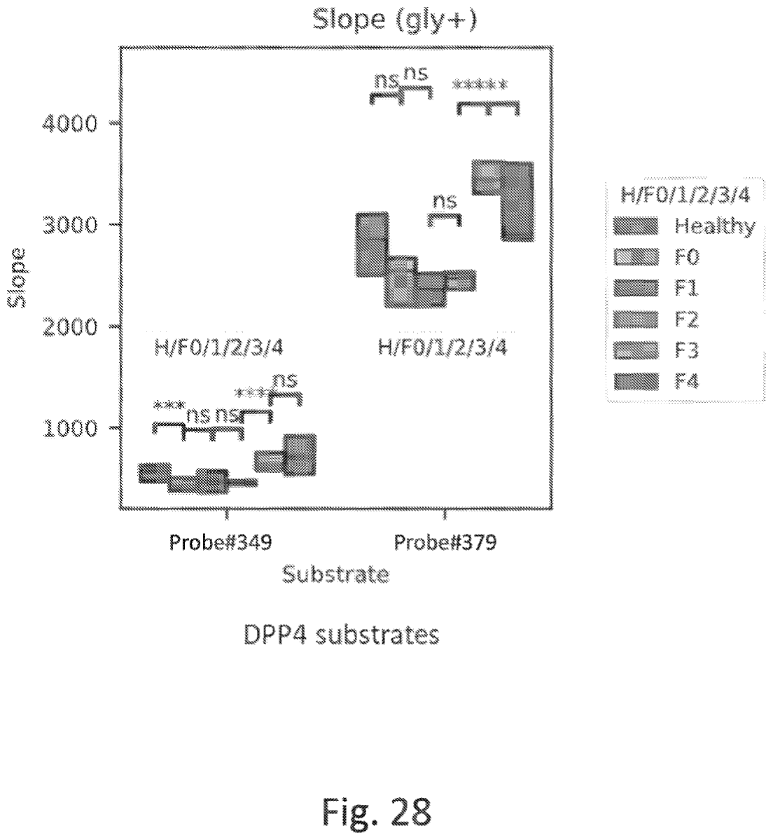

FIG. 28 further demonstrates that the application is not only able to differentiate between healthy and NASH human samples, but it is, surprisingly, also able to differentiate between early-stage (F0-F2) and late-stage (F3+) NASH. The entire F0-F4 data set contains 100 NASH samples, and the experiment was conducted using the method from Example 1.

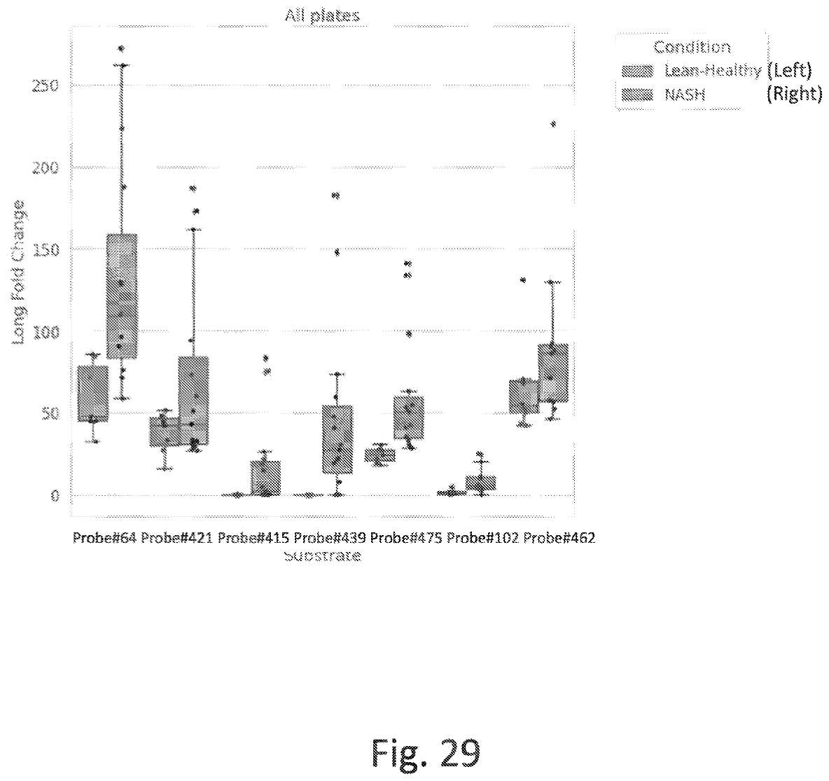

As shown in FIG. 29, multiple probes of the present application are able to differentiate between healthy and NASH samples in humans—this multiplicity furnishes a lower false-positive rate when testing samples.

This experiment demonstrates the application is highly adept at differentiating between healthy and NASH (and different fibrosis stages of NASH) in a non-invasive manner in human subjects.

### Example 6: Mechanism of Function of Liquid Biopsy

In this experiment, the specific protease cleaved by a specific probe is determined in order to show the specificity of the application regarding the disease differences it detects. This experiment also shows that protease activity, not abundance, is the driving factor in the application's determination of disease-markers in a sample.

For this experiment, all plasma samples were prepared individually and diluted 1/10e in PBS with inhibitor added directly to the samples. Inhibitor was prepared at 15× concentration to final. Substrates were diluted in DI water at 18 uM, such that the final concentration on the plate would be 6 uM. All samples were prepared such that their last dilution on the plate would not affect the desired final concentration. 10 ul of each individual sample was pipetted into their corresponding wells, and the plate was then spun down in the centrifuge at 1500 RPM for 30 seconds to coat the bottom of each well with the sample. 5 ul of the 18 uM substrate solution was pipetted into each well being used on a 384 well plate, and then the plate was spun down in the centrifuge at 1500 rpm for 30 seconds. The plate was placed immediately in the plate reader at 37° C. for a 30-minute-long fluorescence kinetic read at 485/535 extended gain.

To assess the proteolytic cleavage pattern of Probe #102, samples were tested using a pool of broad inhibitors for serine, cysteine, threonine, MMP and aspartic protease family members (broad inhibitor) to assess general protease activity, AEBSF for serine proteases, E64 for cysteine proteases, CTSi for broad cathepsin inhibition of cathepsins L, S, K and B, or specific inhibitors for cathepsin K (L00625), for cathepsin L (SID) or cathepsin B (CA074).

All E64 (broad cysteine), SID (CTSL) and the CTSi (CTSL, S, K, B) inhibitors decreased NASH signal significantly with less decrease in signal for healthy, indicating that the nature of the decrease in signal was disease specific. When using the broad inhibitor or E64, we observed a greater than 6-fold decrease in the RFU signal contrast between NASH and healthy samples, indicating that a cysteine protease was responsible for the disease contrast. Broad cathepsin inhibitor CTSi decreased NASH by 47% while only decreasing healthy by 18%, demonstrating that a cathepsin was responsible for the disease contrast. A specific cathepsin inhibitor for CTSL (SID) decreased NASH by 60% while only decreasing healthy by 33%. Both NASH and healthy decreased with the addition of the serine inhibitor, AEBSF. NASH was inhibited 65%, while healthy was inhibited at 60%. The similar decrease in RFU for both NASH and healthy indicates that the AEBSF signal being sensed by Probe #102 is not a significant contributor to the disease specific signal and of a background nature.

Specific inhibitors for cathepsin K and B, L006235 and CA074, respectively, did not significantly decrease signal for NASH or healthy samples.

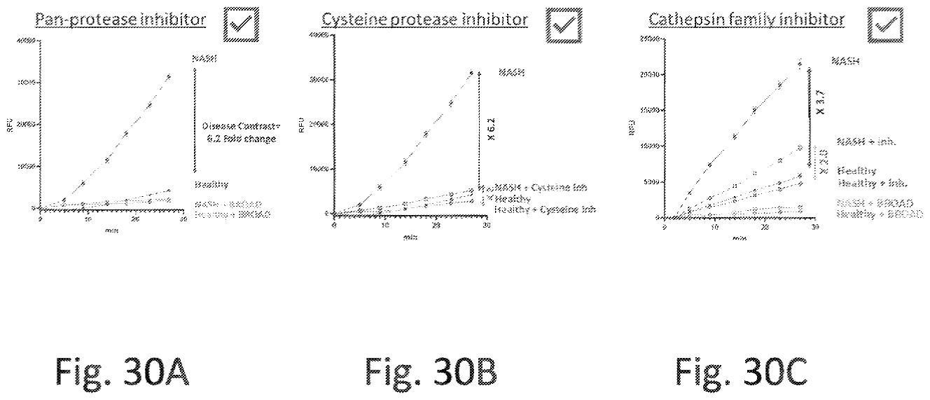

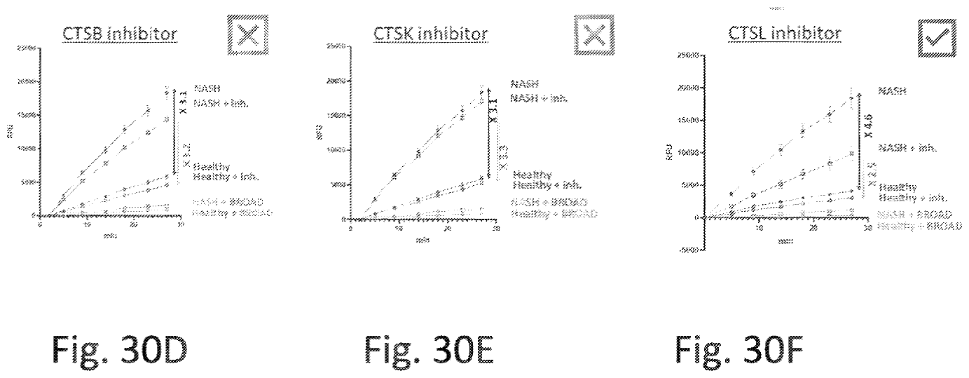

FIG. 30A demonstrates Probe #102 in combination with broad protease inhibitors to show that Probe #102 specifically contacts a protease in order to determine the difference between healthy and NASH samples. FIG. 30B shows that Probe #102 contacts a cysteine protease, and FIG. 30C further limits this to a cathepsin family protease. FIG. 30D-F test individual cathepsins to show that Probe #102 specifically responds to the activity of cathepsin L (CTSL), a NASH-related protease. Thus, cathepsin L activity is responsible for the disease vs. healthy differences in protease activity in samples as recognized by the application.

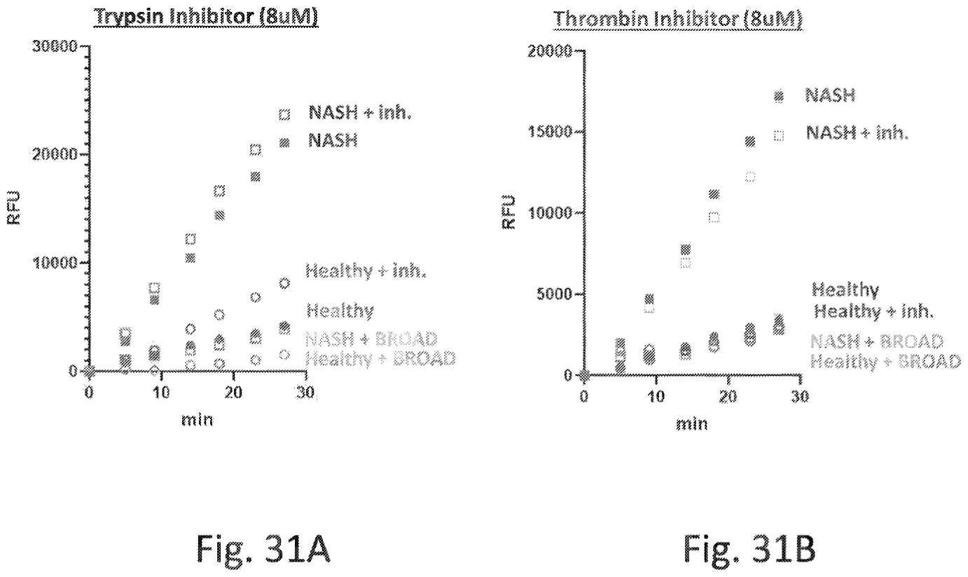

As shown in FIG. 31A-B, the application's discrimination between healthy and NASH tissue is not caused by either trypsin or thrombin, both promiscuous proteases that are constantly present in blood.

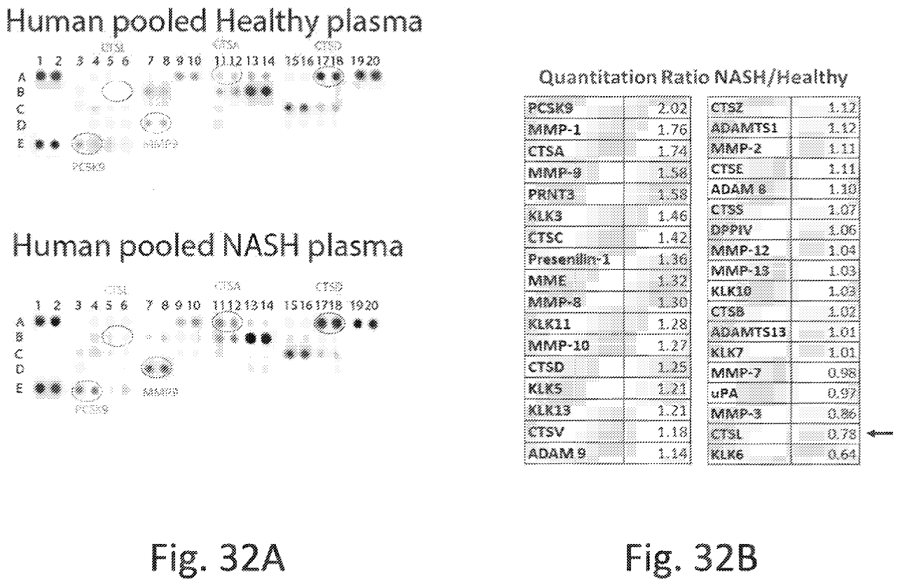

As shown in FIG. 32A-B, protease activity is the true measure of disease, rather than protease quantity. This corroborates the previous determination in mice that activity is more important than abundance as previously seen in Example 2 and as previously shown in FIG. 19.

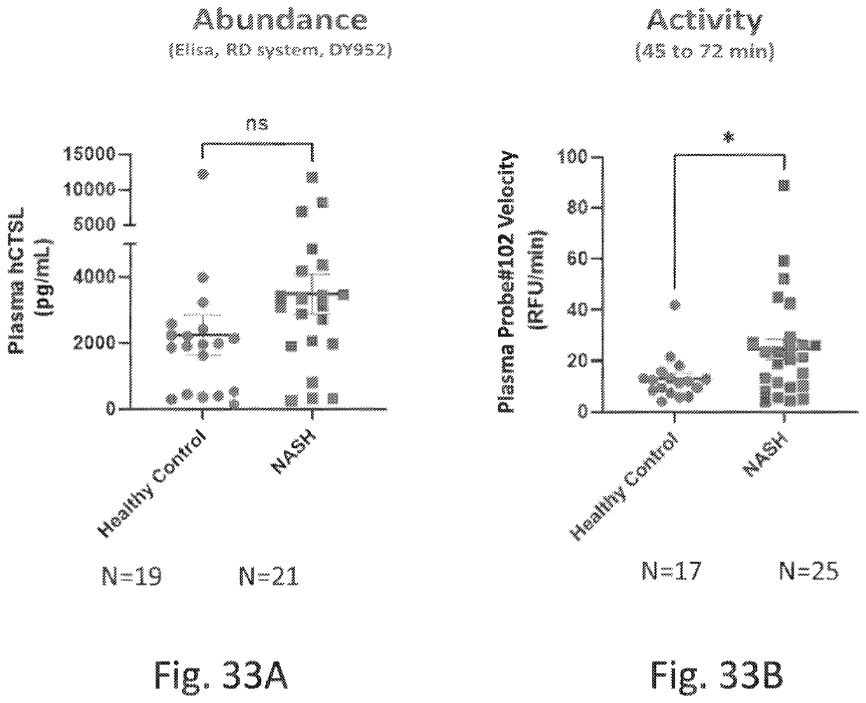

More specifically, FIG. 33 demonstrates that although CTSL is equally abundant in both healthy and NASH human samples, CTSL activity is different between these two sample populations.

The application is shown to function by measuring the activity levels, rather than the abundance of specific disease-related proteases, to give an accurate determination of a specific disease in a sample.

### Example 7: Liquid Biopsy Applications Toward COVID Diagnosis

In this example, the application is directed toward diagnosing COVID.

Initial experiments with COVID used K2 EDTA and Lithium Heparin collected plasma. Samples were thawed on ice from storage in −80° C. and were then diluted to 10% in PBS. After the samples were prepared, the volume was split in half and broad protease inhibitors were added to one tube-100× dilution final, 67× in the tube. 10 μL of each sample were placed into a well in a 96-well plate, and the plates were stored on ice. Substrates were prepared at 18 uM in ddH2O using 1 mM stock prepared in DMF. 5 μL of substrate were added to each well. The 96-well plates were spun down at 1000 RPM for <30 seconds. The plates were read on Biotek Synergy H1 plate reader, Ex/Em=485/535 with a cycling time of 4 mins 30 seconds using a kinetic read, extended dynamic range for 1 hour.

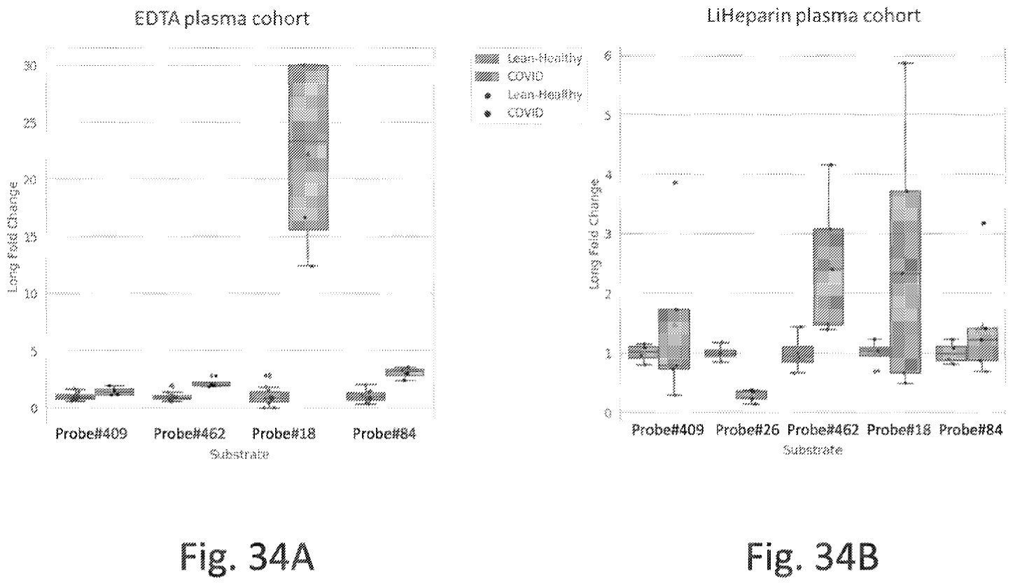

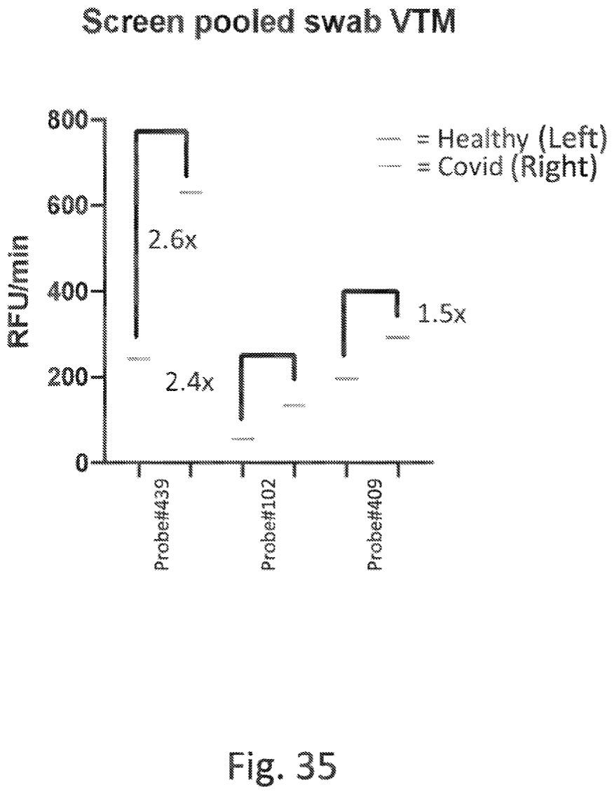

As shown in FIG. 34A-B, multiple sensors demonstrated differential cleavage between COVID and healthy samples. Probe #462, Probe #18 and Probe #84 demonstrated contrast in both sets and Probe #409, the SARS CoV2 coronavirus substrate, showed modest contrast in the K2 EDTA samples.

As shown in FIG. 35, COVID positive and COVID negative swabs (as determined by PCR at the clinical site) were combined with LBx sensors to determine if protease activity can be sensed ex vivo using swabs.

Samples were thawed on ice and then diluted to 10% in DPBS (neutral pH 7.4, Gibco). Where required, samples were pooled according to condition with equal volumes of each sample per condition and then subsequently diluted in DPBS. After the samples are prepared, the volume was split in half and broad protease inhibitors were added to 1 tube-100× dilution final, 67× in the tube. 10 μL of each sample was added into the corresponding wells of a 96-well plate, and the plates were stored on ice. Substrates were prepared at 18 uM in ddH2O using 1 mM stock prepared in DMF. 5 μL of substrate was added to each sample in the 96-well plate, and the plates were spun down at 1000× rpm for <30 seconds. Plates were read on a Biotek Synergy H1 plate reader, Ex/Em=485/535 with a cycling time of 4 mins 30 seconds using a kinetic read, extended dynamic range for 2 hours.

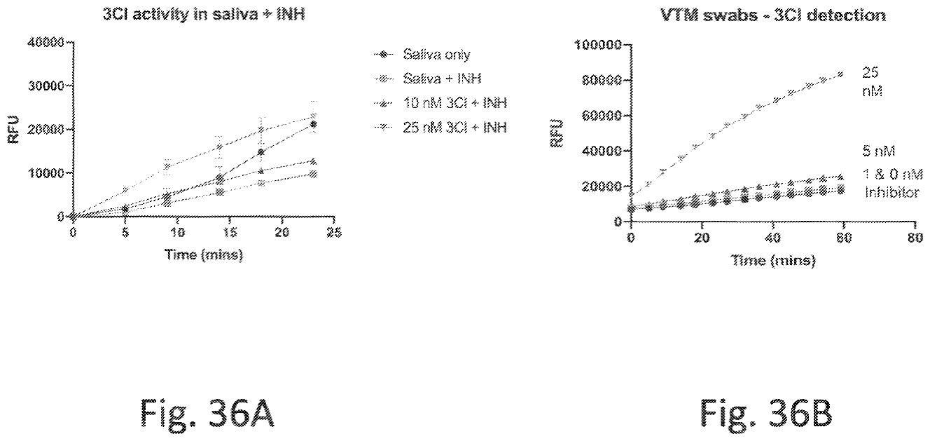

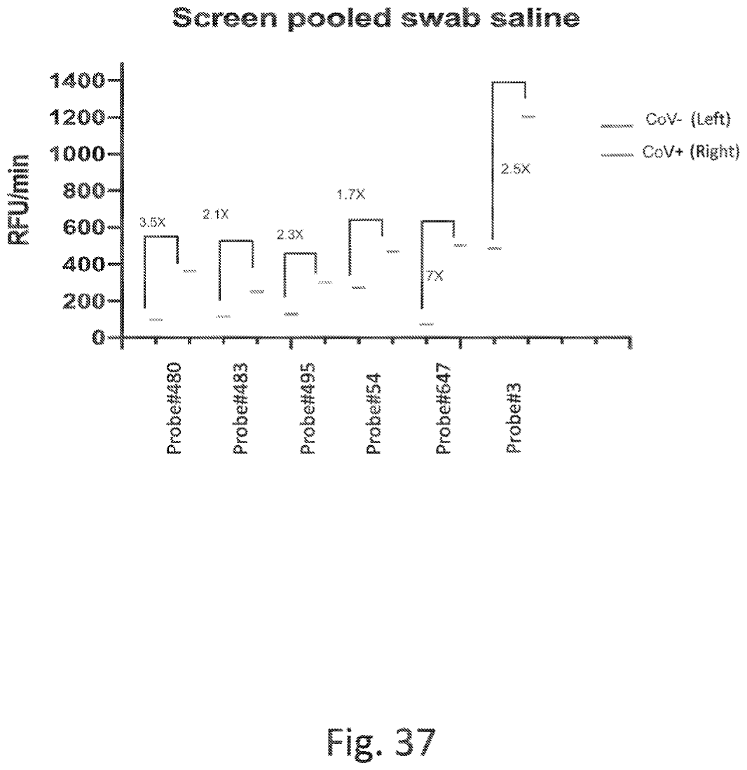

FIGS. 36A-B shows both swabs and saliva samples treated with viral transport media (VTM), which contains some proteases in the serum after contact with the probes of the application. However, when swabs were tested using the method from experiment 1 using a saline media instead of VTM, as shown in FIG. 37, clear differences could be seen between COVID- and COVID+ samples (as determined by clinical PCR testing). The saline media swabs give superior protease activity signal compared to the VTM swabs as they were collected in saline media with no additives. This shows the application has broad applicability across biofluids.

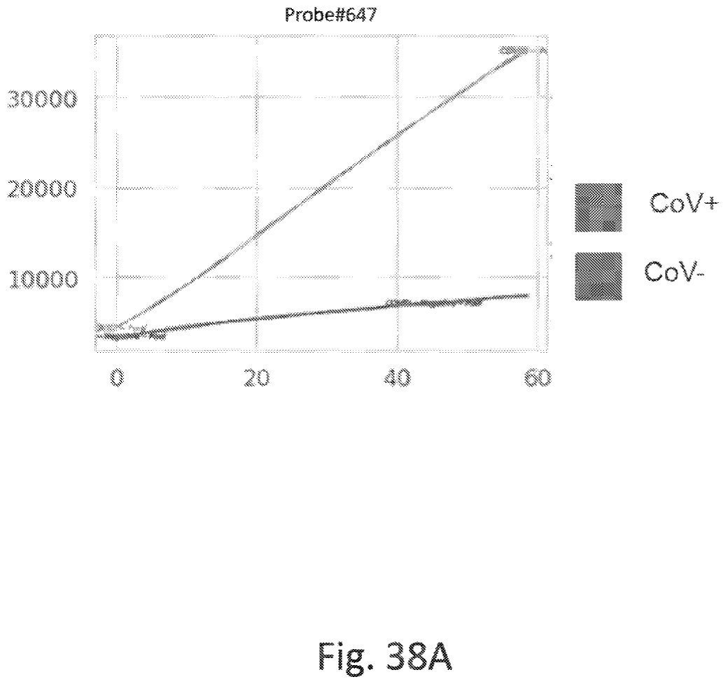

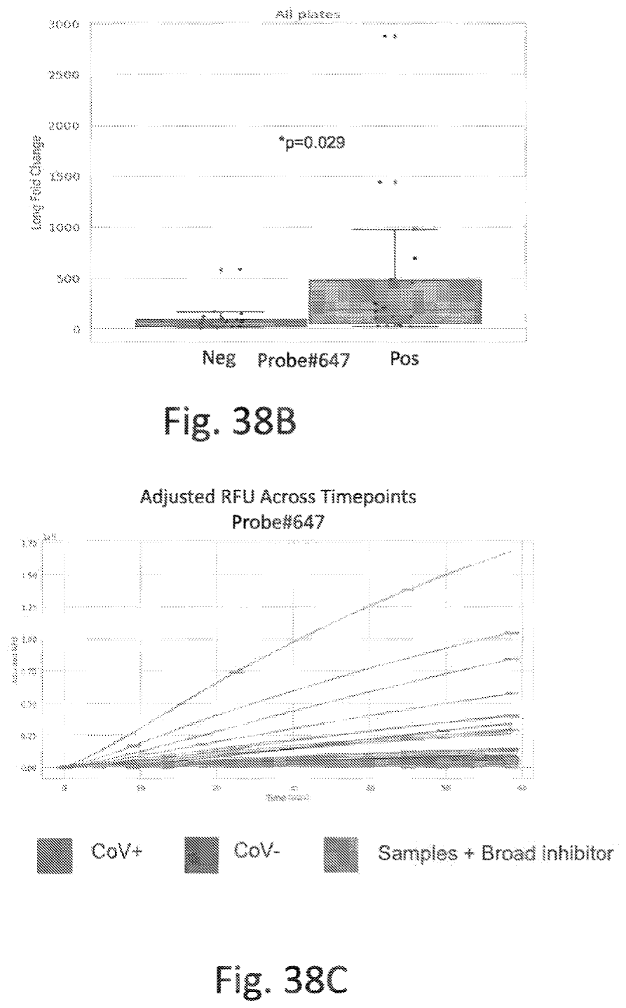

The specific probe, Probe #647, was shown to be a key differentiator between COVID+ and COVID-samples, as shown in FIG. 38A-C.

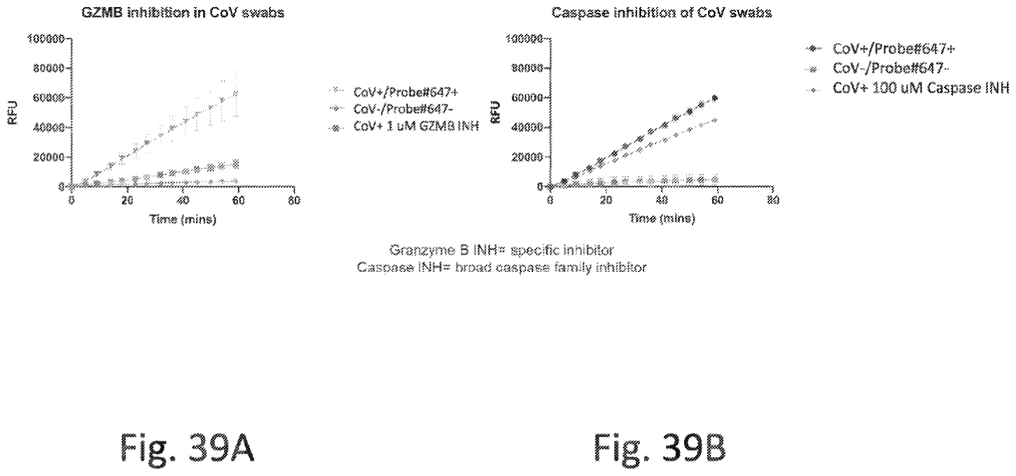

As shown in FIGS. 39A-B, Probe #647 signal measures the activity of protease Granzyme B to differentiate between healthy and COVID samples. Granzyme B is a protease that is linked to other autoimmune diseases and viral infections, showing the application can be applied to a wide range of disease biology.

Biotin and Probe #647 were conjugated by dissolving stock Probe #647 powder at 2 mM in 50/50 DMF/PBS. Biotin-Maleimide was reconstituted from powder at 100 mM and diluted to the following concentrations-2 mM, 3 mM and 6 mM in PBS. Three reaction mixtures were created with the following molar equivalents: 1) 1:1-10 uL to 10 uL 2 mM Biotin+2 mM Probe #647, 2) 1:1.5-10 to 10 uL 3 mM Biotin+2 mM Probe #647, and 3) 1:3-10 to 10 uL 6 mM Biotin+2 mM Probe #647. Once mixed, these were inverted on a Hula sample mixer for 2 hours at room temperature. Once the conjugation reactions were completed, recombinant proteases and samples were tested using 100 nM recombinant Granzyme B with 6 uM Probe #647-Biotin conjugate from above 3 reactions. These were then incubated for multiple time points: 0 mins, 5 minutes, 30 minutes, 1 hour, and optionally overnight. They were then diluted up 1:20 and paper strips were dipped into the mixture and the paper strip was read visually. Once the activity was confirmed using recombinant proteases, results were confirmed in strong COVID+ saline swab samples and COVID-saline swab samples (as determined by clinical PCR testing). 10 μL of dilute saline swab sample was combined with 5 uL Probe #647-Biotin conjugate and incubated for multiple time points: 0 hours and 2 hours. Post-reaction, the sample was diluted 1:20 and read visually with the paper strip.

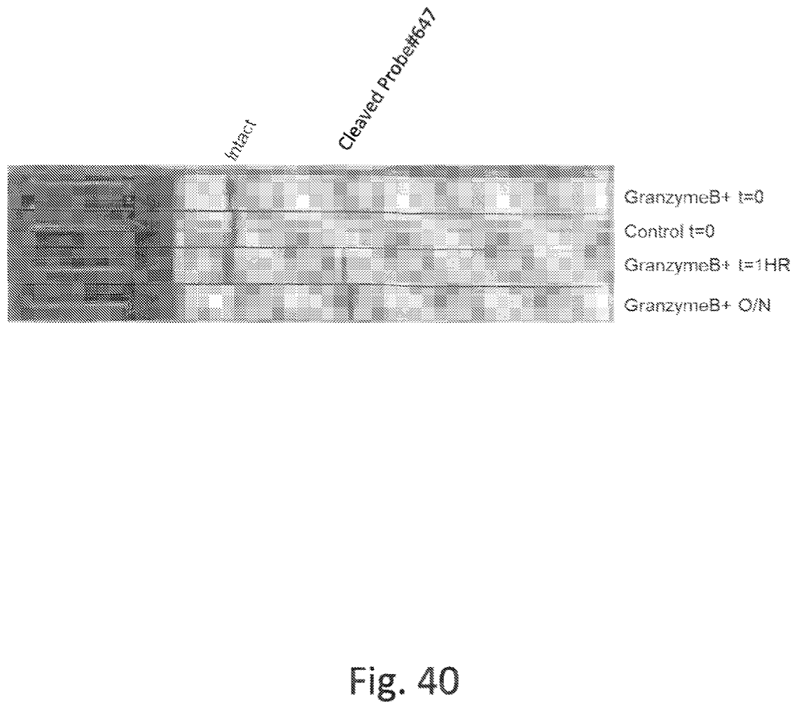

The use of a paper strip test to monitor Granzyme B activity using the probes of the application is shown in FIG. 40. This point of care test for the detection of protease cleavage of a biotin-tagged 5FAM sensor has implications for disease monitoring and response in real-time.

### Example 8: Liquid Biopsy Applications Towards Pancreatic Ductal Adenocarcinoma

In this example, the application is directed toward diagnosing pancreatic ductal adenocarcinoma (PDAC).

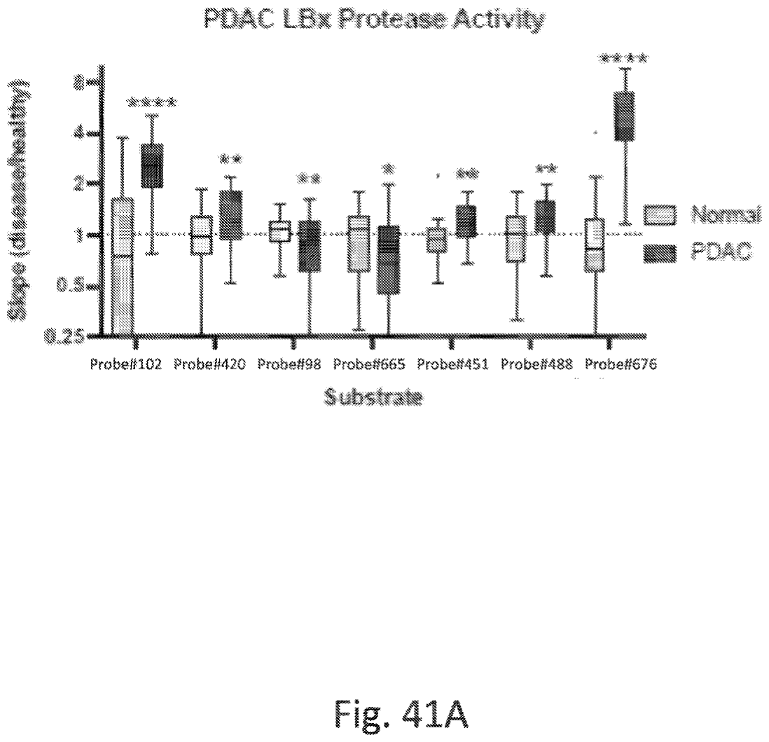

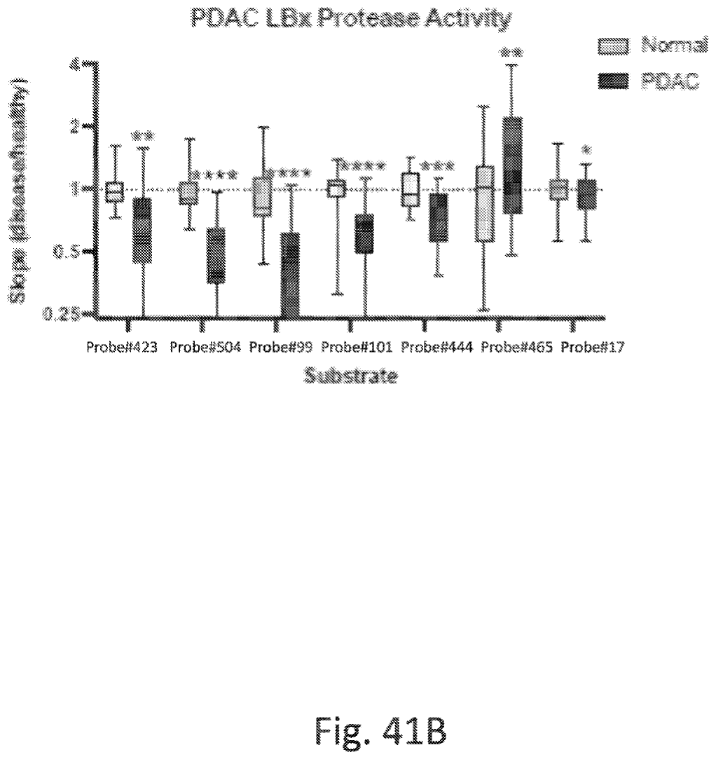

As shown in FIG. 41A-B, when human plasma is contacted with the probes of the application using the method from Experiment 1, one can distinguish between the protease activity of healthy and PDAC human plasma samples.

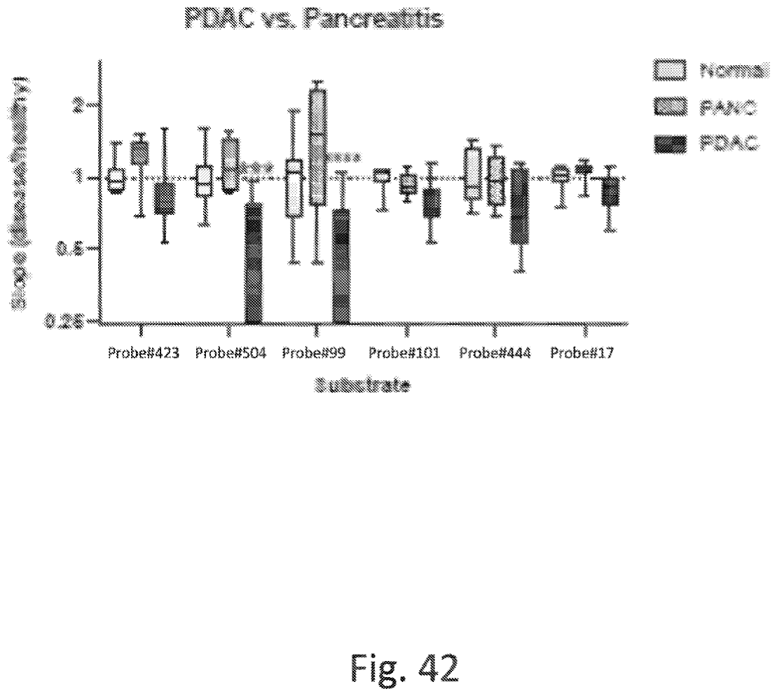

Furthermore, as shown in FIG. 42, the probes are able to differentiate among healthy, PDAC, and pancreatitis samples.

This experiment continues to show that there is broad applicability for the application regarding different types of diseases that have different protease biology.

### Example 9: Probes with a Fluorescent Reporter Will Accurately Measure NASH-Related Protease Activity Levels in Mice

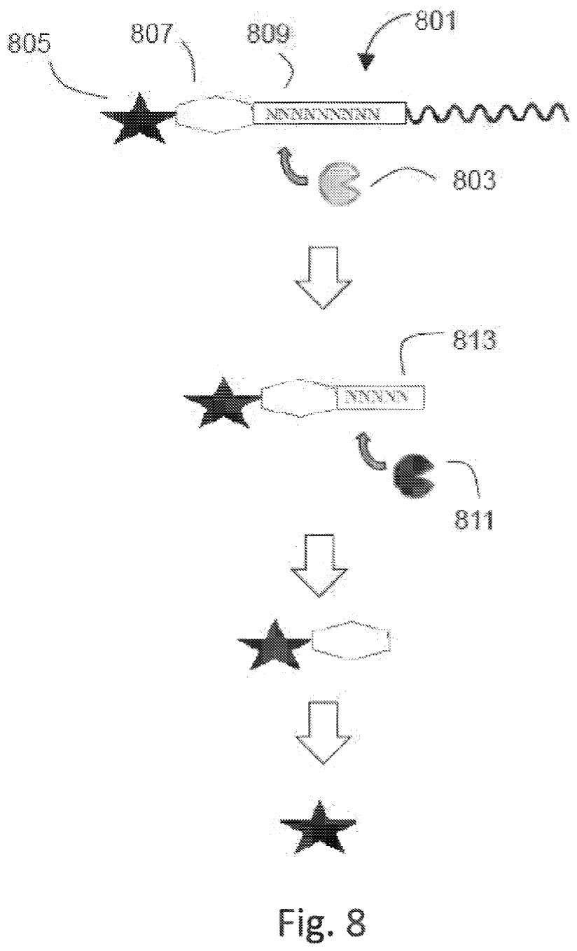

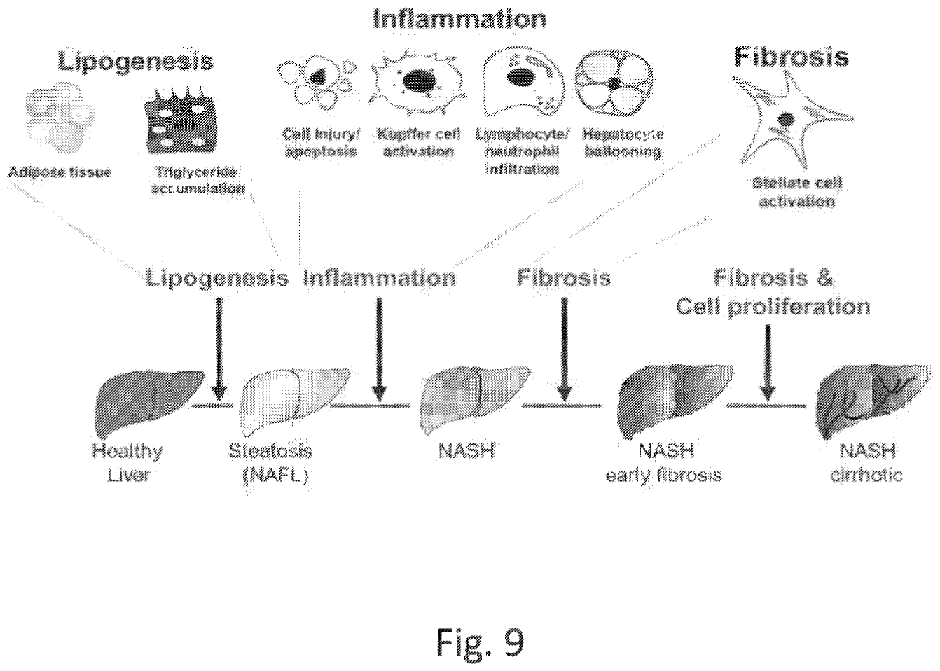

In this prophetic experiment, probes of the present disclosure that include a precipitating fluorescent reporter and a protease substrate cleavable by an endoprotease, like the probes discussed in FIG. 8, will be able to accurately measure the activity levels of NASH-related proteases in healthy mice and NASH mice.

The probes will be engineered such that the protease substrate could be cleaved by a protease such as endoprotease caspase 8, thereby resulting in a second protease substrate linked to a precipitating fluorescent reporter by an auto-immolative spacer. Alternatively, the second protease substrate could be cleaved by the endoprotease CTSD.

Spiking the plasma samples with an excess of CTSD would not result in a measured increase in caspase 8 activity. Thus, in the absence of caspase 8 to cleave the protease substrate, the second substrate will be unavailable for cleavage by CTSD, which will ultimately prevent precipitation of the fluorescent reporter.

However, upon addition of small concentrations of caspase 8 to the fluid sample, a strong signal will be detected by the precipitating fluorophores. Thus, caspase 8 will be able to cleave the protease substrate, thereby resulting in the second protease substrate, which will be cleaved by CTSD. This ultimately will lead to dissociation of the spacer from the precipitating fluorescent reporter, thereby resulting in a fluorescent signal.

Plasma samples with probes having distinguishable precipitating fluorescent reporters will be pooled after incubation with caspase 8 and CTSD. Individually, the plasma samples will be taken from either healthy mice or those with NASH to determine the differences between healthy and NASH samples through detection of caspase 8.

### Example 10: Detecting Alternative Enzymes

In this experiment, measurement of alternative enzymes' activities for disease detection is explored. Different enzyme classes include peroxidases, lipases, esterases, phospholipases, amylase etc.

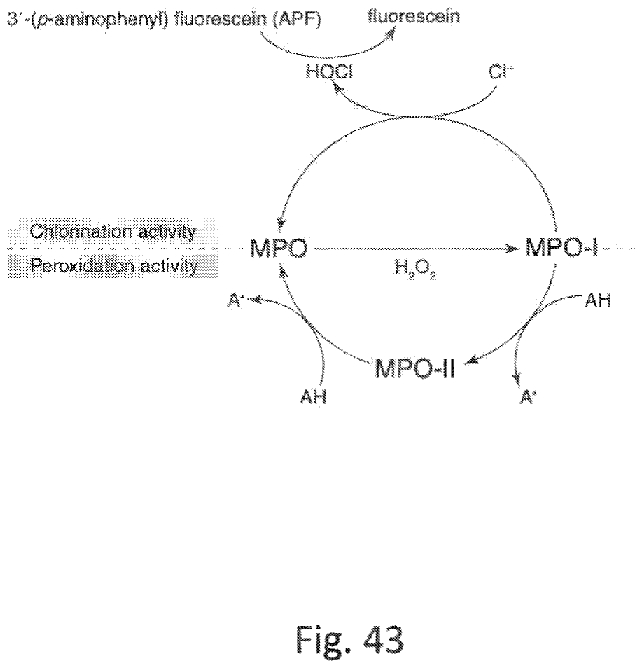

FIG. 43 shows a schematic diagram for detection of Chlorination and peroxidation activity of MPO using the EnzChek® Myeloperoxidase Activity Assay Kit. AH represents the nonfluorescent Amplex® UltraRed substrate, and A represents its fluorescent oxidation product. Hydrogen peroxide converts MPO to MPO-I and MPO is inactive without the presence of hydrogen peroxide. Amplex® UltraRed is then oxidized by MPO-I and creates the fluorescent oxidation product A which can be read at Ex/Em=530/590.

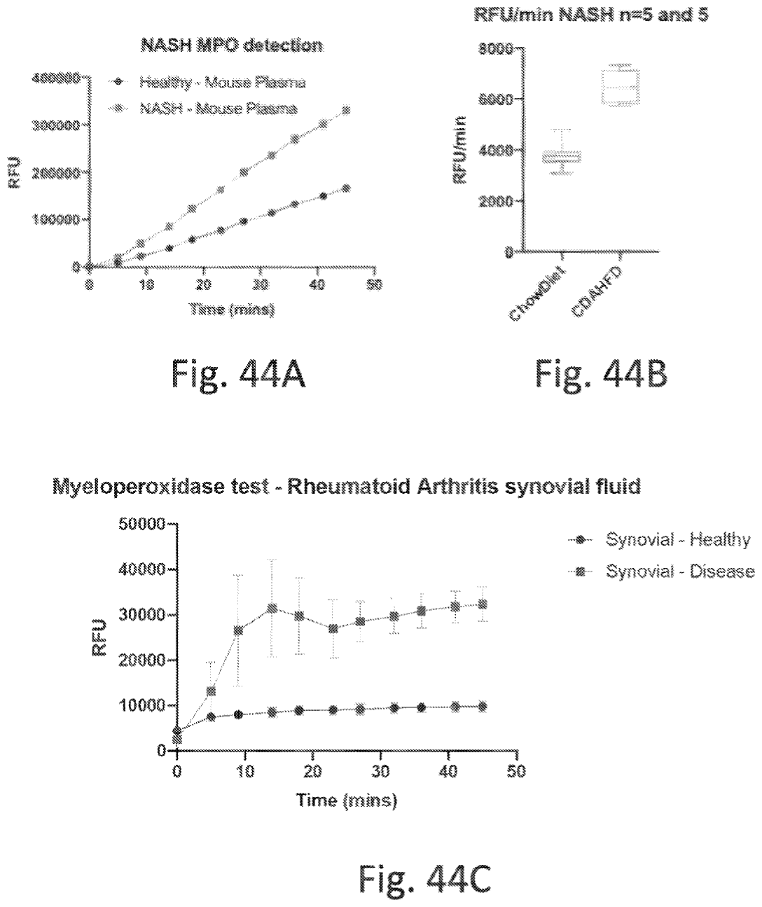

FIG. 44A-C shows the results for detecting peroxidases. FIG. 44A shows that MPO activities are different between healthy mice and mice with NASH. FIG. 44B shows that MPO activities are different between mice fed on a standard ChowDiet (CD), and mice fed on a choline-deficient, L-amino acid-defined, high-fat diet (CDAHFD). FIG. 44C shows that MPO activities are different between healthy subjects and subjects with rheumatoid arthritis. This result shows that we are capable of detecting differential activity in NASH in plasma and rheumatoid arthritis in human pools in synovial fluid.

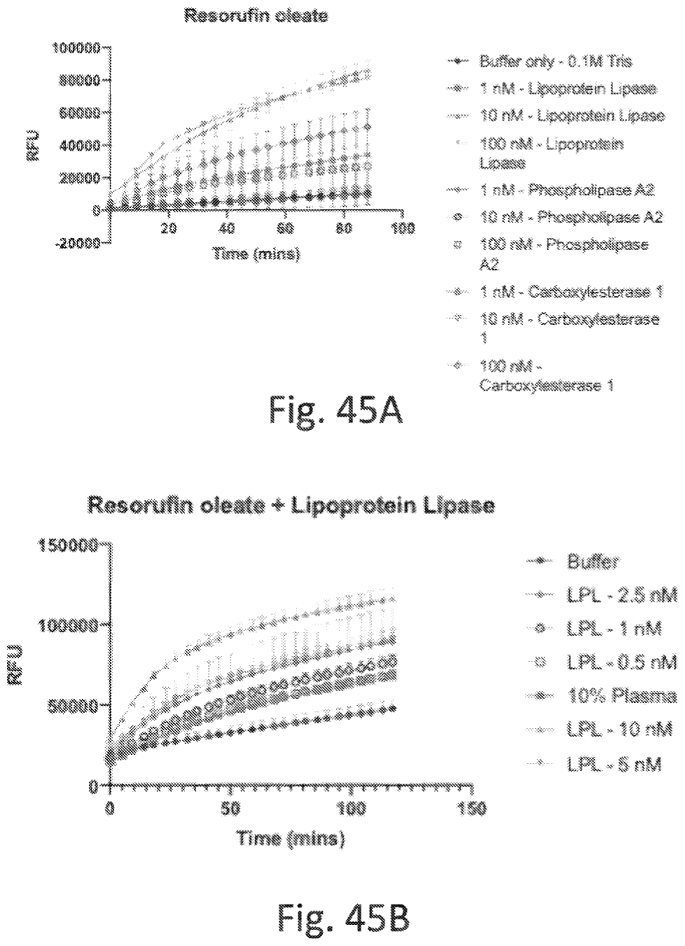

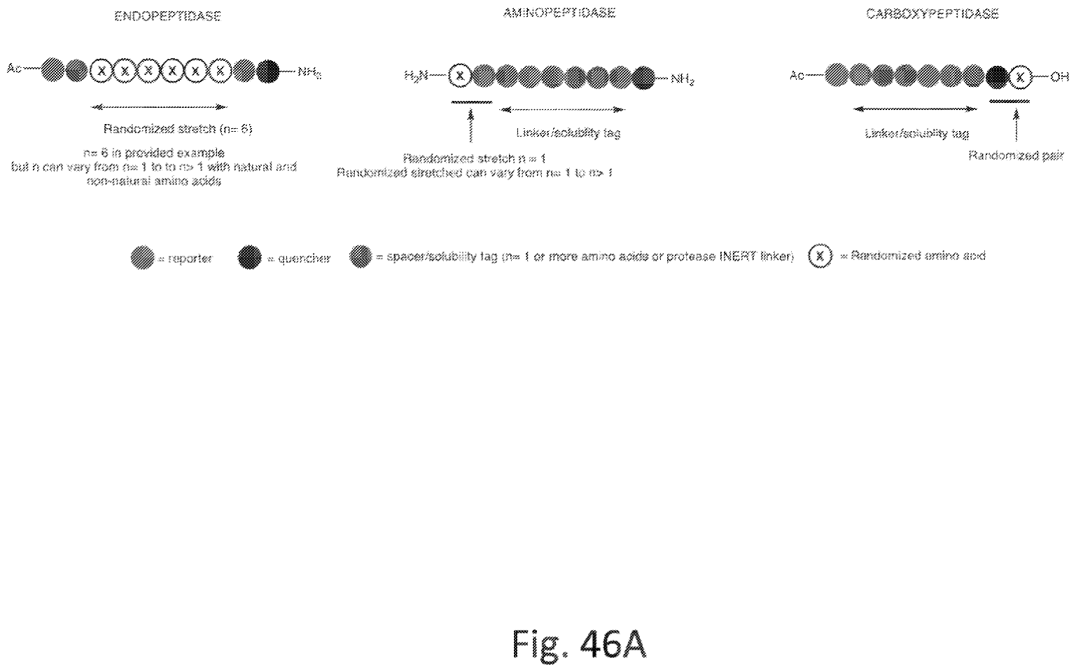

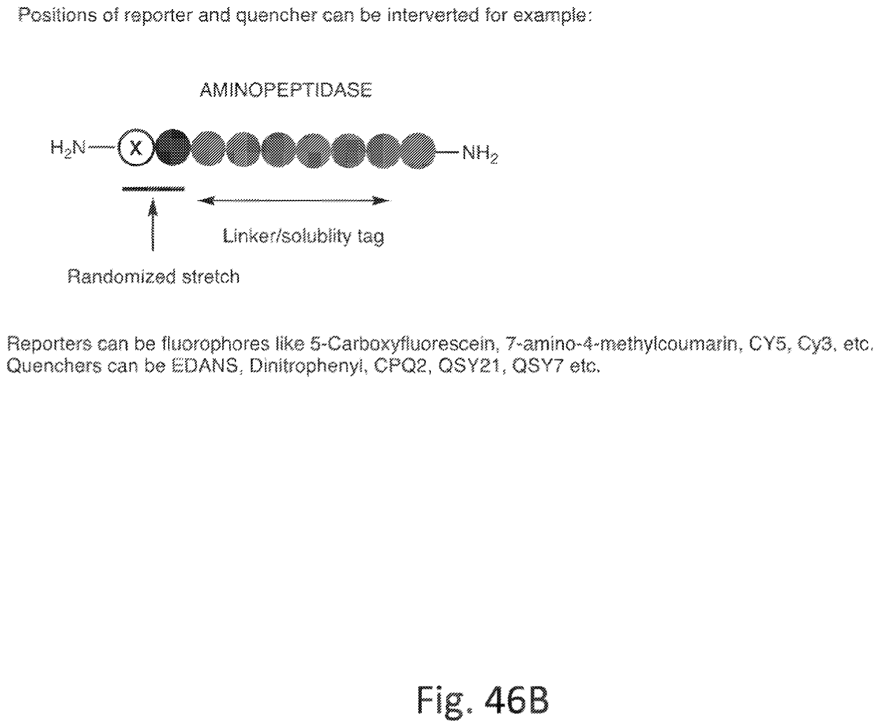

FIG. 45A-B shows the pooled results of spiked recombinant protease in human plasma using resorufin oleate as substrate. FIG. 46A shows result of 3 recombinant enzymes-carboxylesterase 1, phospholipase A2 and lipoprotein lipase. FIG. 46B shows the result of various concentrations of lipoprotein lipase. This result demonstrates that Resorufin oleate and butyrate were promising for detection of broad range of enzymes.

### Example 11: Combinatorial Use of Liquid Biopsy to Diagnose NASH

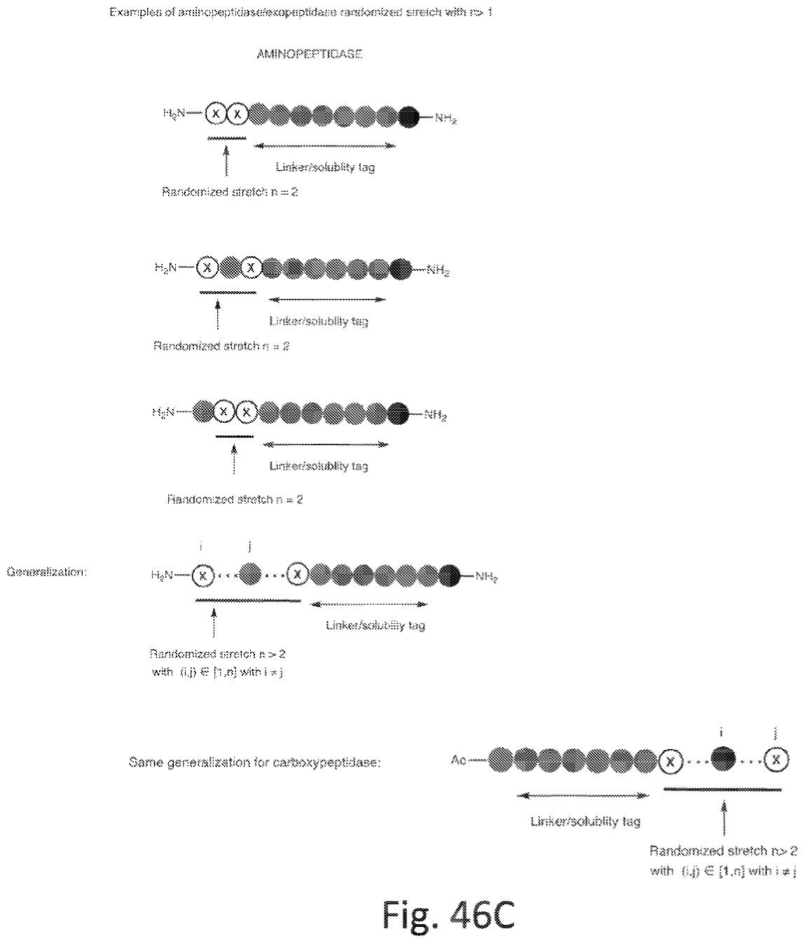

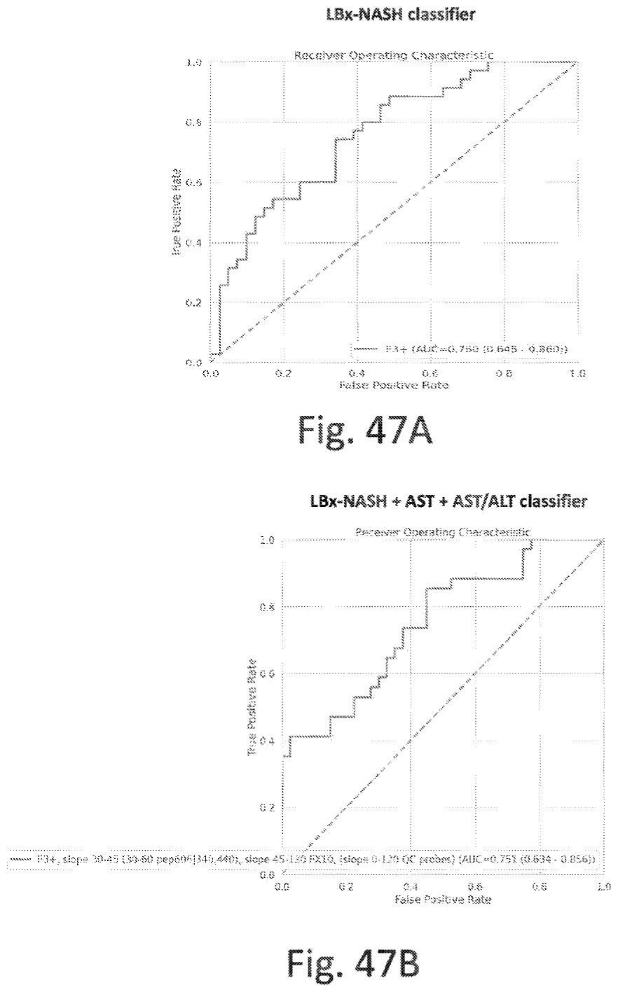

In this example, human plasma was contacted with the probes of the application using the method from Experiment 1 whereby the experiment detected protease activity levels of either healthy or NASH samples. This information was input into an algorithm along with other patient information such as clinical test results, genetic profile information, molecular profile information, and demographic characteristics. The algorithm determined the differences between healthy and NASH samples. The algorithm further determined the differences between different stages of NASH samples. Results are shown in FIG. 47A-B.

### Example 12: Combinatorial Use of Liquid Biopsy and Liver Test Probes to Diagnose NASH

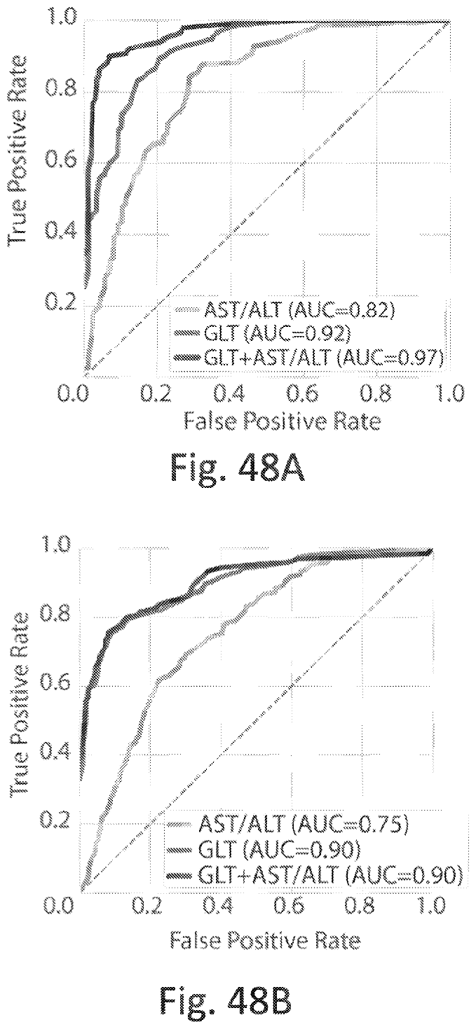

In this example, mouse plasma was contacted with the probes of the application using the method from Experiment 1 whereby the experiment detected protease activity levels of either healthy or NASH samples (GLT). This information was input into an algorithm along with other patient information such as the results of AST tests and ALT tests. The algorithm determined the differences between healthy and NASH samples, with better differentiation when using the GLT alone (FIG. 48A-B).

### Example 13: Combinatorial Use of Liquid Biopsy, Liver Test Probes, and Enhanced Liver Fibrosis Scores to Diagnose NASH

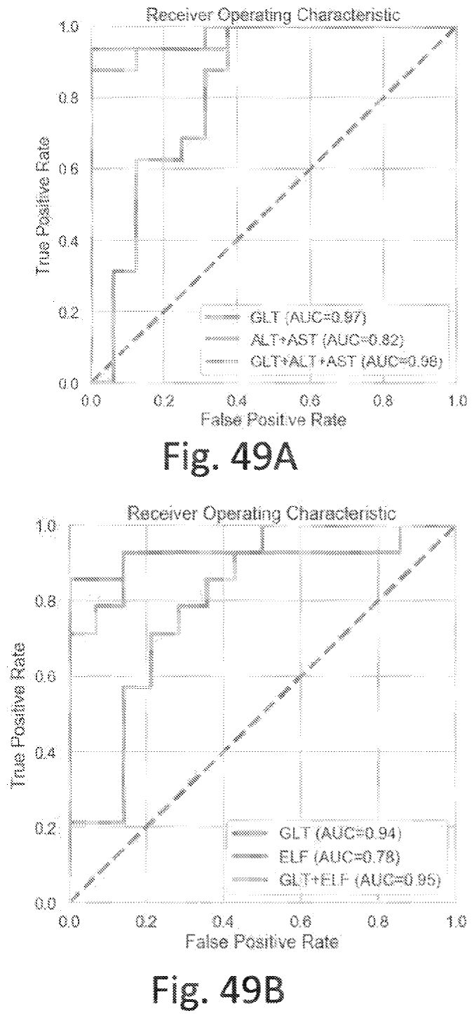

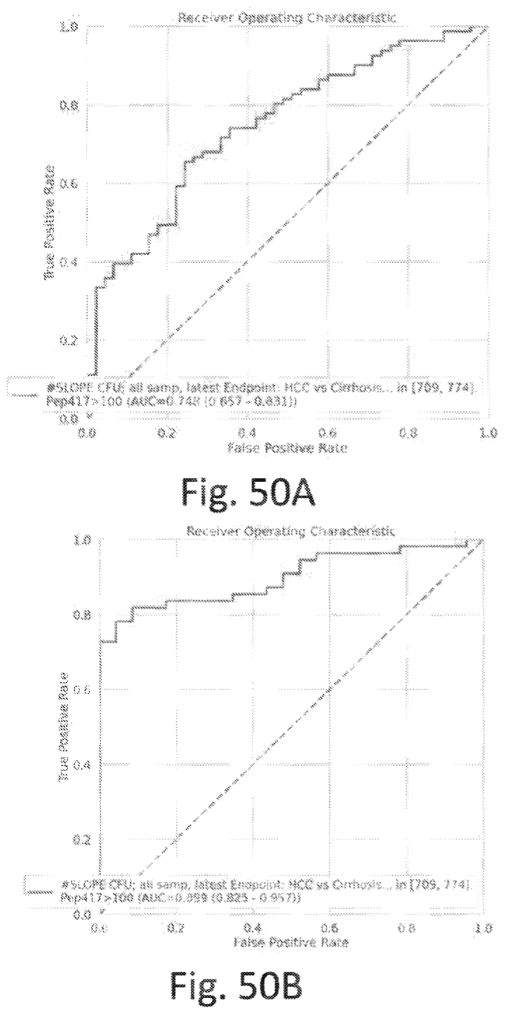

In this example, rat plasma was contacted with the probes of the application using the method from Experiment 1 whereby the experiment detected protease activity levels of either healthy or NASH samples (GLT). This information was input into an algorithm along with other subject information such as Enhanced Liver Fibrosis (ELF) score. The algorithm determined the differences between healthy and NASH samples (FIG. 49A-B).

### Example 14: Plasma Protease Activity and Alpha Feto-Protein (AFP) to Detect HCC

HCC surveillance using ultrasound can be combined with detection of Alpha-Feto-Protein (AFP). In this experiment, AFP detection was combined with a protease biosensor biomarker panel to evaluate the efficacy of the combination treatment to distinguish patients with HCC from patients with cirrhosis without HCC.

Retrospective plasma samples were obtained from patients diagnosed with HCC (cases) and those with cirrhosis without HCC (controls). Protease biosensor cleavage was assayed from plasma by fluorimetry. The relative signal was used for classification by regularized logistic regression using 100 cross-validation (80% train, 20% validation splits). The maximum point from the Youden's J index in the training set was used to the define the optimal cut-off, which was used to calculate sensitivity and specificity for HCC detection in the validation sets. AFP was measured in plasma samples for all patients using a standard ELISA assay. AFP thresholds at both 20 ng/ml and 10 ng/ml were evaluated in this analysis.

Plasma protease activity and AFP levels were assessed in 54 HCC cases (76% male, mean age 56 years, 65% HBV, 82% TNM stage 1 or 2) and 23 cirrhosis controls (35% male, mean age 58 years, 100% NASH etiology). Our panel of protease biosensors had a high accuracy for differentiating patients with HCC vs those with cirrhosis (AUC 0.87 [95% CI 0.78-0.94]). The sensitivity and specificity of the assay, at its pre-specified cut-off, were 0.79 and 0.77, respectively. The sensitivity and specificity of AFP at a cut-off of 20 ng/ml were 0.44 and 1.00, respectively. Of note, the assay was able to detect HCC in 68% (22 of 30) of patients with AFP<20 ng/mL. Finally, a combined classifier of the assay and AFP>20 ng/ml had an AUC of 0.90 [95% CI 0.82-0.96] for differentiating HCC from cirrhosis. The sensitivity and specificity of the combined classifier for HCC detection were 0.82 and 0.82, respectively. Similar results were observed when the AFP threshold was adjusted to the more stringent cutoff of 10 ng/ml.

As seen in FIG. 10A-B, the protease biosensor panel assay was able to correctly classify as HCC 68% (17 out 25) of patients with low AFP levels (AFP<10 ng/ml). Moreover, classification by regularized logistic regression with 100-fold cross-validation (80% train, 20% validation splits) using the protease biosensor panel assay in the challenging subset of patients with low AFP levels (AFP, 10 ng/ml) showed an AUC of 0.75. Finally, a combined classifier of the protease biosensor panel assay and AFP had an AUC of 0.90 [CI 0.83-0.96] for differentiating HCC from cirrhosis in this cohort of patients (FIG. 10B). Using the Youden's cut-off, sensitivity and specificity for HCC detection of this combined classifier of protease biosensor panel plus AFP were 0.84 and 0.87, respectively. These combined results demonstrate the utility of using novel non-invasive biosensors measuring protease activity independently or in combination with AFP to accurately differentiate HCC from cirrhosis.

While preferred embodiments of the present disclosure have been shown and described herein, it will be obvious to those skilled in the art that such embodiments are provided by way of example only. It is not intended that the present disclosure be limited by the specific examples provided within the specification. While the present disclosure has been described with reference to the aforementioned specification, the descriptions and illustrations of the embodiments herein are not meant to be construed in a limiting sense. Numerous variations, changes, and substitutions will now occur to those skilled in the art without departing from the present disclosure. Furthermore, it shall be understood that all aspects of the present disclosure are not limited to the specific depictions, configurations or relative proportions set forth herein which depend upon a variety of conditions and variables. It should be understood that various alternatives to the embodiments of the present disclosure described herein may be employed in practicing the present disclosure. It is therefore contemplated that the present disclosure shall also cover any such alternatives, modifications, variations or equivalents. It is intended that the following claims define the scope of the present disclosure and that methods and structures within the scope of these claims and their equivalents be covered thereby.

What is claimed is:

1. A method comprising:
contacting a body fluid sample from a subject with a synthetic molecule,
wherein said synthetic molecule comprises a cleavable linker and a reporter, and
wherein an agent from body fluid sample cleaves said cleavable linker, thereby releasing said reporter;
detecting said released reporter; and
inputting data from (a) said detection and (b) a parameter that indicates a disease, physiological or clinical condition of said subject into an algorithm, wherein said parameter is not associated with said detectable signal.

2. The method of claim 1, further comprising determining and monitoring a disease, a condition, or a change in condition of said subject based on an output from said algorithm.

3. The method of claim 1 or claim 2, wherein said algorithm comprises a supervised or unsupervised Machine Learning or Deep Learning algorithm, Logistic Regression, Naive Bayes, Support Vector Machine, Decision Tree, Random Forest, Gradient Boosting, Regularizing Gradient Boosting, K-Nearest Neighbors, a continuous regression approach, Ridge Regression, Kernel Ridge Regression, Support Vector Regression, deep learning approach, Neural Networks, Convolutional Neural Network (CNNs), Recurrent Neural Networks (RNNs), Long Short Term Memory Networks (LSTMs), Generative Models, Generative Adversarial Networks (GANs), Deep Belief Networks (DBNs), Feedforward Neural Networks, Autoencoders, Radial Basis Function Networks (RBFNs), Multilayer Perceptrons (MLPs), Stochastic Neural Networks, or any combination thereof.

4. The method of any one of claims 1-3, wherein said parameter comprises a result from a clinical test of said subject, a result from a genetic test of said subject, a result from a protein test of said subject, a demographic characteristic of said subject, a clinical characteristic of said subject, a digital profile of said subject, or a combination thereof.

5. The method of claim 4, wherein said clinical test comprises a tissue biopsy, a blood test, a test for proteins, a test for metabolites, a test for a circulating analyte, a test on any bodily fluid, a test for microbiome, a test for biomarkers, a test for malignancy or tumor, a test for liver function, a test for lipids, or any combination thereof.

6. The method of claim 5, wherein said blood test comprises an enhanced liver fibrosis (ELF) test or any components thereof.

7. The method of claim 5, wherein said circulating analyte comprises a polypeptide, a protein, a glycoprotein, a cytokine, a hormone, or a combination thereof.

8. The method of claim 4, wherein said genetic test comprises an analysis of a nucleic acid, a genomic DNA, a mitochondrial DNA (mtDNA), a mRNA, a microRNA (miRNA), a long non-coding RNA (lncRNA), a snoRNA, rRNA, tRNA, a circulating cell-free DNA (cfDNA), a circulating tumor DNA (ctDNA), a cell-free RNA (cfRNA), a cell-free total nucleic acid (cfTNA), an exosomal nucleic acid, a microbial or viral nucleic acid, a DNA methylation marker, or any combination thereof.

9. The method of claim 4, wherein said protein test comprises an analysis of a post-translational modification, total protein, proteomics, protein ratios or any combination thereof.

10. The method of claim 9, wherein said post-translational modification comprises phosphorylation, glycosylation, ubiquitination, nitrosylation, methylation, acetylation, lipidation, hydroxylation, proteolysis, or any combination thereof.

11. The method of claim 4, wherein said demographic characteristic comprises age, gender, race, ethnicity, education, occupation, income, geographic area, or any combination thereof.

12. The method of claim 4, wherein said clinical characteristic comprises disease characteristics, symptoms and severity/stage of a disease or condition, body mass index (BMI), age at diagnosis, type 2 diabetes, metabolic syndrome, MELD score, alcohol or other toxin consumption, dyslipidemia, genetic risk profile (PNPLA-3), liver stiffness, controlled attenuation parameter (CAP), or any combination thereof.

13. The method of claim 4, wherein said digital profile comprises a digital measurement or data set collected by a wearable device.

14. The method of claim 13, wherein said wearable device comprises a smart watch, a smart clothing, a smart jewelry, a fitness tracker, an implantable device, a head-mounted display, or any combination thereof.

15. The method of claim 13, wherein said digital measurement comprises heart rate, brainwave, muscle bio-signals, sleep patterns and duration, speed, cadence, distance traveled, calories burned, respiration rate, skin conductivity, biomarkers from a bodily fluid, or any combination thereof.

16. The method of claim 1, wherein said parameter comprises at least two parameters.

17. The method of claim 1, wherein said parameter comprises at least five parameters.

18. The method of claim 1, wherein said parameter comprises at least ten parameters.

19. The method of claim 1, wherein said parameter comprises at least twenty parameters.

20. The method of claim 1, wherein said parameter comprises at least fifty parameters.

21. The method of any one of claims 1 to 20, further comprising:
contacting a second body fluid sample from the subject with a second synthetic molecule,
wherein said second synthetic molecule comprises a second cleavable linker and a second reporter, and
wherein said a second agent from said second body fluid sample cleaves said second cleavable linker, thereby releasing said second reporter from said second synthetic molecule,
detecting said released second reporter.

22. The method of claim 21, wherein said second body fluid sample is different from said body fluid sample.

23. The method of claim 21, wherein said second cleavable linker is different from said cleavable linker.

24. The method of claim 1, wherein said disease, physiological or clinical condition comprises liver disease, cancer, organ transplant rejection, infectious diseases, allergic diseases, an autoimmune disease, a chronic or acute inflammation, or an Alzheimer's disease, neurologic disease, or any other protease related disease.

25. The method of claim 24, wherein said liver disease comprises a Non-alcoholic steatohepatitis (NASH), a non-alcoholic fatty liver disease (NAFLD), a toxin mediated liver injury, a viral hepatitis, a fulminant hepatitis, an alcoholic hepatitis, an autoimmune hepatitis, a cirrhosis of the liver, a hepatocellular carcinoma (HCC), a primary biliary cholangitis (PBC), a cholangiocarcinoma, a primary sclerosing cholangitis, an acute or chronic rejection of a transplanted liver, a drug induced liver injury, an inherited liver disease, or a combination thereof.

26. The method of any one of claims 1-25, wherein said body fluid sample is selected from the group consisting of blood, plasma, serum, bone marrow fluid, lymphatic fluid, bile, amniotic fluid, mucosal fluid, saliva, urine, cerebrospinal fluid, spinal fluid, synovial fluid, ascitic fluid, semen, ductal aspirate, feces, stool, vaginal effluent, lachrymal fluid, tissue lysate, and patient-derived cell line supernatant.

27. The method of any one of claims 1-25, wherein said body fluid sample comprises a rinse fluid, a conditioning media or buffer, a swab viral transport media, a saline, a culture media, or a cell culture supernatant.

28. The method of claim 27, wherein said rinse fluid is selected from the group consisting of a mouthwash rinse, a bronchioalveolar rinse, a lavage fluid, a hair wash rinse, a nasal spray effluent, a swab of any bodily surface, orifice, organ structure or solid tumor biopsies applied to saline or any media or any derivatives thereof.

29. The method of any one of claims 1-28, wherein said agent is selected from the group consisting of a oxidoreductase, a transferase, a hydrolase, a lyase, a isomerase, a ligase, a protease (peptidase), a hydrolase, an esterase, a β-glycosidase, a phospholipase and a phosphodiesterase, peroxidase, lipase, amylase a nucleophilic reagent, a reducing reagent, a electrophilic/acidic reagent, an organometallic/metal catalyst, an oxidizing reagent, a hydroxyl ion, a thiols nucleophile, a nitrogen nucleophile, a sodium dithionite, and a sodium periodate.

30. The method of claim 29, wherein said agent comprises a protease.

31. The method of claim 30, wherein said protease comprises an endopeptidase or an exopeptidase.

32. The method of claim 30, wherein said protease is selected from the group consisting of an A20 (TNFa-induced protein 3), an abhydrolase domain containing 4, an abhydrolase domain containing 12, an abhydrolase domain containing 12B, an abhydrolase domain containing 13, an acrosin, an acylaminoacyl-peptidase, a disintegrin and metalloproteinase (ADAM), an ADAM1a, an ADAM2 (Fertilin-b), an ADAM3B, an ADAM4, an ADAM4B, an ADAM5, an ADAM6, an ADAM7, an ADAM8, an ADAM9, an ADAM10, an ADAM11, an ADAM12 metalloprotease, an ADAM15, an ADAM17, an ADAM18, an ADAM19, an ADAM20, an ADAM21, an ADAM22, an ADAM23, an ADAM28, an ADAM29, an ADAM30, an ADAM32, an ADAM33, a disintegrin and metalloproteinase with thrombospondin motifs (ADAMTS), an ADAMTS1, an ADAMTS2, an ADAMTS3, an ADAMTS4, an ADAMTS5/11, an ADAMTS6, an ADAMTS7, an ADAMTS8, an ADAMTS9, an ADAMTS10, an ADAMTS12, an ADAMTS13, an ADAMTS14, an ADAMTS15, an ADAMTS16, an ADAMTS17, an ADAMTS18, an ADAMTS19, an ADAMTS20, an adipocyte-enh. binding protein 1, an Afg3-like protein 1, an Afg3-like protein 2, an airway-trypsin-like protease, an aminoacylase, an aminopeptidase A, an aminopeptidase B, an aminopeptidase B-like 1, an aminopeptidase MAMS/L-RAP, an aminopeptidase N, an aminopeptidase O, an aminopeptidase P homologue, an aminopeptidase P1, an aminopeptidase PILS, an aminopeptidase Q, an aminopeptidase-like 1, an AMSH/STAMBP, an AMSH-LP/STAMBPL1, an angiotensin-converting enzyme 1 (ACE1), an angiotensin-converting enzyme 2 (ACE2), an angiotensin-converting enzyme 3 (ACE3), an anionic trypsin (II), an apolipoprotein (a), an archaemetzincin-1, an archaemetzincin-2, an aspartoacylase, an aspartoacylase-3, an aspartyl aminopeptidase, an ataxin-3, an ataxin-3 like, an ATP/GTP binding protein 1, an ATP/GTP binding protein-like 2, an ATP/GTP binding protein-like 3, an ATP/GTP binding protein-like 4, an ATP/GTP binding protein-like 5, an ATP23 peptidase, an autophagin-1, an autophagin-2, an autophagin-3, an autophagin-4, an azurocidin, a beta lactamase, a beta-secretase 1, a beta-secretase 2, a bleomycin hydrolase, a brain serine proteinase 2, a BRCC36 (BRCA2-containing complex, sub 3), a calpain, a calpain 1, a calpain 2, a calpain 3, a calpain 4, a calpain 5, a calpain 6, a calpain 7, a calpain 7-like, a calpain 8, a calpain 9, a calpain 10, a calpain 11, a calpain 12, a calpain 13, a calpain 14, a calpain 15 (Solh protein), a cysteine protease, a carboxypeptidase A1, a carboxypeptidase A2, a carboxypeptidase A3, a carboxypeptidase A4, a carboxypeptidase A5, a carboxypeptidase A6, a carboxypeptidase B, a carboxypeptidase D, a carboxypeptidase E, a carboxypeptidase M, a carboxypeptidase N, a carboxypeptidase O, a carboxypeptidase U, a carboxypeptidase X1, a carboxypeptidase X2, a carboxypeptidase Z, a carnosine dipeptidase 1, a carnosine dipeptidase 2, a caspase recruitment domain family, member 8, a caspase, a caspase-1, a caspase-2, a caspase-3, a caspase-4/11, a caspase-5, a caspase-6, a caspase-7, a caspase-8, a caspase-9, a caspase-10, a caspase-12, a caspase-14, a caspase-14-like, a casper/FLIP, a cathepsin, a cathepsin A (CTSA), a cathepsin B (CTSB), a cathepsin C (CTSC), a cathepsin D (CTSD), a cathepsin E (CTSE), a cathepsin F, a cathepsin G, a cathepsin H (CTSH), a cathepsin K (CTSK), a cathepsin L (CTSL), a cathepsin L2, a cathepsin O, a cathepsin S (CTSS), a cathepsin V (CTSV), a cathepsin W, a cathepsin Z (CTSZ), a cationic trypsin, a cezanne/OTU domain containing 7B, a cezanne-2, a CGI-58, a chymase, a chymopasin, a chymosin, a chymotrypsin B, a chymotrypsin C, a coagulation factor IXa, a coagulation factor VIIa, a coagulation factor Xa, a coagulation factor XIa, a coagulation factor XIIa, a collagenase 1, a collagenase 2, a collagenase 3, a complement protease C1r serine protease, a complement protease C1s serine protease, a complement C1r-homolog, a complement component 2, a complement component C1ra, a complement component C1sa, a complement factor B, a complement factor D, a complement factor D-like, a complement factor I, a COPS6, a corin, a CSN5 (JAB1), a cylindromatosis protein, a cytosol alanyl aminopep.-like 1, a cytosol alanyl aminopeptidase, a DDI-related protease, a DECYSIN, a Der1-like domain family, member 1, a Der1-like domain family, member 2, a Der1-like domain family, member 3, a DESC1 protease, a desert hedgehog protein, a desumoylating isopeptidase 1, a desumoylating isopeptidase 2, a dihydroorotase, a dihydropyrimidinase, a dihydropyrimidinase-related protein 1, a dihydropyrimidinase-related protein 2, a dihydropyrimidinase-related protein 3, a dihydropyrimidinase-related protein 4, a dihydropyrimidinase-related protein 5, a DINE peptidase, a dipeptidyl peptidase (DPP), a dipeptidyl peptidase (DPP1), a dipeptidyl-peptidase 4 (DPP4), a dipeptidyl-peptidase 6 (DPP6), a dipeptidyl-peptidase 8 (DPP8), a dipeptidyl-peptidase 9 (DPP9), a dipeptidyl-peptidase II, a dipeptidyl-peptidase III, a dipeptidyl-peptidase 10 (DPP10), a DJ-1, a DNA-damage inducible protein, a DNA-damage inducible protein 2, a DUB-1, a DUB-2, a DUB2a, a DUB2a-like, a DUB2a-like2, a DUB6, or a combination thereof.

33. The method of claim 30, wherein said protease is selected from the group consisting of a T cell protease, a complement protease, a fibrosis protease, and an inflammation-related protease.

34. The method of any one of claims 1-33, wherein said cleavable linker comprises a peptide, a carbohydrate, a nucleic acid, a lipid, an ester, a glycoside, a phospholipid, a phosphodiester, a nucleophile/base sensitive linker, a reduction sensitive linker, an electrophile/acid sensitive linker, a metal cleavable linker, an oxidation sensitive linker, or a combination thereof.

35. The method of claim 34, wherein said cleavable linker is a peptide.

36. The method of claim 34, wherein said peptide comprises an amino acid sequence selected from the group consisting of SEQ ID Nos: 1-677 or a sequence comprising a mimetic of any one of SEQ ID Nos: 1-677.

37. The method of claim 36, wherein said mimetic comprises a beta amino acid or a peptoid.

38. The method of claim 37, wherein said amino acid comprises a chemical modification.

39. The method of claim 38, wherein said chemical modification comprises phosphorylation, alkylation, arylation, amination, amidation, sulfonylation, halogenation, borylation, glycosylation, cyclization, linearization, hydration, hydrogenation, nitration, nitrosylation, reduction, oxidation, esterification, hydrolysis, dephosphorylation, dealkylation, dearylation, deamination, deamidation, desulfonylation, dehalogenation, deborylation, deglycosylation, decyclization, delinearization, dehydration, dehydrogenation, denitration, denitrosylation, deesterification, dehydrolysis or any combination thereof.

40. The method of claim 1, wherein said cleavable linker is directly connected to said reporter through a covalent bond.

41. The method of claim 1, wherein said reporter comprises a fluorescent label, a mass tag, a chromophore, an electrochemically active molecule, a bio-Layer interferometry or surface plasmon resonance detectable molecule, a precipitating substance, a mass spectrometry and liquid chromatography substrate, a magnetically active molecule, a gel forming and/or viscosity changing molecule, an immunoassay detectable molecule, a cell-based amplification detectable or a nucleic acid barcode, or any combination thereof.

42. The method of claim 41, wherein said reporter comprises a fluorescent label.

43. The method of claim 42, wherein said fluorescent label is selected from a group consisting of a 5-carboxyfluorescein (5-FAM), a 7-amino-4-carbamoylmethylcoumarin (ACC), a 7-Amino-4-methylcoumarin (AMC), a 2-Aminobenzoyl (Abz), a Cy7, a Cy5, a Cy3, and a (5-((2-Aminoethyl)amino)naphthalene-1-sulfonic acid) (EDANS).

44. The method of claim 42 or claim 43, wherein said synthetic molecule comprises a fluorescent quencher.

45. The method of claim 44, wherein said fluorescent quencher is selected from the group consisting of BHQ0, BHQ1, BHQ2, BHQ3, BBQ650, ATTO 540Q, ATTO 580Q, ATTO 612Q, CPQ2, QSY-21, QSY-35, QSY-7, QSY-9, DABCYL (4-([4′-dimethylamino)phenyl] azo)benzoyl), Dnp (2,4-dinitrophenyl), and Eclipse.

46. The method of claim 44 or claim 45, wherein said fluorescent quencher is directly connected to said cleavable linker through a covalent bond.

47. The method of any one of claims 1-46, wherein said synthetic molecule comprises a carrier.

48. The method of claim 47, wherein said carrier comprises a native protein, a labeled protein, a synthetic protein, a synthetic chemical polymer of precisely known chemical composition or with a distribution around a mean molecular weight, an oligonucleotide, a phosphorodiamidate morpholino oligomer (PMO), a foldamer, a lipid, a lipid micelle, a nanoparticle, a solid support made of polystyrene, polypropylene or any other type of plastic, or any combination thereof.

49. The method of claim 1, wherein said detection comprises fluorescent detection, spectroscopic detection, mass spectrometry, immunological detection, DNA unique molecular identifier barcode detection, imaging detection, or any combination thereof.

50. The method of claim 49, wherein said fluorescent detection is fluorescence resonance energy transfer (FRET).

51. The method of any one of claims 1-50, wherein the contacting is conducted ex vivo.

52. The method of claim 1, wherein said cleavable linker comprises a nucleic acid.

53. The method of claim 1, wherein said subject comprises a mammal.

54. The method of claim 53, wherein said mammal comprises a human.


SEQUENCE LISTING (1298 sequences)

SEQ ID NO: 1 - protein, 5 aa, synthetic construct
SGRSG

SEQ ID NO: 2 - protein, 6 aa, synthetic construct
PGPREG

SEQ ID NO: 3 - protein, 8 aa, synthetic construct
IEPDSGSQ

SEQ ID NO: 4 - protein, 9 aa, synthetic construct
VVADSSMES

SEQ ID NO: 5 - protein, 4 aa, synthetic construct
PTSY

SEQ ID NO: 6 - protein, 4 aa, synthetic construct
YRFK

SEQ ID NO: 7 - protein, 4 aa, synthetic construct
KVPL

SEQ ID NO: 8 - protein, 5 aa, synthetic construct
VDVAD

SEQ ID NO: 9 - protein, 4 aa, synthetic construct
LETD

SEQ ID NO: 10 - protein, 4 aa, synthetic construct
LEHD

SEQ ID NO: 11 - protein, 4 aa, synthetic construct
REQD

SEQ ID NO: 12 - protein, 4 aa, synthetic construct
DEVD

SEQ ID NO: 13 - protein, 4 aa, synthetic construct
VEID

SEQ ID NO: 14 - protein, 6 aa, synthetic construct
VQVDGW

SEQ ID NO: 15 - protein, 6 aa, synthetic construct
YEVDGW

SEQ ID NO: 16 - protein, 4 aa, synthetic construct
LEVD

SEQ ID NO: 17 - protein, 4 aa, synthetic construct
IEVE

SEQ ID NO: 18 - protein, 4 aa, synthetic construct
AAPV

SEQ ID NO: 19 - protein, 4 aa, synthetic construct
FFKF

SEQ ID NO: 20 - protein, 7 aa, synthetic construct
GRRGKGG

SEQ ID NO: 21 - protein, 4 aa, synthetic construct
VKKR

SEQ ID NO: 22 - protein, 7 aa, synthetic construct
  SITE 4: note = p-Nitro phenylalanine
FAAFFVL

SEQ ID NO: 23 - skipped

SEQ ID NO: 24 - protein, 5 aa, synthetic construct
KQKLR

SEQ ID NO: 25 - protein, 8 aa, synthetic construct
RPPGFSAF

SEQ ID NO: 26-27 - skipped (2 entries)

SEQ ID NO: 28 - protein, 5 aa, synthetic construct
LPLGL

SEQ ID NO: 29 - protein, 5 aa, synthetic construct
KPLGL

SEQ ID NO: 30 - protein, 6 aa, synthetic construct
  SITE 1: note = Gamma aminobutyric acid
XPQGLE

SEQ ID NO: 31 - protein, 6 aa, synthetic construct
PKPLAL

SEQ ID NO: 32 - protein, 7 aa, synthetic construct
GPSGIHV

SEQ ID NO: 33 - protein, 12 aa, synthetic construct
WAHRTTFYRRGA

SEQ ID NO: 34 - protein, 8 aa, synthetic construct
WKLRSSKQ

SEQ ID NO: 35 - skipped

SEQ ID NO: 36 - protein, 5 aa, synthetic construct
SYRIF

SEQ ID NO: 37 - skipped

SEQ ID NO: 38 - protein, 8 aa, synthetic construct
TAFRSAYG

SEQ ID NO: 39 - protein, 9 aa, synthetic construct
WAAFRFSQA

SEQ ID NO: 40-41 - skipped (2 entries)

SEQ ID NO: 42 - protein, 7 aa, synthetic construct
KLRSSKQ

SEQ ID NO: 43 - protein, 4 aa, synthetic construct
YASR

SEQ ID NO: 44 - protein, 10 aa, synthetic construct
RFAQAQQQLP

SEQ ID NO: 45 - protein, 8 aa, synthetic construct
KPAKFFRL

SEQ ID NO: 46 - protein, 9 aa, synthetic construct
  SITE 6: note = L-Homophenylalanine
PRAAAFTSP

SEQ ID NO: 47 - protein, 10 aa, synthetic construct
VGPQRFSGAP

SEQ ID NO: 48 - protein, 9 aa, synthetic construct
  SITE 7: note = L-Homophenylalanine
FFLAQAFRS

SEQ ID NO: 49 - protein, 6 aa, synthetic construct
PLAQAV

SEQ ID NO: 50 - protein, 8 aa, synthetic construct
RTAAVFRP

SEQ ID NO: 51 - protein, 13 aa, synthetic construct
DVQEFRGVTAVIR

SEQ ID NO: 52 - protein, 10 aa, synthetic construct
TEGEARGSVI

SEQ ID NO: 53 - skipped

SEQ ID NO: 54 - protein, 7 aa, synthetic construct
PLFAERK

SEQ ID NO: 55 - protein, 4 aa, synthetic construct
LLVY

SEQ ID NO: 56 - protein, 8 aa, synthetic construct
QQKRKIVL

SEQ ID NO: 57 - protein, 8 aa, synthetic construct
ASHLGLAR

SEQ ID NO: 58 - protein, 8 aa, synthetic construct
LPSRSSKI

SEQ ID NO: 59 - protein, 8 aa, synthetic construct
STGRNGFK

SEQ ID NO: 60 - protein, 8 aa, synthetic construct
SLLRSEET

SEQ ID NO: 61 - protein, 8 aa, synthetic construct
HRGRTLEI

SEQ ID NO: 62 - protein, 8 aa, synthetic construct
YLGRSYKV

SEQ ID NO: 63 - protein, 8 aa, synthetic construct
EKQRIIGG

SEQ ID NO: 64 - protein, 8 aa, synthetic construct
QRQRIIGG

SEQ ID NO: 65 - protein, 6 aa, synthetic construct
LQRIYK

SEQ ID NO: 66 - protein, 8 aa, synthetic construct
SLGRKIQI

SEQ ID NO: 67 - protein, 13 aa, synthetic construct
HAAPRSADIQIDI

SEQ ID NO: 68 - skipped

SEQ ID NO: 69 - protein, 4 aa, synthetic construct
SLGR

SEQ ID NO: 70 - protein, 4 aa, synthetic construct
GLQR

SEQ ID NO: 71 - protein, 8 aa, synthetic construct
SVARTLLV

SEQ ID NO: 72 - protein, 5 aa, synthetic construct
GRIFG

SEQ ID NO: 73 - skipped

SEQ ID NO: 74 - protein, 5 aa, synthetic construct
GFSPY

SEQ ID NO: 75 - protein, 8 aa, synthetic construct
WELRHAGH

SEQ ID NO: 76 - protein, 9 aa, synthetic construct
RQSRIVGGE

SEQ ID NO: 77 - protein, 8 aa, synthetic construct
EQAVYQTI

SEQ ID NO: 78 - protein, 11 aa, synthetic construct
VAYSGENTFGF

SEQ ID NO: 79 - skipped

SEQ ID NO: 80 - protein, 4 aa, synthetic construct
ATAD

SEQ ID NO: 81 - protein, 8 aa, synthetic construct
RPLESNAV

SEQ ID NO: 82 - protein, 7 aa, synthetic construct
RPLGLAR

SEQ ID NO: 83 - protein, 4 aa, synthetic construct
AAFF

SEQ ID NO: 84 - protein, 7 aa, synthetic construct
RVKRGLA

SEQ ID NO: 85 - skipped

SEQ ID NO: 86 - protein, 18 aa, synthetic construct
  SITE 4: note = D-Methionine
  SITE 5: note = D-Glutamic Acid
  SITE 8: note = D-Asparagine
  SITE 9: note = D-Aspartic Acid
  SITE 10: note = D-Asparagine
  SITE 11: note = D-Glutamic Acid
  SITE 12: note = D-Glutamic Acid
  SITE 16: note = D-Serine
  SITE 18: note = D-Arginine
CGGMEGVNDNEEGFFSAR

SEQ ID NO: 87 - protein, 8 aa, synthetic construct
GPQGIWGQ

SEQ ID NO: 88 - protein, 7 aa, synthetic construct
GLVPRGS

SEQ ID NO: 89 - protein, 6 aa, synthetic construct
GPVGLI

SEQ ID NO: 90 - protein, 8 aa, synthetic construct
GPWGIWGQ

SEQ ID NO: 91 - protein, 9 aa, synthetic construct
GPVPLSLVM

SEQ ID NO: 92 - protein, 7 aa, synthetic construct
  SITE 2: note = D-Phenylalanine
  SITE 3: note = Piperidine carboxylic acid
GFXRSGG

SEQ ID NO: 93 - protein, 6 aa, synthetic construct
PLGMRG

SEQ ID NO: 94 - protein, 6 aa, synthetic construct
PLGMRG

SEQ ID NO: 95 - protein, 6 aa, synthetic construct
  SITE 2: note = L-cyclohexylalanine
  SITE 4: note = L- Methyl cysteine
PXGCHA

SEQ ID NO: 96 - protein, 9 aa, synthetic construct
RPLALWESQ

SEQ ID NO: 97 - protein, 10 aa, synthetic construct
SGKGPRQITA

SEQ ID NO: 98 - protein, 10 aa, synthetic construct
SGPLFYSVTA

SEQ ID NO: 99 - protein, 9 aa, synthetic construct
SGRIFLRTA

SEQ ID NO: 100 - protein, 10 aa, synthetic construct
SGRSENIRTA

SEQ ID NO: 101 - protein, 5 aa, synthetic construct
GSGGS

SEQ ID NO: 102 - protein, 10 aa, synthetic construct
KPILFFRLKG

SEQ ID NO: 103 - protein, 6 aa, synthetic construct
  SITE 6: note = Norleucine
AWESRX

SEQ ID NO: 104 - protein, 6 aa, synthetic construct
  SITE 6: note = Norleucine
NEKSGX

SEQ ID NO: 105 - protein, 6 aa, synthetic construct
NATIVY

SEQ ID NO: 106 - protein, 6 aa, synthetic construct
DPFVVS

SEQ ID NO: 107 - protein, 6 aa, synthetic construct
  SITE 3: note = Norleucine
FHXFTK

SEQ ID NO: 108 - protein, 6 aa, synthetic construct
  SITE 1: note = Norleucine
XNWHKH

SEQ ID NO: 109 - protein, 6 aa, synthetic construct
FARRWG

SEQ ID NO: 110 - protein, 6 aa, synthetic construct
PGKWSK

SEQ ID NO: 111 - protein, 6 aa, synthetic construct
YEEAQP

SEQ ID NO: 112 - protein, 6 aa, synthetic construct
YGAIKK

SEQ ID NO: 113 - protein, 6 aa, synthetic construct
  SITE 3: note = Norleucine
TSXEGY

SEQ ID NO: 114 - protein, 6 aa, synthetic construct
PNNFGS

SEQ ID NO: 115 - protein, 6 aa, synthetic construct
EDTRNT

SEQ ID NO: 116 - protein, 6 aa, synthetic construct
KDLEQS

SEQ ID NO: 117 - protein, 6 aa, synthetic construct
AALHND

SEQ ID NO: 118 - protein, 6 aa, synthetic construct
ADSFFK

SEQ ID NO: 119 - protein, 6 aa, synthetic construct
ITFWRA

SEQ ID NO: 120 - protein, 6 aa, synthetic construct
  SITE 4: note = Norleucine
LSDXRL

SEQ ID NO: 121 - protein, 6 aa, synthetic construct
EVGWTY

SEQ ID NO: 122 - protein, 6 aa, synthetic construct
  SITE 6: note = Norleucine
IAFRQX

SEQ ID NO: 123 - protein, 6 aa, synthetic construct
  SITE 6: note = Norleucine
YNIHTX

SEQ ID NO: 124 - protein, 6 aa, synthetic construct
  SITE 1: note = Norleucine
XLWANH

SEQ ID NO: 125 - protein, 6 aa, synthetic construct
LYSVQV

SEQ ID NO: 126 - protein, 6 aa, synthetic construct
  SITE 4: note = Norleucine
SHIXSN

SEQ ID NO: 127 - protein, 6 aa, synthetic construct
KLLIDV

SEQ ID NO: 128 - protein, 6 aa, synthetic construct
  SITE 2: note = Norleucine
EXGVFD

SEQ ID NO: 129 - protein, 6 aa, synthetic construct
HQAYTL

SEQ ID NO: 130 - protein, 6 aa, synthetic construct
YVRKIQ

SEQ ID NO: 131 - protein, 6 aa, synthetic construct
DRENSP

SEQ ID NO: 132 - protein, 6 aa, synthetic construct
KYDKPR

SEQ ID NO: 133 - protein, 6 aa, synthetic construct
RPWKQL

SEQ ID NO: 134 - protein, 6 aa, synthetic construct
APLQRY

SEQ ID NO: 135 - protein, 6 aa, synthetic construct
  SITE 6: note = Norleucine
YQGQKX

SEQ ID NO: 136 - protein, 6 aa, synthetic construct
GRISSI

SEQ ID NO: 137 - protein, 6 aa, synthetic construct
HSLTNV

SEQ ID NO: 138 - protein, 6 aa, synthetic construct
EWDFPE

SEQ ID NO: 139 - protein, 6 aa, synthetic construct
  SITE 4: note = Norleucine
YLAXDG

SEQ ID NO: 140 - protein, 6 aa, synthetic construct
  SITE 4: note = Norleucine
FIYXPT

SEQ ID NO: 141 - protein, 6 aa, synthetic construct
GHETWV

SEQ ID NO: 142 - protein, 6 aa, synthetic construct
DYIGDE

SEQ ID NO: 143 - protein, 6 aa, synthetic construct
AGTAHP

SEQ ID NO: 144 - protein, 6 aa, synthetic construct
  SITE 2: note = Norleucine
VXTEIW

SEQ ID NO: 145 - protein, 6 aa, synthetic construct
PDDWQN

SEQ ID NO: 146 - protein, 6 aa, synthetic construct
GLNQEY

SEQ ID NO: 147 - protein, 6 aa, synthetic construct
YRDAVA

SEQ ID NO: 148 - protein, 6 aa, synthetic construct
TGPKGN

SEQ ID NO: 149 - protein, 6 aa, synthetic construct
DHVPQI

SEQ ID NO: 150 - protein, 6 aa, synthetic construct
NKEPIL

SEQ ID NO: 151 - protein, 6 aa, synthetic construct
  SITE 4: note = Norleucine
VWNXVH

SEQ ID NO: 152 - protein, 6 aa, synthetic construct
PVIIEH

SEQ ID NO: 153 - protein, 6 aa, synthetic construct
FQTDNL

SEQ ID NO: 154 - protein, 6 aa, synthetic construct
  SITE 3: note = Norleucine
RFXHGI

SEQ ID NO: 155 - protein, 6 aa, synthetic construct
YAERTT

SEQ ID NO: 156 - protein, 6 aa, synthetic construct
NRGELP

SEQ ID NO: 157 - protein, 6 aa, synthetic construct
HHYFNY

SEQ ID NO: 158 - protein, 6 aa, synthetic construct
STPYYH

SEQ ID NO: 159 - protein, 6 aa, synthetic construct
WFYPSA

SEQ ID NO: 160 - protein, 6 aa, synthetic construct
SEFLFS

SEQ ID NO: 161 - protein, 6 aa, synthetic construct
WYKTQY

SEQ ID NO: 162 - protein, 6 aa, synthetic construct
VTHLKV

SEQ ID NO: 163 - protein, 6 aa, synthetic construct
INGGFS

SEQ ID NO: 164 - protein, 6 aa, synthetic construct
TVLGLD

SEQ ID NO: 165 - protein, 6 aa, synthetic construct
  SITE 5: note = Norleucine
SYWPXQ

SEQ ID NO: 166 - protein, 6 aa, synthetic construct
ASQQHR

SEQ ID NO: 167 - protein, 6 aa, synthetic construct
KNPAKA

SEQ ID NO: 168 - protein, 6 aa, synthetic construct
  SITE 1: note = Norleucine
XYWLVE

SEQ ID NO: 169 - protein, 6 aa, synthetic construct
SWWIFE

SEQ ID NO: 170 - protein, 6 aa, synthetic construct
VNYEQD

SEQ ID NO: 171 - protein, 6 aa, synthetic construct
  SITE 4: note = Norleucine
HFFXAE

SEQ ID NO: 172 - protein, 6 aa, synthetic construct
DIPPHW

SEQ ID NO: 173 - protein, 6 aa, synthetic construct
  SITE 5: note = Norleucine
VDQWXW

SEQ ID NO: 174 - protein, 6 aa, synthetic construct
  SITE 5: note = Norleucine
LRSLXK

SEQ ID NO: 175 - protein, 6 aa, synthetic construct
  SITE 1: note = Norleucine
  SITE 2: note = Norleucine
XXIRHA

SEQ ID NO: 176 - protein, 6 aa, synthetic construct
HDVKFI

SEQ ID NO: 177 - protein, 6 aa, synthetic construct
KRVQFL

SEQ ID NO: 178 - protein, 6 aa, synthetic construct
  SITE 3: note = Norleucine
RDXYAE

SEQ ID NO: 179 - protein, 6 aa, synthetic construct
  SITE 2: note = Norleucine
LXIYFE

SEQ ID NO: 180 - protein, 6 aa, synthetic construct
LRTKQS

SEQ ID NO: 181 - protein, 6 aa, synthetic construct
WHGQQY

SEQ ID NO: 182 - protein, 6 aa, synthetic construct
GPEGTI

SEQ ID NO: 183 - protein, 6 aa, synthetic construct
ELDPIP

SEQ ID NO: 184 - protein, 6 aa, synthetic construct
GRAADF

SEQ ID NO: 185 - protein, 6 aa, synthetic construct
HFIDYI

SEQ ID NO: 186 - protein, 6 aa, synthetic construct
  SITE 2: note = Norleucine
  SITE 3: note = Norleucine
SXXRVH

SEQ ID NO: 187 - protein, 6 aa, synthetic construct
SFRKII

SEQ ID NO: 188 - protein, 6 aa, synthetic construct
  SITE 4: note = Norleucine
TYEXFS

SEQ ID NO: 189 - protein, 6 aa, synthetic construct
HLLGFY

SEQ ID NO: 190 - protein, 6 aa, synthetic construct
  SITE 1: note = Norleucine
XWTALT

SEQ ID NO: 191 - protein, 6 aa, synthetic construct
  SITE 4: note = Norleucine
IWNXVY

SEQ ID NO: 192 - protein, 6 aa, synthetic construct
RRNPLW

SEQ ID NO: 193 - protein, 6 aa, synthetic construct
RWYGGI

SEQ ID NO: 194 - protein, 6 aa, synthetic construct
KTGDAR

SEQ ID NO: 195 - protein, 6 aa, synthetic construct
NYWEAN

SEQ ID NO: 196 - protein, 6 aa, synthetic construct
  SITE 1: note = Norleucine
XQFDTS

SEQ ID NO: 197 - protein, 6 aa, synthetic construct
KRGAVE

SEQ ID NO: 198 - protein, 6 aa, synthetic construct
SLKPTE

SEQ ID NO: 199 - protein, 6 aa, synthetic construct
ENDRLP

SEQ ID NO: 200 - protein, 6 aa, synthetic construct
NSYQVQ

SEQ ID NO: 201 - protein, 6 aa, synthetic construct
YPKEYL

SEQ ID NO: 202 - protein, 6 aa, synthetic construct
INNKWQ

SEQ ID NO: 203 - protein, 6 aa, synthetic construct
  SITE 1: note = Norleucine
XEFQGW

SEQ ID NO: 204 - protein, 6 aa, synthetic construct
PVRSTN

SEQ ID NO: 205 - protein, 6 aa, synthetic construct
SQAIKV

SEQ ID NO: 206 - protein, 6 aa, synthetic construct
  SITE 3: note = Norleucine
WAXLYH

SEQ ID NO: 207 - protein, 6 aa, synthetic construct
ISWIHA

SEQ ID NO: 208 - protein, 5 aa, synthetic construct
AHDIV

SEQ ID NO: 209 - protein, 6 aa, synthetic construct
RHNVAS

SEQ ID NO: 210 - protein, 6 aa, synthetic construct
SVFVIE

SEQ ID NO: 211 - protein, 6 aa, synthetic construct
FAKYYK

SEQ ID NO: 212 - protein, 6 aa, synthetic construct
PYNTLQ

SEQ ID NO: 213 - protein, 6 aa, synthetic construct
  SITE 1: note = Norleucine
  SITE 6: note = Norleucine
XDWGHX

SEQ ID NO: 214 - protein, 6 aa, synthetic construct
SNREWF

SEQ ID NO: 215 - protein, 6 aa, synthetic construct
GKSEHT

SEQ ID NO: 216 - protein, 6 aa, synthetic construct
  SITE 3: note = Norleucine
FPXTDQ

SEQ ID NO: 217 - protein, 6 aa, synthetic construct
  SITE 6: note = Norleucine
WSKFWX

SEQ ID NO: 218 - protein, 6 aa, synthetic construct
RFTRPH

SEQ ID NO: 219 - protein, 6 aa, synthetic construct
  SITE 4: note = Norleucine
QETXKD

SEQ ID NO: 220 - protein, 6 aa, synthetic construct
HWWDVL

SEQ ID NO: 221 - protein, 6 aa, synthetic construct
  SITE 5: note = Norleucine
FNLVXS

SEQ ID NO: 222 - protein, 6 aa, synthetic construct
SAWRQR

SEQ ID NO: 223 - protein, 6 aa, synthetic construct
TFHIFL

SEQ ID NO: 224 - protein, 6 aa, synthetic construct
WPQHVK

SEQ ID NO: 225 - protein, 6 aa, synthetic construct
  SITE 3: note = Norleucine
LIXHKN

SEQ ID NO: 226 - protein, 6 aa, synthetic construct
QDLEQP

SEQ ID NO: 227 - protein, 6 aa, synthetic construct
  SITE 5: note = Norleucine
HQKKXP

SEQ ID NO: 228 - protein, 6 aa, synthetic construct
GVTWLN

SEQ ID NO: 229 - protein, 6 aa, synthetic construct
AGEPFK

SEQ ID NO: 230 - protein, 6 aa, synthetic construct
  SITE 3: note = Norleucine
SRXATT

SEQ ID NO: 231 - protein, 6 aa, synthetic construct
  SITE 4: note = Norleucine
LAFXNH

SEQ ID NO: 232 - protein, 6 aa, synthetic construct
PPSGLS

SEQ ID NO: 233 - protein, 6 aa, synthetic construct
YTHSSP

SEQ ID NO: 234 - protein, 6 aa, synthetic construct
DGSHYR

SEQ ID NO: 235 - protein, 6 aa, synthetic construct
  SITE 2: note = Norleucine
YXGNGY

SEQ ID NO: 236 - protein, 6 aa, synthetic construct
DSITVS

SEQ ID NO: 237 - protein, 6 aa, synthetic construct
QTPNIQ

SEQ ID NO: 238 - protein, 6 aa, synthetic construct
KLFFGY

SEQ ID NO: 239 - protein, 6 aa, synthetic construct
TQNFNW

SEQ ID NO: 240 - protein, 6 aa, synthetic construct
YSDHEV

SEQ ID NO: 241 - protein, 6 aa, synthetic construct
RYVVPA

SEQ ID NO: 242 - protein, 6 aa, synthetic construct
ILHRIR

SEQ ID NO: 243 - protein, 6 aa, synthetic construct
  SITE 6: note = Norleucine
ESDNQX

SEQ ID NO: 244 - protein, 6 aa, synthetic construct
  SITE 6: note = Norleucine
YDDKGX

SEQ ID NO: 245 - protein, 6 aa, synthetic construct
  SITE 4: note = Norleucine
QLSXVW

SEQ ID NO: 246 - protein, 6 aa, synthetic construct
  SITE 6: note = Norleucine
PGGERX

SEQ ID NO: 247 - protein, 6 aa, synthetic construct
WKHHPD

SEQ ID NO: 248 - protein, 6 aa, synthetic construct
QWVDED

SEQ ID NO: 249 - protein, 6 aa, synthetic construct
NAYNEI

SEQ ID NO: 250 - protein, 6 aa, synthetic construct
EEKAPR

SEQ ID NO: 251 - protein, 6 aa, synthetic construct
PWQIGK

SEQ ID NO: 252 - protein, 6 aa, synthetic construct
IAQVGN

SEQ ID NO: 253 - protein, 6 aa, synthetic construct
  SITE 2: note = Norleucine
VXRQSE

SEQ ID NO: 254 - protein, 6 aa, synthetic construct
TERVDA

SEQ ID NO: 255 - protein, 6 aa, synthetic construct
WLRWRL

SEQ ID NO: 256 - protein, 6 aa, synthetic construct
WKTKGQ

SEQ ID NO: 257 - protein, 6 aa, synthetic construct
QSNGDV

SEQ ID NO: 258 - protein, 6 aa, synthetic construct
TLFYAL

SEQ ID NO: 259 - protein, 6 aa, synthetic construct
TVTLNP

SEQ ID NO: 260 - protein, 6 aa, synthetic construct
YAFGRK

SEQ ID NO: 261 - protein, 6 aa, synthetic construct
DYNYWD

SEQ ID NO: 262 - protein, 6 aa, synthetic construct
EWHEII

SEQ ID NO: 263 - protein, 6 aa, synthetic construct
QKAAWD

SEQ ID NO: 264 - protein, 6 aa, synthetic construct
DNTSAD

SEQ ID NO: 265 - protein, 6 aa, synthetic construct
HEGEYV

SEQ ID NO: 266 - protein, 6 aa, synthetic construct
WSPSFK

SEQ ID NO: 267 - protein, 6 aa, synthetic construct
HDEHWT

SEQ ID NO: 268 - protein, 6 aa, synthetic construct
  SITE 4: note = Norleucine
YVWXRD

SEQ ID NO: 269 - protein, 6 aa, synthetic construct
  SITE 1: note = Norleucine
  SITE 4: note = Norleucine
XDPXKF

SEQ ID NO: 270 - protein, 6 aa, synthetic construct
  SITE 1: note = Norleucine
  SITE 3: note = Norleucine
XRXFWD

SEQ ID NO: 271 - protein, 6 aa, synthetic construct
  SITE 6: note = Norleucine
DIAITX

SEQ ID NO: 272 - protein, 6 aa, synthetic construct
  SITE 3: note = Norleucine
PIXRFH

SEQ ID NO: 273 - protein, 6 aa, synthetic construct
VWQGYI

SEQ ID NO: 274 - protein, 6 aa, synthetic construct
  SITE 3: note = Norleucine
KKXSNP

SEQ ID NO: 275 - protein, 6 aa, synthetic construct
GHPLSP

SEQ ID NO: 276 - protein, 6 aa, synthetic construct
VRQHKP

SEQ ID NO: 277 - protein, 6 aa, synthetic construct
AQNFYR

SEQ ID NO: 278 - protein, 6 aa, synthetic construct
VAGKSI

SEQ ID NO: 279 - protein, 6 aa, synthetic construct
LVGQVN

SEQ ID NO: 280 - protein, 6 aa, synthetic construct
QVKHFT

SEQ ID NO: 281 - protein, 6 aa, synthetic construct
QKSVVS

SEQ ID NO: 282 - protein, 6 aa, synthetic construct
  SITE 2: note = Norleucine
YXQEWL

SEQ ID NO: 283 - protein, 6 aa, synthetic construct
  SITE 2: note = Norleucine
GXYIDE

SEQ ID NO: 284 - protein, 6 aa, synthetic construct
NAGSKF

SEQ ID NO: 285 - protein, 6 aa, synthetic construct
EFVHNP

SEQ ID NO: 286 - protein, 6 aa, synthetic construct
  SITE 3: note = Norleucine
WEXVKI

SEQ ID NO: 287 - protein, 6 aa, synthetic construct
WVGASH

SEQ ID NO: 288 - protein, 6 aa, synthetic construct
  SITE 6: note = Norleucine
ITTLYX

SEQ ID NO: 289 - protein, 6 aa, synthetic construct
GHIDEY

SEQ ID NO: 290 - protein, 6 aa, synthetic construct
  SITE 3: note = Norleucine
KVXDYG

SEQ ID NO: 291 - protein, 6 aa, synthetic construct
  SITE 6: note = Norleucine
QEKQTX

SEQ ID NO: 292 - protein, 6 aa, synthetic construct
EVGHEA

SEQ ID NO: 293 - protein, 6 aa, synthetic construct
AWEGQY

SEQ ID NO: 294 - protein, 6 aa, synthetic construct
FLVQWT

SEQ ID NO: 295 - protein, 6 aa, synthetic construct
SKWGYW

SEQ ID NO: 296 - protein, 6 aa, synthetic construct
  SITE 5: note = Norleucine
TWISXQ

SEQ ID NO: 297 - protein, 6 aa, synthetic construct
VIDKDF

SEQ ID NO: 298 - protein, 6 aa, synthetic construct
VKFAIY

SEQ ID NO: 299 - protein, 6 aa, synthetic construct
  SITE 4: note = Norleucine
HNQXKS

SEQ ID NO: 300 - protein, 6 aa, synthetic construct
  SITE 6: note = Norleucine
QYVFFX

SEQ ID NO: 301 - protein, 6 aa, synthetic construct
  SITE 6: note = Norleucine
YNPREX

SEQ ID NO: 302 - protein, 6 aa, synthetic construct
  SITE 4: note = Norleucine
KHGXPE

SEQ ID NO: 303 - protein, 6 aa, synthetic construct
WSREYW

SEQ ID NO: 304 - protein, 6 aa, synthetic construct
IDRVDK

SEQ ID NO: 305 - protein, 9 aa, synthetic construct
  SITE 8: note = Lysine modified with a CPQ2 quencher
  SITE 9: note = Hydroxy Leucine
GDRENSPKL

SEQ ID NO: 306 - protein, 9 aa, synthetic construct
  SITE 9: note = Hydroxy Lysine modified with a CPQ2 quencher
GDRENSPLK

SEQ ID NO: 307 - protein, 8 aa, synthetic construct
  SITE 7: note = Lysine modified with a CPQ2 quencher
  SITE 8: note = Hydroxy Glutamine
NAGSKFKQ

SEQ ID NO: 308 - protein, 8 aa, synthetic construct
  SITE 8: note = Hydroxy Lysine modified with a CPQ2 quencher
NAGSKFQK

SEQ ID NO: 309 - protein, 9 aa, synthetic construct
  SITE 8: note = Lysine modified with a CPQ2 quencher
  SITE 9: note = Hydroxy Valine
GHLLGFYKV

SEQ ID NO: 310 - protein, 9 aa, synthetic construct
  SITE 9: note = Hydroxy Lysine modified with a CPQ2 quencher
GHLLGFYVK

SEQ ID NO: 311 - protein, 9 aa, synthetic construct
  SITE 7: note = Norleucine
  SITE 8: note = Lysine modified with a CPQ2 quencher
  SITE 9: note = Hydroxy Norleucine
GQEKQTXKX

SEQ ID NO: 312 - protein, 9 aa, synthetic construct
  SITE 7: note = Norleucine
  SITE 8: note = Norleucine
  SITE 9: note = Hydroxy Lysine modified with a CPQ2 quencher
GQEKQTXXK

SEQ ID NO: 313 - protein, 10 aa, synthetic construct
  SITE 1: note = D-Lysine
  SITE 9: note = Lysine modified with a CPQ2 quencher
  SITE 10: note = Hydroxy Tryptophan
KGDPFVVSKW

SEQ ID NO: 314 - protein, 10 aa, synthetic construct
  SITE 1: note = D-Lysine
  SITE 10: note = Hydroxy Lysine modified with a CPQ2 quencher
KGDPFVVSWK

SEQ ID NO: 315 - protein, 8 aa, synthetic construct
  SITE 7: note = Lysine modified with a CPQ2 quencher
  SITE 8: note = Hydroxy Arginine
NAYNEIKR

SEQ ID NO: 316 - protein, 8 aa, synthetic construct
  SITE 8: note = Hydroxy Lysine modified with a CPQ2 quencher
NAYNEIRK

SEQ ID NO: 317 - protein, 8 aa, synthetic construct
  SITE 2: note = Norleucine
  SITE 7: note = Lysine modified with a CPQ2 quencher
  SITE 8: note = Hydroxy Asparagine
VXRQSEKN

SEQ ID NO: 318 - protein, 8 aa, synthetic construct
  SITE 2: note = Norleucine
  SITE 8: note = Hydroxy Lysine modified with a CPQ2 quencher
VXRQSENK

SEQ ID NO: 319 - protein, 8 aa, synthetic construct
  SITE 6: note = Norleucine
  SITE 7: note = Lysine modified with a CPQ2 quencher
  SITE 8: note = Hydroxy Isoleucine
YNPREXKI

SEQ ID NO: 320 - protein, 8 aa, synthetic construct
  SITE 6: note = Norleucine
  SITE 8: note = Hydroxy Lysine modified with a CPQ2 quencher
YNPREXIK

SEQ ID NO: 321 - protein, 8 aa, synthetic construct
  SITE 7: note = Lysine modified with a CPQ2 quencher
  SITE 8: note = Hydroxy Lysine
EFVHNPKK

SEQ ID NO: 322 - protein, 8 aa, synthetic construct
  SITE 8: note = Hydroxy Lysine modified with a CPQ2 quencher
EFVHNPKK

SEQ ID NO: 323 - protein, 8 aa, synthetic construct
  SITE 7: note = Lysine modified with a CPQ2 quencher
  SITE 8: note = Hydroxy Histidine
KRVQFLKH

SEQ ID NO: 324 - protein, 8 aa, synthetic construct
  SITE 8: note = Hydroxy Lysine modified with a CPQ2 quencher
KRVQFLHK

SEQ ID NO: 325 - protein, 8 aa, synthetic construct
  SITE 3: note = Norleucine
  SITE 7: note = Lysine modified with a CPQ2 quencher
  SITE 8: note = Hydroxy Glycine
LIXHKNKG

SEQ ID NO: 326 - protein, 8 aa, synthetic construct
  SITE 3: note = Norleucine
  SITE 8: note = Hydroxy Lysine modified with a CPQ2 quencher
LIXHKNGK

SEQ ID NO: 327 - protein, 8 aa, synthetic construct
  SITE 3: note = Norleucine
  SITE 7: note = Lysine modified with a CPQ2 quencher
  SITE 8: note = Hydroxy Serine
WAXLYHKS

SEQ ID NO: 328 - protein, 8 aa, synthetic construct
  SITE 3: note = Norleucine
  SITE 8: note = Hydroxy Lysine modified with a CPQ2 quencher
WAXLYHSK

SEQ ID NO: 329 - protein, 8 aa, synthetic construct
  SITE 7: note = Lysine modified with a CPQ2 quencher
  SITE 8: note = Hydroxy Tyrosine
AHDIVNKY

SEQ ID NO: 330 - protein, 8 aa, synthetic construct
  SITE 8: note = Hydroxy Lysine modified with a CPQ2 quencher
AHDIVNYK

SEQ ID NO: 331 - protein, 8 aa, synthetic construct
  SITE 7: note = Lysine modified with a CPQ2 quencher
  SITE 8: note = Hydroxy Proline
SVFVIEKP

SEQ ID NO: 332 - protein, 8 aa, synthetic construct
  SITE 8: note = Hydroxy Lysine modified with a CPQ2 quencher
SVFVIEPK

SEQ ID NO: 333 - protein, 8 aa, synthetic construct
  SITE 7: note = Lysine modified with a CPQ2 quencher
  SITE 8: note = Hydroxy Glutamic Acid
PPSGLSKE

SEQ ID NO: 334 - protein, 8 aa, synthetic construct
  SITE 8: note = Hydroxy Lysine modified with a CPQ2 quencher
PPSGLSEK

SEQ ID NO: 335 - protein, 8 aa, synthetic construct
  SITE 7: note = Lysine modified with a CPQ2 quencher
  SITE 8: note = Hydroxy Phenylalanine
RWYGGIKF

SEQ ID NO: 336 - protein, 8 aa, synthetic construct
  SITE 8: note = Hydroxy Lysine modified with a CPQ2 quencher
RWYGGIFK

SEQ ID NO: 337 - protein, 8 aa, synthetic construct
  SITE 6: note = Norleucine
  SITE 7: note = Lysine modified with a CPQ2 quencher
  SITE 8: note = Hydroxy Aspartic Acid
QYVFFXKD

SEQ ID NO: 338 - protein, 8 aa, synthetic construct
  SITE 6: note = Norleucine
  SITE 8: note = Hydroxy Lysine modified with a CPQ2 quencher
QYVFFXDK

SEQ ID NO: 339 - protein, 8 aa, synthetic construct
  SITE 7: note = Lysine modified with a CPQ2 quencher
  SITE 8: note = Hydroxy Threonine
FAKYYKKT

SEQ ID NO: 340 - protein, 8 aa, synthetic construct
  SITE 8: note = Hydroxy Lysine modified with a CPQ2 quencher
FAKYYKTK

SEQ ID NO: 341 - protein, 8 aa, synthetic construct
  SITE 7: note = Lysine modified with a CPQ2 quencher
  SITE 8: note = Hydroxy Alanine
QVKHFTKA

SEQ ID NO: 342 - protein, 8 aa, synthetic construct
  SITE 8: note = Hydroxy Lysine modified with a CPQ2 quencher
QVKHFTAK

SEQ ID NO: 343 - skipped

SEQ ID NO: 344 - protein, 8 aa, synthetic construct
  SITE 1: note = Histidine Amide
  SITE 2: note = Carboxy-fluorescein-L-lysine
HKDRENSP

SEQ ID NO: 345 - protein, 8 aa, synthetic construct
  SITE 1: note = Carboxy-fluorescein-L-lysine Amide
KHDRENSP

SEQ ID NO: 346 - protein, 8 aa, synthetic construct
  SITE 1: note = Tryptophan Amide
  SITE 2: note = Carboxy-fluorescein-L-lysine
WKNAGSKF

SEQ ID NO: 347 - protein, 8 aa, synthetic construct
  SITE 1: note = Carboxy-fluorescein-L-lysine Amide
KWNAGSKF

SEQ ID NO: 348 - protein, 8 aa, synthetic construct
  SITE 1: note = Serine Amide
  SITE 2: note = Carboxy-fluorescein-L-lysine
SKHLLGFY

SEQ ID NO: 349 - protein, 8 aa, synthetic construct
  SITE 1: note = Carboxy-fluorescein-L-lysine Amide
KSHLLGFY

SEQ ID NO: 350 - protein, 8 aa, synthetic construct
  SITE 1: note = Lysine Amide
  SITE 2: note = Carboxy-fluorescein-L-lysine
  SITE 8: note = Norleucine
KKQEKQTX

SEQ ID NO: 351 - protein, 8 aa, synthetic construct
  SITE 1: note = Carboxy-fluorescein-L-lysine Amide
  SITE 8: note = Norleucine
KKQEKQTX

SEQ ID NO: 352 - protein, 8 aa, synthetic construct
  SITE 1: note = Glycine Amide
  SITE 2: note = Carboxy-fluorescein-L-lysine
GKDPFVVS

SEQ ID NO: 353 - protein, 8 aa, synthetic construct
  SITE 1: note = Carboxy-fluorescein-L-lysine Amide
KGDPFVVS

SEQ ID NO: 354 - protein, 8 aa, synthetic construct
  SITE 1: note = Proline Amide
  SITE 2: note = Carboxy-fluorescein-L-lysine
PKNAYNEI

SEQ ID NO: 355 - protein, 8 aa, synthetic construct
  SITE 1: note = Carboxy-fluorescein-L-lysine Amide
KPNAYNEI

SEQ ID NO: 356 - protein, 8 aa, synthetic construct
  SITE 1: note = Aspartic Acid Amide
  SITE 2: note = Carboxy-fluorescein-L-lysine
  SITE 4: note = Norleucine
DKVXRQSE

SEQ ID NO: 357 - protein, 8 aa, synthetic construct
  SITE 1: note = Carboxy-fluorescein-L-lysine Amide
  SITE 4: note = Norleucine
KDVXRQSE

SEQ ID NO: 358 - protein, 8 aa, synthetic construct
  SITE 1: note = Glutamic Acid Amide
  SITE 2: note = Carboxy-fluorescein-L-lysine
  SITE 8: note = Norleucine
EKYNPREX

SEQ ID NO: 359 - protein, 8 aa, synthetic construct
  SITE 1: note = Carboxy-fluorescein-L-lysine Amide
  SITE 8: note = Norleucine
KEYNPREX

SEQ ID NO: 360 - protein, 8 aa, synthetic construct
  SITE 1: note = Threonine Amide
  SITE 2: note = Carboxy-fluorescein-L-lysine
TKEFVHNP

SEQ ID NO: 361 - protein, 8 aa, synthetic construct
  SITE 1: note = Carboxy-fluorescein-L-lysine Amide
KTEFVHNP

SEQ ID NO: 362 - protein, 8 aa, synthetic construct
  SITE 1: note = Glutamine Amide
  SITE 2: note = Carboxy-fluorescein-L-lysine
QKKRVQFL

SEQ ID NO: 363 - protein, 8 aa, synthetic construct
  SITE 1: note = Carboxy-fluorescein-L-lysine Amide
KQKRVQFL

SEQ ID NO: 364 - protein, 8 aa, synthetic construct
  SITE 1: note = Tyrosine Amide
  SITE 2: note = Carboxy-fluorescein-L-lysine
  SITE 5: note = Norleucine
YKLIXHKN

SEQ ID NO: 365 - protein, 8 aa, synthetic construct
  SITE 1: note = Carboxy-fluorescein-L-lysine Amide
  SITE 5: note = Norleucine
KYLIXHKN

SEQ ID NO: 366 - protein, 8 aa, synthetic construct
  SITE 1: note = Phenylalanine Amide
  SITE 2: note = Carboxy-fluorescein-L-lysine
  SITE 5: note = Norleucine
FKWAXLYH

SEQ ID NO: 367 - protein, 8 aa, synthetic construct
  SITE 1: note = Carboxy-fluorescein-L-lysine Amide
  SITE 5: note = Norleucine
KFWAXLYH

SEQ ID NO: 368 - protein, 8 aa, synthetic construct
  SITE 1: note = Isoleucine Amide
  SITE 2: note = Carboxy-fluorescein-L-lysine
IKAHDIVN

SEQ ID NO: 369 - protein, 8 aa, synthetic construct
  SITE 1: note = Carboxy-fluorescein-L-lysine Amide
KIAHDIVN

SEQ ID NO: 370 - protein, 8 aa, synthetic construct
  SITE 1: note = Valine Amide
  SITE 2: note = Carboxy-fluorescein-L-lysine
VKSVFVIE

SEQ ID NO: 371 - protein, 8 aa, synthetic construct
  SITE 1: note = Carboxy-fluorescein-L-lysine Amide
KVSVFVIE

SEQ ID NO: 372 - protein, 8 aa, synthetic construct
  SITE 1: note = Norleucine Amide
  SITE 2: note = Carboxy-fluorescein-L-lysine
XKPPSGLS

SEQ ID NO: 373 - protein, 8 aa, synthetic construct
  SITE 1: note = Carboxy-fluorescein-L-lysine Amide
  SITE 2: note = Norleucine
KXPPSGLS

SEQ ID NO: 374 - protein, 8 aa, synthetic construct
  SITE 1: note = Leucine Amide
  SITE 2: note = Carboxy-fluorescein-L-lysine
LKRWYGGI

SEQ ID NO: 375 - protein, 8 aa, synthetic construct
  SITE 1: note = Carboxy-fluorescein-L-lysine Amide
KLRWYGGI

SEQ ID NO: 376 - protein, 8 aa, synthetic construct
  SITE 1: note = Asparagine Amide
  SITE 2: note = Carboxy-fluorescein-L-lysine
  SITE 8: note = Norleucine
NKQYVFFX

SEQ ID NO: 377 - protein, 8 aa, synthetic construct
  SITE 1: note = Carboxy-fluorescein-L-lysine Amide
  SITE 8: note = Norleucine
KNQYVFFX

SEQ ID NO: 378 - protein, 8 aa, synthetic construct
  SITE 1: note = Alanine Amide
  SITE 2: note = Carboxy-fluorescein-L-lysine
AKFAKYYK

SEQ ID NO: 379 - protein, 8 aa, synthetic construct
  SITE 1: note = Carboxy-fluorescein-L-lysine Amide
KAFAKYYK

SEQ ID NO: 380 - protein, 8 aa, synthetic construct
  SITE 1: note = Arginine Amide
  SITE 2: note = Carboxy-fluorescein-L-lysine
RKQVKHFT

SEQ ID NO: 381 - protein, 8 aa, synthetic construct
  SITE 1: note = Carboxy-fluorescein-L-lysine Amide
KRQVKHFT

SEQ ID NO: 382 - skipped

SEQ ID NO: 383 - protein, 9 aa, synthetic construct
  SITE 1: note = D-Lysine
  SITE 2: note = D-Proline
  SITE 3: note = D-Isoleucine
  SITE 4: note = D-Leucine
  SITE 5: note = D-Phenylalanine
  SITE 6: note = D-Phenylalanine
  SITE 7: note = D-Arginine
  SITE 8: note = D-Leucine
  SITE 9: note = D-Lysine
KPILFFRLK

SEQ ID NO: 384-405 - skipped (22 entries)

SEQ ID NO: 406 - protein, 4 aa, synthetic construct
LRGG

SEQ ID NO: 407 - protein, 5 aa, synthetic construct
RLRGG

SEQ ID NO: 408 - protein, 9 aa, synthetic construct
RELNGGAPI

SEQ ID NO: 409 - protein, 11 aa, synthetic construct
TSAVLQSGFRK

SEQ ID NO: 410 - protein, 11 aa, synthetic construct
SGVTFQGKFKK

SEQ ID NO: 411 - protein, 4 aa, synthetic construct
AAFA

SEQ ID NO: 412 - protein, 9 aa, synthetic construct
HGDQMAQKS

SEQ ID NO: 413 - protein, 6 aa, synthetic construct
GPLGMR

SEQ ID NO: 414 - protein, 10 aa, synthetic construct
  SITE 7: note = L-homophenylalanine
FFLAQAFRSK

SEQ ID NO: 415 - protein, 15 aa, synthetic construct
AHAVSRIRIYLLPAK

SEQ ID NO: 416 - protein, 7 aa, synthetic construct
PLALWAR

SEQ ID NO: 417 - protein, 7 aa, synthetic construct
  SITE 4: note = S-para-methoxybenzyl cysteine
PLACWAR

SEQ ID NO: 418 - protein, 7 aa, synthetic construct
APRWIQD

SEQ ID NO: 419 - protein, 9 aa, synthetic construct
LREQQRLKS

SEQ ID NO: 420 - protein, 12 aa, synthetic construct
EFPIYVFLPAKK

SEQ ID NO: 421 - protein, 9 aa, synthetic construct
GAANLVRGG

SEQ ID NO: 422 - protein, 8 aa, synthetic construct
GYAELRMG

SEQ ID NO: 423 - protein, 9 aa, synthetic construct
AAGAMFLEA

SEQ ID NO: 424 - protein, 13 aa, synthetic construct
LGGSGQRGRKALE

SEQ ID NO: 425 - protein, 13 aa, synthetic construct
LGGSGHYGRSGLE

SEQ ID NO: 426 - protein, 4 aa, synthetic construct
YGRS

SEQ ID NO: 427 - protein, 5 aa, synthetic construct
FRGRK

SEQ ID NO: 428 - protein, 8 aa, synthetic construct
DRRKKLTQ

SEQ ID NO: 429 - protein, 6 aa, synthetic construct
HPGGPQ

SEQ ID NO: 430 - protein, 7 aa, synthetic construct
KLRFSKQ

SEQ ID NO: 431 - protein, 8 aa, synthetic construct
AIKFFSAQ

SEQ ID NO: 432 - protein, 8 aa, synthetic construct
AIKFFVRQ

SEQ ID NO: 433 - protein, 9 aa, synthetic construct
RPPGFSAFK

SEQ ID NO: 434 - protein, 6 aa, synthetic construct
FAPQLS

SEQ ID NO: 435 - protein, 6 aa, synthetic construct
FAAQMA

SEQ ID NO: 436 - protein, 6 aa, synthetic construct
GMPANQ

SEQ ID NO: 437 - protein, 8 aa, synthetic construct
LSGRSDNH

SEQ ID NO: 438 - protein, 10 aa, synthetic construct
MAALITRPDF

SEQ ID NO: 439 - protein, 10 aa, synthetic construct
MAAAITRPRF

SEQ ID NO: 440 - protein, 10 aa, synthetic construct
MAALIVRPDL

SEQ ID NO: 441 - protein, 9 aa, synthetic construct
TSGPNQEQE

SEQ ID NO: 442 - protein, 9 aa, synthetic construct
TAGPNQEQE

SEQ ID NO: 443 - protein, 7 aa, synthetic construct
GPGPNQA

SEQ ID NO: 444 - protein, 8 aa, synthetic construct
ASGPAGPA

SEQ ID NO: 445 - protein, 9 aa, synthetic construct
ERGETGPSG

SEQ ID NO: 446 - protein, 8 aa, synthetic construct
VSQELGQR

SEQ ID NO: 447 - protein, 9 aa, synthetic construct
TGPPGYPTG

SEQ ID NO: 448 - protein, 7 aa, synthetic construct
TRLPVYQ

SEQ ID NO: 449 - protein, 8 aa, synthetic construct
RQARVVGG

SEQ ID NO: 450 - protein, 8 aa, synthetic construct
RQRRVVGG

SEQ ID NO: 451 - protein, 8 aa, synthetic construct
RQARAVGG

SEQ ID NO: 452 - protein, 8 aa, synthetic construct
RKRRGSRG

SEQ ID NO: 453 - protein, 8 aa, synthetic construct
KQSRKFVP

SEQ ID NO: 454 - protein, 5 aa, synthetic construct
VTGRS

SEQ ID NO: 455 - protein, 6 aa, synthetic construct
LKSRVK

SEQ ID NO: 456 - protein, 10 aa, synthetic construct
GIGAVLKVLT

SEQ ID NO: 457 - protein, 10 aa, synthetic construct
GLPALISWIK

SEQ ID NO: 458 - protein, 9 aa, synthetic construct
SEVNLDAEF

SEQ ID NO: 459 - protein, 13 aa, synthetic construct
EEKPICFFRLGKE

SEQ ID NO: 460 - protein, 13 aa, synthetic construct
EEKPILFFRLGKE

SEQ ID NO: 461 - protein, 8 aa, synthetic construct
APSSVIAA

SEQ ID NO: 462 - protein, 8 aa, synthetic construct
KKAKRNAL

SEQ ID NO: 463 - protein, 10 aa, synthetic construct
WTNTSANYNL

SEQ ID NO: 464 - protein, 4 aa, synthetic construct
RVRR

SEQ ID NO: 465 - protein, 5 aa, synthetic construct
ERTKR

SEQ ID NO: 466 - protein, 12 aa, synthetic construct
RYQIKPLKSTDE

SEQ ID NO: 467 - protein, 11 aa, synthetic construct
  SITE 8: note = L-homophenylalanine
WELRHQAFRSK

SEQ ID NO: 468 - protein, 12 aa, synthetic construct
  SITE 6: note = L- Methyl cysteine
SGAFKCLKDGAG

SEQ ID NO: 469 - protein, 6 aa, synthetic construct
YVADGW

SEQ ID NO: 470 - protein, 6 aa, synthetic construct
WEHDGW

SEQ ID NO: 471 - protein, 7 aa, synthetic construct
YVADAPV

SEQ ID NO: 472 - protein, 7 aa, synthetic construct
RPPGFSA

SEQ ID NO: 473 - protein, 7 aa, synthetic construct
GSPAFLA

SEQ ID NO: 474 - protein, 7 aa, synthetic construct
AGFSLPA

SEQ ID NO: 475 - protein, 10 aa, synthetic construct
RWHTVGLRWE

SEQ ID NO: 476 - skipped

SEQ ID NO: 477 - protein, 10 aa, synthetic construct
RWPPMGLPWE

SEQ ID NO: 478 - protein, 6 aa, synthetic construct
RPKPVE

SEQ ID NO: 479 - protein, 4 aa, synthetic construct
IETD

SEQ ID NO: 480 - protein, 7 aa, synthetic construct
VGPDFGR

SEQ ID NO: 481 - protein, 9 aa, synthetic construct
GIEFDSGGC

SEQ ID NO: 482 - protein, 7 aa, synthetic construct
GDFLRRV

SEQ ID NO: 483 - skipped

SEQ ID NO: 484 - protein, 10 aa, synthetic construct
YATWSMIAAH

SEQ ID NO: 485 - protein, 10 aa, synthetic construct
VIMWRLTVGT

SEQ ID NO: 486 - protein, 10 aa, synthetic construct
RRVLALQQEL

SEQ ID NO: 487 - protein, 10 aa, synthetic construct
LATWPLSGLW

SEQ ID NO: 488 - protein, 10 aa, synthetic construct
NTPNWLVNAV

SEQ ID NO: 489 - protein, 13 aa, synthetic construct
SPLAQAVRSSSRK

SEQ ID NO: 490 - protein, 10 aa, synthetic construct
QMPGRLSMAF

SEQ ID NO: 491 - protein, 5 aa, synthetic construct
PLGLR

SEQ ID NO: 492 - protein, 10 aa, synthetic construct
QRANSIRVTW

SEQ ID NO: 493 - protein, 5 aa, synthetic construct
PLAVR

SEQ ID NO: 494 - protein, 10 aa, synthetic construct
LLAVPAANTV

SEQ ID NO: 495 - protein, 8 aa, synthetic construct
GPQGLRGQ

SEQ ID NO: 496 - protein, 10 aa, synthetic construct
RTGLYLYNST

SEQ ID NO: 497 - protein, 14 aa, synthetic construct
RKKLTQSKFVGGAE

SEQ ID NO: 498 - protein, 4 aa, synthetic construct
KHYR

SEQ ID NO: 499 - skipped

SEQ ID NO: 500 - protein, 7 aa, synthetic construct
PRPFNYL

SEQ ID NO: 501 - protein, 7 aa, synthetic construct
APFEMSA

SEQ ID NO: 502 - protein, 7 aa, synthetic construct
APFEFSA

SEQ ID NO: 503 - protein, 6 aa, synthetic construct
PLGFRV

SEQ ID NO: 504 - protein, 8 aa, synthetic construct
RPLALWRS

SEQ ID NO: 505 - protein, 9 aa, synthetic construct
RPLALEESQ

SEQ ID NO: 506 - protein, 9 aa, synthetic construct
RPLALWRSQ

SEQ ID NO: 507 - protein, 11 aa, synthetic construct
RNALAVERTAS

SEQ ID NO: 508 - protein, 8 aa, synthetic construct
RPKPQQFW

SEQ ID NO: 509 - protein, 10 aa, synthetic construct
SGSNPYKYTA

SEQ ID NO: 510 - protein, 10 aa, synthetic construct
SGSNPYGYTA

SEQ ID NO: 511 - protein, 10 aa, synthetic construct
SGTLSELHTA

SEQ ID NO: 512 - protein, 10 aa, synthetic construct
SGTISHLHTA

SEQ ID NO: 513 - protein, 13 aa, synthetic construct
  SITE 3: note = L-Ornithine
  SITE 8: note = L-homophenylalanine
SGXRSHPFTLYTA

SEQ ID NO: 514 - protein, 13 aa, synthetic construct
  SITE 3: note = L-Ornithine
  SITE 8: note = L-homophenylalanine
SGXRSHGFFLYTA

SEQ ID NO: 515 - protein, 10 aa, synthetic construct
SGESLAYYTA

SEQ ID NO: 516 - protein, 10 aa, synthetic construct
SGHMHAALTA

SEQ ID NO: 517 - protein, 8 aa, synthetic construct
  SITE 5: note = D-isoleucine
ILSRIVGG

SEQ ID NO: 518 - protein, 8 aa, synthetic construct
  SITE 4: note = D-arginine
  SITE 5: note = D-isoleucine
  SITE 6: note = D-valine
ILSRIVGG

SEQ ID NO: 519 - protein, 8 aa, synthetic construct
RQRRALEK

SEQ ID NO: 520 - protein, 8 aa, synthetic construct
KPISLISS

SEQ ID NO: 521 - protein, 8 aa, synthetic construct
QKGRYKQE

SEQ ID NO: 522 - protein, 8 aa, synthetic construct
GPLGLRSW

SEQ ID NO: 523 - protein, 8 aa, synthetic construct
GPLGVRGK

SEQ ID NO: 524 - protein, 7 aa, synthetic construct
  SITE 2: note = D-Phenylalanine
GFPRSGG

SEQ ID NO: 525-550 - skipped (26 entries)

SEQ ID NO: 551 - protein, 4 aa, synthetic construct
WEHD

SEQ ID NO: 552-554 - skipped (3 entries)

SEQ ID NO: 555 - protein, 4 aa, synthetic construct
AAPM

SEQ ID NO: 556 - protein, 4 aa, synthetic construct
AEPF

SEQ ID NO: 557-566 - skipped (10 entries)

SEQ ID NO: 567 - protein, 4 aa, synthetic construct
AAPV

SEQ ID NO: 568-569 - skipped (2 entries)

SEQ ID NO: 570 - protein, 5 aa, synthetic construct
GPLGP

SEQ ID NO: 571 - protein, 5 aa, synthetic construct
KQKER

SEQ ID NO: 572 - protein, 4 aa, synthetic construct
RVRR

SEQ ID NO: 573 - protein, 4 aa, synthetic construct
IEGR

SEQ ID NO: 574 - skipped

SEQ ID NO: 575 - protein, 4 aa, synthetic construct
AAPV

SEQ ID NO: 576 - protein, 8 aa, synthetic construct
RPFHLLVY

SEQ ID NO: 577 - protein, 7 aa, synthetic construct
  SITE 1: note = Amino-n-butyric acid
  SITE 4: note = Guamidine-L-phenylalanine
XWSFTVF

SEQ ID NO: 578 - protein, 6 aa, synthetic construct
HSSKLQ

SEQ ID NO: 579 - skipped

SEQ ID NO: 580 - protein, 8 aa, synthetic construct
  SITE 7: note = Dinitrobenzylation of lysine
  SITE 8: note = Hydroxy Leucine
DRENSPKL

SEQ ID NO: 581 - protein, 10 aa, synthetic construct
  SITE 1: note = D-Lysine
  SITE 2: note = D-Lysine
  SITE 10: note = Dinitrobenzylation of Hydroxy lysine
KKDRENSPLK

SEQ ID NO: 582 - protein, 8 aa, synthetic construct
  SITE 7: note = Dinitrobenzylation of lysine
  SITE 8: note = Hydroxy Glutamine
NAGSKFKQ

SEQ ID NO: 583 - protein, 8 aa, synthetic construct
  SITE 8: note = Dinitrobenzylation of Hydroxy lysine
NAGSKFQK

SEQ ID NO: 584 - protein, 8 aa, synthetic construct
  SITE 7: note = Dinitrobenzylation of lysine
  SITE 8: note = Hydroxy Valine
HLLGFYKV

SEQ ID NO: 585 - protein, 8 aa, synthetic construct
  SITE 8: note = Dinitrobenzylation of Hydroxy lysine
HLLGFYVK

SEQ ID NO: 586 - protein, 8 aa, synthetic construct
  SITE 6: note = Norleucine
  SITE 7: note = Dinitrobenzylation of lysine
  SITE 8: note = Hydroxy Norleucine
QEKQTXKX

SEQ ID NO: 587 - protein, 8 aa, synthetic construct
  SITE 6: note = Norleucine
  SITE 7: note = Norleucine
  SITE 8: note = Dinitrobenzylation of Hydroxy lysine
QEKQTXXK

SEQ ID NO: 588 - protein, 8 aa, synthetic construct
  SITE 7: note = Dinitrobenzylation of lysine
  SITE 8: note = Hydroxy Tryptophan
DPFVVSKW

SEQ ID NO: 589 - protein, 8 aa, synthetic construct
  SITE 8: note = Dinitrobenzylation of Hydroxy lysine
DPFVVSWK

SEQ ID NO: 590 - protein, 8 aa, synthetic construct
  SITE 7: note = Dinitrobenzylation of lysine
  SITE 8: note = Hydroxy Arginine
NAYNEIKR

SEQ ID NO: 591 - protein, 8 aa, synthetic construct
  SITE 8: note = Dinitrobenzylation of Hydroxy lysine
NAYNEIRK

SEQ ID NO: 592 - protein, 8 aa, synthetic construct
  SITE 2: note = Norleucine
  SITE 7: note = Dinitrobenzylation of lysine
  SITE 8: note = Hydroxy Asparagine
VXRQSEKN

SEQ ID NO: 593 - protein, 8 aa, synthetic construct
  SITE 2: note = Norleucine
  SITE 8: note = Dinitrobenzylation of Hydroxy lysine
VXRQSENK

SEQ ID NO: 594 - protein, 8 aa, synthetic construct
  SITE 6: note = Norleucine
  SITE 7: note = Dinitrobenzylation of lysine
  SITE 8: note = Hydroxy Isoleucine
YNPREXKI

SEQ ID NO: 595 - protein, 8 aa, synthetic construct
  SITE 6: note = Norleucine
  SITE 8: note = Dinitrobenzylation of Hydroxy lysine
YNPREXIK

SEQ ID NO: 596 - protein, 8 aa, synthetic construct
  SITE 7: note = Dinitrobenzylation of lysine
  SITE 8: note = Hydroxy Lysine
EFVHNPKK

SEQ ID NO: 597 - protein, 8 aa, synthetic construct
  SITE 8: note = Dinitrobenzylation of Hydroxy lysine
EFVHNPKK

SEQ ID NO: 598 - protein, 8 aa, synthetic construct
  SITE 7: note = Dinitrobenzylation of lysine
  SITE 8: note = Hydroxy Histidine
KRVQFLKH

SEQ ID NO: 599 - protein, 8 aa, synthetic construct
  SITE 8: note = Dinitrobenzylation of Hydroxy lysine
KRVQFLHK

SEQ ID NO: 600 - protein, 8 aa, synthetic construct
  SITE 3: note = Norleucine
  SITE 7: note = Dinitrobenzylation of lysine
  SITE 8: note = Hydroxy Glycine
LIXHKNKG

SEQ ID NO: 601 - protein, 8 aa, synthetic construct
  SITE 3: note = Norleucine
  SITE 8: note = Dinitrobenzylation of Hydroxy lysine
LIXHKNGK

SEQ ID NO: 602 - protein, 8 aa, synthetic construct
  SITE 3: note = Norleucine
  SITE 7: note = Dinitrobenzylation of lysine
  SITE 8: note = Hydroxy Serine
WAXLYHKS

SEQ ID NO: 603 - protein, 8 aa, synthetic construct
  SITE 3: note = Norleucine
  SITE 8: note = Dinitrobenzylation of Hydroxy lysine
WAXLYHSK

SEQ ID NO: 604 - protein, 8 aa, synthetic construct
  SITE 7: note = Dinitrobenzylation of lysine
  SITE 8: note = Hydroxy Tyrosine
AHDIVNKY

SEQ ID NO: 605 - protein, 8 aa, synthetic construct
  SITE 8: note = Dinitrobenzylation of Hydroxy lysine
AHDIVNYK

SEQ ID NO: 606 - protein, 8 aa, synthetic construct
  SITE 7: note = Dinitrobenzylation of lysine
  SITE 8: note = Hydroxy Proline
SVFVIEKP

SEQ ID NO: 607 - protein, 8 aa, synthetic construct
  SITE 8: note = Dinitrobenzylation of Hydroxy lysine
SVFVIEPK

SEQ ID NO: 608 - protein, 8 aa, synthetic construct
  SITE 7: note = Dinitrobenzylation of lysine
  SITE 8: note = Hydroxy Glutamic Acid
PPSGLSKE

SEQ ID NO: 609 - protein, 8 aa, synthetic construct
  SITE 8: note = Dinitrobenzylation of Hydroxy lysine
PPSGLSEK

SEQ ID NO: 610 - protein, 8 aa, synthetic construct
  SITE 7: note = Dinitrobenzylation of lysine
  SITE 8: note = Hydroxy Phenylalanine
RWYGGIKF

SEQ ID NO: 611 - protein, 8 aa, synthetic construct
  SITE 8: note = Dinitrobenzylation of Hydroxy lysine
RWYGGIFK

SEQ ID NO: 612 - protein, 8 aa, synthetic construct
  SITE 6: note = Norleucine
  SITE 7: note = Dinitrobenzylation of lysine
  SITE 8: note = Hydroxy Aspartic Acid
QYVFFXKD

SEQ ID NO: 613 - protein, 8 aa, synthetic construct
  SITE 6: note = Norleucine
  SITE 8: note = Dinitrobenzylation of Hydroxy lysine
QYVFFXDK

SEQ ID NO: 614 - protein, 8 aa, synthetic construct
  SITE 7: note = Dinitrobenzylation of lysine
  SITE 8: note = Hydroxy Threonine
FAKYYKKT

SEQ ID NO: 615 - protein, 8 aa, synthetic construct
  SITE 8: note = Dinitrobenzylation of Hydroxy lysine
FAKYYKTK

SEQ ID NO: 616 - protein, 8 aa, synthetic construct
  SITE 7: note = Dinitrobenzylation of lysine
  SITE 8: note = Hydroxy Alanine
QVKHFTKA

SEQ ID NO: 617 - protein, 8 aa, synthetic construct
  SITE 8: note = Dinitrobenzylation of Hydroxy lysine
QVKHFTAK

SEQ ID NO: 618 - protein, 7 aa, synthetic construct
  SITE 7: note = Dinitrobenzylation of Hydroxy lysine
YVADAPK

SEQ ID NO: 619 - protein, 7 aa, synthetic construct
KGISSQY

SEQ ID NO: 620 - protein, 7 aa, synthetic construct
ALPALQN

SEQ ID NO: 621 - protein, 5 aa, synthetic construct
HRFRG

SEQ ID NO: 622 - protein, 9 aa, synthetic construct
APEEIMDQQ

SEQ ID NO: 623 - protein, 7 aa, synthetic construct
SRKSQQY

SEQ ID NO: 624 - protein, 7 aa, synthetic construct
SKGRSLI

SEQ ID NO: 625 - protein, 7 aa, synthetic construct
FAQSIPK

SEQ ID NO: 626 - protein, 7 aa, synthetic construct
RQRRVVG

SEQ ID NO: 627 - protein, 8 aa, synthetic construct
ERGETGPS

SEQ ID NO: 628 - protein, 6 aa, synthetic construct
ASGPSS

SEQ ID NO: 629 - protein, 4 aa, synthetic construct
YRFR

SEQ ID NO: 630 - protein, 7 aa, synthetic construct
KLFSSKQ

SEQ ID NO: 631 - protein, 5 aa, synthetic construct
IVPRG

SEQ ID NO: 632 - protein, 8 aa, synthetic construct
IRRSSYFK

SEQ ID NO: 633 - protein, 6 aa, synthetic construct
  SITE 1: note = Benzyl-L-histidine
  SITE 2: note = L-tert-leucine
  SITE 6: note = L-methionine-sulfoxide
HXPSDM

SEQ ID NO: 634 - protein, 8 aa, synthetic construct
  SITE 1: note = Norvaline
  SITE 4: note = L-octahydroindole-2-carboxylic acid
XIEXDFGR

SEQ ID NO: 635-637 - skipped (3 entries)

SEQ ID NO: 638 - protein, 6 aa, synthetic construct
  SITE 1: note = Benzyl-L-histidine
  SITE 2: note = L-tert-leucine
  SITE 6: note = L-methionine-sulfoxide
HXPSDM

SEQ ID NO: 639-643 - skipped (5 entries)

SEQ ID NO: 644 - protein, 6 aa, synthetic construct
  SITE 4: note = Guamidine-L-phenylalanine
FVTFSW

SEQ ID NO: 645-646 - skipped (2 entries)

SEQ ID NO: 647 - protein, 7 aa, synthetic construct
AIEPDSG

SEQ ID NO: 648 - protein, 7 aa, synthetic construct
AIEFDSG

SEQ ID NO: 649 - protein, 7 aa, synthetic construct
AAEAISD

SEQ ID NO: 650 - protein, 8 aa, synthetic construct
AGGAQMGA

SEQ ID NO: 651 - protein, 8 aa, synthetic construct
AQPDALNV

SEQ ID NO: 652 - protein, 8 aa, synthetic construct
ATDVTTTP

SEQ ID NO: 653 - protein, 8 aa, synthetic construct
DIVTVANA

SEQ ID NO: 654 - protein, 8 aa, synthetic construct
DLGLKSVP

SEQ ID NO: 655 - protein, 8 aa, synthetic construct
DVMASNKR

SEQ ID NO: 656 - protein, 8 aa, synthetic construct
ESDELNTI

SEQ ID NO: 657 - protein, 8 aa, synthetic construct
FHPLHSKI

SEQ ID NO: 658 - protein, 7 aa, synthetic construct
HARLVHV

SEQ ID NO: 659 - protein, 8 aa, synthetic construct
HIANVERV

SEQ ID NO: 660 - protein, 8 aa, synthetic construct
KAAATQKK

SEQ ID NO: 661 - protein, 8 aa, synthetic construct
LATASTMD

SEQ ID NO: 662 - protein, 7 aa, synthetic construct
LGPKGQT

SEQ ID NO: 663 - protein, 8 aa, synthetic construct
LSLPETGE

SEQ ID NO: 664 - protein, 8 aa, synthetic construct
NLAGILKE

SEQ ID NO: 665 - protein, 8 aa, synthetic construct
NPGMSEPV

SEQ ID NO: 666 - protein, 7 aa, synthetic construct
PFGCHAK

SEQ ID NO: 667 - protein, 7 aa, synthetic construct
PLGLRWW

SEQ ID NO: 668 - protein, 8 aa, synthetic construct
QMGVMQGV

SEQ ID NO: 669 - protein, 8 aa, synthetic construct
QTCKCSCK

SEQ ID NO: 670 - protein, 8 aa, synthetic construct
QWAGLVEK

SEQ ID NO: 671 - protein, 8 aa, synthetic construct
RPAVMTSP

SEQ ID NO: 672 - protein, 8 aa, synthetic construct
TLRELHLD

SEQ ID NO: 673 - protein, 8 aa, synthetic construct
TPPPSQGK

SEQ ID NO: 674 - protein, 8 aa, synthetic construct
TSEDLVVQ

SEQ ID NO: 675 - protein, 8 aa, synthetic construct
VWAAEAIS

SEQ ID NO: 676-677 - skipped (2 entries)

SEQ ID NO: 678 - protein, 8 aa, synthetic construct
  SITE 8: note = Lysine modified with a CPQ2 quencher
GSGRSGGK

SEQ ID NO: 679 - protein, 9 aa, synthetic construct
  SITE 9: note = Lysine modified with a CPQ2 quencher
GPGPREGGK

SEQ ID NO: 680 - protein, 11 aa, synthetic construct
  SITE 11: note = Lysine modified with a CPQ2 quencher
GIEPDSGSQGK

SEQ ID NO: 681 - protein, 12 aa, synthetic construct
  SITE 12: note = Lysine modified with a CPQ2 quencher
GVVADSSMESGK

SEQ ID NO: 682 - protein, 7 aa, synthetic construct
  SITE 7: note = Lysine modified with a CPQ2 quencher
GPTSYGK

SEQ ID NO: 683 - protein, 7 aa, synthetic construct
  SITE 7: note = Lysine modified with a CPQ2 quencher
GYRFKGK

SEQ ID NO: 684 - protein, 7 aa, synthetic construct
  SITE 7: note = Lysine modified with a CPQ2 quencher
GKVPLGK

SEQ ID NO: 685 - protein, 8 aa, synthetic construct
  SITE 8: note = Lysine modified with a CPQ2 quencher
GVDVADGK

SEQ ID NO: 686 - protein, 7 aa, synthetic construct
  SITE 7: note = Lysine modified with a CPQ2 quencher
GLETDGK

SEQ ID NO: 687 - protein, 7 aa, synthetic construct
  SITE 7: note = Lysine modified with a CPQ2 quencher
GLEHDGK

SEQ ID NO: 688 - protein, 7 aa, synthetic construct
  SITE 7: note = Lysine modified with a CPQ2 quencher
GREQDGK

SEQ ID NO: 689 - protein, 7 aa, synthetic construct
  SITE 7: note = Lysine modified with a CPQ2 quencher
GDEVDGK

SEQ ID NO: 690 - protein, 7 aa, synthetic construct
  SITE 7: note = Lysine modified with a CPQ2 quencher
GVEIDGK

SEQ ID NO: 691 - protein, 9 aa, synthetic construct
  SITE 9: note = Lysine modified with a CPQ2 quencher
GVQVDGWGK

SEQ ID NO: 692 - protein, 9 aa, synthetic construct
  SITE 9: note = Lysine modified with a CPQ2 quencher
GYEVDGWGK

SEQ ID NO: 693 - protein, 7 aa, synthetic construct
  SITE 7: note = Lysine modified with a CPQ2 quencher
GLEVDGK

SEQ ID NO: 694 - protein, 7 aa, synthetic construct
  SITE 7: note = Lysine modified with a CPQ2 quencher
GIEVEGK

SEQ ID NO: 695 - protein, 7 aa, synthetic construct
  SITE 7: note = Lysine modified with a CPQ2 quencher
GAAPVGK

SEQ ID NO: 696 - protein, 7 aa, synthetic construct
  SITE 7: note = Lysine modified with a CPQ2 quencher
GFFKFGK

SEQ ID NO: 697 - protein, 10 aa, synthetic construct
  SITE 10: note = Lysine modified with a CPQ2 quencher
GGRRGKGGGK

SEQ ID NO: 698 - protein, 7 aa, synthetic construct
  SITE 7: note = Lysine modified with a CPQ2 quencher
GVKKRGK

SEQ ID NO: 699 - protein, 10 aa, synthetic construct
  SITE 5: note = p-Nitro phenylalanine
  SITE 10: note = Lysine modified with a CPQ2 quencher
GFAAFFVLGK

SEQ ID NO: 700 - protein, 6 aa, synthetic construct
  SITE 6: note = Lysine modified with a CPQ2 quencher
GVVRGK

SEQ ID NO: 701 - protein, 8 aa, synthetic construct
  SITE 8: note = Lysine modified with a CPQ2 quencher
GKQKLRGK

SEQ ID NO: 702 - protein, 11 aa, synthetic construct
  SITE 11: note = Lysine modified with a CPQ2 quencher
GRPPGFSAFGK

SEQ ID NO: 703 - protein, 6 aa, synthetic construct
  SITE 6: note = Lysine modified with a CPQ2 quencher
GGPRGK

SEQ ID NO: 704 - protein, 5 aa, synthetic construct
  SITE 5: note = Lysine modified with a CPQ2 quencher
GFRGK

SEQ ID NO: 705 - protein, 8 aa, synthetic construct
  SITE 8: note = Lysine modified with a CPQ2 quencher
GLPLGLGK

SEQ ID NO: 706 - protein, 8 aa, synthetic construct
  SITE 8: note = Lysine modified with a CPQ2 quencher
GKPLGLGK

SEQ ID NO: 707 - protein, 9 aa, synthetic construct
  SITE 2: note = Gamma aminobutyric acid
  SITE 9: note = Lysine modified with a CPQ2 quencher
GXPQGLEGK

SEQ ID NO: 708 - protein, 9 aa, synthetic construct
  SITE 9: note = Lysine modified with a CPQ2 quencher
GPKPLALGK

SEQ ID NO: 709 - protein, 10 aa, synthetic construct
  SITE 10: note = Lysine modified with a CPQ2 quencher
GGPSGIHVGK

SEQ ID NO: 710 - protein, 15 aa, synthetic construct
  SITE 15: note = Lysine modified with a CPQ2 quencher
GWAHRTTFYRRGAGK

SEQ ID NO: 711 - protein, 11 aa, synthetic construct
  SITE 11: note = Lysine modified with a CPQ2 quencher
GWKLRSSKQGK

SEQ ID NO: 712 - protein, 6 aa, synthetic construct
  SITE 6: note = Lysine modified with a CPQ2 quencher
GPFRGK

SEQ ID NO: 713 - protein, 8 aa, synthetic construct
  SITE 8: note = Lysine modified with a CPQ2 quencher
GSYRIFGK

SEQ ID NO: 714 - protein, 6 aa, synthetic construct
  SITE 6: note = Lysine modified with a CPQ2 quencher
GRPYGK

SEQ ID NO: 715 - protein, 11 aa, synthetic construct
  SITE 11: note = Lysine modified with a CPQ2 quencher
GTAFRSAYGGK

SEQ ID NO: 716 - protein, 12 aa, synthetic construct
  SITE 12: note = Lysine modified with a CPQ2 quencher
GWAAFRFSQAGK

SEQ ID NO: 717 - protein, 6 aa, synthetic construct
  SITE 6: note = Lysine modified with a CPQ2 quencher
GVPRGK

SEQ ID NO: 718 - protein, 10 aa, synthetic construct
  SITE 10: note = Lysine modified with a CPQ2 quencher
GKLRSSKQGK

SEQ ID NO: 719 - protein, 7 aa, synthetic construct
  SITE 7: note = Lysine modified with a CPQ2 quencher
GYASRGK

SEQ ID NO: 720 - protein, 13 aa, synthetic construct
  SITE 13: note = Lysine modified with a CPQ2 quencher
GRFAQAQQQLPGK

SEQ ID NO: 721 - protein, 11 aa, synthetic construct
  SITE 11: note = Lysine modified with a CPQ2 quencher
GKPAKFFRLGK

SEQ ID NO: 722 - protein, 12 aa, synthetic construct
  SITE 7: note = L-Homophenylalanine
  SITE 12: note = Lysine modified with a CPQ2 quencher
GPRAAAFTSPGK

SEQ ID NO: 723 - protein, 13 aa, synthetic construct
  SITE 13: note = Lysine modified with a CPQ2 quencher
GVGPQRFSGAPGK

SEQ ID NO: 724 - protein, 12 aa, synthetic construct
  SITE 8: note = L-Homophenylalanine
  SITE 12: note = Lysine modified with a CPQ2 quencher
GFFLAQAFRSGK

SEQ ID NO: 725 - protein, 9 aa, synthetic construct
  SITE 9: note = Lysine modified with a CPQ2 quencher
GPLAQAVGK

SEQ ID NO: 726 - protein, 11 aa, synthetic construct
  SITE 11: note = Lysine modified with a CPQ2 quencher
GRTAAVFRPGK

SEQ ID NO: 727 - protein, 16 aa, synthetic construct
  SITE 16: note = Lysine modified with a CPQ2 quencher
GDVQEFRGVTAVIRGK

SEQ ID NO: 728 - protein, 13 aa, synthetic construct
  SITE 13: note = Lysine modified with a CPQ2 quencher
GTEGEARGSVIGK

SEQ ID NO: 729 - protein, 6 aa, synthetic construct
  SITE 2: note = D-Leucine
  SITE 6: note = Lysine modified with a CPQ2 quencher
GLTRGK

SEQ ID NO: 730 - protein, 10 aa, synthetic construct
  SITE 10: note = Lysine modified with a CPQ2 quencher
GPLFAERKGK

SEQ ID NO: 731 - protein, 7 aa, synthetic construct
  SITE 7: note = Lysine modified with a CPQ2 quencher
GLLVYGK

SEQ ID NO: 732 - protein, 11 aa, synthetic construct
  SITE 11: note = Lysine modified with a CPQ2 quencher
GQQKRKIVLGK

SEQ ID NO: 733 - protein, 11 aa, synthetic construct
  SITE 11: note = Lysine modified with a CPQ2 quencher
GASHLGLARGK

SEQ ID NO: 734 - protein, 11 aa, synthetic construct
  SITE 11: note = Lysine modified with a CPQ2 quencher
GLPSRSSKIGK

SEQ ID NO: 735 - protein, 11 aa, synthetic construct
  SITE 11: note = Lysine modified with a CPQ2 quencher
GSTGRNGFKGK

SEQ ID NO: 736 - protein, 11 aa, synthetic construct
  SITE 11: note = Lysine modified with a CPQ2 quencher
GSLLRSEETGK

SEQ ID NO: 737 - protein, 11 aa, synthetic construct
  SITE 11: note = Lysine modified with a CPQ2 quencher
GHRGRTLEIGK

SEQ ID NO: 738 - protein, 11 aa, synthetic construct
  SITE 11: note = Lysine modified with a CPQ2 quencher
GYLGRSYKVGK

SEQ ID NO: 739 - protein, 11 aa, synthetic construct
  SITE 11: note = Lysine modified with a CPQ2 quencher
GEKQRIIGGGK

SEQ ID NO: 740 - protein, 11 aa, synthetic construct
  SITE 11: note = Lysine modified with a CPQ2 quencher
GQRQRIIGGGK

SEQ ID NO: 741 - protein, 9 aa, synthetic construct
  SITE 9: note = Lysine modified with a CPQ2 quencher
GLQRIYKGK

SEQ ID NO: 742 - protein, 11 aa, synthetic construct
  SITE 11: note = Lysine modified with a CPQ2 quencher
GSLGRKIQIGK

SEQ ID NO: 743 - protein, 16 aa, synthetic construct
  SITE 16: note = Lysine modified with a CPQ2 quencher
GHAAPRSADIQIDIGK

SEQ ID NO: 744 - protein, 6 aa, synthetic construct
  SITE 6: note = Lysine modified with a CPQ2 quencher
GFGRGK

SEQ ID NO: 745 - protein, 7 aa, synthetic construct
  SITE 7: note = Lysine modified with a CPQ2 quencher
GSLGRGK

SEQ ID NO: 746 - protein, 7 aa, synthetic construct
  SITE 7: note = Lysine modified with a CPQ2 quencher
GGLQRGK

SEQ ID NO: 747 - protein, 11 aa, synthetic construct
  SITE 11: note = Lysine modified with a CPQ2 quencher
GSVARTLLVGK

SEQ ID NO: 748 - protein, 8 aa, synthetic construct
  SITE 8: note = Lysine modified with a CPQ2 quencher
GGRIFGGK

SEQ ID NO: 749 - protein, 6 aa, synthetic construct
  SITE 6: note = Lysine modified with a CPQ2 quencher
GAPKGK

SEQ ID NO: 750 - protein, 8 aa, synthetic construct
  SITE 8: note = Lysine modified with a CPQ2 quencher
GGFSPYGK

SEQ ID NO: 751 - protein, 11 aa, synthetic construct
  SITE 11: note = Lysine modified with a CPQ2 quencher
GWELRHAGHGK

SEQ ID NO: 752 - protein, 12 aa, synthetic construct
  SITE 12: note = Lysine modified with a CPQ2 quencher
GRQSRIVGGEGK

SEQ ID NO: 753 - protein, 11 aa, synthetic construct
  SITE 11: note = Lysine modified with a CPQ2 quencher
GEQAVYQTIGK

SEQ ID NO: 754 - protein, 14 aa, synthetic construct
  SITE 14: note = Lysine modified with a CPQ2 quencher
GVAYSGENTFGFGK

SEQ ID NO: 755 - protein, 6 aa, synthetic construct
  SITE 6: note = Lysine modified with a CPQ2 quencher
GGGRGK

SEQ ID NO: 756 - protein, 7 aa, synthetic construct
  SITE 7: note = Lysine modified with a CPQ2 quencher
GATADGK

SEQ ID NO: 757 - protein, 11 aa, synthetic construct
  SITE 11: note = Lysine modified with a CPQ2 quencher
GRPLESNAVGK

SEQ ID NO: 758 - protein, 10 aa, synthetic construct
  SITE 10: note = Lysine modified with a CPQ2 quencher
GRPLGLARGK

SEQ ID NO: 759 - protein, 7 aa, synthetic construct
  SITE 7: note = Lysine modified with a CPQ2 quencher
GAAFFGK

SEQ ID NO: 760 - protein, 10 aa, synthetic construct
  SITE 10: note = Lysine modified with a CPQ2 quencher
GRVKRGLAGK

SEQ ID NO: 761 - protein, 6 aa, synthetic construct
  SITE 6: note = Lysine modified with a CPQ2 quencher
GAALGK

SEQ ID NO: 762 - protein, 20 aa, synthetic construct
  SITE 4: note = D-Methionine
  SITE 5: note = D-Glutamic Acid
  SITE 8: note = D-Asparagine
  SITE 9: note = D-Aspartic Acid
  SITE 10: note = D-Asparagine
  SITE 11: note = D-Glutamic Acid
  SITE 12: note = D-Glutamic Acid
  SITE 16: note = D-Serine
  SITE 18: note = D-Arginine
  SITE 20: note = Lysine modified with a CPQ2 quencher
CGGMEGVNDNEEGFFSARGK

SEQ ID NO: 763 - protein, 10 aa, synthetic construct
  SITE 10: note = Lysine modified with a CPQ2 quencher
GGPQGIWGQK

SEQ ID NO: 764 - protein, 10 aa, synthetic construct
  SITE 10: note = Lysine modified with a CPQ2 quencher
GGLVPRGSGK

SEQ ID NO: 765 - protein, 9 aa, synthetic construct
  SITE 9: note = Lysine modified with a CPQ2 quencher
GGPVGLIGK

SEQ ID NO: 766 - protein, 11 aa, synthetic construct
  SITE 11: note = Lysine modified with a CPQ2 quencher
GGPWGIWGQGK

SEQ ID NO: 767 - protein, 11 aa, synthetic construct
  SITE 11: note = Lysine modified with a CPQ2 quencher
GGPVPLSLVMK

SEQ ID NO: 768 - protein, 10 aa, synthetic construct
  SITE 3: note = D-Phenylalanine
  SITE 4: note = Piperidine carboxylic acid
  SITE 10: note = Lysine modified with a CPQ2 quencher
GGFXRSGGGK

SEQ ID NO: 769 - protein, 10 aa, synthetic construct
  SITE 3: note = D-Phenylalanine
  SITE 4: note = Piperidine carboxylic acid
  SITE 10: note = Lysine modified with a CPQ2 quencher
GGFXKSGGGK

SEQ ID NO: 770 - protein, 9 aa, synthetic construct
  SITE 9: note = Lysine modified with a CPQ2 quencher
GPLGMRGGK

SEQ ID NO: 771 - protein, 9 aa, synthetic construct
  SITE 3: note = L-cyclohexylalanine
  SITE 5: note = L- Methyl cysteine
  SITE 9: note = Lysine modified with a CPQ2 quencher
GPXGCHAGK

SEQ ID NO: 772 - protein, 12 aa, synthetic construct
  SITE 12: note = Lysine modified with a CPQ2 quencher
GRPLALWESQGK

SEQ ID NO: 773 - protein, 11 aa, synthetic construct
  SITE 11: note = Lysine modified with a CPQ2 quencher
SGKGPRQITAK

SEQ ID NO: 774 - protein, 11 aa, synthetic construct
  SITE 11: note = Lysine modified with a CPQ2 quencher
SGPLFYSVTAK

SEQ ID NO: 775 - protein, 10 aa, synthetic construct
  SITE 10: note = Lysine modified with a CPQ2 quencher
SGRIFLRTAK

SEQ ID NO: 776 - protein, 11 aa, synthetic construct
  SITE 11: note = Lysine modified with a CPQ2 quencher
SGRSENIRTAK

SEQ ID NO: 777 - protein, 7 aa, synthetic construct
  SITE 7: note = Lysine modified with a CPQ2 quencher
GGSGGSK

SEQ ID NO: 778 - protein, 12 aa, synthetic construct
  SITE 12: note = Lysine modified with a CPQ2 quencher
GKPILFFRLKGK

SEQ ID NO: 779 - protein, 9 aa, synthetic construct
  SITE 7: note = Norleucine
  SITE 9: note = Lysine Amide modified with a CPQ2 quencher
GAWESRXGK

SEQ ID NO: 780 - protein, 9 aa, synthetic construct
  SITE 7: note = Norleucine
  SITE 9: note = Lysine Amide modified with a CPQ2 quencher
GNEKSGXGK

SEQ ID NO: 781 - protein, 9 aa, synthetic construct
  SITE 9: note = Lysine modified with a CPQ2 quencher
GNATIVYGK

SEQ ID NO: 782 - protein, 9 aa, synthetic construct
  SITE 9: note = Lysine modified with a CPQ2 quencher
GDPFVVSGK

SEQ ID NO: 783 - protein, 9 aa, synthetic construct
  SITE 4: note = Norleucine
  SITE 9: note = Lysine modified with a CPQ2 quencher
GFHXFTKGK

SEQ ID NO: 784 - protein, 9 aa, synthetic construct
  SITE 2: note = Norleucine
  SITE 9: note = Lysine Amide modified with a CPQ2 quencher
GXNWHKHGK

SEQ ID NO: 785 - protein, 9 aa, synthetic construct
  SITE 9: note = Lysine modified with a CPQ2 quencher
GFARRWGGK

SEQ ID NO: 786 - protein, 9 aa, synthetic construct
  SITE 9: note = Lysine modified with a CPQ2 quencher
GPGKWSKGK

SEQ ID NO: 787 - protein, 9 aa, synthetic construct
  SITE 9: note = Lysine modified with a CPQ2 quencher
GYEEAQPGK

SEQ ID NO: 788 - protein, 9 aa, synthetic construct
  SITE 9: note = Lysine modified with a CPQ2 quencher
GYGAIKKGK

SEQ ID NO: 789 - protein, 9 aa, synthetic construct
  SITE 4: note = Norleucine
  SITE 9: note = Lysine modified with a CPQ2 quencher
GTSXEGYGK

SEQ ID NO: 790 - protein, 9 aa, synthetic construct
  SITE 9: note = Lysine modified with a CPQ2 quencher
GPNNFGSGK

SEQ ID NO: 791 - protein, 9 aa, synthetic construct
  SITE 9: note = Lysine Amide modified with a CPQ2 quencher
GEDTRNTGK

SEQ ID NO: 792 - protein, 9 aa, synthetic construct
  SITE 9: note = Lysine Amide modified with a CPQ2 quencher
GKDLEQSGK

SEQ ID NO: 793 - protein, 9 aa, synthetic construct
  SITE 9: note = Lysine modified with a CPQ2 quencher
GAALHNDGK

SEQ ID NO: 794 - protein, 9 aa, synthetic construct
  SITE 9: note = Lysine Amide modified with a CPQ2 quencher
GADSFFKGK

SEQ ID NO: 795 - protein, 9 aa, synthetic construct
  SITE 9: note = Lysine Amide modified with a CPQ2 quencher
GITFWRAGK

SEQ ID NO: 796 - protein, 9 aa, synthetic construct
  SITE 5: note = Norleucine
  SITE 9: note = Lysine Amide modified with a CPQ2 quencher
GLSDXRLGK

SEQ ID NO: 797 - protein, 9 aa, synthetic construct
  SITE 9: note = Lysine modified with a CPQ2 quencher
GEVGWTYGK

SEQ ID NO: 798 - protein, 9 aa, synthetic construct
  SITE 7: note = Norleucine
  SITE 9: note = Lysine Amide modified with a CPQ2 quencher
GIAFRQXGK

SEQ ID NO: 799 - protein, 9 aa, synthetic construct
  SITE 7: note = Norleucine
  SITE 9: note = Lysine modified with a CPQ2 quencher
GYNIHTXGK

SEQ ID NO: 800 - protein, 9 aa, synthetic construct
  SITE 2: note = Norleucine
  SITE 9: note = Lysine modified with a CPQ2 quencher
GXLWANHGK

SEQ ID NO: 801 - protein, 9 aa, synthetic construct
  SITE 9: note = Lysine modified with a CPQ2 quencher
GLYSVQVGK

SEQ ID NO: 802 - protein, 9 aa, synthetic construct
  SITE 5: note = Norleucine
  SITE 9: note = Lysine modified with a CPQ2 quencher
GSHIXSNGK

SEQ ID NO: 803 - protein, 9 aa, synthetic construct
  SITE 9: note = Lysine Amide modified with a CPQ2 quencher
GKLLIDVGK

SEQ ID NO: 804 - protein, 9 aa, synthetic construct
  SITE 3: note = Norleucine
  SITE 9: note = Lysine modified with a CPQ2 quencher
GEXGVFDGK

SEQ ID NO: 805 - protein, 9 aa, synthetic construct
  SITE 9: note = Lysine modified with a CPQ2 quencher
GHQAYTLGK

SEQ ID NO: 806 - protein, 9 aa, synthetic construct
  SITE 9: note = Lysine modified with a CPQ2 quencher
GYVRKIQGK

SEQ ID NO: 807 - protein, 9 aa, synthetic construct
  SITE 9: note = Lysine Amide modified with a CPQ2 quencher
GDRENSPGK

SEQ ID NO: 808 - protein, 9 aa, synthetic construct
  SITE 9: note = Lysine Amide modified with a CPQ2 quencher
GKYDKPRGK

SEQ ID NO: 809 - protein, 9 aa, synthetic construct
  SITE 9: note = Lysine modified with a CPQ2 quencher
GRPWKQLGK

SEQ ID NO: 810 - protein, 9 aa, synthetic construct
  SITE 9: note = Lysine Amide modified with a CPQ2 quencher
GAPLQRYGK

SEQ ID NO: 811 - protein, 9 aa, synthetic construct
  SITE 7: note = Norleucine
  SITE 9: note = Lysine Amide modified with a CPQ2 quencher
GYQGQKXGK

SEQ ID NO: 812 - protein, 9 aa, synthetic construct
  SITE 9: note = Lysine Amide modified with a CPQ2 quencher
GGRISSIGK

SEQ ID NO: 813 - protein, 9 aa, synthetic construct
  SITE 9: note = Lysine modified with a CPQ2 quencher
GHSLTNVGK

SEQ ID NO: 814 - protein, 9 aa, synthetic construct
  SITE 9: note = Lysine modified with a CPQ2 quencher
GEWDFPEGK

SEQ ID NO: 815 - protein, 9 aa, synthetic construct
  SITE 5: note = Norleucine
  SITE 9: note = Lysine modified with a CPQ2 quencher
GYLAXDGGK

SEQ ID NO: 816 - protein, 9 aa, synthetic construct
  SITE 5: note = Norleucine
  SITE 9: note = Lysine modified with a CPQ2 quencher
GFIYXPTGK

SEQ ID NO: 817 - protein, 9 aa, synthetic construct
  SITE 9: note = Lysine modified with a CPQ2 quencher
GGHETWVGK

SEQ ID NO: 818 - protein, 9 aa, synthetic construct
  SITE 9: note = Lysine modified with a CPQ2 quencher
GDYIGDEGK

SEQ ID NO: 819 - protein, 9 aa, synthetic construct
  SITE 9: note = Lysine modified with a CPQ2 quencher
GAGTAHPGK

SEQ ID NO: 820 - protein, 9 aa, synthetic construct
  SITE 3: note = Norleucine
  SITE 9: note = Lysine modified with a CPQ2 quencher
GVXTEIWGK

SEQ ID NO: 821 - protein, 9 aa, synthetic construct
  SITE 9: note = Lysine modified with a CPQ2 quencher
GPDDWQNGK

SEQ ID NO: 822 - protein, 9 aa, synthetic construct
  SITE 9: note = Lysine modified with a CPQ2 quencher
GGLNQEYGK

SEQ ID NO: 823 - protein, 9 aa, synthetic construct
  SITE 9: note = Lysine Amide modified with a CPQ2 quencher
GYRDAVAGK

SEQ ID NO: 824 - protein, 9 aa, synthetic construct
  SITE 9: note = Lysine Amide modified with a CPQ2 quencher
GTGPKGNGK

SEQ ID NO: 825 - protein, 9 aa, synthetic construct
  SITE 9: note = Lysine modified with a CPQ2 quencher
GDHVPQIGK

SEQ ID NO: 826 - protein, 9 aa, synthetic construct
  SITE 9: note = Lysine Amide modified with a CPQ2 quencher
GNKEPILGK

SEQ ID NO: 827 - protein, 9 aa, synthetic construct
  SITE 5: note = Norleucine
  SITE 9: note = Lysine modified with a CPQ2 quencher
GVWNXVHGK

SEQ ID NO: 828 - protein, 9 aa, synthetic construct
  SITE 9: note = Lysine modified with a CPQ2 quencher
GPVIIEHGK

SEQ ID NO: 829 - protein, 9 aa, synthetic construct
  SITE 9: note = Lysine modified with a CPQ2 quencher
GFQTDNLGK

SEQ ID NO: 830 - protein, 9 aa, synthetic construct
  SITE 4: note = Norleucine
  SITE 9: note = Lysine modified with a CPQ2 quencher
GRFXHGIGK

SEQ ID NO: 831 - protein, 9 aa, synthetic construct
  SITE 9: note = Lysine Amide modified with a CPQ2 quencher
GYAERTTGK

SEQ ID NO: 832 - protein, 9 aa, synthetic construct
  SITE 9: note = Lysine Amide modified with a CPQ2 quencher
GNRGELPGK

SEQ ID NO: 833 - protein, 9 aa, synthetic construct
  SITE 9: note = Lysine modified with a CPQ2 quencher
GHHYFNYGK

SEQ ID NO: 834 - protein, 9 aa, synthetic construct
  SITE 9: note = Lysine modified with a CPQ2 quencher
GSTPYYHGK

SEQ ID NO: 835 - protein, 9 aa, synthetic construct
  SITE 9: note = Lysine modified with a CPQ2 quencher
GWFYPSAGK

SEQ ID NO: 836 - protein, 9 aa, synthetic construct
  SITE 9: note = Lysine modified with a CPQ2 quencher
GSEFLFSGK

SEQ ID NO: 837 - protein, 9 aa, synthetic construct
  SITE 9: note = Lysine Amide modified with a CPQ2 quencher
GWYKTQYGK

SEQ ID NO: 838 - protein, 9 aa, synthetic construct
  SITE 9: note = Lysine modified with a CPQ2 quencher
GVTHLKVGK

SEQ ID NO: 839 - protein, 9 aa, synthetic construct
  SITE 9: note = Lysine modified with a CPQ2 quencher
GINGGFSGK

SEQ ID NO: 840 - protein, 9 aa, synthetic construct
  SITE 9: note = Lysine modified with a CPQ2 quencher
GTVLGLDGK

SEQ ID NO: 841 - protein, 9 aa, synthetic construct
  SITE 6: note = Norleucine
  SITE 9: note = Lysine modified with a CPQ2 quencher
GSYWPXQGK

SEQ ID NO: 842 - protein, 9 aa, synthetic construct
  SITE 9: note = Lysine modified with a CPQ2 quencher
GASQQHRGK

SEQ ID NO: 843 - protein, 9 aa, synthetic construct
  SITE 9: note = Lysine modified with a CPQ2 quencher
GKNPAKAGK

SEQ ID NO: 844 - protein, 9 aa, synthetic construct
  SITE 2: note = Norleucine
  SITE 9: note = Lysine modified with a CPQ2 quencher
GXYWLVEGK

SEQ ID NO: 845 - protein, 9 aa, synthetic construct
  SITE 9: note = Lysine modified with a CPQ2 quencher
GSWWIFEGK

SEQ ID NO: 846 - protein, 9 aa, synthetic construct
  SITE 9: note = Lysine modified with a CPQ2 quencher
GVNYEQDGK

SEQ ID NO: 847 - protein, 9 aa, synthetic construct
  SITE 5: note = Norleucine
  SITE 9: note = Lysine modified with a CPQ2 quencher
GHFFXAEGK

SEQ ID NO: 848 - protein, 9 aa, synthetic construct
  SITE 9: note = Lysine modified with a CPQ2 quencher
GDIPPHWGK

SEQ ID NO: 849 - protein, 9 aa, synthetic construct
  SITE 6: note = Norleucine
  SITE 9: note = Lysine modified with a CPQ2 quencher
GVDQWXWGK

SEQ ID NO: 850 - protein, 9 aa, synthetic construct
  SITE 6: note = Norleucine
  SITE 9: note = Lysine modified with a CPQ2 quencher
GLRSLXKGK

SEQ ID NO: 851 - protein, 9 aa, synthetic construct
  SITE 2: note = Norleucine
  SITE 3: note = Norleucine
  SITE 9: note = Lysine modified with a CPQ2 quencher
GXXIRHAGK

SEQ ID NO: 852 - protein, 9 aa, synthetic construct
  SITE 9: note = Lysine modified with a CPQ2 quencher
GHDVKFIGK

SEQ ID NO: 853 - protein, 9 aa, synthetic construct
  SITE 9: note = Lysine modified with a CPQ2 quencher
GKRVQFLGK

SEQ ID NO: 854 - protein, 9 aa, synthetic construct
  SITE 4: note = Norleucine
  SITE 9: note = Lysine Amide modified with a CPQ2 quencher
GRDXYAEGK

SEQ ID NO: 855 - protein, 9 aa, synthetic construct
  SITE 3: note = Norleucine
  SITE 9: note = Lysine modified with a CPQ2 quencher
GLXIYFEGK

SEQ ID NO: 856 - protein, 9 aa, synthetic construct
  SITE 9: note = Lysine modified with a CPQ2 quencher
GLRTKQSGK

SEQ ID NO: 857 - protein, 9 aa, synthetic construct
  SITE 9: note = Lysine modified with a CPQ2 quencher
GWHGQQYGK

SEQ ID NO: 858 - protein, 9 aa, synthetic construct
  SITE 9: note = Lysine modified with a CPQ2 quencher
GGPEGTIGK

SEQ ID NO: 859 - protein, 9 aa, synthetic construct
  SITE 9: note = Lysine modified with a CPQ2 quencher
GELDPIPGK

SEQ ID NO: 860 - protein, 9 aa, synthetic construct
  SITE 9: note = Lysine Amide modified with a CPQ2 quencher
GGRAADFGK

SEQ ID NO: 861 - protein, 9 aa, synthetic construct
  SITE 9: note = Lysine modified with a CPQ2 quencher
GHFIDYIGK

SEQ ID NO: 862 - protein, 9 aa, synthetic construct
  SITE 3: note = Norleucine
  SITE 4: note = Norleucine
  SITE 9: note = Lysine modified with a CPQ2 quencher
GSXXRVHGK

SEQ ID NO: 863 - protein, 9 aa, synthetic construct
  SITE 9: note = Lysine modified with a CPQ2 quencher
GSFRKIIGK

SEQ ID NO: 864 - protein, 9 aa, synthetic construct
  SITE 5: note = Norleucine
  SITE 9: note = Lysine modified with a CPQ2 quencher
GTYEXFSGK

SEQ ID NO: 865 - protein, 9 aa, synthetic construct
  SITE 9: note = Lysine modified with a CPQ2 quencher
GHLLGFYGK

SEQ ID NO: 866 - protein, 9 aa, synthetic construct
  SITE 2: note = Norleucine
  SITE 9: note = Lysine modified with a CPQ2 quencher
GXWTALTGK

SEQ ID NO: 867 - protein, 9 aa, synthetic construct
  SITE 5: note = Norleucine
  SITE 9: note = Lysine modified with a CPQ2 quencher
GIWNXVYGK

SEQ ID NO: 868 - protein, 9 aa, synthetic construct
  SITE 9: note = Lysine modified with a CPQ2 quencher
GRRNPLWGK

SEQ ID NO: 869 - protein, 9 aa, synthetic construct
  SITE 9: note = Lysine Amide modified with a CPQ2 quencher
GRWYGGIGK

SEQ ID NO: 870 - protein, 9 aa, synthetic construct
  SITE 9: note = Lysine modified with a CPQ2 quencher
GKTGDARGK

SEQ ID NO: 871 - protein, 9 aa, synthetic construct
  SITE 9: note = Lysine modified with a CPQ2 quencher
GNYWEANGK

SEQ ID NO: 872 - protein, 9 aa, synthetic construct
  SITE 2: note = Norleucine
  SITE 9: note = Lysine modified with a CPQ2 quencher
GXQFDTSGK

SEQ ID NO: 873 - protein, 9 aa, synthetic construct
  SITE 9: note = Lysine modified with a CPQ2 quencher
GKRGAVEGK

SEQ ID NO: 874 - protein, 9 aa, synthetic construct
  SITE 9: note = Lysine Amide modified with a CPQ2 quencher
GSLKPTEGK

SEQ ID NO: 875 - protein, 9 aa, synthetic construct
  SITE 9: note = Lysine Amide modified with a CPQ2 quencher
GENDRLPGK

SEQ ID NO: 876 - protein, 9 aa, synthetic construct
  SITE 9: note = Lysine modified with a CPQ2 quencher
GNSYQVQGK

SEQ ID NO: 877 - protein, 9 aa, synthetic construct
  SITE 9: note = Lysine Amide modified with a CPQ2 quencher
GYPKEYLGK

SEQ ID NO: 878 - protein, 9 aa, synthetic construct
  SITE 9: note = Lysine Amide modified with a CPQ2 quencher
GINNKWQGK

SEQ ID NO: 879 - protein, 9 aa, synthetic construct
  SITE 2: note = Norleucine
  SITE 9: note = Lysine modified with a CPQ2 quencher
GXEFQGWGK

SEQ ID NO: 880 - protein, 9 aa, synthetic construct
  SITE 9: note = Lysine Amide modified with a CPQ2 quencher
GPVRSTNGK

SEQ ID NO: 881 - protein, 9 aa, synthetic construct
  SITE 9: note = Lysine Amide modified with a CPQ2 quencher
GSQAIKVGK

SEQ ID NO: 882 - protein, 9 aa, synthetic construct
  SITE 4: note = Norleucine
  SITE 9: note = Lysine modified with a CPQ2 quencher
GWAXLYHGK

SEQ ID NO: 883 - protein, 9 aa, synthetic construct
  SITE 9: note = Lysine modified with a CPQ2 quencher
GISWIHAGK

SEQ ID NO: 884 - protein, 9 aa, synthetic construct
  SITE 9: note = Lysine modified with a CPQ2 quencher
GAHDIVNGK

SEQ ID NO: 885 - protein, 9 aa, synthetic construct
  SITE 9: note = Lysine modified with a CPQ2 quencher
GRHNVASGK

SEQ ID NO: 886 - protein, 9 aa, synthetic construct
  SITE 9: note = Lysine modified with a CPQ2 quencher
GSVFVIEGK

SEQ ID NO: 887 - protein, 9 aa, synthetic construct
  SITE 9: note = Lysine modified with a CPQ2 quencher
GFAKYYKGK

SEQ ID NO: 888 - protein, 9 aa, synthetic construct
  SITE 9: note = Lysine modified with a CPQ2 quencher
GPYNTLQGK

SEQ ID NO: 889 - protein, 9 aa, synthetic construct
  SITE 2: note = Norleucine
  SITE 7: note = Norleucine
  SITE 9: note = Lysine modified with a CPQ2 quencher
GXDWGHXGK

SEQ ID NO: 890 - protein, 9 aa, synthetic construct
  SITE 9: note = Lysine Amide modified with a CPQ2 quencher
GSNREWFGK

SEQ ID NO: 891 - protein, 9 aa, synthetic construct
  SITE 9: note = Lysine modified with a CPQ2 quencher
GGKSEHTGK

SEQ ID NO: 892 - protein, 9 aa, synthetic construct
  SITE 4: note = Norleucine
  SITE 9: note = Lysine modified with a CPQ2 quencher
GFPXTDQGK

SEQ ID NO: 893 - protein, 9 aa, synthetic construct
  SITE 7: note = Norleucine
  SITE 9: note = Lysine modified with a CPQ2 quencher
GWSKFWXGK

SEQ ID NO: 894 - protein, 9 aa, synthetic construct
  SITE 9: note = Lysine Amide modified with a CPQ2 quencher
GRFTRPHGK

SEQ ID NO: 895 - protein, 9 aa, synthetic construct
  SITE 5: note = Norleucine
  SITE 9: note = Lysine Amide modified with a CPQ2 quencher
GQETXKDGK

SEQ ID NO: 896 - protein, 9 aa, synthetic construct
  SITE 9: note = Lysine modified with a CPQ2 quencher
GHWWDVLGK

SEQ ID NO: 897 - protein, 9 aa, synthetic construct
  SITE 6: note = Norleucine
  SITE 9: note = Lysine modified with a CPQ2 quencher
GFNLVXSGK

SEQ ID NO: 898 - protein, 9 aa, synthetic construct
  SITE 9: note = Lysine modified with a CPQ2 quencher
GSAWRQRGK

SEQ ID NO: 899 - protein, 9 aa, synthetic construct
  SITE 9: note = Lysine modified with a CPQ2 quencher
GTFHIFLGK

SEQ ID NO: 900 - protein, 9 aa, synthetic construct
  SITE 9: note = Lysine modified with a CPQ2 quencher
GWPQHVKGK

SEQ ID NO: 901 - protein, 9 aa, synthetic construct
  SITE 4: note = Norleucine
  SITE 9: note = Lysine modified with a CPQ2 quencher
GLIXHKNGK

SEQ ID NO: 902 - protein, 9 aa, synthetic construct
  SITE 9: note = Lysine modified with a CPQ2 quencher
GQDLEQPGK

SEQ ID NO: 903 - protein, 9 aa, synthetic construct
  SITE 6: note = Norleucine
  SITE 9: note = Lysine Amide modified with a CPQ2 quencher
GHQKKXPGK

SEQ ID NO: 904 - protein, 9 aa, synthetic construct
  SITE 9: note = Lysine modified with a CPQ2 quencher
GGVTWLNGK

SEQ ID NO: 905 - protein, 9 aa, synthetic construct
  SITE 9: note = Lysine Amide modified with a CPQ2 quencher
GAGEPFKGK

SEQ ID NO: 906 - protein, 9 aa, synthetic construct
  SITE 4: note = Norleucine
  SITE 9: note = Lysine Amide modified with a CPQ2 quencher
GSRXATTGK

SEQ ID NO: 907 - protein, 9 aa, synthetic construct
  SITE 5: note = Norleucine
  SITE 9: note = Lysine modified with a CPQ2 quencher
GLAFXNHGK

SEQ ID NO: 908 - protein, 9 aa, synthetic construct
  SITE 9: note = Lysine modified with a CPQ2 quencher
GPPSGLSGK

SEQ ID NO: 909 - protein, 9 aa, synthetic construct
  SITE 9: note = Lysine modified with a CPQ2 quencher
GYTHSSPGK

SEQ ID NO: 910 - protein, 9 aa, synthetic construct
  SITE 9: note = Lysine modified with a CPQ2 quencher
GDGSHYRGK

SEQ ID NO: 911 - protein, 9 aa, synthetic construct
  SITE 3: note = Norleucine
  SITE 9: note = Lysine modified with a CPQ2 quencher
GYXGNGYGK

SEQ ID NO: 912 - protein, 9 aa, synthetic construct
  SITE 9: note = Lysine modified with a CPQ2 quencher
GDSITVSGK

SEQ ID NO: 913 - protein, 9 aa, synthetic construct
  SITE 9: note = Lysine modified with a CPQ2 quencher
GQTPNIQGK

SEQ ID NO: 914 - protein, 9 aa, synthetic construct
  SITE 9: note = Lysine Amide modified with a CPQ2 quencher
GKLFFGYGK

SEQ ID NO: 915 - protein, 9 aa, synthetic construct
  SITE 9: note = Lysine modified with a CPQ2 quencher
GTQNFNWGK

SEQ ID NO: 916 - protein, 9 aa, synthetic construct
  SITE 9: note = Lysine modified with a CPQ2 quencher
GYSDHEVGK

SEQ ID NO: 917 - protein, 9 aa, synthetic construct
  SITE 9: note = Lysine Amide modified with a CPQ2 quencher
GRYVVPAGK

SEQ ID NO: 918 - protein, 9 aa, synthetic construct
  SITE 9: note = Lysine Amide modified with a CPQ2 quencher
GILHRIRGK

SEQ ID NO: 919 - protein, 9 aa, synthetic construct
  SITE 7: note = Norleucine
  SITE 9: note = Lysine modified with a CPQ2 quencher
GESDNQXGK

SEQ ID NO: 920 - protein, 9 aa, synthetic construct
  SITE 7: note = Norleucine
  SITE 9: note = Lysine Amide modified with a CPQ2 quencher
GYDDKGXGK

SEQ ID NO: 921 - protein, 9 aa, synthetic construct
  SITE 5: note = Norleucine
  SITE 9: note = Lysine modified with a CPQ2 quencher
GQLSXVWGK

SEQ ID NO: 922 - protein, 9 aa, synthetic construct
  SITE 7: note = Norleucine
  SITE 9: note = Lysine Amide modified with a CPQ2 quencher
GPGGERXGK

SEQ ID NO: 923 - protein, 9 aa, synthetic construct
  SITE 9: note = Lysine Amide modified with a CPQ2 quencher
GWKHHPDGK

SEQ ID NO: 924 - protein, 9 aa, synthetic construct
  SITE 9: note = Lysine modified with a CPQ2 quencher
GQWVDEDGK

SEQ ID NO: 925 - protein, 9 aa, synthetic construct
  SITE 9: note = Lysine modified with a CPQ2 quencher
GNAYNEIGK

SEQ ID NO: 926 - protein, 9 aa, synthetic construct
  SITE 9: note = Lysine modified with a CPQ2 quencher
GEEKAPRGK

SEQ ID NO: 927 - protein, 9 aa, synthetic construct
  SITE 9: note = Lysine Amide modified with a CPQ2 quencher
GPWQIGKGK

SEQ ID NO: 928 - protein, 9 aa, synthetic construct
  SITE 9: note = Lysine modified with a CPQ2 quencher
GIAQVGNGK

SEQ ID NO: 929 - protein, 9 aa, synthetic construct
  SITE 3: note = Norleucine
  SITE 9: note = Lysine Amide modified with a CPQ2 quencher
GVXRQSEGK

SEQ ID NO: 930 - protein, 9 aa, synthetic construct
  SITE 9: note = Lysine Amide modified with a CPQ2 quencher
GTERVDAGK

SEQ ID NO: 931 - protein, 9 aa, synthetic construct
  SITE 9: note = Lysine modified with a CPQ2 quencher
GWLRWRLGK

SEQ ID NO: 932 - protein, 9 aa, synthetic construct
  SITE 9: note = Lysine modified with a CPQ2 quencher
GWKTKGQGK

SEQ ID NO: 933 - protein, 9 aa, synthetic construct
  SITE 9: note = Lysine modified with a CPQ2 quencher
GQSNGDVGK

SEQ ID NO: 934 - protein, 9 aa, synthetic construct
  SITE 9: note = Lysine modified with a CPQ2 quencher
GTLFYALGK

SEQ ID NO: 935 - protein, 9 aa, synthetic construct
  SITE 9: note = Lysine modified with a CPQ2 quencher
GTVTLNPGK

SEQ ID NO: 936 - protein, 9 aa, synthetic construct
  SITE 9: note = Lysine modified with a CPQ2 quencher
GYAFGRKGK

SEQ ID NO: 937 - protein, 9 aa, synthetic construct
  SITE 9: note = Lysine modified with a CPQ2 quencher
GDYNYWDGK

SEQ ID NO: 938 - protein, 9 aa, synthetic construct
  SITE 9: note = Lysine modified with a CPQ2 quencher
GEWHEIIGK

SEQ ID NO: 939 - protein, 9 aa, synthetic construct
  SITE 9: note = Lysine Amide modified with a CPQ2 quencher
GQKAAWDGK

SEQ ID NO: 940 - protein, 9 aa, synthetic construct
  SITE 9: note = Lysine modified with a CPQ2 quencher
GDNTSADGK

SEQ ID NO: 941 - protein, 9 aa, synthetic construct
  SITE 9: note = Lysine modified with a CPQ2 quencher
GHEGEYVGK

SEQ ID NO: 942 - protein, 9 aa, synthetic construct
  SITE 9: note = Lysine Amide modified with a CPQ2 quencher
GWSPSFKGK

SEQ ID NO: 943 - protein, 9 aa, synthetic construct
  SITE 9: note = Lysine modified with a CPQ2 quencher
GHDEHWTGK

SEQ ID NO: 944 - protein, 9 aa, synthetic construct
  SITE 5: note = Norleucine
  SITE 9: note = Lysine Amide modified with a CPQ2 quencher
GYVWXRDGK

SEQ ID NO: 945 - protein, 9 aa, synthetic construct
  SITE 2: note = Norleucine
  SITE 5: note = Norleucine
  SITE 9: note = Lysine Amide modified with a CPQ2 quencher
GXDPXKFGK

SEQ ID NO: 946 - protein, 9 aa, synthetic construct
  SITE 2: note = Norleucine
  SITE 4: note = Norleucine
  SITE 9: note = Lysine Amide modified with a CPQ2 quencher
GXRXFWDGK

SEQ ID NO: 947 - protein, 9 aa, synthetic construct
  SITE 7: note = Norleucine
  SITE 9: note = Lysine modified with a CPQ2 quencher
GDIAITXGK

SEQ ID NO: 948 - protein, 9 aa, synthetic construct
  SITE 4: note = Norleucine
  SITE 9: note = Lysine modified with a CPQ2 quencher
GPIXRFHGK

SEQ ID NO: 949 - protein, 9 aa, synthetic construct
  SITE 9: note = Lysine modified with a CPQ2 quencher
GVWQGYIGK

SEQ ID NO: 950 - protein, 9 aa, synthetic construct
  SITE 4: note = Norleucine
  SITE 9: note = Lysine modified with a CPQ2 quencher
GKKXSNPGK

SEQ ID NO: 951 - protein, 9 aa, synthetic construct
  SITE 9: note = Lysine modified with a CPQ2 quencher
GGHPLSPGK

SEQ ID NO: 952 - protein, 9 aa, synthetic construct
  SITE 9: note = Lysine Amide modified with a CPQ2 quencher
GVRQHKPGK

SEQ ID NO: 953 - protein, 9 aa, synthetic construct
  SITE 9: note = Lysine Amide modified with a CPQ2 quencher
GAQNFYRGK

SEQ ID NO: 954 - protein, 9 aa, synthetic construct
  SITE 9: note = Lysine Amide modified with a CPQ2 quencher
GVAGKSIGK

SEQ ID NO: 955 - protein, 9 aa, synthetic construct
  SITE 9: note = Lysine modified with a CPQ2 quencher
GLVGQVNGK

SEQ ID NO: 956 - protein, 9 aa, synthetic construct
  SITE 9: note = Lysine modified with a CPQ2 quencher
GQVKHFTGK

SEQ ID NO: 957 - protein, 9 aa, synthetic construct
  SITE 9: note = Lysine Amide modified with a CPQ2 quencher
GQKSVVSGK

SEQ ID NO: 958 - protein, 9 aa, synthetic construct
  SITE 3: note = Norleucine
  SITE 9: note = Lysine modified with a CPQ2 quencher
GYXQEWLGK

SEQ ID NO: 959 - protein, 9 aa, synthetic construct
  SITE 3: note = Norleucine
  SITE 9: note = Lysine modified with a CPQ2 quencher
GGXYIDEGK

SEQ ID NO: 960 - protein, 9 aa, synthetic construct
  SITE 9: note = Lysine Amide modified with a CPQ2 quencher
GNAGSKFGK

SEQ ID NO: 961 - protein, 9 aa, synthetic construct
  SITE 9: note = Lysine modified with a CPQ2 quencher
GEFVHNPGK

SEQ ID NO: 962 - protein, 9 aa, synthetic construct
  SITE 4: note = Norleucine
  SITE 9: note = Lysine Amide modified with a CPQ2 quencher
GWEXVKIGK

SEQ ID NO: 963 - protein, 9 aa, synthetic construct
  SITE 9: note = Lysine modified with a CPQ2 quencher
GWVGASHGK

SEQ ID NO: 964 - protein, 9 aa, synthetic construct
  SITE 7: note = Norleucine
  SITE 9: note = Lysine modified with a CPQ2 quencher
GITTLYXGK

SEQ ID NO: 965 - protein, 9 aa, synthetic construct
  SITE 9: note = Lysine modified with a CPQ2 quencher
GGHIDEYGK

SEQ ID NO: 966 - protein, 9 aa, synthetic construct
  SITE 4: note = Norleucine
  SITE 9: note = Lysine Amide modified with a CPQ2 quencher
GKVXDYGGK

SEQ ID NO: 967 - protein, 9 aa, synthetic construct
  SITE 7: note = Norleucine
  SITE 9: note = Lysine Amide modified with a CPQ2 quencher
GQEKQTXGK

SEQ ID NO: 968 - protein, 9 aa, synthetic construct
  SITE 9: note = Lysine modified with a CPQ2 quencher
GEVGHEAGK

SEQ ID NO: 969 - protein, 9 aa, synthetic construct
  SITE 9: note = Lysine modified with a CPQ2 quencher
GAWEGQYGK

SEQ ID NO: 970 - protein, 9 aa, synthetic construct
  SITE 9: note = Lysine modified with a CPQ2 quencher
GFLVQWTGK

SEQ ID NO: 971 - protein, 9 aa, synthetic construct
  SITE 9: note = Lysine Amide modified with a CPQ2 quencher
GSKWGYWGK

SEQ ID NO: 972 - protein, 9 aa, synthetic construct
  SITE 6: note = Norleucine
  SITE 9: note = Lysine modified with a CPQ2 quencher
GTWISXQGK

SEQ ID NO: 973 - protein, 9 aa, synthetic construct
  SITE 9: note = Lysine Amide modified with a CPQ2 quencher
GVIDKDFGK

SEQ ID NO: 974 - protein, 9 aa, synthetic construct
  SITE 9: note = Lysine Amide modified with a CPQ2 quencher
GVKFAIYGK

SEQ ID NO: 975 - protein, 9 aa, synthetic construct
  SITE 5: note = Norleucine
  SITE 9: note = Lysine modified with a CPQ2 quencher
GHNQXKSGK

SEQ ID NO: 976 - protein, 9 aa, synthetic construct
  SITE 7: note = Norleucine
  SITE 9: note = Lysine modified with a CPQ2 quencher
GQYVFFXGK

SEQ ID NO: 977 - protein, 9 aa, synthetic construct
  SITE 7: note = Norleucine
  SITE 9: note = Lysine Amide modified with a CPQ2 quencher
GYNPREXGK

SEQ ID NO: 978 - protein, 9 aa, synthetic construct
  SITE 5: note = Norleucine
  SITE 9: note = Lysine modified with a CPQ2 quencher
GKHGXPEGK

SEQ ID NO: 979 - protein, 9 aa, synthetic construct
  SITE 9: note = Lysine Amide modified with a CPQ2 quencher
GWSREYWGK

SEQ ID NO: 980 - protein, 9 aa, synthetic construct
  SITE 9: note = Lysine modified with a CPQ2 quencher
GIDRVDKGK

SEQ ID NO: 981 - protein, 11 aa, synthetic construct
  SITE 1: note = D-Lysine
  SITE 2: note = D-Lysine
  SITE 10: note = Lysine modified with a CPQ2 quencher
  SITE 11: note = Hydroxy Leucine
KKGDRENSPKL

SEQ ID NO: 982 - protein, 11 aa, synthetic construct
  SITE 1: note = D-Lysine
  SITE 2: note = D-Lysine
  SITE 11: note = Hydroxy Lysine modified with a CPQ2 quencher
KKGDRENSPLK

SEQ ID NO: 983 - protein, 9 aa, synthetic construct
  SITE 8: note = Lysine modified with a CPQ2 quencher
  SITE 9: note = Hydroxy Glutamine
GNAGSKFKQ

SEQ ID NO: 984 - protein, 9 aa, synthetic construct
  SITE 9: note = Hydroxy Lysine modified with a CPQ2 quencher
GNAGSKFQK

SEQ ID NO: 985 - protein, 11 aa, synthetic construct
  SITE 1: note = D-Lysine
  SITE 2: note = D-Lysine
  SITE 10: note = Lysine modified with a CPQ2 quencher
  SITE 11: note = Hydroxy Valine
KKGHLLGFYKV

SEQ ID NO: 986 - protein, 11 aa, synthetic construct
  SITE 1: note = D-Lysine
  SITE 2: note = D-Lysine
  SITE 11: note = Hydroxy Lysine modified with a CPQ2 quencher
KKGHLLGFYVK

SEQ ID NO: 987 - protein, 11 aa, synthetic construct
  SITE 1: note = D-Lysine
  SITE 2: note = D-Lysine
  SITE 9: note = Norleucine
  SITE 10: note = Lysine modified with a CPQ2 quencher
  SITE 11: note = Hydroxy Norleucine
KKGQEKQTXKX

SEQ ID NO: 988 - protein, 11 aa, synthetic construct
  SITE 1: note = D-Lysine
  SITE 2: note = D-Lysine
  SITE 9: note = Norleucine
  SITE 10: note = Norleucine
  SITE 11: note = Hydroxy Lysine modified with a CPQ2 quencher
KKGQEKQTXXK

SEQ ID NO: 989 - protein, 10 aa, synthetic construct
  SITE 1: note = D-Lysine
  SITE 9: note = Lysine modified with a CPQ2 quencher
  SITE 10: note = Hydroxy Tryptophan
KGDPFVVSKW

SEQ ID NO: 990 - protein, 10 aa, synthetic construct
  SITE 1: note = D-Lysine
  SITE 10: note = Hydroxy Lysine modified with a CPQ2 quencher
KGDPFVVSWK

SEQ ID NO: 991 - protein, 9 aa, synthetic construct
  SITE 8: note = Lysine modified with a CPQ2 quencher
  SITE 9: note = Hydroxy Arginine
GNAYNEIKR

SEQ ID NO: 992 - protein, 9 aa, synthetic construct
  SITE 9: note = Hydroxy Lysine modified with a CPQ2 quencher
GNAYNEIRK

SEQ ID NO: 993 - protein, 9 aa, synthetic construct
  SITE 3: note = Norleucine
  SITE 8: note = Lysine modified with a CPQ2 quencher
  SITE 9: note = Hydroxy Asparagine
GVXRQSEKN

SEQ ID NO: 994 - protein, 9 aa, synthetic construct
  SITE 3: note = Norleucine
  SITE 9: note = Lysine modified with a CPQ2 quencher
GVXRQSENK

SEQ ID NO: 995 - protein, 9 aa, synthetic construct
  SITE 7: note = Norleucine
  SITE 8: note = Lysine modified with a CPQ2 quencher
  SITE 9: note = Hydroxy Isoleucine
GYNPREXKI

SEQ ID NO: 996 - protein, 9 aa, synthetic construct
  SITE 7: note = Norleucine
  SITE 9: note = Hydroxy Lysine modified with a CPQ2 quencher
GYNPREXIK

SEQ ID NO: 997 - protein, 10 aa, synthetic construct
  SITE 1: note = D-Lysine
  SITE 9: note = Lysine modified with a CPQ2 quencher
  SITE 10: note = Hydroxy Lysine
KGEFVHNPKK

SEQ ID NO: 998 - protein, 10 aa, synthetic construct
  SITE 1: note = D-Lysine
  SITE 10: note = Hydroxy Lysine modified with a CPQ2 quencher
KGEFVHNPKK

SEQ ID NO: 999 - protein, 9 aa, synthetic construct
  SITE 8: note = Lysine modified with a CPQ2 quencher
  SITE 9: note = Hydroxy Histidine
GKRVQFLKH

SEQ ID NO: 1000 - protein, 9 aa, synthetic construct
  SITE 9: note = Hydroxy Lysine modified with a CPQ2 quencher
GKRVQFLHK

SEQ ID NO: 1001 - protein, 10 aa, synthetic construct
  SITE 1: note = D-Lysine
  SITE 5: note = Norleucine
  SITE 9: note = Lysine modified with a CPQ2 quencher
  SITE 10: note = Hydroxy Glycine
KGLIXHKNKG

SEQ ID NO: 1002 - protein, 10 aa, synthetic construct
  SITE 1: note = D-Lysine
  SITE 5: note = Norleucine
  SITE 10: note = Hydroxy Lysine modified with a CPQ2 quencher
KGLIXHKNGK

SEQ ID NO: 1003 - protein, 11 aa, synthetic construct
  SITE 1: note = D-Lysine
  SITE 2: note = D-Lysine
  SITE 6: note = Norleucine
  SITE 10: note = Lysine modified with a CPQ2 quencher
  SITE 11: note = Hydroxy Serine
KKGWAXLYHKS

SEQ ID NO: 1004 - protein, 11 aa, synthetic construct
  SITE 1: note = D-Lysine
  SITE 2: note = D-Lysine
  SITE 6: note = Norleucine
  SITE 11: note = Hydroxy Lysine modified with a CPQ2 quencher
KKGWAXLYHSK

SEQ ID NO: 1005 - protein, 11 aa, synthetic construct
  SITE 1: note = D-Lysine
  SITE 2: note = D-Lysine
  SITE 10: note = Lysine modified with a CPQ2 quencher
  SITE 11: note = Hydroxy Tyrosine
KKGAHDIVNKY

SEQ ID NO: 1006 - protein, 11 aa, synthetic construct
  SITE 1: note = D-Lysine
  SITE 2: note = D-Lysine
  SITE 11: note = Hydroxy Lysine modified with a CPQ2 quencher
KKGAHDIVNYK

SEQ ID NO: 1007 - protein, 10 aa, synthetic construct
  SITE 1: note = D-Lysine
  SITE 9: note = Lysine modified with a CPQ2 quencher
  SITE 10: note = Hydroxy Proline
KGSVFVIEKP

SEQ ID NO: 1008 - protein, 10 aa, synthetic construct
  SITE 1: note = D-Lysine
  SITE 10: note = Hydroxy Lysine modified with a CPQ2 quencher
KGSVFVIEPK

SEQ ID NO: 1009 - protein, 10 aa, synthetic construct
  SITE 1: note = D-Lysine
  SITE 9: note = Lysine modified with a CPQ2 quencher
  SITE 10: note = Hydroxy Glutamic Acid
KGPPSGLSKE

SEQ ID NO: 1010 - protein, 10 aa, synthetic construct
  SITE 1: note = D-Lysine
  SITE 10: note = Hydroxy Lysine modified with a CPQ2 quencher
KGPPSGLSEK

SEQ ID NO: 1011 - protein, 11 aa, synthetic construct
  SITE 1: note = D-Lysine
  SITE 2: note = D-Lysine
  SITE 10: note = Lysine modified with a CPQ2 quencher
  SITE 11: note = Hydroxy Phenylalanine
KKGRWYGGIKF

SEQ ID NO: 1012 - protein, 11 aa, synthetic construct
  SITE 1: note = D-Lysine
  SITE 2: note = D-Lysine
  SITE 11: note = Hydroxy Lysine modified with a CPQ2 quencher
KKGRWYGGIFK

SEQ ID NO: 1013 - protein, 10 aa, synthetic construct
  SITE 1: note = D-Lysine
  SITE 8: note = Norleucine
  SITE 9: note = Lysine modified with a CPQ2 quencher
  SITE 10: note = Hydroxy Aspartic Acid
KGQYVFFXKD

SEQ ID NO: 1014 - protein, 10 aa, synthetic construct
  SITE 1: note = D-Lysine
  SITE 8: note = Norleucine
  SITE 10: note = Hydroxy Lysine modified with a CPQ2 quencher
KGQYVFFXDK

SEQ ID NO: 1015 - protein, 10 aa, synthetic construct
  SITE 1: note = D-Lysine
  SITE 9: note = Lysine modified with a CPQ2 quencher
  SITE 10: note = Hydroxy Threonine
KGFAKYYKKT

SEQ ID NO: 1016 - protein, 10 aa, synthetic construct
  SITE 1: note = D-Lysine
  SITE 10: note = Hydroxy Lysine modified with a CPQ2 quencher
KGFAKYYKTK

SEQ ID NO: 1017 - protein, 10 aa, synthetic construct
  SITE 1: note = D-Lysine
  SITE 9: note = Lysine modified with a CPQ2 quencher
  SITE 10: note = Hydroxy Alanine
KGQVKHFTKA

SEQ ID NO: 1018 - protein, 10 aa, synthetic construct
  SITE 1: note = D-Lysine
  SITE 10: note = Hydroxy Lysine modified with a CPQ2 quencher
KGQVKHFTAK

SEQ ID NO: 1019 - protein, 10 aa, synthetic construct
  SITE 1: note = Histidine Amide
  SITE 2: note = Carboxy-fluorescein-L-lysine
  SITE 10: note = Lysine Amide modified with a CPQ2 quencher
HKDRENSPGK

SEQ ID NO: 1020 - protein, 10 aa, synthetic construct
  SITE 1: note = Carboxy-fluorescein-L-lysine Amide
  SITE 10: note = Lysine Amide modified with a CPQ2 quencher
KHDRENSPGK

SEQ ID NO: 1021 - protein, 11 aa, synthetic construct
  SITE 1: note = Tryptophan Amide
  SITE 2: note = Carboxy-fluorescein-L-lysine
  SITE 10: note = D-Lysine
  SITE 11: note = Lysine Amide modified with a CPQ2 quencher
WKNAGSKFGKK

SEQ ID NO: 1022 - protein, 11 aa, synthetic construct
  SITE 1: note = Carboxy-fluorescein-L-lysine Amide
  SITE 10: note = D-Lysine
  SITE 11: note = Lysine Amide modified with a CPQ2 quencher
KWNAGSKFGKK

SEQ ID NO: 1023 - protein, 11 aa, synthetic construct
  SITE 1: note = Serine Amide
  SITE 2: note = Carboxy-fluorescein-L-lysine
  SITE 10: note = D-Lysine
  SITE 11: note = Lysine Amide modified with a CPQ2 quencher
SKHLLGFYGKK

SEQ ID NO: 1024 - protein, 11 aa, synthetic construct
  SITE 1: note = Carboxy-fluorescein-L-lysine Amide
  SITE 10: note = D-Lysine
  SITE 11: note = Lysine Amide modified with a CPQ2 quencher
KSHLLGFYGKK

SEQ ID NO: 1025 - protein, 10 aa, synthetic construct
  SITE 1: note = Lysine Amide
  SITE 2: note = Carboxy-fluorescein-L-lysine
  SITE 8: note = Norleucine
  SITE 10: note = Lysine Amide modified with a CPQ2 quencher
KKQEKQTXGK

SEQ ID NO: 1026 - protein, 10 aa, synthetic construct
  SITE 1: note = Carboxy-fluorescein-L-lysine Amide
  SITE 8: note = Norleucine
  SITE 10: note = Lysine Amide modified with a CPQ2 quencher
KKQEKQTXGK

SEQ ID NO: 1027 - protein, 10 aa, synthetic construct
  SITE 1: note = Glycine Amide
  SITE 2: note = Carboxy-fluorescein-L-lysine
  SITE 10: note = Lysine Amide modified with a CPQ2 quencher
GKDPFVVSGK

SEQ ID NO: 1028 - protein, 10 aa, synthetic construct
  SITE 1: note = Carboxy-fluorescein-L-lysine Amide
  SITE 10: note = Lysine Amide modified with a CPQ2 quencher
KGDPFVVSGK

SEQ ID NO: 1029 - protein, 10 aa, synthetic construct
  SITE 1: note = Proline Amide
  SITE 2: note = Carboxy-fluorescein-L-lysine
  SITE 10: note = Lysine Amide modified with a CPQ2 quencher
PKNAYNEIGK

SEQ ID NO: 1030 - protein, 10 aa, synthetic construct
  SITE 1: note = Carboxy-fluorescein-L-lysine Amide
  SITE 10: note = Lysine Amide modified with a CPQ2 quencher
KPNAYNEIGK

SEQ ID NO: 1031 - protein, 11 aa, synthetic construct
  SITE 1: note = Aspartic Acid Amide
  SITE 2: note = Carboxy-fluorescein-L-lysine
  SITE 4: note = Norleucine
  SITE 10: note = D-Lysine
  SITE 11: note = Lysine Amide modified with a CPQ2 quencher
DKVXRQSEGKK

SEQ ID NO: 1032 - protein, 11 aa, synthetic construct
  SITE 1: note = Carboxy-fluorescein-L-lysine Amide
  SITE 4: note = Norleucine
  SITE 10: note = D-Lysine
  SITE 11: note = Lysine Amide modified with a CPQ2 quencher
KDVXRQSEGKK

SEQ ID NO: 1033 - protein, 11 aa, synthetic construct
  SITE 1: note = Glutamic Acid Amide
  SITE 2: note = Carboxy-fluorescein-L-lysine
  SITE 8: note = Norleucine
  SITE 10: note = D-Lysine
  SITE 11: note = Lysine Amide modified with a CPQ2 quencher
EKYNPREXGKK

SEQ ID NO: 1034 - protein, 11 aa, synthetic construct
  SITE 1: note = Carboxy-fluorescein-L-lysine Amide
  SITE 8: note = Norleucine
  SITE 10: note = D-Lysine
  SITE 11: note = Lysine Amide modified with a CPQ2 quencher
KEYNPREXGKK

SEQ ID NO: 1035 - protein, 11 aa, synthetic construct
  SITE 1: note = Threonine Amide
  SITE 2: note = Carboxy-fluorescein-L-lysine
  SITE 10: note = D-Lysine
  SITE 11: note = Lysine Amide modified with a CPQ2 quencher
TKEFVHNPGKK

SEQ ID NO: 1036 - protein, 11 aa, synthetic construct
  SITE 1: note = Carboxy-fluorescein-L-lysine Amide
  SITE 10: note = D-Lysine
  SITE 11: note = Lysine Amide modified with a CPQ2 quencher
KTEFVHNPGKK

SEQ ID NO: 1037 - protein, 10 aa, synthetic construct
  SITE 1: note = Glutamine Amide
  SITE 2: note = Carboxy-fluorescein-L-lysine
  SITE 10: note = Lysine Amide modified with a CPQ2 quencher
QKKRVQFLGK

SEQ ID NO: 1038 - protein, 10 aa, synthetic construct
  SITE 1: note = Carboxy-fluorescein-L-lysine Amide
  SITE 10: note = Lysine Amide modified with a CPQ2 quencher
KQKRVQFLGK

SEQ ID NO: 1039 - protein, 10 aa, synthetic construct
  SITE 1: note = Tyrosine Amide
  SITE 2: note = Carboxy-fluorescein-L-lysine
  SITE 5: note = Norleucine
  SITE 10: note = Lysine Amide modified with a CPQ2 quencher
YKLIXHKNGK

SEQ ID NO: 1040 - protein, 10 aa, synthetic construct
  SITE 1: note = Carboxy-fluorescein-L-lysine Amide
  SITE 5: note = Norleucine
  SITE 10: note = Lysine Amide modified with a CPQ2 quencher
KYLIXHKNGK

SEQ ID NO: 1041 - protein, 11 aa, synthetic construct
  SITE 1: note = Phenylalanine Amide
  SITE 2: note = Carboxy-fluorescein-L-lysine
  SITE 5: note = Norleucine
  SITE 10: note = D-Lysine
  SITE 11: note = Lysine Amide modified with a CPQ2 quencher
FKWAXLYHGKK

SEQ ID NO: 1042 - protein, 11 aa, synthetic construct
  SITE 1: note = Carboxy-fluorescein-L-lysine Amide
  SITE 5: note = Norleucine
  SITE 10: note = D-Lysine
  SITE 11: note = Lysine Amide modified with a CPQ2 quencher
KFWAXLYHGKK

SEQ ID NO: 1043 - protein, 11 aa, synthetic construct
  SITE 1: note = Isoleucine Amide
  SITE 2: note = Carboxy-fluorescein-L-lysine
  SITE 10: note = D-Lysine
  SITE 11: note = Lysine Amide modified with a CPQ2 quencher
IKAHDIVNGKK

SEQ ID NO: 1044 - protein, 11 aa, synthetic construct
  SITE 1: note = Carboxy-fluorescein-L-lysine Amide
  SITE 10: note = D-Lysine
  SITE 11: note = Lysine Amide modified with a CPQ2 quencher
KIAHDIVNGKK

SEQ ID NO: 1045 - protein, 10 aa, synthetic construct
  SITE 1: note = Valine Amide
  SITE 2: note = Carboxy-fluorescein-L-lysine
  SITE 10: note = Lysine Amide modified with a CPQ2 quencher
VKSVFVIEGK

SEQ ID NO: 1046 - protein, 10 aa, synthetic construct
  SITE 1: note = Carboxy-fluorescein-L-lysine Amide
  SITE 10: note = Lysine Amide modified with a CPQ2 quencher
KVSVFVIEGK

SEQ ID NO: 1047 - protein, 10 aa, synthetic construct
  SITE 1: note = Norleucine Amide
  SITE 2: note = Carboxy-fluorescein-L-lysine
  SITE 10: note = Lysine Amide modified with a CPQ2 quencher
XKPPSGLSGK

SEQ ID NO: 1048 - protein, 10 aa, synthetic construct
  SITE 1: note = Carboxy-fluorescein-L-lysine Amide
  SITE 2: note = Norleucine
  SITE 10: note = Lysine Amide modified with a CPQ2 quencher
KXPPSGLSGK

SEQ ID NO: 1049 - protein, 11 aa, synthetic construct
  SITE 1: note = Leucine Amide
  SITE 2: note = Carboxy-fluorescein-L-lysine
  SITE 10: note = D-Lysine
  SITE 11: note = Lysine Amide modified with a CPQ2 quencher
LKRWYGGIGKK

SEQ ID NO: 1050 - protein, 11 aa, synthetic construct
  SITE 1: note = Carboxy-fluorescein-L-lysine Amide
  SITE 10: note = D-Lysine
  SITE 11: note = Lysine Amide modified with a CPQ2 quencher
KLRWYGGIGKK

SEQ ID NO: 1051 - protein, 10 aa, synthetic construct
  SITE 1: note = Asparagine Amide
  SITE 2: note = Carboxy-fluorescein-L-lysine
  SITE 8: note = Norleucine
  SITE 10: note = Lysine Amide modified with a CPQ2 quencher
NKQYVFFXGK

SEQ ID NO: 1052 - protein, 10 aa, synthetic construct
  SITE 1: note = Carboxy-fluorescein-L-lysine Amide
  SITE 8: note = Norleucine
  SITE 10: note = Lysine Amide modified with a CPQ2 quencher
KNQYVFFXGK

SEQ ID NO: 1053 - protein, 10 aa, synthetic construct
  SITE 1: note = Alanine Amide
  SITE 2: note = Carboxy-fluorescein-L-lysine
  SITE 10: note = Lysine Amide modified with a CPQ2 quencher
AKFAKYYKGK

SEQ ID NO: 1054 - protein, 10 aa, synthetic construct
  SITE 1: note = Carboxy-fluorescein-L-lysine Amide
  SITE 10: note = Lysine Amide modified with a CPQ2 quencher
KAFAKYYKGK

SEQ ID NO: 1055 - protein, 10 aa, synthetic construct
  SITE 1: note = Arginine Amide
  SITE 2: note = Carboxy-fluorescein-L-lysine
  SITE 10: note = Lysine Amide modified with a CPQ2 quencher
RKQVKHFTGK

SEQ ID NO: 1056 - protein, 10 aa, synthetic construct
  SITE 1: note = Carboxy-fluorescein-L-lysine Amide
  SITE 10: note = Lysine Amide modified with a CPQ2 quencher
KRQVKHFTGK

SEQ ID NO: 1057 - protein, 4 aa, synthetic construct
  SITE 1: note = Carboxy-fluorescein-L-lysine Amide
  SITE 4: note = Lysine Amide modified with a CPQ2 quencher
KPPK

SEQ ID NO: 1058 - protein, 12 aa, synthetic construct
  SITE 2: note = D-Lysine
  SITE 3: note = D-Proline
  SITE 4: note = D-Isoleucine
  SITE 5: note = D-Leucine
  SITE 6: note = D-Phenylalanine
  SITE 7: note = D-Phenylalanine
  SITE 8: note = D-Arginine
  SITE 9: note = D-Leucine
  SITE 10: note = D-Lysine
  SITE 12: note = Lysine modified with a CPQ2 quencher
GKPILFFRLKGK

SEQ ID NO: 1059 - protein, 5 aa, synthetic construct
ZLRGG

SEQ ID NO: 1060 - protein, 8 aa, synthetic construct
  SITE 8: note = Lysine modified with a CPQ2 quencher
GRLRGGGK

SEQ ID NO: 1061 - protein, 12 aa, synthetic construct
  SITE 12: note = Lysine modified with a CPQ2 quencher
GRELNGGAPIGK

SEQ ID NO: 1062 - protein, 14 aa, synthetic construct
  SITE 14: note = Lysine modified with a CPQ2 quencher
GTSAVLQSGFRKGK

SEQ ID NO: 1063 - protein, 14 aa, synthetic construct
  SITE 14: note = Lysine modified with a CPQ2 quencher
GSGVTFQGKFKKGK

SEQ ID NO: 1064 - protein, 7 aa, synthetic construct
  SITE 7: note = Lysine modified with a CPQ2 quencher
GAAFAGK

SEQ ID NO: 1065 - protein, 11 aa, synthetic construct
  SITE 11: note = Lysine modified with a CPQ2 quencher
GHGDQMAQKSK

SEQ ID NO: 1066 - protein, 9 aa, synthetic construct
  SITE 9: note = Lysine modified with a CPQ2 quencher
GGPLGMRGK

SEQ ID NO: 1067 - protein, 12 aa, synthetic construct
  SITE 8: note = L-homophenylalanine
  SITE 12: note = Lysine modified with a CPQ2 quencher
GFFLAQAFRSKK

SEQ ID NO: 1068 - protein, 17 aa, synthetic construct
  SITE 17: note = Lysine modified with a CPQ2 quencher
GAHAVSRIRIYLLPAKK

SEQ ID NO: 1069 - protein, 9 aa, synthetic construct
  SITE 9: note = Lysine modified with a CPQ2 quencher
GPLALWARK

SEQ ID NO: 1070 - protein, 9 aa, synthetic construct
  SITE 5: note = S-para-methoxybenzyl cysteine
  SITE 9: note = Lysine modified with a CPQ2 quencher
GPLACWARK

SEQ ID NO: 1071 - protein, 9 aa, synthetic construct
  SITE 9: note = Lysine modified with a CPQ2 quencher
GAPRWIQDK

SEQ ID NO: 1072 - protein, 11 aa, synthetic construct
  SITE 11: note = Lysine modified with a CPQ2 quencher
GLREQQRLKSK

SEQ ID NO: 1073 - protein, 14 aa, synthetic construct
  SITE 14: note = Lysine modified with a CPQ2 quencher
GEFPIYVFLPAKKK

SEQ ID NO: 1074 - protein, 11 aa, synthetic construct
  SITE 11: note = Lysine modified with a CPQ2 quencher
GGAANLVRGGK

SEQ ID NO: 1075 - protein, 11 aa, synthetic construct
  SITE 11: note = Lysine modified with a CPQ2 quencher
GGYAELRMGGK

SEQ ID NO: 1076 - protein, 11 aa, synthetic construct
  SITE 11: note = Lysine modified with a CPQ2 quencher
GAAGAMFLEAK

SEQ ID NO: 1077 - protein, 16 aa, synthetic construct
  SITE 16: note = Lysine modified with a CPQ2 quencher
GLGGSGQRGRKALEGK

SEQ ID NO: 1078 - protein, 16 aa, synthetic construct
  SITE 16: note = Lysine modified with a CPQ2 quencher
GLGGSGHYGRSGLEGK

SEQ ID NO: 1079 - protein, 7 aa, synthetic construct
  SITE 7: note = Lysine modified with a CPQ2 quencher
GYGRSGK

SEQ ID NO: 1080 - protein, 8 aa, synthetic construct
  SITE 8: note = Lysine modified with a CPQ2 quencher
GFRGRKGK

SEQ ID NO: 1081 - protein, 11 aa, synthetic construct
  SITE 11: note = Lysine modified with a CPQ2 quencher
GDRRKKLTQGK

SEQ ID NO: 1082 - protein, 9 aa, synthetic construct
  SITE 9: note = Lysine modified with a CPQ2 quencher
GHPGGPQGK

SEQ ID NO: 1083 - protein, 10 aa, synthetic construct
  SITE 10: note = Lysine modified with a CPQ2 quencher
GKLRFSKQGK

SEQ ID NO: 1084 - protein, 11 aa, synthetic construct
  SITE 11: note = Lysine modified with a CPQ2 quencher
GAIKFFSAQGK

SEQ ID NO: 1085 - protein, 11 aa, synthetic construct
  SITE 11: note = Lysine modified with a CPQ2 quencher
GAIKFFVRQGK

SEQ ID NO: 1086 - protein, 12 aa, synthetic construct
  SITE 12: note = Lysine modified with a CPQ2 quencher
GRPPGFSAFKGK

SEQ ID NO: 1087 - protein, 9 aa, synthetic construct
  SITE 9: note = Lysine modified with a CPQ2 quencher
GFAPQLSGK

SEQ ID NO: 1088 - protein, 9 aa, synthetic construct
  SITE 9: note = Lysine modified with a CPQ2 quencher
GFAAQMAGK

SEQ ID NO: 1089 - protein, 9 aa, synthetic construct
  SITE 9: note = Lysine modified with a CPQ2 quencher
GGMPANQGK

SEQ ID NO: 1090 - protein, 11 aa, synthetic construct
  SITE 11: note = Lysine modified with a CPQ2 quencher
GLSGRSDNHGK

SEQ ID NO: 1091 - protein, 13 aa, synthetic construct
  SITE 13: note = Lysine modified with a CPQ2 quencher
GMAALITRPDFGK

SEQ ID NO: 1092 - protein, 13 aa, synthetic construct
  SITE 13: note = Lysine modified with a CPQ2 quencher
GMAAAITRPRFGK

SEQ ID NO: 1093 - protein, 13 aa, synthetic construct
  SITE 13: note = Lysine modified with a CPQ2 quencher
GMAALIVRPDLGK

SEQ ID NO: 1094 - protein, 12 aa, synthetic construct
  SITE 12: note = Lysine modified with a CPQ2 quencher
GTSGPNQEQEGK

SEQ ID NO: 1095 - protein, 12 aa, synthetic construct
  SITE 12: note = Lysine modified with a CPQ2 quencher
GTAGPNQEQEGK

SEQ ID NO: 1096 - protein, 10 aa, synthetic construct
  SITE 10: note = Lysine modified with a CPQ2 quencher
GGPGPNQAGK

SEQ ID NO: 1097 - protein, 11 aa, synthetic construct
  SITE 11: note = Lysine modified with a CPQ2 quencher
GASGPAGPAGK

SEQ ID NO: 1098 - protein, 12 aa, synthetic construct
  SITE 12: note = Lysine modified with a CPQ2 quencher
GERGETGPSGGK

SEQ ID NO: 1099 - protein, 11 aa, synthetic construct
  SITE 11: note = Lysine modified with a CPQ2 quencher
GVSQELGQRGK

SEQ ID NO: 1100 - protein, 12 aa, synthetic construct
  SITE 12: note = Lysine modified with a CPQ2 quencher
GTGPPGYPTGGK

SEQ ID NO: 1101 - protein, 10 aa, synthetic construct
  SITE 10: note = Lysine modified with a CPQ2 quencher
GTRLPVYQGK

SEQ ID NO: 1102 - protein, 11 aa, synthetic construct
  SITE 11: note = Lysine modified with a CPQ2 quencher
GRQARVVGGGK

SEQ ID NO: 1103 - protein, 11 aa, synthetic construct
  SITE 11: note = Lysine modified with a CPQ2 quencher
GRQRRVVGGGK

SEQ ID NO: 1104 - protein, 11 aa, synthetic construct
  SITE 11: note = Lysine modified with a CPQ2 quencher
GRQARAVGGGK

SEQ ID NO: 1105 - protein, 11 aa, synthetic construct
  SITE 11: note = Lysine modified with a CPQ2 quencher
GRKRRGSRGGK

SEQ ID NO: 1106 - protein, 11 aa, synthetic construct
  SITE 11: note = Lysine modified with a CPQ2 quencher
GKQSRKFVPGK

SEQ ID NO: 1107 - protein, 8 aa, synthetic construct
  SITE 8: note = Lysine modified with a CPQ2 quencher
GVTGRSGK

SEQ ID NO: 1108 - protein, 9 aa, synthetic construct
  SITE 9: note = Lysine modified with a CPQ2 quencher
GLKSRVKGK

SEQ ID NO: 1109 - protein, 13 aa, synthetic construct
  SITE 13: note = Lysine modified with a CPQ2 quencher
GGIGAVLKVLTGK

SEQ ID NO: 1110 - protein, 13 aa, synthetic construct
  SITE 13: note = Lysine modified with a CPQ2 quencher
GGLPALISWIKGK

SEQ ID NO: 1111 - protein, 12 aa, synthetic construct
  SITE 12: note = Lysine modified with a CPQ2 quencher
GSEVNLDAEFGK

SEQ ID NO: 1112 - protein, 16 aa, synthetic construct
  SITE 16: note = Lysine modified with a CPQ2 quencher
GEEKPICFFRLGKEGK

SEQ ID NO: 1113 - protein, 16 aa, synthetic construct
  SITE 16: note = Lysine modified with a CPQ2 quencher
GEEKPILFFRLGKEGK

SEQ ID NO: 1114 - protein, 11 aa, synthetic construct
  SITE 11: note = Lysine modified with a CPQ2 quencher
GAPSSVIAAGK

SEQ ID NO: 1115 - protein, 11 aa, synthetic construct
  SITE 11: note = Lysine modified with a CPQ2 quencher
GKKAKRNALGK

SEQ ID NO: 1116 - protein, 13 aa, synthetic construct
  SITE 13: note = Lysine modified with a CPQ2 quencher
GWTNTSANYNLGK

SEQ ID NO: 1117 - protein, 7 aa, synthetic construct
  SITE 7: note = Lysine modified with a CPQ2 quencher
GRVRRGK

SEQ ID NO: 1118 - protein, 8 aa, synthetic construct
  SITE 8: note = Lysine modified with a CPQ2 quencher
GERTKRGK

SEQ ID NO: 1119 - protein, 15 aa, synthetic construct
  SITE 15: note = Lysine modified with a CPQ2 quencher
GRYQIKPLKSTDEGK

SEQ ID NO: 1120 - protein, 14 aa, synthetic construct
  SITE 9: note = L-homophenylalanine
  SITE 14: note = Lysine modified with a CPQ2 quencher
GWELRHQAFRSKGK

SEQ ID NO: 1121 - protein, 15 aa, synthetic construct
  SITE 7: note = L- Methyl cysteine
  SITE 15: note = Lysine modified with a CPQ2 quencher
GSGAFKCLKDGAGGK

SEQ ID NO: 1122 - protein, 9 aa, synthetic construct
  SITE 9: note = Lysine modified with a CPQ2 quencher
GYVADGWGK

SEQ ID NO: 1123 - protein, 9 aa, synthetic construct
  SITE 9: note = Lysine modified with a CPQ2 quencher
GWEHDGWGK

SEQ ID NO: 1124 - protein, 10 aa, synthetic construct
  SITE 10: note = Lysine modified with a CPQ2 quencher
GYVADAPVGK

SEQ ID NO: 1125 - protein, 10 aa, synthetic construct
  SITE 10: note = Lysine modified with a CPQ2 quencher
GRPPGFSAGK

SEQ ID NO: 1126 - protein, 10 aa, synthetic construct
  SITE 10: note = Lysine modified with a CPQ2 quencher
GGSPAFLAGK

SEQ ID NO: 1127 - protein, 10 aa, synthetic construct
  SITE 10: note = Lysine modified with a CPQ2 quencher
GAGFSLPAGK

SEQ ID NO: 1128 - protein, 13 aa, synthetic construct
  SITE 13: note = Lysine modified with a CPQ2 quencher
GRWHTVGLRWEGK

SEQ ID NO: 1129 - protein, 6 aa, synthetic construct
  SITE 6: note = Lysine modified with a CPQ2 quencher
GLEQGK

SEQ ID NO: 1130 - protein, 13 aa, synthetic construct
  SITE 13: note = Lysine modified with a CPQ2 quencher
GRWPPMGLPWEGK

SEQ ID NO: 1131 - protein, 9 aa, synthetic construct
  SITE 9: note = Lysine modified with a CPQ2 quencher
GRPKPVEGK

SEQ ID NO: 1132 - protein, 7 aa, synthetic construct
  SITE 7: note = Lysine modified with a CPQ2 quencher
GIETDGK

SEQ ID NO: 1133 - protein, 10 aa, synthetic construct
  SITE 10: note = Lysine modified with a CPQ2 quencher
GVGPDFGRGK

SEQ ID NO: 1134 - protein, 12 aa, synthetic construct
  SITE 12: note = Lysine modified with a CPQ2 quencher
GGIEFDSGGCGK

SEQ ID NO: 1135 - protein, 10 aa, synthetic construct
  SITE 10: note = Lysine modified with a CPQ2 quencher
GGDFLRRVGK

SEQ ID NO: 1136 - protein, 6 aa, synthetic construct
  SITE 6: note = Lysine modified with a CPQ2 quencher
GAALGK

SEQ ID NO: 1137 - protein, 13 aa, synthetic construct
  SITE 13: note = Lysine modified with a CPQ2 quencher
GYATWSMIAAHGK

SEQ ID NO: 1138 - protein, 13 aa, synthetic construct
  SITE 13: note = Lysine modified with a CPQ2 quencher
GVIMWRLTVGTGK

SEQ ID NO: 1139 - protein, 13 aa, synthetic construct
  SITE 13: note = Lysine modified with a CPQ2 quencher
GRRVLALQQELGK

SEQ ID NO: 1140 - protein, 13 aa, synthetic construct
  SITE 13: note = Lysine modified with a CPQ2 quencher
GLATWPLSGLWGK

SEQ ID NO: 1141 - protein, 13 aa, synthetic construct
  SITE 13: note = Lysine modified with a CPQ2 quencher
GNTPNWLVNAVGK

SEQ ID NO: 1142 - protein, 16 aa, synthetic construct
  SITE 16: note = Lysine modified with a CPQ2 quencher
GSPLAQAVRSSSRKGK

SEQ ID NO: 1143 - protein, 13 aa, synthetic construct
  SITE 13: note = Lysine modified with a CPQ2 quencher
GQMPGRLSMAFGK

SEQ ID NO: 1144 - protein, 8 aa, synthetic construct
  SITE 8: note = Lysine modified with a CPQ2 quencher
GPLGLRGK

SEQ ID NO: 1145 - protein, 13 aa, synthetic construct
  SITE 13: note = Lysine modified with a CPQ2 quencher
GQRANSIRVTWGK

SEQ ID NO: 1146 - protein, 8 aa, synthetic construct
  SITE 8: note = Lysine modified with a CPQ2 quencher
GPLAVRGK

SEQ ID NO: 1147 - protein, 13 aa, synthetic construct
  SITE 13: note = Lysine modified with a CPQ2 quencher
GLLAVPAANTVGK

SEQ ID NO: 1148 - protein, 11 aa, synthetic construct
  SITE 11: note = Lysine modified with a CPQ2 quencher
GGPQGLRGQGK

SEQ ID NO: 1149 - protein, 13 aa, synthetic construct
  SITE 13: note = Lysine modified with a CPQ2 quencher
GRTGLYLYNSTGK

SEQ ID NO: 1150 - protein, 17 aa, synthetic construct
  SITE 17: note = Lysine modified with a CPQ2 quencher
GRKKLTQSKFVGGAEGK

SEQ ID NO: 1151 - protein, 7 aa, synthetic construct
  SITE 7: note = Lysine modified with a CPQ2 quencher
GKHYRGK

SEQ ID NO: 1152 - protein, 6 aa, synthetic construct
  SITE 6: note = Lysine modified with a CPQ2 quencher
GQARGK

SEQ ID NO: 1153 - protein, 10 aa, synthetic construct
  SITE 10: note = Lysine modified with a CPQ2 quencher
GPRPFNYLGK

SEQ ID NO: 1154 - protein, 10 aa, synthetic construct
  SITE 10: note = Lysine modified with a CPQ2 quencher
GAPFEMSAGK

SEQ ID NO: 1155 - protein, 10 aa, synthetic construct
  SITE 10: note = Lysine modified with a CPQ2 quencher
GAPFEFSAGK

SEQ ID NO: 1156 - protein, 9 aa, synthetic construct
  SITE 9: note = Lysine modified with a CPQ2 quencher
GPLGFRVGK

SEQ ID NO: 1157 - protein, 11 aa, synthetic construct
  SITE 11: note = Lysine modified with a CPQ2 quencher
GRPLALWRSGK

SEQ ID NO: 1158 - protein, 12 aa, synthetic construct
  SITE 12: note = Lysine modified with a CPQ2 quencher
GRPLALEESQGK

SEQ ID NO: 1159 - protein, 12 aa, synthetic construct
  SITE 12: note = Lysine modified with a CPQ2 quencher
GRPLALWRSQGK

SEQ ID NO: 1160 - protein, 14 aa, synthetic construct
  SITE 14: note = Lysine modified with a CPQ2 quencher
GRNALAVERTASGK

SEQ ID NO: 1161 - protein, 11 aa, synthetic construct
  SITE 11: note = Lysine modified with a CPQ2 quencher
GRPKPQQFWGK

SEQ ID NO: 1162 - protein, 11 aa, synthetic construct
  SITE 11: note = Lysine modified with a CPQ2 quencher
SGSNPYKYTAK

SEQ ID NO: 1163 - protein, 11 aa, synthetic construct
  SITE 11: note = Lysine modified with a CPQ2 quencher
SGSNPYGYTAK

SEQ ID NO: 1164 - protein, 11 aa, synthetic construct
  SITE 11: note = Lysine modified with a CPQ2 quencher
SGTLSELHTAK

SEQ ID NO: 1165 - protein, 11 aa, synthetic construct
  SITE 11: note = Lysine modified with a CPQ2 quencher
SGTISHLHTAK

SEQ ID NO: 1166 - protein, 14 aa, synthetic construct
  SITE 3: note = L-Ornithine
  SITE 8: note = L-homophenylalanine
  SITE 14: note = Lysine modified with a CPQ2 quencher
SGXRSHPFTLYTAK

SEQ ID NO: 1167 - protein, 14 aa, synthetic construct
  SITE 3: note = L-Ornithine
  SITE 8: note = L-homophenylalanine
  SITE 14: note = Lysine modified with a CPQ2 quencher
SGXRSHGFFLYTAK

SEQ ID NO: 1168 - protein, 11 aa, synthetic construct
  SITE 11: note = Lysine modified with a CPQ2 quencher
SGESLAYYTAK

SEQ ID NO: 1169 - protein, 11 aa, synthetic construct
  SITE 11: note = Lysine modified with a CPQ2 quencher
SGHMHAALTAK

SEQ ID NO: 1170 - protein, 11 aa, synthetic construct
  SITE 6: note = D-isoleucine
  SITE 11: note = Lysine modified with a CPQ2 quencher
GILSRIVGGGK

SEQ ID NO: 1171 - protein, 11 aa, synthetic construct
  SITE 5: note = D-arginine
  SITE 6: note = D-isoleucine
  SITE 7: note = D-valine
  SITE 11: note = Lysine modified with a CPQ2 quencher
GILSRIVGGGK

SEQ ID NO: 1172 - protein, 11 aa, synthetic construct
  SITE 11: note = Lysine modified with a CPQ2 quencher
GRQRRALEKGK

SEQ ID NO: 1173 - protein, 11 aa, synthetic construct
  SITE 11: note = Lysine modified with a CPQ2 quencher
GKPISLISSGK

SEQ ID NO: 1174 - protein, 11 aa, synthetic construct
  SITE 11: note = Lysine modified with a CPQ2 quencher
GQKGRYKQEGK

SEQ ID NO: 1175 - protein, 10 aa, synthetic construct
  SITE 10: note = Lysine modified with a CPQ2 quencher
GGPLGLRSWK

SEQ ID NO: 1176 - protein, 10 aa, synthetic construct
  SITE 10: note = Lysine modified with a CPQ2 quencher
GGPLGVRGKK

SEQ ID NO: 1177 - protein, 10 aa, synthetic construct
  SITE 3: note = D-Phenylalanine
  SITE 10: note = Lysine modified with a CPQ2 quencher
GGFPRSGGGK

SEQ ID NO: 1178 - protein, 4 aa, synthetic construct
  SITE 1: note = Acetyl Tryptophan
WEHD

SEQ ID NO: 1179 - protein, 4 aa, synthetic construct
AAPM

SEQ ID NO: 1180 - protein, 4 aa, synthetic construct
AEPF

SEQ ID NO: 1181 - protein, 4 aa, synthetic construct
AAPV

SEQ ID NO: 1182 - protein, 5 aa, synthetic construct
GPLGP

SEQ ID NO: 1183 - protein, 5 aa, synthetic construct
  SITE 1: note = Acetyl Lysine
KQKLR

SEQ ID NO: 1184 - protein, 4 aa, synthetic construct
  SITE 1: note = Butyloxycarbonyl Arginine
RVRR

SEQ ID NO: 1185 - protein, 4 aa, synthetic construct
  SITE 1: note = Butyloxycarbonyl Isoleucine
IEGR

SEQ ID NO: 1186 - protein, 4 aa, synthetic construct
AAPV

SEQ ID NO: 1187 - protein, 8 aa, synthetic construct
RPFHLLVY

SEQ ID NO: 1188 - protein, 7 aa, synthetic construct
  SITE 1: note = Amino-n-butyric acid
  SITE 4: note = Guamidine-L-phenylalanine
XWSFTVF

SEQ ID NO: 1189 - protein, 6 aa, synthetic construct
  SITE 1: note = Morpholinocarbonyl histidine
HSSKLQ

SEQ ID NO: 1190 - protein, 10 aa, synthetic construct
  SITE 1: note = D-Lysine
  SITE 2: note = D-Lysine
  SITE 9: note = Dinitrobenzylation of lysine
KKDRENSPKL

SEQ ID NO: 1191 - protein, 10 aa, synthetic construct
  SITE 1: note = D-Lysine
  SITE 2: note = D-Lysine
  SITE 10: note = Dinitrobenzylation of lysine
KKDRENSPLK

SEQ ID NO: 1192 - protein, 8 aa, synthetic construct
  SITE 7: note = Dinitrobenzylation of lysine
NAGSKFKQ

SEQ ID NO: 1193 - protein, 8 aa, synthetic construct
  SITE 8: note = Dinitrobenzylation of lysine
NAGSKFQK

SEQ ID NO: 1194 - protein, 10 aa, synthetic construct
  SITE 1: note = D-Lysine
  SITE 2: note = D-Lysine
  SITE 9: note = Dinitrobenzylation of lysine
KKHLLGFYKV

SEQ ID NO: 1195 - protein, 10 aa, synthetic construct
  SITE 1: note = D-Lysine
  SITE 2: note = D-Lysine
  SITE 10: note = Dinitrobenzylation of lysine
KKHLLGFYVK

SEQ ID NO: 1196 - protein, 10 aa, synthetic construct
  SITE 1: note = D-Lysine
  SITE 2: note = D-Lysine
  SITE 8: note = Norleucine
  SITE 9: note = Dinitrobenzylation of lysine
  SITE 10: note = Norleucine
KKQEKQTXKX

SEQ ID NO: 1197 - protein, 10 aa, synthetic construct
  SITE 1: note = D-Lysine
  SITE 2: note = D-Lysine
  SITE 8: note = Norleucine
  SITE 9: note = Norleucine
  SITE 10: note = Dinitrobenzylation of lysine
KKQEKQTXXK

SEQ ID NO: 1198 - protein, 9 aa, synthetic construct
  SITE 1: note = D-Lysine
  SITE 8: note = Dinitrobenzylation of lysine
KDPFVVSKW

SEQ ID NO: 1199 - protein, 9 aa, synthetic construct
  SITE 1: note = D-Lysine
  SITE 9: note = Dinitrobenzylation of lysine
KDPFVVSWK

SEQ ID NO: 1200 - protein, 8 aa, synthetic construct
  SITE 7: note = Dinitrobenzylation of lysine
NAYNEIKR

SEQ ID NO: 1201 - protein, 8 aa, synthetic construct
  SITE 8: note = Dinitrobenzylation of lysine
NAYNEIRK

SEQ ID NO: 1202 - protein, 8 aa, synthetic construct
  SITE 2: note = Norleucine
  SITE 7: note = Dinitrobenzylation of lysine
VXRQSEKN

SEQ ID NO: 1203 - protein, 8 aa, synthetic construct
  SITE 2: note = Norleucine
  SITE 8: note = Dinitrobenzylation of lysine
VXRQSENK

SEQ ID NO: 1204 - protein, 8 aa, synthetic construct
  SITE 6: note = Norleucine
  SITE 7: note = Dinitrobenzylation of lysine
YNPREXKI

SEQ ID NO: 1205 - protein, 8 aa, synthetic construct
  SITE 6: note = Norleucine
  SITE 8: note = Dinitrobenzylation of lysine
YNPREXIK

SEQ ID NO: 1206 - protein, 9 aa, synthetic construct
  SITE 1: note = D-Lysine
  SITE 8: note = Dinitrobenzylation of lysine
KEFVHNPKK

SEQ ID NO: 1207 - protein, 9 aa, synthetic construct
  SITE 1: note = D-Lysine
  SITE 9: note = Dinitrobenzylation of lysine
KEFVHNPKK

SEQ ID NO: 1208 - protein, 8 aa, synthetic construct
  SITE 7: note = Dinitrobenzylation of lysine
KRVQFLKH

SEQ ID NO: 1209 - protein, 8 aa, synthetic construct
  SITE 8: note = Dinitrobenzylation of lysine
KRVQFLHK

SEQ ID NO: 1210 - protein, 9 aa, synthetic construct
  SITE 1: note = D-Lysine
  SITE 4: note = Norleucine
  SITE 8: note = Dinitrobenzylation of lysine
KLIXHKNKG

SEQ ID NO: 1211 - protein, 9 aa, synthetic construct
  SITE 1: note = D-Lysine
  SITE 4: note = Norleucine
  SITE 9: note = Dinitrobenzylation of lysine
KLIXHKNGK

SEQ ID NO: 1212 - protein, 10 aa, synthetic construct
  SITE 1: note = D-Lysine
  SITE 2: note = D-Lysine
  SITE 5: note = Norleucine
  SITE 9: note = Dinitrobenzylation of lysine
KKWAXLYHKS

SEQ ID NO: 1213 - protein, 10 aa, synthetic construct
  SITE 1: note = D-Lysine
  SITE 2: note = D-Lysine
  SITE 5: note = Norleucine
  SITE 10: note = Dinitrobenzylation of lysine
KKWAXLYHSK

SEQ ID NO: 1214 - protein, 10 aa, synthetic construct
  SITE 1: note = D-Lysine
  SITE 2: note = D-Lysine
  SITE 9: note = Dinitrobenzylation of lysine
KKAHDIVNKY

SEQ ID NO: 1215 - protein, 10 aa, synthetic construct
  SITE 1: note = D-Lysine
  SITE 2: note = D-Lysine
  SITE 10: note = Dinitrobenzylation of lysine
KKAHDIVNYK

SEQ ID NO: 1216 - protein, 9 aa, synthetic construct
  SITE 1: note = D-Lysine
  SITE 8: note = Dinitrobenzylation of lysine
KSVFVIEKP

SEQ ID NO: 1217 - protein, 9 aa, synthetic construct
  SITE 1: note = D-Lysine
  SITE 9: note = Dinitrobenzylation of lysine
KSVFVIEPK

SEQ ID NO: 1218 - protein, 9 aa, synthetic construct
  SITE 1: note = D-Lysine
  SITE 8: note = Dinitrobenzylation of lysine
KPPSGLSKE

SEQ ID NO: 1219 - protein, 9 aa, synthetic construct
  SITE 1: note = D-Lysine
  SITE 9: note = Dinitrobenzylation of lysine
KPPSGLSEK

SEQ ID NO: 1220 - protein, 10 aa, synthetic construct
  SITE 1: note = D-Lysine
  SITE 2: note = D-Lysine
  SITE 9: note = Dinitrobenzylation of lysine
KKRWYGGIKF

SEQ ID NO: 1221 - protein, 10 aa, synthetic construct
  SITE 1: note = D-Lysine
  SITE 2: note = D-Lysine
  SITE 10: note = Dinitrobenzylation of lysine
KKRWYGGIFK

SEQ ID NO: 1222 - protein, 9 aa, synthetic construct
  SITE 1: note = D-Lysine
  SITE 7: note = Norleucine
  SITE 8: note = Dinitrobenzylation of lysine
KQYVFFXKD

SEQ ID NO: 1223 - protein, 9 aa, synthetic construct
  SITE 1: note = D-Lysine
  SITE 7: note = Norleucine
  SITE 9: note = Dinitrobenzylation of lysine
KQYVFFXDK

SEQ ID NO: 1224 - protein, 9 aa, synthetic construct
  SITE 1: note = D-Lysine
  SITE 8: note = Dinitrobenzylation of lysine
KFAKYYKKT

SEQ ID NO: 1225 - protein, 9 aa, synthetic construct
  SITE 1: note = D-Lysine
  SITE 9: note = Dinitrobenzylation of lysine
KFAKYYKTK

SEQ ID NO: 1226 - protein, 9 aa, synthetic construct
  SITE 1: note = D-Lysine
  SITE 8: note = Dinitrobenzylation of lysine
KQVKHFTKA

SEQ ID NO: 1227 - protein, 9 aa, synthetic construct
  SITE 1: note = D-Lysine
  SITE 9: note = Dinitrobenzylation of lysine
KQVKHFTAK

SEQ ID NO: 1228 - protein, 8 aa, synthetic construct
  SITE 1: note = D-Lysine
  SITE 8: note = Dinitrobenzylation of lysine
KYVADAPK

SEQ ID NO: 1229 - protein, 9 aa, synthetic construct
  SITE 9: note = Dinitrobenzylation of lysine amide
GKGISSQYK

SEQ ID NO: 1230 - protein, 9 aa, synthetic construct
  SITE 9: note = Dinitrobenzylation of lysine
GALPALQNK

SEQ ID NO: 1231 - protein, 7 aa, synthetic construct
  SITE 7: note = Dinitrobenzylation of lysine amide
GHRFRGK

SEQ ID NO: 1232 - protein, 11 aa, synthetic construct
  SITE 11: note = Dinitrobenzylation of lysine
GAPEEIMDQQK

SEQ ID NO: 1233 - protein, 9 aa, synthetic construct
  SITE 9: note = Dinitrobenzylation of lysine amide
GSRKSQQYK

SEQ ID NO: 1234 - protein, 10 aa, synthetic construct
  SITE 10: note = Dinitrobenzylation of lysine amide
GSKGRSLIGK

SEQ ID NO: 1235 - protein, 9 aa, synthetic construct
  SITE 9: note = Dinitrobenzylation of lysine
GFAQSIPKK

SEQ ID NO: 1236 - protein, 10 aa, synthetic construct
  SITE 10: note = Dinitrobenzylation of lysine amide
GRQRRVVGGK

SEQ ID NO: 1237 - protein, 11 aa, synthetic construct
  SITE 11: note = Dinitrobenzylation of lysine amide
GERGETGPSGK

SEQ ID NO: 1238 - protein, 9 aa, synthetic construct
  SITE 9: note = Dinitrobenzylation of lysine
GASGPSSGK

SEQ ID NO: 1239 - protein, 7 aa, synthetic construct
  SITE 7: note = Dinitrobenzylation of lysine amide
GYRFRGK

SEQ ID NO: 1240 - protein, 9 aa, synthetic construct
  SITE 9: note = Dinitrobenzylation of lysine amide
GKLFSSKQK

SEQ ID NO: 1241 - protein, 7 aa, synthetic construct
  SITE 7: note = Dinitrobenzylation of lysine amide
GIVPRGK

SEQ ID NO: 1242 - protein, 10 aa, synthetic construct
  SITE 10: note = Dinitrobenzylation of lysine amide
GIRRSSYFKK

SEQ ID NO: 1243 - protein, 10 aa, synthetic construct
  SITE 2: note = Benzyl-L-histidine
  SITE 3: note = L-tert-leucine
  SITE 7: note = L-methionine-sulfoxide
  SITE 9: note = Dinitrobenzylation of lysine
GHXPSDMGKG

SEQ ID NO: 1244 - protein, 9 aa, synthetic construct
  SITE 1: note = Norvaline
  SITE 4: note = L-octahydroindole-2-carboxylic acid
  SITE 9: note = Dinitrobenzylation of Lysine amide
XIEXDFGRK

SEQ ID NO: 1245 - protein, 4 aa, synthetic construct
  SITE 1: note = Acetyl Histidine
  SITE 2: note = D-threonine
  SITE 3: note = 2,3,4,5,6-pentafluoro-L-penylalanine
HTFR

SEQ ID NO: 1246 - protein, 10 aa, synthetic construct
  SITE 2: note = Benzyl-L-histidine
  SITE 3: note = L-tert-leucine
  SITE 7: note = L-methionine-sulfoxide
  SITE 9: note = Dinitrobenzylation of lysine
GHXPSDMGKG

SEQ ID NO: 1247 - protein, 7 aa, synthetic construct
  SITE 2: note = 2,3-diaminopropionic acid
  SITE 3: note = L-Ornithine
  SITE 4: note = 3-chloro-L-phenylalanine
  SITE 5: note = S-para-methoxybenzyl cysteine
  SITE 7: note = Dinitrobenzylation of lysine amide
GXXFCGK

SEQ ID NO: 1248 - protein, 7 aa, synthetic construct
  SITE 2: note = L-cyclohexylalanine
  SITE 4: note = Homoserine
  SITE 7: note = Dinitrobenzylation of lysine amide
GXLXRGK

SEQ ID NO: 1249 - protein, 7 aa, synthetic construct
  SITE 4: note = Guamidine-L-phenylalanine
  SITE 7: note = Dinitrobenzylation of lysine amide
FVTFSWK

SEQ ID NO: 1250 - protein, 7 aa, synthetic construct
  SITE 2: note = Homocyclohexylalnine
  SITE 3: note = Phenylalanine derivative with a guanidine group in the para position
  SITE 4: note = L-octahydroindole-2-carboxylic acid
  SITE 7: note = Dinitrobenzylation of lysine amide
GXFXRGK

SEQ ID NO: 1251 - protein, 10 aa, synthetic construct
  SITE 10: note = Lysine modified with a CPQ2 quencher
GAIEPDSGGK

SEQ ID NO: 1252 - protein, 14 aa, synthetic construct
  SITE 10: note = Lysine modified with a CPQ2 quencher
  SITE 11: note = D-Lysine
  SITE 12: note = D-Lysine
  SITE 14: note = Cysteine Amide
GAIEFDSGGKKKGC

SEQ ID NO: 1253 - protein, 13 aa, synthetic construct
  SITE 11: note = Lysine modified with a CPQ2 quencher
  SITE 12: note = D-Lysine
  SITE 13: note = D-Lysine
GGAAEAISDAKKK

SEQ ID NO: 1254 - protein, 13 aa, synthetic construct
  SITE 11: note = Lysine modified with a CPQ2 quencher
  SITE 12: note = D-Lysine
  SITE 13: note = D-Lysine
GGAGGAQMGAKKK

SEQ ID NO: 1255 - protein, 13 aa, synthetic construct
  SITE 11: note = Lysine modified with a CPQ2 quencher
  SITE 12: note = D-Lysine
  SITE 13: note = D-Lysine
GGAQPDALNVKKK

SEQ ID NO: 1256 - protein, 13 aa, synthetic construct
  SITE 11: note = Lysine modified with a CPQ2 quencher
  SITE 12: note = D-Lysine
  SITE 13: note = D-Lysine
GGATDVTTTPKKK

SEQ ID NO: 1257 - protein, 13 aa, synthetic construct
  SITE 11: note = Lysine modified with a CPQ2 quencher
  SITE 12: note = D-Lysine
  SITE 13: note = D-Lysine
GGDIVTVANAKKK

SEQ ID NO: 1258 - protein, 13 aa, synthetic construct
  SITE 11: note = Lysine modified with a CPQ2 quencher
  SITE 12: note = D-Lysine
  SITE 13: note = D-Lysine
GGDLGLKSVPKKK

SEQ ID NO: 1259 - protein, 13 aa, synthetic construct
  SITE 11: note = Lysine modified with a CPQ2 quencher
  SITE 12: note = D-Lysine
  SITE 13: note = D-Lysine
GGDVMASNKRKKK

SEQ ID NO: 1260 - protein, 13 aa, synthetic construct
  SITE 11: note = Lysine modified with a CPQ2 quencher
  SITE 12: note = D-Lysine
  SITE 13: note = D-Lysine
GGESDELNTIKKK

SEQ ID NO: 1261 - protein, 13 aa, synthetic construct
  SITE 11: note = Lysine modified with a CPQ2 quencher
  SITE 12: note = D-Lysine
  SITE 13: note = D-Lysine
GGFHPLHSKIKKK

SEQ ID NO: 1262 - protein, 13 aa, synthetic construct
  SITE 11: note = Lysine modified with a CPQ2 quencher
  SITE 12: note = D-Lysine
  SITE 13: note = D-Lysine
GGGHARLVHVKKK

SEQ ID NO: 1263 - protein, 13 aa, synthetic construct
  SITE 11: note = Lysine modified with a CPQ2 quencher
  SITE 12: note = D-Lysine
  SITE 13: note = D-Lysine
GGHIANVERVKKK

SEQ ID NO: 1264 - protein, 13 aa, synthetic construct
  SITE 11: note = Lysine modified with a CPQ2 quencher
  SITE 12: note = D-Lysine
  SITE 13: note = D-Lysine
GGKAAATQKKKKK

SEQ ID NO: 1265 - protein, 13 aa, synthetic construct
  SITE 11: note = Lysine modified with a CPQ2 quencher
  SITE 12: note = D-Lysine
  SITE 13: note = D-Lysine
GGLATASTMDKKK

SEQ ID NO: 1266 - protein, 13 aa, synthetic construct
  SITE 11: note = Lysine modified with a CPQ2 quencher
  SITE 12: note = D-Lysine
  SITE 13: note = D-Lysine
GGLGPKGQTGKKK

SEQ ID NO: 1267 - protein, 13 aa, synthetic construct
  SITE 11: note = Lysine modified with a CPQ2 quencher
  SITE 12: note = D-Lysine
  SITE 13: note = D-Lysine
GGLSLPETGEKKK

SEQ ID NO: 1268 - protein, 13 aa, synthetic construct
  SITE 11: note = Lysine modified with a CPQ2 quencher
  SITE 12: note = D-Lysine
  SITE 13: note = D-Lysine
GGNLAGILKEKKK

SEQ ID NO: 1269 - protein, 13 aa, synthetic construct
  SITE 11: note = Lysine modified with a CPQ2 quencher
  SITE 12: note = D-Lysine
  SITE 13: note = D-Lysine
GGNPGMSEPVKKK

SEQ ID NO: 1270 - protein, 12 aa, synthetic construct
  SITE 10: note = Lysine modified with a CPQ2 quencher
  SITE 11: note = D-Lysine
  SITE 12: note = D-Lysine
GGPFGCHAKKKK

SEQ ID NO: 1271 - protein, 12 aa, synthetic construct
  SITE 10: note = Lysine modified with a CPQ2 quencher
  SITE 11: note = D-Lysine
  SITE 12: note = D-Lysine
GGPLGLRWWKKK

SEQ ID NO: 1272 - protein, 13 aa, synthetic construct
  SITE 11: note = Lysine modified with a CPQ2 quencher
  SITE 12: note = D-Lysine
  SITE 13: note = D-Lysine
GGQMGVMQGVKKK

SEQ ID NO: 1273 - protein, 13 aa, synthetic construct
  SITE 11: note = Lysine modified with a CPQ2 quencher
  SITE 12: note = D-Lysine
  SITE 13: note = D-Lysine
GGQTCKCSCKKKK

SEQ ID NO: 1274 - protein, 13 aa, synthetic construct
  SITE 11: note = Lysine modified with a CPQ2 quencher
  SITE 12: note = D-Lysine
  SITE 13: note = D-Lysine
GGQWAGLVEKKKK

SEQ ID NO: 1275 - protein, 13 aa, synthetic construct
  SITE 11: note = Lysine modified with a CPQ2 quencher
  SITE 12: note = D-Lysine
  SITE 13: note = D-Lysine
GGRPAVMTSPKKK

SEQ ID NO: 1276 - protein, 13 aa, synthetic construct
  SITE 11: note = Lysine modified with a CPQ2 quencher
  SITE 12: note = D-Lysine
  SITE 13: note = D-Lysine
GGTLRELHLDKKK

SEQ ID NO: 1277 - protein, 13 aa, synthetic construct
  SITE 11: note = Lysine modified with a CPQ2 quencher
  SITE 12: note = D-Lysine
  SITE 13: note = D-Lysine
GGTPPPSQGKKKK

SEQ ID NO: 1278 - protein, 13 aa, synthetic construct
  SITE 11: note = Lysine modified with a CPQ2 quencher
  SITE 12: note = D-Lysine
  SITE 13: note = D-Lysine
GGTSEDLVVQKKK

SEQ ID NO: 1279 - protein, 13 aa, synthetic construct
  SITE 11: note = Lysine modified with a CPQ2 quencher
  SITE 12: note = D-Lysine
  SITE 13: note = D-Lysine
GGVWAAEAISKKK

SEQ ID NO: 1280 - protein, 9 aa, synthetic construct
  VARIANT 4: note = R or K
  SITE 9: note = Lysine Amide modified with a CPQ2 quencher
GWYKTQYGK

SEQ ID NO: 1281 - protein, 9 aa, synthetic construct
  VARIANT 4: note = R or K
  SITE 9: note = Lysine modified with a CPQ2 quencher
GFARRWGGK

SEQ ID NO: 1282 - protein, 9 aa, synthetic construct
  VARIANT 4: note = F, Y, L, or W
  SITE 6: note = Norleucine
  SITE 9: note = Lysine modified with a CPQ2 quencher
GSYWPXQGK

SEQ ID NO: 1283 - protein, 9 aa, synthetic construct
  VARIANT 4: note = F or Y
  SITE 5: note = Norleucine
  SITE 9: note = Lysine modified with a CPQ2 quencher
GFIYXPTGK

SEQ ID NO: 1284 - protein, 9 aa, synthetic construct
  SITE 9: note = Lysine Amide modified with a CPQ2 quencher
GTGPKGNGK

SEQ ID NO: 1285 - protein, 9 aa, synthetic construct
  VARIANT 4: note = F or K
  VARIANT 5: note = K or F
  SITE 7: note = Norleucine
  SITE 9: note = Lysine modified with a CPQ2 quencher
GWSKFWXGK

SEQ ID NO: 1286 - protein, 9 aa, synthetic construct
  VARIANT 4: note = G or D
  VARIANT 5: note = D or G
  SITE 9: note = Lysine modified with a CPQ2 quencher
GKTGDARGK

SEQ ID NO: 1287 - protein, 9 aa, synthetic construct
  VARIANT 4: note = P or L
  VARIANT 5: note = L or P
  SITE 9: note = Lysine modified with a CPQ2 quencher
GGHPLSPGK

SEQ ID NO: 1288 - protein, 9 aa, synthetic construct
  VARIANT 3: note = I or D
  VARIANT 4: note = D, T, I, or V
  SITE 9: note = Lysine Amide modified with a CPQ2 quencher
GVIDKDFGK

SEQ ID NO: 1289 - protein, 9 aa, synthetic construct
  VARIANT 5: note = R or K
  SITE 9: note = Lysine modified with a CPQ2 quencher
GFARRWGGK

SEQ ID NO: 1290 - protein, 9 aa, synthetic construct
  VARIANT 4: note = R or S
  VARIANT 5: note = S or R
  SITE 9: note = Lysine Amide modified with a CPQ2 quencher
GPVRSTNGK

SEQ ID NO: 1291 - protein, 9 aa, synthetic construct
  VARIANT 2: note = E or D
  VARIANT 4: note = D or E
  SITE 9: note = Lysine Amide modified with a CPQ2 quencher
GENDRLPGK

SEQ ID NO: 1292 - protein, 9 aa, synthetic construct
  VARIANT 4: note = V or D
  VARIANT 7: note = D or V
  SITE 9: note = Lysine modified with a CPQ2 quencher
GQWVDEDGK

SEQ ID NO: 1293 - protein, 10 aa, synthetic construct
  SITE 1: note = D-Lysine
  SITE 9: note = Lysine modified with a CPQ2 quencher
  VARIANT 10: note = Hydroxy Lysine or Hydroxy Arginine
KGEFVHNPKK

SEQ ID NO: 1294 - protein, 9 aa, synthetic construct
  SITE 8: note = Lysine modified with a CPQ2 quencher
  VARIANT 9: note = Hydroxy Arginine, Hydroxy Lysine or Hydroxy Histidine
GNAYNEIKR

SEQ ID NO: 1295 - protein, 11 aa, synthetic construct
  VARIANT 1: note = Tryptophan Amide, Glycine Amide, or Phenylalanine Amide
  SITE 2: note = Carboxy-fluorescein-L-lysine
  SITE 10: note = D-Lysine
  SITE 11: note = Lysine Amide modified with a CPQ2 quencher
WKNAGSKFGKK

SEQ ID NO: 1296 - protein, 10 aa, synthetic construct
  VARIANT 1: note = Glutamine Amide or Lysine Amide
  SITE 2: note = Carboxy-fluorescein-L-lysine
  SITE 10: note = Lysine Amide modified with a CPQ2 quencher
QKKRVQFLGK

SEQ ID NO: 1297 - protein, 4 aa, synthetic construct
  SITE 1: note = D-Lysine
  SITE 2: note = D-Lysine
KKGC

SEQ ID NO: 1298 - protein, 4 aa, synthetic construct
  SITE 1: note = D-Lysine
  SITE 2: note = D-Lysine
  SITE 4: note = Cysteine Amide
KKGC